(19) United States
(12) Patent Application Publication
(10) Pub. No.: US 2025/0163091 A1
(43) Pub. Date: May 22, 2025

(54) FUNCTIONALIZED HUMAN MILK OLIGOSACCHARIDES AND METHODS FOR PRODUCING THEM

(71) Applicant: The Regents of the University of California, Oakland, CA (US)

(72) Inventors: Xi CHEN, Davis, CA (US); Yuanyuan BAI, Davis, CA (US); Hai YU, Davis, CA (US); Libo ZHANG, Davis, CA (US); Xiaoxiao YANG, Davis, CA (US)

(21) Appl. No.: 18/729,672

(22) PCT Filed: Jan. 19, 2023
(86) PCT No.: PCT/US2023/060916

§ 371 Date: Jul. 17, 2024

Related U.S. Application Data
(60) Provisional application No. 63/300,989, filed on Jan. 19, 2022.

(51) Int. Cl. C07H 5/06

(52) U.S. Cl. CPC C07H 5/06

(57) ABSTRACT

New derivatives of human milk oligosaccharides and methods for preparing them are disclosed. Also described herein are hST6GALNAC V and hGCNT2-B variants, along with polynucleotides encoding the variants and host cells comprising the polynucleotides. Further provided herein are reaction mixtures comprising the hST6GALNAC V and hGCNT2-B variants.

### CROSS-REFERENCE TO RELATED APPLICATION

The present application claims priority to U.S. Provisional Pat. Appl. No. 63/300,989, filed on Jan. 19, 2022, which application is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under Grant No. U01GM120419 and Grant No. R01GM148568, awarded by the National Institutes of Health. The government has certain rights in the invention.

### REFERENCE TO BIOLOGICAL SEQUENCE DISCLOSURE

The instant application contains a Sequence Listing which has been submitted in 20 .xml format via Patent Center in accordance with 37 C.F.R. §§ 1.831 to 1.834, and is hereby incorporated by reference in its entirety for all purposes. The Sequence Listing written in .xml format is named 1369949.xml, was created on Jan. 19, 2023, and is 31,135 bytes in size.

### BACKGROUND OF THE INVENTION

Industrial production of chemicals has benefited tremendously and will continue to benefit from the incorporation of biocatalysis into its synthetic processes. Biocatalysis is environmentally friendly, sustainable, highly efficient, and selective for the desired molecular transformation. It has an especially high potential in industrial production of carbohydrates and glycoconjugates that are soluble in aqueous solution which is a preferred medium for enzyme-catalyzed reactions. The enzyme stabilization power of high concentration carbohydrate substrates has also been shown, adding another advantage for carbohydrate production by biocatalysis. With the growing demands for carbohydrates as pharmaceuticals, nutraceuticals, and ingredients for foods and health beneficial products, developing biocatalytic strategies for their synthesis will become increasingly important especially if their natural sources are limited and their chemical synthesis is challenging. Human milk oligosaccharides (HMOs) are among the well-suited attractive carbohydrate targets for industrial biocatalytic production.

In addition to lactose, lipids, and proteins, human milk oligosaccharides (HMOs) are a major component of human milk which is the sole food for breast-fed infants in the first few months of their lives. HMOs cannot be digested directly by infants but contribute significantly to the health benefits of breastfeeding. Their roles as prebiotics, decoys to pathogenic bacteria and bacteriostatic agents, gut epithelial cell maturation and immune regulators, nutrients for brain development and their applications as infant formula supplements, potential nutraceuticals and therapeutics are being continuously explored.

More than 150 HMOs structures are known. They are extended from a free reducing end disaccharide, lactose, with β1-3/6-linked N-acetyl-D-glucosamine (GlcNAc) and β1-3/4-linked D-galactose (Gal), and with or without α2-3/6-linked N-acetylneuraminic acid (Neu5Ac) and/or α1-2/3/4-linked _{L}-fucose (Fuc). The progress in exploring the biological functions of HMOs and their applications is highly dependent on the access to structurally defined HMOs in sufficient quantity which is currently limited. However, only a portion of structurally characterized HMOs have been synthesized by chemical, enzymatic and chemoenzymatic, and fermentation approaches. HMOs and their sialylated and/or fucosylated derivatives including those with short chain and long-chain structures with or without branches are challenging targets for chemical synthesis or fermentation production but are well suited for biocatalytic production using purified or partially purified enzymes. With the identification and characterization of an increasing number of glycosyltransferases that are well suited for HMO synthesis and the advances in the development of enzymatic and chemoenzymatic methods, the number of HMOs that have been produced in preparative and multi-gram-scales is increasing. HMO product purification remains a bottleneck to quick accessing HMOs in even multi-gram quantities. Continuous innovation is necessary to overcome the challenges for large-scale production of HMOs.

### SUMMARY OF THE INVENTION

Described herein are compounds of Formula I:

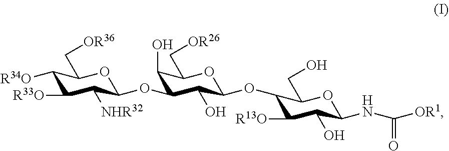

or a salt thereof, wherein R¹ is selected from the group consisting of benzyl and fluorenylmethyl; R¹³ is selected from the group consisting of H and fucosyl; R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R³² is selected from the group consisting of H, acetyl, trifluoroacetyl, Boc, Fmoc; R³⁶ is selected from the group consisting of H and N-acetylneuraminic acid; R³³ and R³⁴ are independently selected from the group consisting of H, fucosyl, and a galactosyl moiety A:

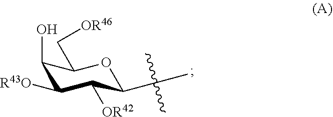

R⁴² is selected from the group consisting of H and fucosyl; R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R⁴³ is selected from the group consisting of a H, a monosaccharide, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; provided that: (i) at least one of R³³ and R³⁴ is fucosyl or galactosyl moiety A; (ii) at least one of R⁴² and R⁴³ is a monosaccharide or an oligosaccharide when (a) R²⁶, R³⁶, and R⁴⁶ are H, and (b) R³³ is galactosyl moiety A and R³⁴ is H or fucosyl, or R³⁴ is galactosyl moiety A and R³³ is H or fucosyl; and (iii) R⁴³ is a monosaccharide or an oligosaccharide when R²⁶, R³⁶, and R⁴⁶ are H, R⁴² is fucosyl, R³³ is galactosyl moiety A, and R³⁴ is H.

Also described are compounds according to Formula II:

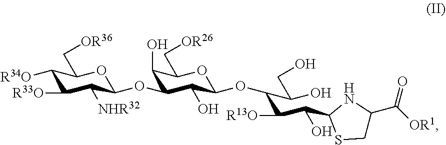

or a salt thereof, wherein: R¹ is selected from the group consisting of benzyl and fluorenylmethyl; R¹³ is selected from the group consisting of H and fucosyl; R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R³² is selected from the group consisting of H, acetyl, trifluoroacetyl, Boc, Fmoc; R³⁶ is selected from the group consisting of H and N-acetylneuraminic acid; R³³ and R³⁴ are independently selected from the group consisting of H, fucosyl, and a galactosyl moiety A:

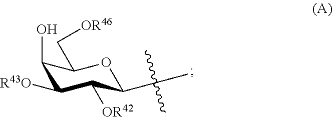

R⁴² is selected from the group consisting of H and fucosyl; R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R⁴³ is selected from the group consisting of a H, a monosaccharide, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid.

Also described are compounds according to Formula III:

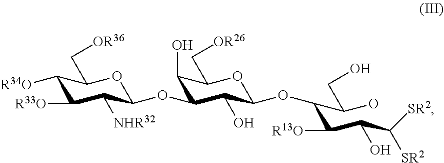

or a salt thereof, wherein: each R² is is independently selected from the group consisting of C1-6 alkyl and optionally substituted C6-14 aryl, or two R² are taken together to form a C1-6 alkylene diradical; R¹³ is selected from the group consisting of H and fucosyl; R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R³² is selected from the group consisting of H, acetyl, trifluoroacetyl, Boc, Fmoc; R³⁶ is selected from the group consisting of H and N-acetylneuraminic acid; R³³ and R³⁴ are independently selected from the group consisting of H, fucosyl, and a galactosyl moiety A:

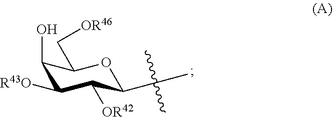

R⁴² is selected from the group consisting of H and fucosyl; R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R⁴³ is selected from the group consisting of a H, a monosaccharide, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid.

Methods for making and using compounds according to Formulas I, II and III and are also described herein.

Also described herein is an hST6GALNAC V variant comprising a polypeptide having at least 80% identity to the amino acid sequence of wild-type hST6GALNAC V. Optionally, the hST6GALNAC V variant comprises a mutation at one or more positions corresponding to G82, A84, V99, H107, G132, S140, Y176, Q184, H190, L191, A201, H207, Q218, T239, F275, and N306 in wild-type hST6GALNAC V. In some cases, the N-terminus of the polypeptide is fused to a maltose binding protein. In some cases, the C-terminus of the polypeptide is fused to a His₆ peptide. Further described herein are hST6GALNAC V variants comprising a polypeptide having at least 80% identity to the amino acid sequence set forth in SEQ ID NO: 5, SEQ ID NO: 6, SEQ ID NO: 7, or SEQ ID NO: 8. Further described herein is a polynucleotide encoding a hST6GALNAC V variant as described herein. A host cell comprising the polynucleotide is also provided herein. Additionally provided herein is a reaction mixture comprising an hST6GALNAC V variant as described herein.

Further provided herein is an hGCNT2-B variant comprising a polypeptide having at least 80% identity to the amino acid sequence set forth in SEQ ID NO: 10. Also provided herein is a polynucleotide encoding an hGCNT2-B variant as described herein. A host cell comprising the polynucleotide is also provided herein. Additionally provided herein is a reaction mixture comprising an hGCNT2-B variant as described herein.

The details of one or more embodiments are set forth in the drawings and the description below. Other features, objects, and advantages will be apparent from the description and drawings, and from the claims.

### BRIEF DESCRIPTION OF THE DRAWINGS

FIG. 1 shows a schematic illustration of the combined substrate and process engineering strategy for chemoenzymatic synthesis and purification of complex human milk oligosaccharides (HMOs) with a free reducing end.

FIG. 2 shows the symbol nomenclatures for LacβNHCbz (1) and the βNHCbz-tagged HMO targets (2-22) synthesized using the MSOPME strategy described herein.

FIG. 3 shows glycosyltransferase acceptor engineering by protecting group-free chemical tagging of lactose for the formation of LacβNHCbz (1) via a β-lactosylamine intermediate.

FIG. 4 shows sugar activation (SA) systems, glycosyltransferases (GTs), and the corresponding one-pot multienzyme (OPME) systems used in either the multistep one-pot multienzyme (MSOPME) strategy or the stepwise one-pot multienzyme (StOPMe) strategy with pre-generation of sugar nucleotides for synthesizing HMOs. Enzymes and abbreviations: BLNahK, Bifidobacterium longum N-acetylhexosamine-1-kinase; PmGlmU, Pasteurella multocida N-acetylglucosamine 1-phosphate uridylyltransferase; PmPpA, Pasteurella multocida inorganic pyrophosphatase; NmLgtA, Neisseria meningitidis β1-3-N-acetylglucosaminyltransferase (GT1a); Hpβ3GlcNAcT, Helicobacter pylori β1-3-N-acetylglucosaminyltransferase (GT1b); β6GlcNAcT, β1-6-N-acetylglucosaminyltransferase (GT1c); SpGalK, Streptococcus pneumoniae galactokinase; BLUSP, Bifidobacterium longum UDP-sugar synthase; Cvβ3GalT, Chromobacterium violaceum β1-3-galactosyltransferase (GT2a); NmLgtB, Neisseria meningitidis β1-4-galactosyltransferase (GT2b); BfFKP, a bifunctional enzyme from Bacteroides fragilis that has both L-fucokinase and GDP-fucose pyrophosphorylase activities; Hm2FT, Helicobacter mustelae α1-2-fucosyltransferase (GT3a); Te2FT, Thermosynechococcus elongatus α1-2-fucosyltransferase; Hp3/4FT, Helicobacter pylori α1-3/4-fucosyltransferase (GT3b); NmCSS, Neisseria meningitidis CMP-sialic acid synthetase; PmST3, Pasteurella multocida α2-3-sialyltransferase 3; PmST1_M144D, Pasteurella multocida α2-3-sialyltransferase 1 M144D mutant (GT4a); Pd2,6ST, Photobacterium damselae α2-6-sialyltransferase (can add Neu5Ac to both terminal and internal Gal residues) (GT4b); Pd2,6ST_A200Y/S232Y, Photobacterium damselae α2-6-sialyltransferase A200Y/S232Y double mutant (a regio-selective α2-6-sialyltransferase for adding Neu5Ac to terminal Gal only) (GT4c); PmST1_P34H/M144V, PmST1P34H/M144V double mutant (a regio-selective α2-6-sialyltransferase for adding Neu5Ac preferably to terminal Gal) (GT4d); hST6GALNAC V, recombinant human ST6GALNAC V expressed in E. coli (GT4e).

FIG. 5 shows catalytic hydrogenation of glycan-βNHCbz (2-47) followed by hydrolysis to form target HMOs (2a-47a) with a free reducing end.

FIG. 6 shows the preparation of benzyl protected L-cysteine (F6.3) and its use in preparing thiazolidine-derivatized lactoside (Lac-thiazolidine F6.4).

FIG. 7A shows the preparation of lactose dithioacetal.

FIG. 7B shows examples of using lactose dithioacetal for preventing fucosylation at the core Glc to achieve site-selective fucosylation in HMOs.

FIG. 8A shows a route employing a GlcNTFA/GlcNH₂ chemoenzymatic synthon strategy for preventing fucosylation at the internal GlcNAc residues to achieve site-selective fucosylation in HMOs.

FIG. 8B shows an alternative route employing a GlcNTFA/GlcNH₂ chemoenzymatic synthon strategy for preventing fucosylation at the internal GlcNAc residues to achieve site-selective fucosylation in HMOs.

FIG. 9A shows the Fmoc protection of GlcNH₂ for preventing fucosylation at the internal GlcNAc residues to achieve site-selective fucosylation in HMOs.

FIG. 9B shows the Boc protection of GlcNH₂ for preventing fucosylation at the internal GlcNAc residues to achieve site-selective fucosylation in HMOs.

FIG. 10 shows possible fucosylation and sialylation sites on the reported HMOs (a-b) containing the LNT (a) or the LNnT (b) backbone; thirty-three HMO synthetic targets (c-g) including sixteen neutral HMOs with or without fucosylation (c), and seventeen sialylated HMOs with those containing a Neu5Ac α2-3 (d) or α2-6-linked to the terminal Gal (e) or α2-6-linked to the internal GlcNAc (f), and FDS-LNT I (g), a complex fucose-containing disialyl HMO that has been found in human milk. Arrows indicate the presence or the absence of the specified glycosidic linkages.

FIGS. 11A and 11B show Enzyme Assembly Synthetic Maps (EASyMaps) for StOPMe systematic synthesis of neutral HMOβNHCbz compounds contain 0-3 fucose residues (3, 5-10, 24-31) on the LNT (11A) or the LNnT (11B) core structure. Structures having not been identified from human milk are underlined. Targets marked with a 4-point star (8 out of 16 structures) can be further sialylated with a Neu5Ac α2-3- and/or α2-6-linked to the terminal Gal by a sialyltransferase. SA4 is carried out together with sialyltransferase in the same step. In comparison, SA1-SA3 are performed before the first glycosylation step of the overall StOPMe process, which is the NmLgtA (GT1a)-catalyzed step for all targets shown. In the EASyMaps for LNTFHepβNHCbz (23) and LNnTFHepβNHCbz (28), the “2×” in front of “SA3” in the last step indicates the equivalents of the donor needed for this glycosylation step. The yields and the amounts of the pure products obtained are also shown.

FIG. 12 shows yields and amounts of NHCbz-tagged sialylated HMOs with a Neu5Ac α2-3/6-linked to the terminal Gal of the tetraose core. The HMO structures that have not been identified from human milk (8 out of 12) are underlined. ^{[a]}Synthesized by StOPMe from LacβNHCbz (1) (100 mg) by following EASyMaps shown in FIGS. 11A and 11B with an additional one-pot two-enzyme (OP2E) sialylation step. [b]Synthesized by a single one-pot two-enzyme (OP2E) sialylation reaction from NHCbz-tagged fucosylated HMO products obtained in FIGS. 11A and 11B.

FIG. 13A shows an EASyMap for StOPMe synthesis of negatively charged HMOs containing the lacto-N-tetraose (LNT) core structure with a Neu5Acα2-6GlcNAc linkage (44-48).

FIG. 13B shows one-pot two-enzyme (OP2E) α2-6-sialylation of LSTaβNHCbz (32) for the formation of DSLNTβNHCbz (46).

FIG. 13C shows one-pot two-enzyme (OP2E) α2-6-sialylation of S-LNF IIβNHCbz (37) for the formation of FDS-LNT IβNHCbz (48).

FIG. 14 shows ¹H and ¹³C NMR spectra of Fucα2Galβ(Fucα4)GlcNAcβ3Galβ4(Fucα3)GlcβNHCbz (LNTFHepβNHCbz, 23).

FIG. 15 shows ¹H and ¹³C NMR spectra of Fucα2GalβGlcNAcβ3Galβ4(Fucα3)GlcβNHCbz (LNDFH-IIIβNHCbz, 24).

FIG. 16 shows ¹H and ¹³C NMR spectra of Galβ(Fucα4)GlcNAcβ3Galβ4GlcβNHCbz (LNFP-IIβNHCbz, 25).

FIG. 17 shows ¹H and ¹³C NMR spectra of Fucα2Galβ3(Fucα4)GlcNAcβ3Galβ4GlcβNHCbz (LNDFH-IβNHCbz, 26).

FIG. 18 shows ¹H and ¹³C NMR spectra of Fucα2Galβ4GlcNAcβ3Galβ4GlcβNHCbz (LNnFP-IβNHCbz, 27).

FIG. 19 shows ¹H and ¹³C NMR spectra of Fucα2Galβ4(Fucα3)GlcNAcβ3Galβ4(Fucα4)GlcβNHCbz (LNTFHepβNHCbz, 28).

FIG. 20 shows ¹H and ¹³C NMR spectra of Fucα2Galβ4GlcNAcβ3Galβ4(Fucα3)GlcβNHCbz (LNnDFH-IIIβNHCbz, 29).

FIG. 21 shows ¹H and ¹³C NMR spectra of Galβ4(Fucα3)GlcNAcβ3Galβ4GlcβNHCbz (LNFP-IIIβNHCbz, 30).

FIG. 22 shows ¹H and ¹³C NMR spectra of Fucα2Galβ4(Fucα3)GlcNAcβ3Galβ4GlcβNHCbz (LNnDFH-IβNHCbz, 31).

FIG. 23 shows ¹H and ¹³C NMR spectra of Fucα2Galβ4(Fucα3)GlcNAcβ3Galβ4GlcβNHCbz (LNnDFH-IβNHCbz, 31).

FIG. 24 shows ¹H and ¹³C NMR spectra of Neu5Acα3Galβ3GlcNAcβ3Galβ4GlcβNHCbz (LSTaβNHCbz, 32).

FIG. 25 shows ¹H and ¹³C NMR spectra of Neu5Acα3GalβGlcNAcβ3Galβ4(Fucα3)GlcβNHCbz (34).

FIG. 26 shows ¹H and ¹³C NMR spectra of Neu5Acα3Galβ(Fucα4)GlcNAcβ3Galβ4(Fucα3)GlcβNHCbz (36).

FIG. 27 shows ¹H and ¹³C NMR spectra of Neu5Acα3Galβ(Fucα4)GlcNAcβ3Galβ4GlcβNHCbz (37).

FIG. 28 shows ¹H and ¹³C NMR spectra of Neu5Acα3Galβ4GlcNAcβ3Galβ4GlcβNHCbz (38).

FIG. 29 shows ¹H and ¹³C NMR spectra of Neu5Acα3Galβ4GlcNAcβ3Galβ4(Fucα3)GlcβNHCbz (40).

FIG. 30 shows ¹H and ¹³C NMR spectra of Neu5Acα3Galβ(Fucα3)GlcNAcβ3Galβ4(Fucα3)GlcβNHCbz (42).

FIG. 31 shows ¹H and ¹³C NMR spectra of Neu5Acα3Galβ4(Fucα3)GlcNAcβ3Galβ4GlcβNHCbz (43).

FIG. 32 shows ¹H and ¹³C NMR spectra of Neu5Acα6Galβ3GlcNAcβ3Galβ4GlcβNHCbz (LSTcβNHCbz, 33).

FIG. 33 shows ¹H and ¹³C NMR spectra of Neu5Acα6Galβ3GlcNAcβ3Galβ4(Fucα3)GlcβNHCbz (35).

FIG. 34 shows ¹H and ¹³C NMR spectra of Neu5Acα6Galβ4GlcNAcβ3Galβ4GlcβNHCbz (39).

FIG. 35 shows ¹H and ¹³C NMR spectra of Neu5Acα6Galβ4GlcNAcβ3Galβ4(Fucα3)GlcβNHCbz (41).

FIG. 36 shows ¹H and ¹³C NMR spectra of Galβ(Neu5Acα6)GlcNAcβ3Galβ4GlcβNHCbz (LSTbβNHCbz, 44).

FIG. 37 shows ¹H and ¹³C NMR spectra of Fucα2Galβ3(Neu5Acα6)GlcNAcβ3Galβ4GlcβNHCbz (45).

FIG. 38 shows ¹H and ¹³C NMR spectra of Neu5Acα3Galβ(Neu5Acα6)GlcNAcβ3Galβ4GlcβNHCbz (DSLNTβNHCbz, 46).

FIG. 39 shows ¹H and ¹³C NMR spectra of Neu5Acα3Galβ(Neu5Acα6)GlcNAcβ3Galβ4(Fucα3)GlcβNHCbz (47).

FIG. 40 shows ¹H and ¹³C NMR spectra of Fucα2Galβ3(Fucα4)GlcNAcβ3Galβ4(Fucα3)Glc (LNTFHep, 23a).

FIG. 41 shows ¹H and ¹³C NMR spectra Fucα2Galβ3GlcNAcβ3Galβ4(Fucα3)Glc (LNDFH-III, 24a).

FIG. 42 shows ¹H and ¹³C NMR spectra of Galβ(Fucα4)GlcNAcβ3Gal β4Glc (LNFP-II, 25a).

FIG. 43 shows ¹H and ¹³C NMR spectra of Fucα2Galβ3(Fucα4)GlcNAcβ3Galβ4Glc (LNDFH-I, 26a).

FIG. 44 shows ¹H and ¹³C NMR spectra of Fucα2Galβ4GlcNAcβ3Galβ4Glc (LNnFP-I, 27a).

FIG. 45 shows ¹H and ¹³C NMR spectra of Fucα2Galβ4(Fucα3)GlcNAcβ3Galβ4(Fucα3)Glc (LNnTFHep, 28a).

FIG. 46 shows ¹H and ¹³C NMR spectra of Fucα2Galβ4GlcNAcβ3Galβ4(Fucα3)Glc (LNnDFH-III, 29a).

FIG. 47 shows ¹H and ¹³C NMR spectra of Gal β4(Fucα3)GlcNAcβ3Gal β4Glc (LNFP-III, 30a).

FIG. 48 shows ¹H and ¹³C NMR spectra of Fucα2Galβ4(Fucα3)GlcNAcβ3Galβ4Glc (LNnDFH-I, 31a).

FIG. 49 shows ¹H and ¹³C NMR spectra of Neu5Acα3Galβ3GlcNAcβ3Galβ4Glc (LSTa, 32a).

FIG. 50 shows ¹H and ¹³C NMR spectra of Neu5Acα3GalβGlcNAcβ3Galβ4(Fucα3)Glc (34a).

FIG. 51 shows ¹H and ¹³C NMR spectra of Neu5Acα3Galβ(Fucα4)GlcNAcβ3Galβ4(Fucα3)Glc (36a).

FIG. 52 shows ¹H and ¹³C NMR spectra of Neu5Acα3Galβ(Fucα4)GlcNAcβ3Galβ4Glc (37a).

FIG. 53 shows ¹H and ¹³C NMR spectra of Neu5Acα3Galβ4GlcNAcβ3Galβ4Glc (38a).

FIG. 54 shows ¹H and ¹³C NMR spectra of Neu5Acα3Galβ4GlcNAcβ3Galβ4(Fucα3)Glc (40a).

FIG. 55 shows ¹H and ¹³C NMR spectra of Neu5Acα3Galβ4(Fucα3)GlcNAcβ3Galβ4(Fucα3)Glc (42a).

FIG. 56 shows ¹H and ¹³C NMR spectra of Neu5Acα3Galβ4(Fucα3)GlcNAcβ3Galβ4Glc (43a).

FIG. 57 shows ¹H and ¹³C NMR spectra of Neu5Acα6Galβ3GlcNAcβ3Galβ4Glc (LSTc, 33a).

FIG. 58 shows ¹H and ¹³C NMR spectra of Neu5Acα6Galβ3GlcNAcβ3Galβ4(Fucα3)Glc (35a).

FIG. 59 shows ¹H and ¹³C NMR spectra of Neu5Acα6Galβ4GlcNAcβ3Galβ4Glc (39a).

FIG. 60 shows ¹H and ¹³C NMR spectra of Neu5Acα6Galβ4GlcNAcβ3Galβ4(Fucα3)Glc (41a).

FIG. 61 shows ¹H and ¹³C NMR spectra of Galβ3(Neu5Acα6)GlcNAcβ3Galβ4Glc (LSTb, 44a).

FIG. 62 shows ¹H and ¹³C NMR spectra of Fucα2Galβ3(Neu5Acα6)GlcNAcβ3Galβ4Glc (45a).

FIG. 63 shows ¹H and ¹³C NMR spectra of Neu5Acα3Galβ(Neu5Acα6)GlcNAcβ3Galβ4Glc (DSLNT, 46a).

FIG. 64 shows ¹H and ¹³C NMR spectra of Neu5Acα3Galβ(Neu5Acα6)GlcNAcβ3Galβ4(Fucα3)Glc (47a).

FIG. 65 shows expression and purification of MBP-Δ25hGCNT2-B-His₆. Lanes: M, protein markers (PageRuler™ Plus Prestained Protein Ladder, 10 to 250 kDa); BI, before induction; AI, after induction; Lys, lysate; PP, purified protein. The calculated MW of MBP_delN25hGCNT2B is 89 kDa. The band marked in an arrow on the right is the target band.

FIG. 66 shows reaction catalyzed by MBP-Δ25hGCNT2-B-His₆ using LNnTβNHCbz (3) as the acceptor substrate and UDP-GlcNAc as the donor substrate.

FIG. 67 shows HRMS analysis data for small-scale assays carried out using MBP-A25hGCNT2-B-His₆. The molecular weight of LNnTβNHCBz+Na is 863.2919, found 863.2857; the molecular weight of target product+Na is 1066.3703, found 1066.3638.

FIG. 68 shows One-pot multienzyme (OPME) reaction catalyzed by MBP-A25hGCNT2-B-His₆ using LNnTβNHCbz (3) as the acceptor substrate with in situ generation of UDP-GlcNAc as the donor substrate with sugar activation system 1 (SA1).

FIG. 69 shows ¹³C NMR spectra comparison of several compounds and the product (Branched LNnTβNHCbz, 49) of the reaction catalyzed by MBP-Δ25hGCNT2-B-His₆ using LNnTβNHCbz (3) as the acceptor substrate.

FIG. 70 shows expression and purification of MBP-Δ50hST6GALNAC V_Design7_ext20-His₆. Lanes: M, protein markers (PageRuler™ Plus Prestained Protein Ladder, 10 to 250 kDa); BI, before induction; AI, after induction; Lys, lysate; PP, purified protein.

FIG. 71A shows HRMS results for the reactions catalyzed by MBP-Δ50hST6GALNAC V_ext20-His₆ for the formation of FDS-LNTβNHCbz (48) from S-LNF-IIβNHCbz (37) after 24 hours.

FIG. 71B shows HRMS results for the reactions catalyzed by MBP-Δ50hST6GALNAC V_ext20-His₆ for the formation of FDS-LNTβNHCbz (48) from S-LNF-IIβNHCbz (37) after 72 hours.

FIG. 72 shows EASyMap for the synthesis of several HMOβNHCbz compounds containing the hexoase pLNnH-core.

FIG. 73 shows ¹H and ¹³C NMR spectra of Galβ4(Fucα3)GlcNAcβ3Galβ4(Fucα3)GlcNAcβ3Galβ4GlcβNHCbz (50).

FIG. 74 shows ¹H and ¹³C NMR spectra of Galβ4GlcNAcβ3Galβ4(Fucα3)GlcNAcβ3Galβ4GlcβNHCbz (51).

FIG. 75 shows ¹H and ¹³C NMR spectra of Galβ4(Fucα3)GlcNAcβ3Galβ4GlcNAcβ3Galβ4GlcβNHCbz (52).

FIG. 76 shows ¹H and ¹³C NMR spectra of Galβ4GlcNAcβ3Galβ4GlcNAcβ3Galβ4(Fucα3)GlcβNHCbz (53).

### DETAILED DESCRIPTION OF THE INVENTION

### I. General

Innovation in process development is essential for applying biocatalysis in industrial and laboratory productions of organic compounds, including beneficial carbohydrates such as human milk oligosaccharides (HMOs). HMOs have attracted increasing attention for exploring their potential applications as key ingredients in products that can improve human health. As described in detail below, strategies that combine substrate and process engineering have now been developed to access HMOs via biocatalysis in an efficient manner. The strategies, referred to herein as multistep one-pot multienzyme (MSOPME) and stepwise one-pot multienzyme (StOPMe) processes, allow the access to target HMOs using a single C18-cartridge purification. Their efficiency has been demonstrated in high-yield (71-92%) synthesis of more than fifty tagged HMOs including fucosylated and/or sialylated HMOs short-chain and long-chain oligosaccharides up to nonaoses in one-pot from lactoside without the isolation of the intermediate oligosaccharides. Gram-scale synthesis of an important HMO, lacto-N-fucopentaose-I (LNFP-I), succeeded with 84% yield. Tag removal was achieved in high efficiency (90-97%) without the need for column purification to produce the desired natural HMOs with a free reducing end. The method can be readily adapted for automation to allow quick access of HMOs as well as other glycans and glycoconjugates.

The development of a glycosyltransferase acceptor substrate tagging strategy for highly efficient one-pot multienzyme (OPME) synthesis of sialosides allows facile purification of the more complex glycoside product from its reaction mixture in a single C18-cartridge-based process. The carboxybenzyl (Cbz) group is easy to install from an inexpensive commercially available material in a protection group-free fashion and is readily removable from the product to form oligosaccharides with a free reducing end. Described herein is the development of strategies that combine process engineering with the substrate-tagging (substrate engineering) and OPME chemoenzymatic synthetic systems for facile chemoenzymatic synthesis and purification of HMO targets including those with fucose and/or sialic acid those with long chain structures. Examples for preparative-scale synthesis of targets up to nonaoses are provided herein and gram-scale synthesis is demonstrated for lacto-N-fucopentaose-I (LNFP-I). The methods can be readily adapted for automation and is suitable for introducing isotope-labeled or structurally modified monosaccharides for the formation of HMOs, other glycans and glycoconjugates, as well as their derivatives.

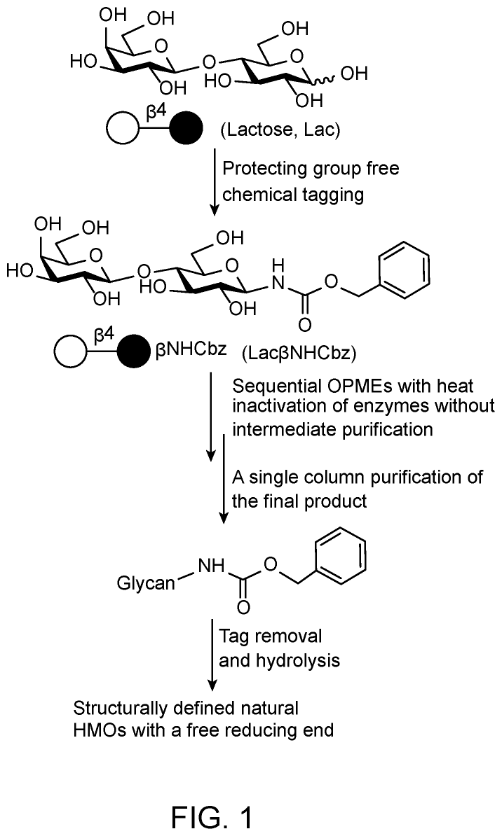

As shown in FIG. 1, the overall strategy involves protecting group free chemical tagging of inexpensive lactose (Lac) for the formation of carboxybenzyl (Cbz) protected β-lactosylamine (LacβNHCbz, 1). Glycosyltransferase-based sequential one-pot multienzyme (OPME) systems are used to extend LacβNHCbz (1) to obtain Cbz-tagged HMOs with in situ generation of the corresponding sugar nucleotide for the glycosyltransferase from a simple and inexpensive monosaccharide to form the desired glycosidic linkage in a regio- and stereospecific manner. Deactivation of enzymes in the reaction mixture after the completion of each OPME glycosylation step prevented uncontrolled polymerization processes. Without the purification of intermediate oligosaccharides, the desired tagged HMOs, even those with long-chain structures, can be obtained in high yields in a true one-pot fashion. The method is referred to herein as a multistep one-pot multienzyme (MSOPME) process. A final step single C18-cartridge purification efficiently provides the pure product containing the hydrophobic Cbz tag. The Cbz tag is easily removed by catalytic hydrogenation followed by spontaneous hydrolysis to provide the desired HMOs with a free reducing end.

To further improve the efficiency, Stepwise One-Pot Multienzyme (StOPMe) strategy with pre-generation of sugar nucleotides is provided herein. In this strategy, sugar nucleotides including uridine 5′-diphosphate-N-acetylglucosamine (UDP-GlcNAc), uridine 5′-diphosphate-galatose (UDP-Gal), and guanosine 5′-diphosphate-L-fucose (GDP-Fuc) are pregenerated using sugar activation (SA) systems before stepwise adding of glycosyltransferases and inactivation of enzymes between glycosyltransferase-catalyzed reaction steps. Compared to MSOPME approach, the StOPMe strategy shortens the glycosyltransferase-catalyzed reaction times and minimize the lost of glycosyltransferase activity during extended reaction time. Enzyme Assembly Synthetic Maps (EASyMaps) are designed to guide the synthesis of these HMOs in a target orientated systematic manner.

### II. Definitions

As used herein, the term “monosaccharide” refers to a sugar having a six-membered carbon backbone (i.e., a hexose) or a nine-carbon backbone (i.e., a nonulosonic acid). Examples of monosaccharides include, but are not limited to, glucose (Glc), galactose (Gal), L-fucose (Fuc), mannose (Man), glucuronic acid (GlcA), iduronic acid (IdoA), and sialic acids such as N-acetylneuraminic acid (Neu5Ac), N-glycoylneuraminic acid (Neu5Gc), 3-keto-3-deoxy-D-glycero-D-galacto-nononic acid (Kdn), and others. Monosaccharides also include hexoses and nonulosonic acid substituted with hydroxy groups, oxo groups, amino groups, acetamido groups, and other functional groups. “Deoxy” monosaccharides refer to monosaccharides having carbon atoms one or more carbon atoms in the hexose backbone having only hydrogen substituents. Monosaccharides also include, but are not limited to, glucosamine (2-amino-2-deoxy-glucose; GlcN), N-acetylglucosamine (2-acetamido-2-deoxy-glucose; GlcNAc), galactosamine (2-amino-2-deoxy-galactose; GalN), N-acetylgalactosamine (2-acetamido-2-deoxy-galactose; GalNAc), mannosamine (2-amino-2-deoxy-mannose; ManN), N-acetylmannosamine (2-acetamido-2-deoxy-mannose; ManNAc), neuraminic acid (Neu), N-trifluoroacetyl neuraminic acid (Neu5AcTFA), and others.

As used herein, the term “oligosaccharide” refers to a compound containing at least two sugars covalently linked together. Oligosaccharides include disaccharides, trisaccharides, tetrasaccharides, pentasaccharides, hexasaccharides, heptasaccharides, octasaccharides, and the like. Covalent linkages for linking sugars generally consist of glycosidic linkages (i.e., C—O—C bonds) formed from the hydroxyl groups of adjacent sugars. Linkages can occur between the 1-carbon (the anomeric carbon) and the 4-carbon of adjacent sugars (i.e., a 1-4 linkage), the 1-carbon (the anomeric carbon) and the 3-carbon of adjacent sugars (i.e., a 1-3 linkage), the 1-carbon (the anomeric carbon) and the 6-carbon of adjacent sugars (i.e., a 1-6 linkage), or the 1-carbon (the anomeric carbon) and the 2-carbon of adjacent sugars (i.e., a 1-2 linkage). A sugar can be linked within an oligosaccharide such that the anomeric carbon is in the α- or β-configuration. The oligosaccharides prepared according to the methods of the invention can also include linkages between carbon atoms other than the 1-, 2-, 3-, 4-, and 6-carbons.

As used herein, the term “isomer” refers to a compound having the same bond structure as a reference compound but having a different three-dimensional arrangement of the bonds. An isomer can be, for example, an enantiomer or a diastereomer.

As used herein, the term “glycoside” refers to a saccharide compound having a moiety “—OR” replacing a hydroxyl group of the parent compound, wherein R is another saccharide (e.g., a monosaccharide, oligosaccharide, or polysaccharide) or a non-saccharide moiety (e.g., a lipid, a protein, a peptide, a linker moiety, a label moiety, etc.). In some embodiments, the moiety —OR in the glycoside replaces the hydroxyl group of the anomeric carbon at the reducing end of the parent saccharide.

A “galactoside” refers to a galactopyranose moiety or a galactofuranose moiety wherein one of the hydroxyl groups of the parent compound is replaced with a moiety —OR as described above. Galactosides include, for example, lactosides (i.e., β-D-galactopyranosyl-(1→4)-D-glucopyranoses).

A “sialoside” refers to a sialic acid moiety wherein one of the hydroxyl groups of the parent compound is replaced with a moiety —OR as described above. Sialic acid is a general term for N- and O-substituted derivatives of neuraminic acid, and includes, but is not limited to, N-acetyl (Neu5Ac) or N-glycolyl (Neu5Gc) substitutions, as well as O-substitutions including acetyl, lactyl, methyl, sulfate and phosphate, among others.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. The term “alkylene” refers to a divalent alkyl radical, wherein the two points of attachment to the diradical are on the same carbon atom or different carbon atoms.

As used herein, the terms “halo” and “halogen” refer to a fluorine, chlorine, bromine, or iodine atom.

As used herein, the term “CMP-sialic acid synthetase” refers to a polypeptide that catalyzes the synthesis of cytidine monophosphate sialic acid (CMP-sialic acid) from cytidine triphosphate (CTP) and sialic acid.

As used herein, the term “sialyltransferase” refers to an enzyme that catalyzes the transfer of a sialic acid to a monosaccharide, an oligosaccharide, or another glycosylated molecule.

As used herein, the term “sialidase” refers to an enzyme that catalyzes the cleavage of a terminal sialic acid from a sialylated target such as an oligosaccharide, a polysaccharide, or a glycosylated protein.

The term “variant,” or “mutant,” in the context of the enzymes in the present disclosure, means a polypeptide, typically recombinant, that comprises one or more amino acid substitutions relative to a corresponding, naturally-occurring or unmodified enzyme.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an _{L}-amino acid and the corresponding _{D}-amino acid).

Naturally-occurring amino acids are those encoded by the genetic code, as well as those amino acids that are later modified, e.g., hydroxyproline, γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission. Nucleotides, as described herein, may also be referred to by their commonly accepted single-letter codes.

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

As used herein, the term “forming a reaction mixture” refers to the process of bringing into contact at least two distinct species such that they mix together and can react, either modifying one of the initial reactants or forming a third distinct species, i.e., a product. It should be appreciated, however, the resulting reaction product can be produced directly from a reaction between the added reagents or from an intermediate from one or more of the added reagents which can be produced in the reaction mixture.

The terms “expression” and “expressed” in the context of a gene refer to the transcriptional and/or translational product of the gene. The level of expression of a DNA molecule in a cell may be determined on the basis of either the amount of corresponding mRNA that is present within the cell or the amount of protein encoded by that DNA produced by the cell.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. For example, recombinant cells express genes that are not found within the native (non-recombinant) form of the cell or express native genes that are otherwise abnormally expressed, under-expressed, or not expressed at all.

The terms “amino acid modification” and “amino acid alteration” refer to a substitution, a deletion, or an insertion of one or more amino acids. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “nucleic acid,” “nucleotide,” and “polynucleotide” refer to deoxyribonucleic acids (DNA) or ribonucleic acids (RNA) and polymers. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), orthologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J Biol. Chem. 260:2605-2608 (1985); and Rossolini et al., Mol. Cell. Probes 8:91-98 (1994)).

The terms “nucleotide sequence encoding a peptide” and “gene” refer to the segment of DNA involved in producing a peptide chain. In addition, a gene will generally include regions preceding and following the coding region (leader and trailer) involved in the transcription/translation of the gene product and the regulation of the transcription/translation. A gene can also include intervening sequences (introns) between individual coding segments (exons). Leaders, trailers, and introns can include regulatory elements that are necessary during the transcription and the translation of a gene (e.g., promoters, terminators, translational regulatory sequences such as ribosome binding sites and internal ribosome entry sites, enhancers, silencers, insulators, boundary elements, replication origins, matrix attachment sites and locus control regions, etc.). A “gene product” can refer to either the mRNA or protein expressed from a particular gene.

“Percentage of sequence identity” is determined by comparing two optimally aligned sequences over a comparison window, wherein the portion of the sequence (e.g., a peptide of the invention) in the comparison window may comprise additions or deletions (i.e., gaps) as compared to the reference sequence which does not comprise additions or deletions, for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

“Identical” and “identity,” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection. These definitions also refer to the complement of a nucleic acid test sequence.

“Similarity” and “percent similarity,” in the context of two or more polypeptide sequences, refer to two or more sequences or subsequences that have a specified percentage of amino acid residues that are either the same or similar as defined by a conservative amino acid substitutions (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% similar over a specified region), when compared and aligned for maximum correspondence over a comparison window, or designated region as measured using one of the following sequence comparison algorithms or by manual alignment and visual inspection. Sequences are “substantially similar” to each other if, for example, they are at least 20%, at least 25%, at least 30%, at least 35%, at least 40%, at least 45%, at least 50%, or at least 55% similar to each other.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

Methods of alignment of sequences for comparison are well-known in the art. Optimal alignment of sequences for comparison can be conducted, which are useful for determining percent sequence identity and sequence similarity, are the BLAST and BLAST 2.0 algorithms, which are described in Altschul et al., (1990) J Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat. Acad. Sci. USA, 90: 5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or peptides are substantially identical is that the peptide encoded by the first nucleic acid is immunologically cross reactive with the antibodies raised against the peptide encoded by the second nucleic acid. Thus, a peptide is typically substantially identical to a second peptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “transfection” and “transfected” refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions or developmental conditions.

A polynucleotide/polypeptide sequence is “heterologous” to an organism or a second polynucleotide/polypeptide sequence if it originates from a different species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

An “expression cassette” refers to a nucleic acid construct, which when introduced into a host cell, results in transcription and/or translation of a RNA or polypeptide, respectively. Antisense constructs or sense constructs that are not or cannot be translated are expressly included by this definition. One of skill will recognize that the inserted polynucleotide sequence need not be identical, but may be only substantially similar to a sequence of the gene from which it was derived.

The terms “vector” and “recombinant expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain.

As used herein, the term “glycosyltransferase” refers to a polypeptide that catalyzes the formation of an oligosaccharide from a nucleotide-sugar an acceptor sugar. Nucleotide-sugars include, but are not limited to, nucleotide diphosphate sugars (NDP-sugars) and nucleotide monophosphate sugars (NMP-sugars) such as a cytidine monophosphate sugar (CMP-sugar). In general, a glycosyltransferase catalyzes the transfer of the monosaccharide moiety of an NDP-sugar or CMP-sugar to a hydroxyl group of the acceptor sugar. The covalent linkage between the monosaccharide and the acceptor sugar can be a 1-3 linkage, a 1-4 linkage, a 1-6-linkage, a 1-2 linkage, a 2-3 linkage, a 2-6 linkage, a 2-8 linkage, or a 2-9 linkage as described above. The linkage may be in the α- or β-configuration with respect to the anomeric carbon of the monosaccharide. Other types of linkages may be formed by the glycosyltransferases in the methods of the invention. Glycosyltransferases include, but are not limited to, heparosan synthases (HSs), glucosaminyltransferases, N-acetylglucosaminyltransferases, glucosyltransferasess, glucuronyltransferases, and sialyltransferases.

As used herein, the term “oligosaccharide” refers to a compound containing at least two monosaccharides covalently linked together. Oligosaccharides include disaccharides, trisaccharides, tetrasaccharides, pentasaccharides, hexasaccharides, heptasaccharides, octasaccharides, and the like. Covalent linkages generally consist of glycosidic linkages (i.e., C—O—C bonds) formed from the hydroxyl groups of adjacent sugars. Linkages can occur between the 1-carbon and the 4-carbon of adjacent sugars (i.e., a 1-4 linkage), the 1-carbon and the 3-carbon of adjacent sugars (i.e., a 1-3 linkage), the 1-carbon and the 6-carbon of adjacent sugars (i.e., a 1-6 linkage), or the 1-carbon and the 2-carbon of adjacent sugars (i.e., a 1-2 linkage). Linkages can occur between the 2-carbon and the 3-carbon of adjacent sugars (i.e., a 2-3 linkage), the 2-carbon and the 6-carbon of adjacent sugars (i.e., a 2-6 linkage), the 2-carbon and the 8-carbon of adjacent sugars (i.e., a 2-8 linkage), or the 2-carbon and the 9-carbon of adjacent sugars (i.e., a 2-9 linkage). A sugar can be linked within an oligosaccharide such that the anomeric carbon is in the α- or β-configuration. The oligosaccharides prepared according to the methods of the invention can also include linkages between carbon atoms other than the 1-, 2-, 3-, 4-, and 6-carbons or the 2-, 3-, 6-, 8-, and 9-carbons.

“Acceptor glycoside” or “glycosylation acceptor” refers to a substance (e.g., a glycosylated amino acid, a glycosylated protein, an oligosaccharide, or a polysaccharide) containing a sphingosine moiety that accepts a sugar moiety from a donor substrate.

As used herein, the term “kinase” refers to a polypeptide that catalyzes the covalent addition of a phosphate group to a substrate. The substrate for a kinase used in the methods of the invention is generally a sugar as defined above, and a phosphate group is added to the anomeric carbon (i.e. the “1,” position) of the sugar. The product of the reaction is a sugar-1-phosphate. Kinases include, but are not limited to, N-acetylhexosamine 1-kinases (NahKs), glucuronokinases (GlcAKs), glucokinases (GlcKs), galactokinases (GalKs), monosaccharide-1-kinases, and xylulokinases. Certain kinases utilize nucleotide triphosphates, including adenosine-5′-triphosphate (ATP) as substrates.

As used herein, the term “dehydrogenase” refers to a polypeptide that catalyzes the oxidation of a primary alcohol. In general, the dehyrogenases used in the methods of the invention convert the hydroxymethyl group of a hexose (i.e. the C6-OH moiety) to a carboxylic acid. Dehydrogenases useful in the methods of the invention include, but are not limited to, UDP-glucose dehydrogenases (Ugds).

As used herein, the term “nucleotide-sugar pyrophosphorylase” refers to a polypeptide that catalyzes the conversion of a sugar-1-phosphate to a UDP-sugar. In general, a uridine-5′-monophosphate moiety is transferred from uridine-5′-triphosphate to the sugar-1-phosphate to form the UDP-sugar. Examples of nucleotide-sugar pyrophosphorylases include glucosamine uridylyltransferases (GlmUs) and glucose-1-phosphate uridylyltransferases (GalUs). Nucleotide-sugar pyrophosphorylases also include promiscuous UDP-sugar pyrophosphorylases, termed “USPs,” that can catalyze the conversion of various sugar-1-phosphates to UDP-sugars including UDP-Glc, UDP-GlcNAc, UDP-GlcNH₂, UDP-Gal, UDP-GalNAc, UDP-GalNH₂, UDP-Man, UDP-ManNAc, UDP-ManNH₂, UDP-GlcA, UDP-IdoA, UDP-GalA, and their substituted analogs.

As used herein, the term “pyrophosphatase” (abbreviated as PpA) refers to a polypeptide that catalyzes the conversion of pyrophosphate (i.e., P₂O₇⁴⁻, HP₂O₇³⁻, H₂P₂O₇²⁻, H₃P₂O₇⁻) to two molar equivalents of inorganic phosphate (i.e., PO₄³⁻, HPO₄²⁻, H₂PO₄⁻).

An amino acid residue “corresponding to an amino acid residue [X] in [specified sequence,” or an amino acid substitution “corresponding to an amino acid substitution [X] in [specified sequence]” refers to an amino acid in a polypeptide of interest that aligns with the equivalent amino acid of a specified sequence. Generally, as described herein, the amino acid corresponding to a position of a specified polypeptide sequence can be determined using an alignment algorithm such as BLAST.

### III. HMO Compounds

Human milk oligosaccharides (HMOs) are the third major component of human milk. They contribute significantly to the health benefit of breast feeding. The potential of applying HMOs in the development of infant formula additives and therapeutics for treating infant and adult diseases is continuously explored. In addition to the challenges in quantitative analysis of HMOs, difficulties to access HMOs (especially in their structurally pure individual forms) in sufficient amounts hindered the progress of related research and practical applications of HMOs.

Accordingly, one aspect of the disclosure provides compounds of Formula I:

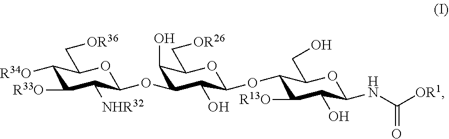

or a salt thereof, wherein:
R¹ is selected from the group consisting of benzyl and fluorenylmethyl;
R¹³ is selected from the group consisting of H and fucosyl;
R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R³² is selected from the group consisting of H, acetyl, trifluoroacetyl, Boc, Fmoc;
R³⁶ is selected from the group consisting of H and N-acetylneuraminic acid;
R³³ and R³⁴ are independently selected from the group consisting of H, fucosyl, and a galactosyl moiety A:

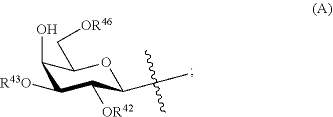

R⁴² is selected from the group consisting of H and fucosyl;
R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R⁴³ is selected from the group consisting of a H, a monosaccharide, and an oligosaccharide comprising 2-10 monosaccharides; and
each monosaccharide is independently selected from the group consisting of glucose, galactose, _{L}-fucose, N-acetylglucosamine, N-trifluoroacetylglucosamine, glucosamine, N-Fmoc-glucosamine, N-Boc-glucosamine, and N-acetylneuraminic acid.

Accordingly, another aspect of the disclosure provides compounds of Formula I:

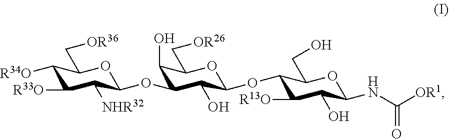

or a salt thereof, as described above wherein:
R¹ is selected from the group consisting of benzyl and fluorenylmethyl;
R¹³ is selected from the group consisting of H and fucosyl;
R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R³² is selected from the group consisting of H, acetyl, trifluoroacetyl, Boc, Fmoc;
R³⁶ is selected from the group consisting of H and N-acetylneuraminic acid;
R³³ and R³⁴ are independently selected from the group consisting of H, fucosyl, and a galactosyl moiety A:

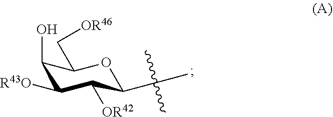

R⁴² is selected from the group consisting of H and fucosyl;
R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R⁴³ is selected from the group consisting of a H, a monosaccharide, and an oligosaccharide comprising 2-10 monosaccharides; and each monosaccharide is independently selected from the group consisting of glucose, galactose, _{L}-fucose, N-acetylglucosamine, N-trifluoroacetylglucosamine, glucosamine, N-Fmoc-glucosamine, N-Boc-glucosamine, and N-acetylneuraminic acid; provided that:
   (i) at least one of R³³ and R³⁴ is fucosyl or galactosyl moiety A;
   (ii) at least one of R⁴² and R⁴³ is a monosaccharide or an oligosaccharide when (a) R²⁶, R³⁶, and R⁴⁶ are H, and
      (b) R³³ is galactosyl moiety A and R³⁴ is H or fucosyl, or
         R³⁴ is galactosyl moiety A and R³³ is H or fucosyl; and
            R⁴³ is a monosaccharide or an oligosaccharide when R²⁶, R³⁶, and R⁴⁶ are H, R⁴² is fucosyl,
            R³³ is galactosyl moiety A, and R³⁴ is H.

In some embodiments, compounds according to Formula I are provided as described above wherein R¹ is benzyl.

In some embodiments, compounds according to Formula I are provided as described above wherein R³³ is galactosyl moiety A.

In some embodiments, compounds according to Formula I are provided as described above wherein R⁴² is H and R⁴³ is a monosaccharide or an oligosaccharide.

In some embodiments, compounds according to Formula I are provided as described above wherein R¹³ is H. In some embodiments, R¹³ is fucosyl.

In some embodiments, compounds according to Formula I are provided as described above wherein the compound according to Formula I is Galβ4GlcβNHCbz, GlcNAcβ3Galβ4GlcβNHCbz, Galβ4GlcNAcβ3Galβ4GlcβNHCbz, GlcNAcβ3Galβ4GlcNAcβ3Galβ4GlcβNHCbz, Galβ4GlcNAcβ3Galβ4(Fucα3)GlcβNHCbz, Galβ4(Fucα3)GlcNAcβ3Galβ4(Fucα3)GlcβNHCbz, Galβ3GlcNAcβ3Galβ4GlcβNHCbz, Fucα2GalβGlcNAcβ3Galβ4GlcβNHCbz, GalβGlcNAcβ3Galβ4(Fucα3)GlcβNHCbz, Galβ(Fucα4)GlcNAcβ3Galβ4(Fucα3)GlcβNHCbz, Galβ4GlcNAcβ3Galβ4GlcNAcβ3Galβ4GlcβNHCbz, GlcNAcβ3Galβ4GlcNAcβ3Galβ4GlcNAcβ3Galβ4GlcβNHCbz, Fucα2Galβ4GlcNAcβ3Galβ4GlcNAcβ3Galβ4GlcβNHCbz, Galβ4(Fucα3)GlcNAcβ3Galβ4(Fucα3)GlcNAcβ3Galβ4(Fucα3)GlcβNHCbz, Neu5Acα3Gal β4GlcNAcβ3Galβ4GlcNAcβ3Gal β4GlcβNHCbz, Neu5Acα6Gal β4GlcNAcβ3Galβ4GlcNAcβ3Galβ4GlcβNHCbz, GalβGlcNAcβ3Galβ4GlcNAcβ3Galβ4GlcβNHCbz, Fucα2GalβGlcNAcβ3Galβ4GlcNAcβ3Galβ4GlcβNHCbz, Galβ(Fucα4)GlcNAcβ3Galβ4(Fucα3)GlcNAcβ3Galβ4(Fucα3)GlcβNHCbz, Galβ4GlcNAcβ3Galβ4GlcNAcβ3Galβ4GlcNAcβ3Galβ4GlcβNHCbz, GalβGlcNAcβ3Galβ4GlcNAcβ3Galβ4GlcNAcβ3Galβ4GlcβNHCbz, Fucα2GalβGlcNAcβ3Galβ4GlcNAcβ3Galβ4GlcNAcβ3Galβ4GlcβNHCbz, Fucα2Galβ(Fucα4)GlcNAcβ3Galβ4(Fucα3)GlcβNHCbz, Fucα2GalβGlcNAcβ3Galβ4(Fucα3)GlcβNHCbz, Galβ(Fucα4)GlcNAcβ3Galβ4GlcβNHCbz, Fucα2Galβ(Fucα4)GlcNAcβ3Galβ4GlcβNHCbz, Fucα2Galβ4GlcNAcβ3Galβ4GlcβNHCbz, Fucα2Galβ4(Fucα3)GlcNAcβ3Galβ4(Fucα3)GlcβNHCbz, Fucα2Galβ4GlcNAcβ3Galβ4(Fucα3)GlcβNHCbz, Galβ4(Fucα3)GlcNAcβ3Galβ4Galβ1βNHCbz, Fucα2Galβ4(Fucα3)GlcNAcβ3Galβ4GlcβNHCbz, Neu5Acα3GalβGlcNAcβ3Galβ4GlcβNHCbz, Neu5Acα6Gal β3GlcNAcβ3Galβ4GlcβNHCbz, Neu5Acα3GalβGlcNAcβ3Galβ4(Fucα3)GlcβNHCbz, Neu5Acα6Gal β3GlcNAcβ3Galβ4(Fucα3)GlcβNHCbz, Neu5Acα3Galβ(Fucα4)GlcNAcβ3Galβ4(Fucα3)GlcβNHCbz, Neu5Acα3Galβ(Fucα4)GlcNAcβ3Galβ4GlcβNHCbz, Neu5Acα3Galβ4GlcNAcβ3Galβ4GlcβNHCbz, Neu5Acα6Galβ4GlcNAcβ3Galβ4GlcβNHCbz, Neu5Acα3Galβ4GlcNAcβ3Galβ4(Fucα3)GlcβNHCbz, Neu5Acα6Galβ4GlcNAcβ3Galβ4(Fucα3)GlcβNHCbz, Neu5Acα3Galβ4(Fucα3)GlcNAcβ3Galβ4(Fucα3)GlcβNHCbz, Neu5Acα3Galβ4(Fucα3)GlcNAcβ3Galβ4GlcβNHCbz, Galβ(Neu5Acα6)GlcNAcβ3Galβ4GlcβNHCbz, Fucα2Galβ(Neu5Acα6)GlcNAcβ3Galβ4GlcβNHCbz, Neu5Acα3Galβ(Neu5Acα6)GlcNAcβ3Galβ4GlcβNHCbz, Neu5Acα3Galβ(Neu5Acα6)GlcNAcβ3Galβ4(Fucα3)GlcβNHCbz, Neu5Acα3Galβ3(Neu5Acα6) (Fucα4)GlcNAcβ3Galβ4GlcβNHCbz, Galβ4GlcNAcβ3(GlcNAcβ6)Galβ4GlcβNHCbz Galβ4(Fucα3)GlcNAcβ3Galβ4(Fucα3)GlcNAcβ3Galβ4GlcβNHCbz, Galβ4GlcNAcβ3Galβ4(Fucα3)GlcNAcβ3Galβ4GlcβNHCbz, Galβ4(Fucα3)GlcNAcβ3Galβ4GlcNAcβ3Galβ4GlcβNHCbz, Galβ4GlcNAcβ3Galβ4GlcNAcβ3Galβ4(Fucα3)GlcβNHCbz, or any salt thereof.

In some embodiments, compounds according to Formula I are provided as described above wherein the compound according to Formula I is GlcNHTFAβ3Galβ4GlcβNHCbz, GlcNH₂Galβ4GlcβNHCbz, Galβ4GlcNAcβ3Galβ4GlcNH₂β3Galβ4GlcβNHCbz, Galβ4GlcNAcβ3Galβ4GlcNHBocβ3Galβ4GlcβNHCbz, Galβ4GlcNAcβ3Galβ4GlcNHFmocβ3Galβ4GlcβNHCbz, Galβ4(Fucα3)GlcNAcβ3Galβ4GlcNHBocβ3Galβ4(Fucα3)GlcβNHCbz, Galβ4(Fucα3)GlcNAcβ3Galβ4GlcNHFmocβ3Galβ4(Fucα3)GlcβNHCbz, Galβ4(Fucα3)GlcNAcβ3Galβ4GlcNAcβ3Galβ4(Fucα3)GlcβNHCbz, GalβGlcNAcβ3Galβ4GlcNH₂β3Galβ4GlcβNHCbz, GalβGlcNAcβ3Galβ4GlcNHBocβ3Galβ4GlcβNHCbz, GalβGlcNAcβ3Galβ4GlcNHFmocβ3Galβ4GlcβNHCbz, Galβ(Fucα4)GlcNAcβ3Galβ4GlcNHBocβ3Galβ4(Fucα3)GlcβNHCbz, Galβ(Fucα4)GlcNAcβ3Galβ4GlcNHFmocβ3Galβ4(Fucα3)GlcβNHCbz, Galβ(Fucα4)GlcNAcβ3Galβ4GlcNAcβ3Galβ4(Fucα3)GlcβNHCbz, or any salt thereof.

Another aspect of the disclosure provides compounds of Formula II:

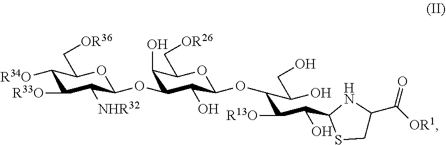

or a salt thereof, wherein:
R¹ is selected from the group consisting of benzyl and fluorenylmethyl;
R¹³ is selected from the group consisting of H and fucosyl;
R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R³² is selected from the group consisting of H, acetyl, trifluoroacetyl, Boc, Fmoc;
R³⁶ is selected from the group consisting of H and N-acetylneuraminic acid;
R³³ and R³⁴ are independently selected from the group consisting of H, fucosyl, and a galactosyl moiety A:

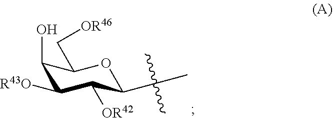

R⁴² is selected from the group consisting of H and fucosyl;
R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R⁴³ is selected from the group consisting of a H, a monosaccharide, and an oligosaccharide comprising 2-10 monosaccharides; and
each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid.

In some embodiments, compounds according to Formula II are provided as described above wherein:
(i) at least one of R³³ and R³⁴ is fucosyl or galactosyl moiety A;
(ii) at least one of R⁴² and R⁴³ is a monosaccharide or an oligosaccharide when
   (a) R²⁶, R³⁶, and R⁴⁶ are H, and
   (b) R³³ is galactosyl moiety A and R³⁴ is H or fucosyl, or R³⁴ is galactosyl moiety A and R³³ is H or fucosyl; and
(iii) R⁴³ is a monosaccharide or an oligosaccharide when R⁴² is fucosyl, R³³ is galactosyl moiety A, and R³⁴ is H.

In some embodiments, compounds according to Formula II are provided as described above wherein R¹ is benzyl.

In some embodiments, compounds according to Formula II are provided as described above wherein R³³ is galactosyl moiety A.

In some embodiments, compounds according to Formula II are provided as described above wherein R⁴² is H and R⁴³ is a monosaccharide or an oligosaccharide.

In some embodiments, compounds according to Formula II are provided as described above wherein R¹³ is H. In some embodiments, R¹³ is fucosyl.

Another aspect of the disclosure provides compounds of Formula III:

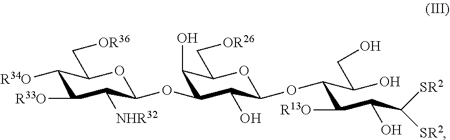

or a salt thereof, wherein:
each R² is is independently selected from the group consisting of C₁-6 alkyl and optionally substituted C₆₋₁₄ aryl, or two R² are taken together to form a C₁₋₆ alkylene diradical;
R¹³ is selected from the group consisting of H and fucosyl;
R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R³² is selected from the group consisting of H, acetyl, trifluoroacetyl, Boc, and Fmoc;
R³⁶ is selected from the group consisting of H and N-acetylneuraminic acid;
R³³ and R³⁴ are independently selected from the group consisting of H, fucosyl, and a galactosyl moiety A:

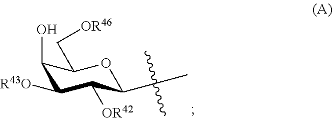

R⁴² is selected from the group consisting of H and fucosyl;
R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R⁴³ is selected from the group consisting of a H, a monosaccharide, and an oligosaccharide comprising 2-10 monosaccharides; and each monosaccharide is independently selected from the group consisting of glucose, galactose, _{L}-fucose, N-acetylglucosamine, N-trifluoroacetylglucosamine, glucosamine, N-Fmoc-glucosamine, N-Boc-glucosamine, and N-acetylneuraminic acid.

In some embodiments, compounds according to Formula III are provided as described above wherein R¹ is benzyl.

In some embodiments, compounds according to Formula III are provided as described above wherein R³³ is galactosyl moiety A.

In some embodiments, compounds according to Formula III are provided as described above wherein R⁴² is H and R⁴³ is a monosaccharide or an oligosaccharide.

In some embodiments, compounds according to Formula III are provided as described above wherein R¹³ is H. In some embodiments, R¹³ is fucosyl.

Described herein, in some embodiments, are HMO compounds having a tetraose core (e.g., HMO compounds of Formula I). Human milk oligosaccharides (HMOs) containing a tetraose core such as lacto-N-tetraose (LNT), lacto-N-neotetraose (LNnT), lacto-N-fucopentaose I (LNFP-I) and II (LNFP-II), lacto-N-difuco-hexaose-I (LNDFH-I), and disialyllacto-N-tetraose (DSLNT) are among the top ten most abundant HMOs in human milk. Different from animals which may contain both N-acetylneuraminic acid (Neu5Ac) and N-glycolylneuraminic acid (Neu5Gc) as the sialic acid forms in their milk, human milk contains only Neu5Ac as the sialic acid form. Overall, a total of thirteen LNT-based and six LNnT-based HMOs have been reported.

Systematic synthesis of a complete library of HMOs containing a tetraose core such as LNT or LNnT had not been previously achieved. The highly efficient methods presented herein allow access, in a systematic manner, to the complete library of tetraose-core-based HMOs including those with different combination of fucosylation and/or sialylation. Described herein is the synthesis of these HMOs in a systematic target orientated manner using a Stepwise One-Pot Multienzyme (StOPMe) strategy with pre-generation of sugar nucleotides. Enzyme Assembly Synthetic Maps (EASyMaps) are designed to guide the synthesis. The challenging Neu5Acα2-6GlcNAc-containing HMOs including DSLNT and a complex disialyl Lewis a structure are accessed using E. coli-expressed human α2-6-sialyltransferase (hST6GALNAC V) and mutants thereof with improved activity.

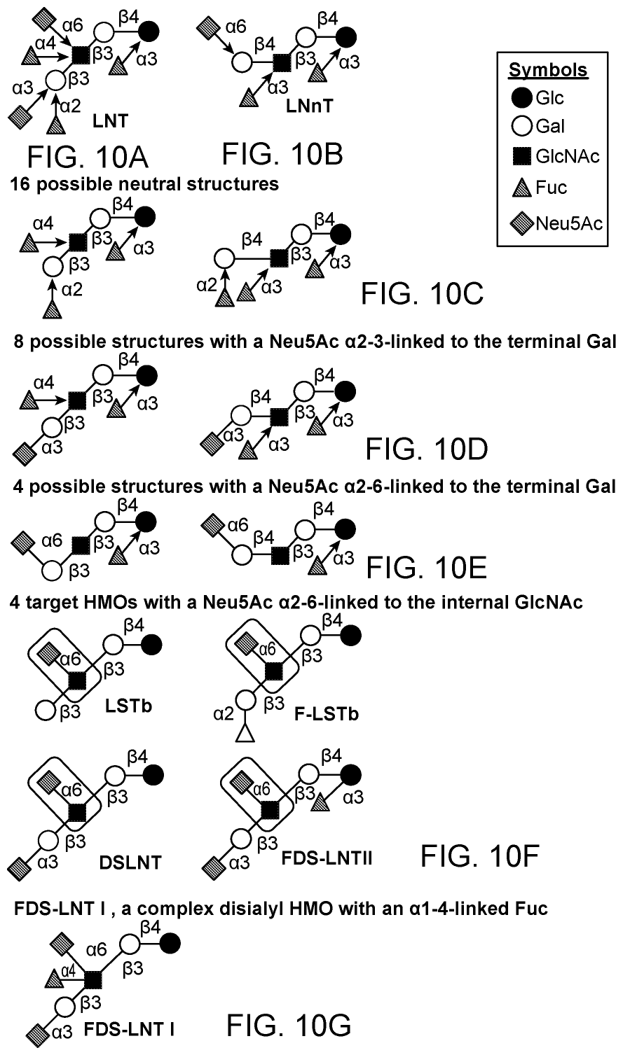

FIG. 10a and FIG. 10b show the reported HMOs with either an LNT core (FIG. 10a) or a LNnT core (FIG. 10b).

Theoretically, a total of 33 HMOs (FIGS. 10c-10g) are possible with the consideration of all possible combination of α1-3 and/or α1-4-fucosylation on GlcNAc or Glc, α1-2-fucosylation or α2-3/6-sialyltion on the terminal Gal, and 5 Neu5Acα2-6GlcNAc-containing HMOs that have been reported.

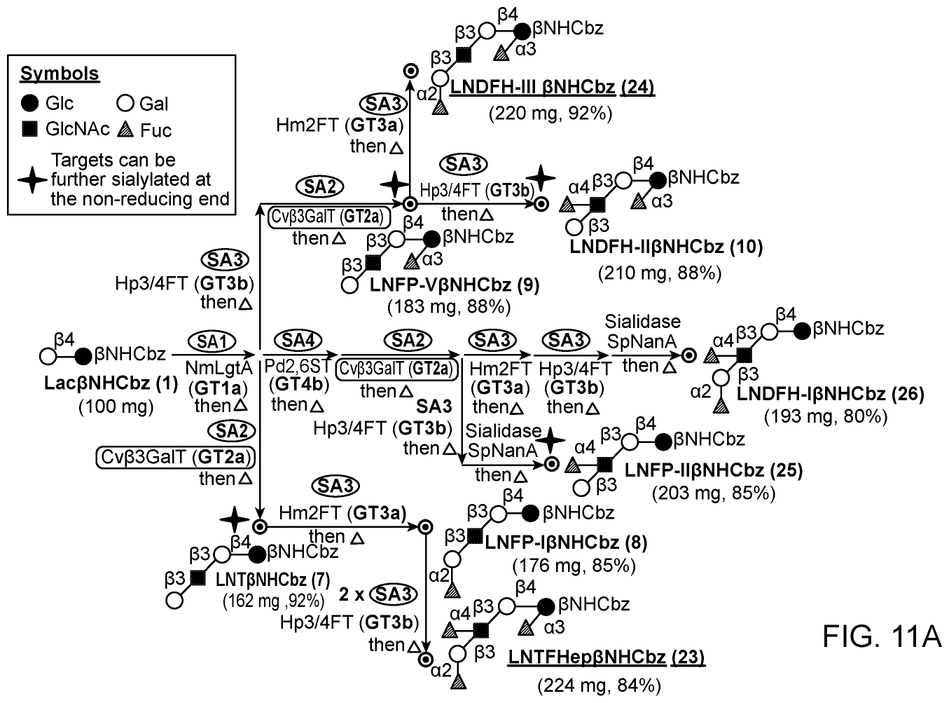

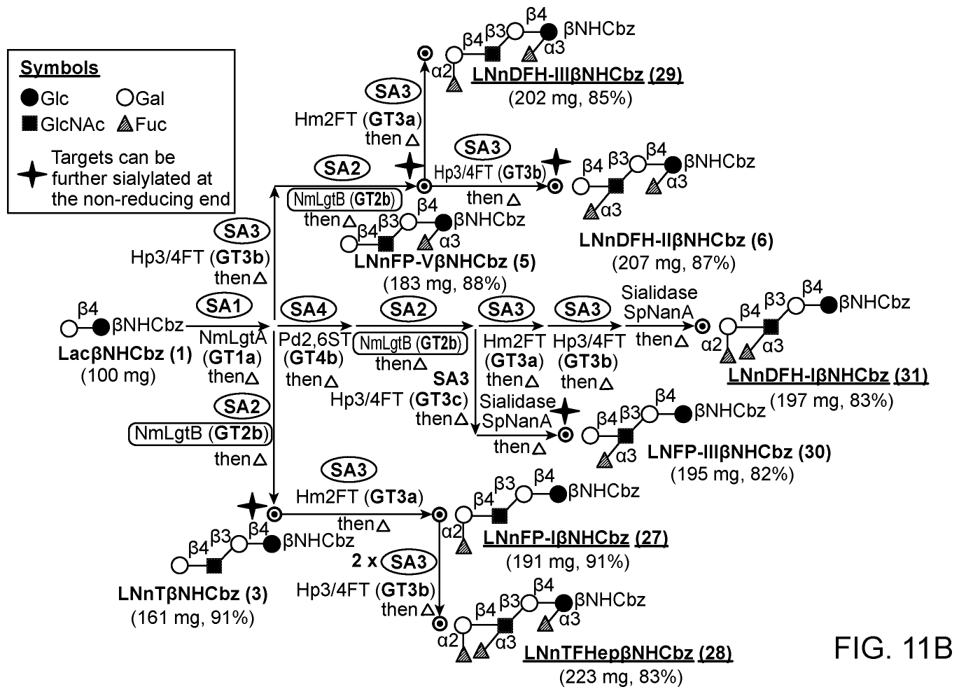

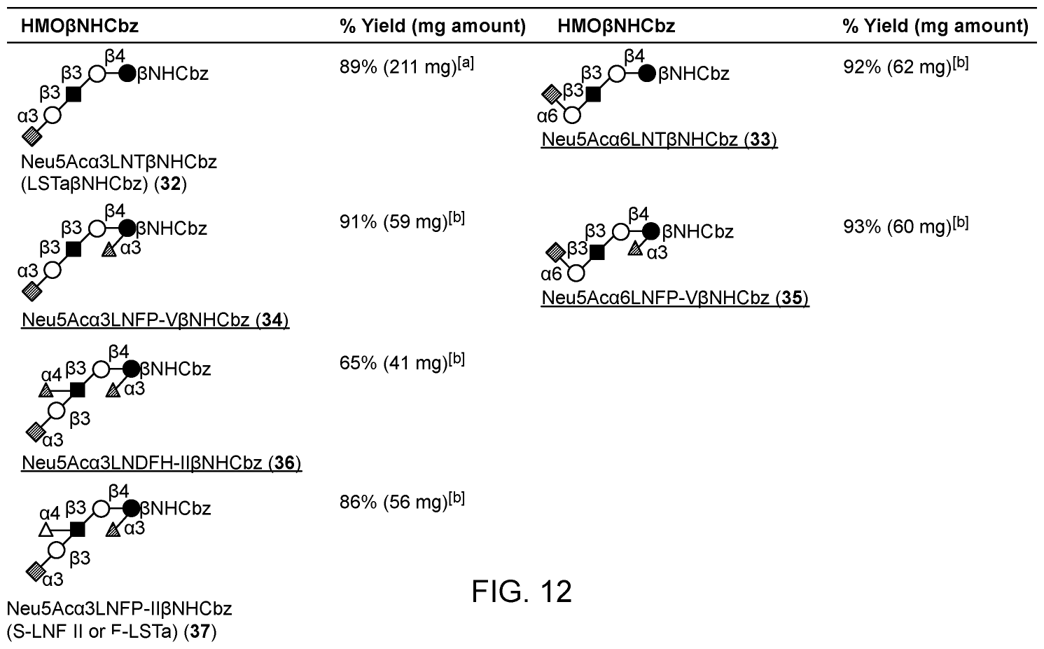

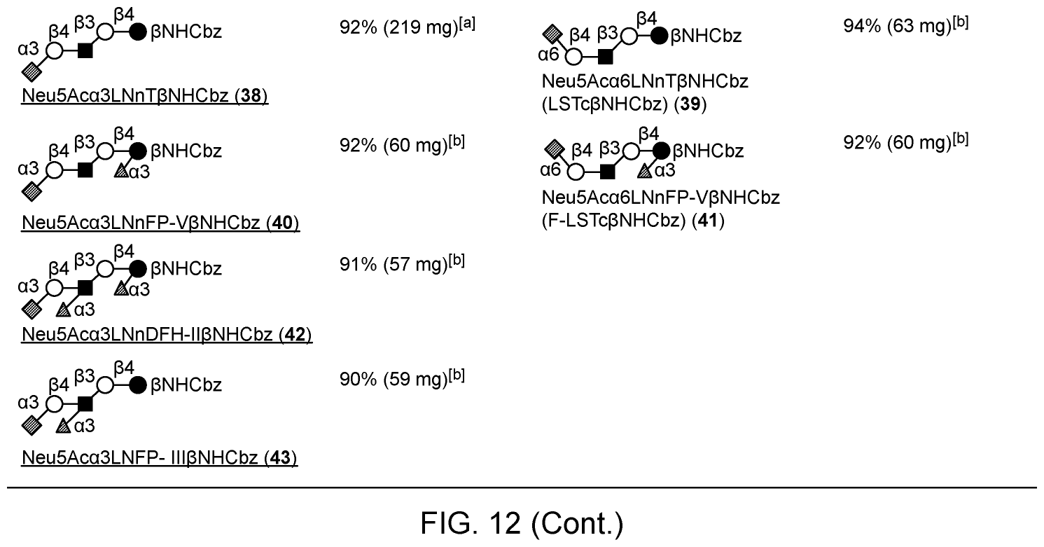

In some embodiments, compounds described herein are HMOs with an LNT or LNnT core. In some embodiments, compounds described herein have a neutral tetraose core, as shown in FIG. 10c. Certain non-limiting examples of HMO compounds described herein with a neutral LNT core are shown in FIG. 11A. Certain non-limiting examples HMO compounds described herein with a neutreal LNnT core are shown in FIG. 11B. In some aspects, the neutral HMO compounds described herein can contain 0-3 fucose residues, as shown in FIGS. 10C, 11A, and 11B.

Although LNnT-based HMOs with a fucose α1-2-linked to the terminal Gal have not been previously identified or isolated, they are possible products of appropriate fucosyltransferases. Therefore, there are a total of sixteen possible neutral HMOs containing a tetraose core of either LNT or LNnT (FIG. 10c). These neutral HMOs contain 0-3 fucose residues with the consideration of the possible presence and the absence of an α1-3-linked fucose at the D-glucose (Glc) in the reducing end, an α1-4-linked (for LNT-based HMOs) or α1-3-linked (for LNnT-based HMOs) fucose at the internal N-acetyl-D-glucosamine (GlcNAc), and an α1-2-linked fucose at the D-galactose (Gal) in the non-reducing end although the latter has not been reported for LNnT-based HMOs yet.

In some embodiments, HMO compounds described herein contain an LNT or an LNnT core, wherein the HMO compounds are negatively charged and monosialylated, and wherein the HMO compounds contain a Neu5Ac α2-3 (FIG. 10d) or α2-6 (FIG. 10e)-linked to the terminal Gal of the tetraose core.

In some embodiments, HMO compounds provided herein are LDNF-IIIβNHCbz, LNFP-VβNHCbz, LNDFH-IIβNHCbz, LNFP-IIβNHCbz, LNDFH-IβNHCbz, LNTβNHCbz, LNFP-IβNHCbz, or LNTFHepβNHCbz.

In some embodiments, HMO compounds provided herein are LNnFP-VβNHCbz, LNnDFH-IIIβNHCbz, LNnDFH-IIβNHCbz, LNFP-IIIβNHCbz, LNnDFH-IβNHCbz, LNnTβNHCbz, LNnFP-IβNHCbz, or LNnTFHepβNHCbz.

In some embodiments, the HMO compounds provided herein with a tetraose core may be sialylated. In some embodiments, the sialylated HMO compounds provided herein are Neu5Acα3LNTβNHCbz(LSTaβNHCbz), Neu5Acα6LNTβNHCbz, Neu5Acα3LNFP-VβNHCbz, Neu5Acα6LNFP-VβNHCbz, Neu5Acα3LNDFH-IIβNHCbz, Neu5Acα3LNFP-IIβNHCbz(S-LNF II or F-LSTa), Neu5Acα3LNnTβNHCbz, Neu5Acα6LNnTβNHCbz (LSTcβNHCbz), Neu5Acα3LNnFP-VβNHCbz, Neu5Acα6LNnFP-VβNHCbz(F-LSTcβNHCbz), Neu5Acα3LNnDFH-IIβNHCbz, or Neu5Acα3LNFP-IIIβNHCbz.

In some embodiments, the HMO compounds provided herein are negatively charged with an LNT core with a Neu5Acα2-6GlcNAc linkage. In some embodiments, the HMO compounds are LSTbβNHCbz, F-LSTbβNHCbz, DSLNTβNHCbz, FDS-LNT-IIβNHCbz, or FDS-LNT IβNHCbz.

In some embodiments, any one of the compounds provided herein may be obtained in isolated form. An isolated HMO compound as provided herein may be between 70% and 100% pure (e.g., 70%, 75%, 80%, 85%, 90%, 95%, or 100% purity).

In some embodiments, the compounds described herein are obtained in at least 50% yield and up to quantitative (100%) yield (e.g., 50% to 99%, 60% to 95%, 70% to 90% or 80 to 85% yield).

### IV. Methods of Preparing HMO Compounds

Described herein are multistep one-pot multienzyme (MSOPME) and stepwise one-pot multienzyme (StOPMe) strategies for enzymatic synthesis of HMO compounds from precursor materials, e.g., from monosaccharides and oligosaccharides. Monosaccharides may include, but are not limited to, GlcNAc, Gal, L-Fuc, Neu5Ac, ManNAc, Neu5Gc, ManNGc, mannose, and Kdn. Oligosaccharides may include, but are not limited to, Lac and LacβOR. As used herein, “one-pot” refers to performing one or multiple steps in the same reaction vessel. Optionally, the methods provided herein are performed without the purification of intermediate oligosaccharides. The methods described herein provide quick access to a variety of HMO compounds and derivatives.

For example, the methods and enzymes described herein can be applied to synthesizing a variety of HMO compounds, including short-chain and long-chain as well as linear and branched HMOs and their sialylated and/or fucosylated derivatives.

Accordingly, one aspect of the disclosure provides a method for preparing a compound according to Formula I, or salt thereof, the method comprising: forming a reaction mixture containing a protected lactosylamine; one or more monosaccharides selected from the group consisting of galactose, fucose, N-acetylglucosamine, and N-acetylneuraminic acid; one or more monosaccharide sugar activation enzymes; one or more nucleotide triphosphates; and one or more glycosyltransferases; and incubating the reaction mixture to form the compound according to Formula I;

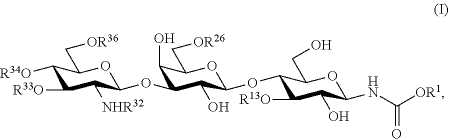

wherein: R¹ is selected from the group consisting of benzyl and fluorenylmethyl; R¹³ is selected from the group consisting of H and fucosyl; R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R³² is selected from the group consisting of H, acetyl, trifluoroacetyl, Boc, Fmoc; R³⁶ is selected from the group consisting of H and N-acetylneuraminic acid; R³³ and R³⁴ are independently selected from the group consisting of H, fucosyl, and a galactosyl moiety A:

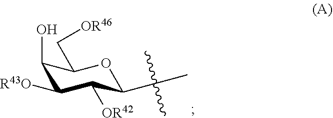

R⁴² is selected from the group consisting of H and fucosyl; R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R⁴³ is selected from the group consisting of a H, a monosaccharide, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid.

In some embodiments, the the reaction mixture of the method for preparing a compound according to Formula I, or salt thereof, comprises the protected lactosylamine. Optionally, the protected lactosylamine is purified prior to introduction to the enzymatic reaction mixture. In some examples, the protected lactosylamine is purified by crystallization. The crystallization can be performed by (i) dissolving crude protected lactosylamine in a solvent mixture at an elevated temperature; (ii) cooling the combined mixture of protected lactosylamine in the solvent mixture to room temperature; (iii) adding pure protected lactosylamine; and (iv) incubating the mixture for a period of time to obtain crystals. Optionally, the solvent mixture comprises water and an alcohol (e.g., methanol, ethanol, propanol, 1-butanol, isobutanol, and the like). Optionally, the solvent mixture comprises a 5:1 to 1:5 ratio (e.g., a 2:1 to 1:2 ratio) of the water to the alcohol.

Another aspect described herein is a method for preparing a compound according to Formula II, or a salt thereof, the method comprising: forming a reaction mixture containing a protected lactosylthiazolidine; one or more monosaccharides selected from the group consisting of galactose, fucose, N-acetylglucosamine, and N-acetylneuraminic acid; one or more monosaccharide sugar activation enzymes; one or more nucleotide triphosphates; and one or more glycosyltransferases; and incubating the reaction mixture to form the compound according to Formula II;

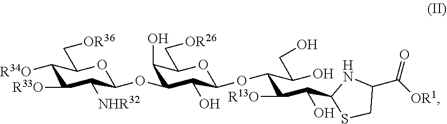

wherein: R¹ is selected from the group consisting of benzyl and fluorenylmethyl; R¹³ is selected from the group consisting of H and fucosyl; R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R³² is selected from the group consisting of H, acetyl, trifluoroacetyl, Boc, Fmoc; R³⁶ is selected from the group consisting of H and N-acetylneuraminic acid; R³³ and R³⁴ are independently selected from the group consisting of H, fucosyl, and a galactosyl moiety A:

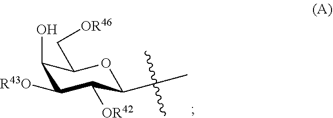

R⁴² is selected from the group consisting of H and fucosyl; R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R⁴³ is selected from the group consisting of a H, a monosaccharide, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid.

Another aspect of the disclosure provides a method for preparing a compound according to Formula III,

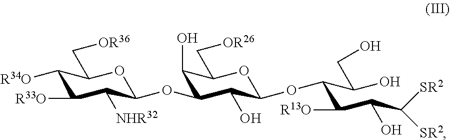

or salt thereof, the method comprising: forming a reaction mixture containing a lactose dithioacetal, one or more monosaccharides selected from the group consisting of galactose, fucose, N-acetylglucosamine, and N-acetylneuraminic acid, one or more monosaccharide sugar activation enzymes, one or more nucleotide triphosphates, and one or more glycosyltransferases; and incubating the reaction mixture to form the compound according to Formula III; wherein each R² is is independently selected from the group consisting of C₁₋₆ alkyl and optionally substituted C₆₋₁₄ aryl, or two R² are taken together to form a C₁₋₆ alkylene diradical; R¹³ is selected from the group consisting of H and fucosyl; R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R³² is selected from the group consisting of H, acetyl, trifluoroacetyl, Boc, Fmoc; R³⁶ is selected from the group consisting of H and N-acetylneuraminic acid; R³³ and R³⁴ are independently selected from the group consisting of H, fucosyl, and a galactosyl moiety A

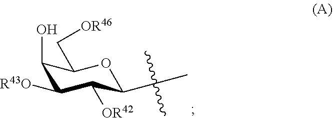

R⁴² is selected from the group consisting of H and fucosyl; R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid; R⁴³ is selected from the group consisting of a H, a monosaccharide, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid.

In some embodiments, the incubation of the reaction mixture of the methods described herein comprises converting the nucleotide triphosphate and the monosaccharide to 1.1-2 molar equivalents of a nucleotide sugar, with respect to the protected lactosylamine, the protected lactosylthiazolidine, or the lactose dithioacetal.

In some embodiments, the reaction mixture of the methods described herein to prepare compounds of Formula I, Formula II, or Formula III, wherein the compounds of Formula I, Formula II, or Formula III comprise one or more fucosyl moieties, comprises one or more of the glycosyltransferases in the reaction mixture wherein the one or more glycosyltransferases are fucosyltransferases.

In some embodiments, two or more of the glycosyltransferase reactions of the methods described herein are conducted without purification and/or isolation of the oligosaccharide intermediates.

In some embodiments, the methods described herein include a purification step to isolate the compounds described herein. The purification step may include C18 cartridge purification. In some embodiments, the compounds described herein may be isolated via reverse-phase chromatography. In some embodiments, wherein the isolated compound is a compound of Formula I, the —NHC(O)OR¹ moiety is converted to a —OH moiety in the isolated compound.

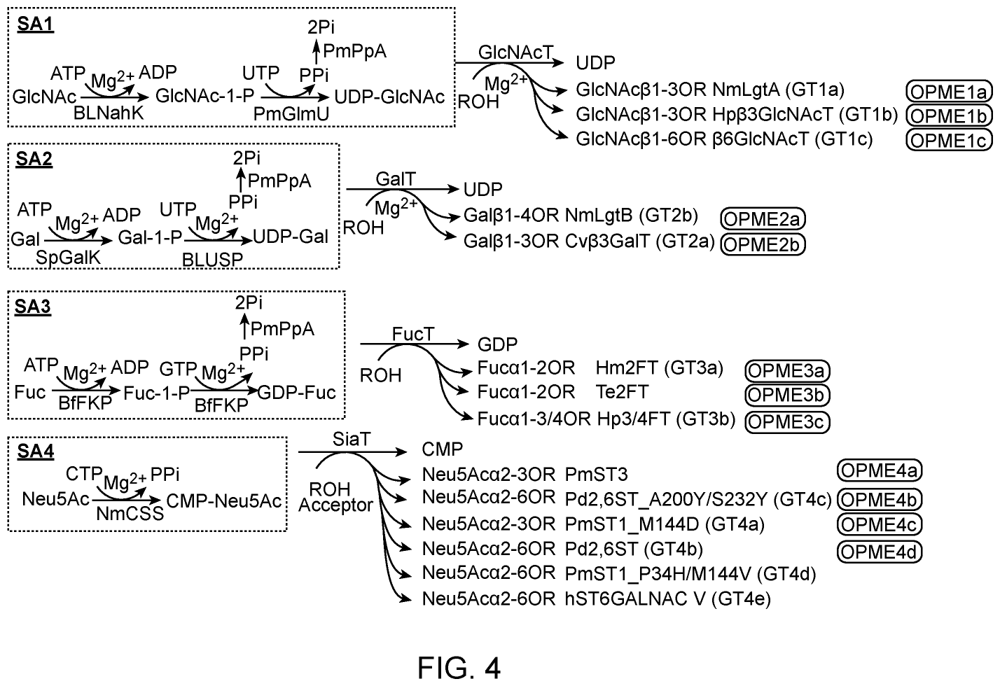

The methods described herein may include one or more sugar activation systems (SA), as shown in FIG. 4. The sugar activation systems described herein may contain one or more enzymes. In some embodiments, the enzymes are glycosyltransferases. Glycosyltransferases in the sugar activation systems described herein use activated sugar nucleotides as donor substrates which can be generated in situ from simple monosaccharides and enzymes involved in the formation of sugar nucleotides. Without being limited by theory, the four sugar activation systems described herein (SA1, SA2, SA3, and SA4, shown in FIG. 4), can be used for in situ-generation of the corresponding sugar nucleotides which can be combined with suitable glycosyltransferases for the formation of the desired glycosidic linkages.

In some embodiments, the enzymes for the formation of sugar nucleotide and the glycosyltransferases used herein are BLNahK, Bifidobacterium longum N-acetylhexosamine-1-kinase; PmGlmU, Pasteurella multocida N-acetylglucosamine 1-phosphate uridylyltransferase; PmPpA, Pasteurella multocida inorganic pyrophosphatase; NmLgtA, Neisseria meningitidis 01-3-N-acetylglucosaminyltransferase; Hpβ3GlcNAcT, Helicobacter pylori 01-3-N-acetylglucosaminyltransferase; β6GlcNAcT, β1-6-N-acetylglucosaminyltransferase; SpGalK, Streptococcus pneumoniae galactokinase; BLUSP, Bifidobacterium longum UDP-sugar synthase; Cvβ3GalT, Chromobacterium violaceum β1-3-galactosyltransferase; NmLgtB, Neisseria meningitidis β1-4-galactosyltransferase; BfFKP, a bifunctional enzyme from Bacteroides fragilis that has both L-fucokinase and GDP-fucose pyrophosphorylase activities; Hm2FT, Helicobacter mustelae α1-2-fucosyltransferase; Te2FT, Thermosynechococcus elongatus α1-2-fucosyltransferase; Hp3/4FT, Helicobacter pylori α1-3/4-fucosyltransferase; NmCSS, Neisseria meningitidis CMP-sialic acid synthetase; PmST3, Pasteurella multocida α2-3-sialyltransferase 3; PmST1_M144D, Pasteurella multocida α2-3-sialyltransferase 1 M144D mutant; Pd2,6ST, Photobacterium damselae α2-6-sialyltransferase (can add Neu5Ac to both terminal and internal Gal residues); Pd2,6ST_A200Y/S232Y, Photobacterium damselae α2-6-sialyltransferase A200Y/S232Y double mutant (a regio-selective α2-6-sialyltransferase for adding Neu5Ac to terminal Gal only); PmST1_β34H/M144V, PmST1 P34H/M144V double mutant (a regio-selective α2-6-sialyltransferase for adding Neu5Ac preferably to terminal Gal); hST6GALNAC V, recombinant human ST6GALNAC V expressed in E. coli; or a combination thereof.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### V. Nucleic Acid and Amino Acid Sequences of Enzymes Described Herein

Also described herein is an hST6GALNAC V variant comprising a polypeptide having at least 80% identity to the amino acid sequence of wild-type hST6GALNAC V. Optionally, the hST6GALNAC V variant comprises a mutation at one or more positions corresponding to G82, A84, V99, H107, G132, 5140, Y176, Q184, H190, L191, A201, H207, Q218, T239, F275, and N306 in wild-type hST6GALNAC V. In some cases, the N-terminus of the polypeptide is fused to a maltose binding protein. In some cases, the C-terminus of the polypeptide is fused to a His₆ peptide. Further described herein are hST6GALNAC V variants comprising a polypeptide having at least 80% identity to the amino acid sequence set forth in SEQ ID NO: 5, SEQ ID NO: 6, SEQ ID NO: 7, or SEQ ID NO: 8. Further described herein is a polynucleotide encoding a hST6GALNAC V variant as described herein. A host cell comprising the polynucleotide is also provided herein. Additionally provided herein is a reaction mixture comprising an hST6GALNAC V variant as described herein.

Further provided herein is an hGCNT2-B variant comprising a polypeptide having at least 80% identity to the amino acid sequence set forth in SEQ ID NO: 10. Also provided herein is a polynucleotide encoding an hGCNT2-B variant as described herein. A host cell comprising the polynucleotide is also provided herein. Additionally provided herein is a reaction mixture comprising an hGCNT2-B variant as described herein.

Provided below in the application are sequences corresponding to hST6GALNAC V and hGCNT2-B variants.

SEQ ID NO: 1 is the nucleic acid sequence of MBP-Δ50hST6GALNAC V-His₆, as provided in the Sequence Listing herein.

SEQ ID NO: 2 is the nucleic acid sequence of MBP-Δ50hST6GALNAC V_V99M-His₆, as provided in the Sequence Listing herein.

SEQ ID NO: 3 is the nucleic acid sequence of MBP-Δ50hST6GALNAC V_ext20-His₆, as provided in the Sequence Listing herein.

SEQ ID NO: 4 is the nucleic acid sequence of MBP-Δ50hST6GALNAC V_V99M_ext20-His₆, as provided in the Sequence Listing herein.

SEQ ID NO: 5 is the amino acid sequence of MBP-Δ50hST6GALNAC V-His₆, as provided in the Sequence Listing herein.

SEQ ID NO: 6 is the amino acid sequence of MBP-Δ50hST6GALNAC V_V99M-His₆, as provided in the Sequence Listing herein.

SEQ ID NO: 7 is the amino acid sequence of MBP-Δ50hST6GALNAC V_ext20-His₆, as provided in the Sequence Listing herein.

SEQ ID NO: 8 is the amino acid sequence of MBP-Δ50hST6GALNAC V_V99M_ext20-His₆, as provided in the Sequence Listing herein.

SEQ ID NO: 9 is the nucleic acid sequence of MBP-Δ25hGCNT2-B-His₆, as provided in the Sequence Listing herein.

SEQ ID NO: 10 is the amino acid sequence of MBP-Δ25hGCNT2-B-His₆, as provided in the Sequence Listing herein.

### Example 1. Materials and Methods for HMO Compounds Prepared Using MSOPME Strategy

Materials. All chemicals were obtained from commercial suppliers and used without further purification. ¹H NMR (600 or 800 MHz) and ¹³C NMR (150 or 200 MHz) spectra were recorded on a Bruker Avance-600 Spectrometer or a Avance-800 Spectrometer. High-resolution electrospray ionization (ESI) mass spectra were recorded using a Thermo Scientific Q Exactive HF Orbitrap Mass Spectrometer at the Mass Spectrometry Facilities in The University of California, Davis. Thin-layer chromatography (TLC, Sorbent Technologies) was performed on silica gel plates using anisaldehyde sugar stain for detection.

Expression and purification of enzymes involved in the synthesis. Recombinant enzymes were expressed and purified as described previously for NmLgtA, Hpβ3GlcNAcT, NmLgtB, Cvβ3GalT, PmST3, Pd2,6ST_A200Y/S232Y, Hm2FT, Hp3/4FT, Te2FT, SpGalK, BLUSP, BLNahK, PmGlmU, NmCSS, BfFKP, PmPpA. See, Lau, Yu, and X. Chen, et al. Chem. Commun. 2010, 46, 6066-6068; Li, Yu, and X. Chen, et al. Bioorg. Med. Chem. 2016, 24, 1696-1705; McArthur, Yu, and X. Chen, et al. ACS Catal. 2019, 9, 10721-10726; Thon, Yu, and X. Chen, et al. Appl. Microbiol. Biotechnol. 2012, 94, 977-985; Xu, Wang, Cao, and Cheng, et al. ACS Catal. 2018, 8, 7222-7227; Xiao and Wang, et al. J. Org. Chem. 2016, 81, 5851-5865; Yu and X. Chen, et al. Chem. Commun. 2017,53, 11012-11015; Zhao, Yu, and X. Chen, et al. Chem. Commun. 2016, 52, 3899-3902; M. Chen and Wang, et al. Carbohydr. Res. 2011, 346, 2421-2425; Muthana, Yu, and X. Chen, et al. Chem. Commun. 2012, 48, 2728-2730; Li, Yu, and X. Chen, et al. Molecules 2011, 16, 6396-6407; Y. Chen, Yu, and X. Chen, et al. Chem. Commun. 2011, 47, 10815-10817; Yu and X. Chen, et al. Bioorg. Med. Chem. 2004, 12, 6427-6435; Yi and X. Chen et al., Proc. Nat. Acad. Sci. U.S.A 2009, 106, 4207-4212; which are incorporated herein by reference in their entireties.

Briefly, E. coli BL21 (DE3) strains harboring the recombinant plasmid with the target gene was cultured in 50 mL Luria-Bertani (LB) media (10 g/L tryptone, 5 g/L yeast extract, and 10 g/L NaCl) containing 0.1 mg/mL ampicillin with rapid shaking (220 rpm) at 37° C. for overnight. Then 15 mL of the overnight cell culture was transferred into 1 L of LB media containing 0.1 mg/mL ampicillin and incubated at 37° C. When the OD_{600 nm} of the cell culture reached 0.6-0.8, isopropyl-1-thio-β-D-galactopyranoside (IPTG, 0.1 mM) was added to induce the expression of the recombinant enzyme. The culture was then incubated at 20° C. with shaking (220 rpm) for 20 h. Cells were collected by centrifugation at 4392×g for 50 mn at 4° C. The cell pellet was re-suspended with lysis buffer (100 mM Tris-HCl buffer, pH 8.0, containing 0.1% Triton X-100). The cells were lysed by sonication with the following conditions: amplitude at 65%, 2 s pulse on and 3 s pulse off for 120 cycles. Cell lysate was obtained by centrifugation at 9016×g for 1 hour at 4° C. The supernatant was collected and loaded to a Ni²⁺-NTA affinity column pre-equilibrated with a binding buffer (50 mM Tris-HCl buffer, pH 7.5, 5 mM imidazole, 0.5 M NaCl). The column was washed with 10 column volumes of binding buffer and 10 column volumes of washing buffer (50 mM Tris-HCl buffer, pH 7.5, 10 mM imidazole, 0.5 M NaCl) and eluted using 10 column volumes of elution buffer (50 mM Tris-HCl buffer, pH 7.5, 200 mM imidazole, 0.5 M NaCl). Fractions containing the target protein were collected. PmST3, SpGalK, BLUSP, BLNahK, PmGlmU, NmCSS and PmPpA were dialyzed against a dialysis buffer (20 mM Tris-HCl buffer, pH 7.5, 10% glycerol) three times and stored at −20° C. 10% glycerol (by volume) was added to un-dialyzed NmLgtA, Hpβ3GlcNAcT, NmLgtB, Cvβ3GalT, Pd2,6ST_A200Y/S232Y, Hm2FT, Hp3/4FT, Te2FT and BfFKP, prior to storage at −20° C.

Mutagenesis to obtain Pd2,6ST_A200Y/S232Y. Plasmid in pET15b vector for expressing an N-terminal His₆-tagged truncated Pd2,6ST (16-497 aa) was used as a mutagenesis template. See, Yu and X. Chen, et al. Angew. Chem. Int. Ed. Engl. 2006, 45, 3938-3944; See, Huynh, Yu, X. Chen, and Fisher, et al. FEBS Lett. 2014, 588, 4720-4729, which are incorporated herein by reference in their entireties. The plasmid was used as the template for polymerase chain reactions (PCRs) to introduce mutation sites following the QuikChange™ site-directed mutagenesis method (Stratagene; La Jolla, CA, USA). The single mutant Pd2,6ST_A200Y was obtained first which was then used as the template to obtain the double mutant Pd2,6ST_A200Y/S232Y. Primers used for generating the A200Y and S232Y mutations are shown below (with mutated sited underlined).

A200Y forward
(SEQ ID NO: 11)
5′-TATACAAATACATATCATGTTTTTAATAATTTACCACCT-3′
A200Y reverse
(SEQ ID NO: 12)
5′-ATTAAAAACATGATATGTATTTGTATAGAACTCTATATT-3′
S232Y forward
(SEQ ID NO: 13)
5′-GATGATGGTTCTTATGAATATGTAAGTT-TATATCAATGG-3′
S232Y reverse
(SEQ ID NO: 14)
5′-ACTTACATATTCATAAGAACCATCATCATACAAAC-TAAT-3′

Polymerase chain reactions (PCRs) were each performed in a 50 μL reaction mixture containing 5 ng of template DNA, 1 M for each of the forward and reverse primers, 5 μL of 10× Phusion© HF buffer, 1 mM dNTP mixture, and 5 units (1 μL) of Phusion® HF DNA polymerase. The reaction mixtures were subjected to 30 cycles of amplifications with an annealing temperature of 55° C. The resulting PCR products were digested with DpnI, purified, and transformed into chemically competent E. coli DH5α cells. The plasmids of selected colonies were purified and sent to Genewiz (South Plainfield, NJ, USA) for sequencing. Positive plasmids were transformed into BL21 (DE3) chemically competent cells. Selected clones were grown for protein expression, purification, characterization, and application in chemoenzymatic synthesis.

### Example 2. Preparative Scale Synthesis and Purification of βNHCbz-Tagged Human Milk Oligosaccharides

Multigram-scale synthesis and purification of LacβNHCbz (1) from lactose. Lactose (5 g, 14.62 mmol) and ammonium bicarbonate (1.3 g, 16.45 mmol) was dissolved in ammonium hydroxide (25 mL) and the solution was incubated at 45-50° C. for 24 hours to form lactosylamine (LacβNH₂). Solvent was removed in vacuo and the residue was dried under vacuum for 3-4 h. To obtain LacβNHCbz, LacβNH₂ (5 g, 14.62 mmol) was dissolved in 650 mL of MeOH (methanol) and benzyl chloroformate (CbzCl, 9.5 g, 55.69 mmol) was added to the reaction mixture in a 1 L round bottom flask submerged in an ice-water bath. The pH of the mixture was adjusted and kept at 8.0-10.0 by adding N,N-diisopropylethylamine. The reaction mixture was then stirred at room temperature for 22 hours. LacβNHCbz was purified by ODS-SM column (140 g, 50 μm, 120 Å, Yamazen) on a CombiFlash® Rf 200i system. Consistently, 75-80% yields were obtained for 3-5 g-scale syntheses. The product formation was monitored by thin-layer chromatography (TLC) and by high-resolution mass spectrometry (HRMS). Ethyl acetate (EtOAc):methanol (MeOH):H₂O=5:1.6:1 (by volume) was used as the developing solvent for TLC. HRMS (ESI-Orbitrap) m/z: [M+Na]⁺ calculated for C₂₀H₂₉NNaO₁₂ 498.1587; found 498.1585.

Single C18-cartridge purification process for MSOPME-synthesized βNHCbz-tagged HMOs (2-22). After the reaction was completed, the reaction mixture was incubated in a boiling water bath for 5 min to denature the enzymes. The mixture was then cooled down to room temperature and centrifuged at 9016×g for 30 min at 4° C. The supernatant was collected. The precipitate was washed twice, each time with 3 mL of H₂O, and the supernatants were combined. The combined supernatant was concentrated by rotavap to reduce the volume to about 3-5 mL which was purified by passing through a ODS-SM column (51 g, 50 μm, 120 Å, Yamazen) on a CombiFlash® Rf 200i system and monitored at 214 nm. The product was eluted with a mixed solvent of acetonitrile and water with a flow rate of 30 mL/min. The eluting program used was the following: Mobile phase A: water (v/v); Mobile phase B: acetonitrile (v/v); 0% B for 8 min followed by gradient 0% to 60% B over 25 min, gradient 60% to 100% B over 3 min, 100% B for 2 min, then 100% to 80% B over 2 min.

Preparative-scale synthesis of GlcNAcβ3Galβ4GlcβNHCbz (LNT-IIβNHCbz) (2) by OPME1a. LacβNHCbz (1, 100 mg, 0.21 mmol), GlcNAc (0.32 mmol), ATP (0.32 mmol), UTP (0.32 mmol) were dissolved in water in a 50 mL centrifuge tube containing Tris-HCl buffer (100 mM, pH 7.5) and MgCl₂ (20 mM). After the addition of BLNahK (1.5 mg), PmGlmU (1.2 mg), NmLgtA (1.8 mg), and PmPpA (1 mg) (OPME1a), water was added to bring the final volume to 21 mL and the concentration of LacβNHCbz (1) was 10 mM. The reaction mixture was incubated at 30° C. with agitation at 180 rpm in an incubator shaker for overnight. The product formation was monitored by thin-layer chromatography (TLC) and by high-resolution mass spectrometry (HRMS). Ethyl acetate (EtOAc):methanol (MeOH):H₂O=5:1.6:1 (by volume) was used as the developing solvent for TLC. After the reaction was completed (22 h), the reaction mixture was incubated in a boiling water bath for 5 min to denature the enzymes. The reaction mixture was then cooled down and the product was purified by following the single C18-cartridge purification procedures described above. LNT-IIβNHCbz (2) was obtained as a white powder (130 mg, 91% yield in 1 step from 100 mg LacβNHCbz). ¹H NMR (800 MHz, D₂O, 30° C.) δ 7.46-7.38 (m, 5H), 5.17 (t, J=9.4 Hz, 2H), 4.81 (d, J=9.3 Hz, 1H), 4.67 (d, J=8.5 Hz, 1H), 4.43 (d, J=7.9 Hz, 1H), 4.13 (d, J=3.3 Hz, 1H), 3.93-3.84 (m, 2H), 3.80-3.52 (m, 12H), 3.49-3.34 (m, 3H), 2.02 (s, 3H). ¹³C NMR (200 MHz, D₂O, 30° C.) δ 174.92, 158.08, 135.99, 128.76, 128.46, 127.80, 102.82, 102.81, 81.85, 81.62, 77.67, 76.41, 76.11, 75.62, 74.99, 74.85, 73.51, 71.37, 69.98, 69.65, 69.20, 68.33, 67.38, 60.94, 60.44, 59.83, 55.62, 22.12. HRMS (ESI-Orbitrap) m/z: [M+Na]⁺ calculated for C₂₈H₄₂N₂NaO₁₇ 701.2381; found 701.2383.

MSOPME preparative-scale synthesis of Galβ4GlcNAcβ3Galβ4GlcβNHCbz (LNnTβNHCbz) (3). The same procedure was carried out as described above (OPME1a) for the preparation of LNT-IIβNHCbz (2) from LacβNHCbz (1). After confirming the completion of the reaction for the formation of LNT-IIβNHCbz (2), the reaction mixture was incubated in a boiling water bath for 5 min and was then cooled down to room temperature. To the reaction mixture in the same tube, Gal (0.32 mmol), ATP (0.32 mmol), and UTP (0.32 mmol) were added and the pH of the reaction was adjusted to 7.5 by adding 4 M NaOH. Enzymes including SpGalK (2.5 mg), BLUSP (2 mg), NmLgtB (3 mg), and PmPpA (1 mg) (OPME2a) were then added without increasing the volume of the reaction mixture significantly. The reaction mixture was incubated at 30° C. in an incubator shaker with agitation at 180 rpm. The product formation was monitored by TLC with EtOAc:MeOH:H₂O=5:2:1 (by volume) as the developing solvent and by HRMS. After the reaction was completed (14 h), the reaction mixture was incubated in a boiling water bath for 5 min to denature the enzymes and then cooled down to room temperature before the product was purified. LNnTβNHCbz (3) was obtained as a white powder (152 mg, 86% yield for two steps from 100 mg LacβNHCbz). ¹H NMR (800 MHz, D₂O, 30° C.) δ 7.45-7.38 (m, 5H), 5.17 (t, J=9.3 Hz, 2H), 4.81 (s, 1H), 4.69 (d, J=8.4 Hz, 1H), 4.46 (d, J=7.9 Hz, 1H), 4.43 (d, J=7.9 Hz, 1H), 4.14 (d, J=3.3 Hz, 1H), 3.96-3.87 (m, 3H), 3.86-3.61 (m, 16H), 3.60-3.50 (m, 3H), 3.43-3.33 (m, 1H), 2.02 (s, 3H). ¹³C NMR (200 MHz, D₂O, 30° C.) δ 174.94, 158.15, 136.05, 128.83, 128.53, 127.87, 102.89, 102.78, 82.01, 81.70, 78.19, 77.74, 76.48, 76.18, 75.38, 75.06, 74.91, 74.59, 72.53, 72.20, 71.44, 71.00, 70.01, 69.27, 68.58, 68.39, 67.45, 61.06, 61.01, 59.89, 55.22, 22.21. HRMS (ESI-Orbitrap) m/z: [M+Na]⁺ calculated for C₃₄H₅₂N₂NaO₂₂ 863.2909; found 863.2896.

MSOPME preparative-scale synthesis of GlcNAcβ3Galβ4GlcNAcβ3Galβ4Glc-βNHCbz (GlcNAc-LNnTβNHCbz) (4). LacβNHCbz (1, 50 mg, 0.105 mmol), GlcNAc (0.16 mmol), ATP (0.16 mmol), and UTP (0.16 mmol) were dissolved in water in a 50 mL centrifuge tube containing Tris-HCl buffer (200 mM, pH 7.5) and MgCl₂ (40 mM). After the addition of BLNahK (1.2 mg), PmGlmU (1 mg), NmLgtA (1.5 mg), and PmPpA (0.5 mg) (OPME1a), water was added to bring the final volume to 5 mL, resulting in a solution containing 20 mM LacβNHCbz (1). The reaction mixture was incubated at 30° C. in an incubator shaker for 18 h with agitation at 180 rpm. Reaction progress was monitored by TLC and by HRMS as described above for LNT-IIβNHCbz (2). After incubating the reaction mixture in a boiling water bath for 5 min and then cooled down, Gal (0.16 mmol), ATP (0.16 mmol), and UTP (0.16 mmol) were added, the pH of the reaction was adjusted to 7.5 by adding 4 M NaOH. SpGalK (1.5 mg), BLUSP (1.2 mg), NmLgtB (1.8 mg), and PmPpA (0.5 mg) (OPME2a) were then added and the reaction mixture was incubated at 30° C. for overnight with agitation at 180 rpm. Reaction progress was monitored by TLC and by HRMS. Once the reaction was completed (14 h), the mixture was incubated in a boiling water bath for 5 min and then cooled down. In the same reaction tube without workup or purification, GlcNAc (0.16 mmol), ATP (0.16 mmol), and UTP (0.16 mmol) were added, and the pH of the reaction was adjusted to 7.5 by adding 4 M NaOH. BLNahK (1 mg), PmGlmU (0.8 mg), Hpβ3GlcNAcT (1.5 mg), and PmPpA (0.5 mg) (OPME1b) were then added. The final concentration of the acceptor in the reaction mixture was kept at around 10 mM and the final volume of the reaction mixture was kept at around 10.5 mL by adding Tris-HCl buffer (100 mM, pH 7.5) and MgCl₂ (20 mM). The reaction mixture was incubated at 30° C. for overnight with agitation at 180 rpm. The product formation was monitored by TLC with EtOAc:MeOH:H₂O=5:2.4:1 (by volume) as the developing solvent and by HRMS. After the reaction was completed (16 h), the reaction mixture was incubated in a boiling water bath for 5 min, cooled down, and purified. GlcNAc-LNnTβNHCbz (4) was obtained as a white powder (91 mg, 83% yield for three steps from 50 mg LacβNHCbz). ¹H NMR (600 MHz, D₂O, 30° C.) δ 7.48-7.39 (m, 5H), 5.18 (d, J=3.5 Hz, 2H), 4.82 (d, J=9.2 Hz, 1H), 4.69 (dd, J=11.7, 8.4 Hz, 2H), 4.47 (d, J=7.9 Hz, 1H), 4.44 (d, J=7.9 Hz, 1H), 4.15 (t, J=3.0 Hz, 2H), 3.97-3.87 (m, 3H), 3.85-3.65 (m, 18H), 3.58 (ddt, J=11.8, 10.4, 5.9 Hz, 4H), 3.51-3.40 (m, 3H), 2.04 (s, 3H), 2.03 (s, 3H). ¹³C NMR (150 MHz, D₂O, 30° C.) δ 174.93, 174.87, 158.08, 135.99, 128.77, 128.47, 128.41, 127.81, 127.67, 102.87, 102.83, 102.71, 81.97, 81.94, 81.62, 78.18, 77.71, 76.13, 75.64, 75.01, 74.86, 74.84, 74.53, 73.54, 72.14, 69.98, 69.95, 69.66, 68.34, 68.29, 67.39, 60.95, 60.93, 60.46, 59.85, 55.64, 55.13, 22.15. HRMS (ESI-Orbitrap) m/z: [M+Na]⁺ calculated for C₄₂H₆₅N₃NaO₂₇ 1066.3703; found 1066.3708.

MSOPME preparative-scale synthesis of Galβ4GlcNAcβ3Galβ4(Fucα3)Glc-βNHCbz (LNnFP-VβNHCbz) (5). LacβNHCbz (1, 50 mg, 0.105 mmol), GlcNAc (0.16 mmol), ATP (0.16 mmol), UTP (0.16 mmol) were dissolved in water in a 50 mL centrifuge tube containing Tris-HCl buffer (200 mM, pH 7.5) and MgCl₂ (40 mM). After the addition of BLNahK (1.2 mg), PmGlmU (1 mg), NmLgtA (1.5 mg), and PmPpA (0.5 mg) (OPME1a), water was added to bring the final volume to 5 mL with the concentration of LacβNHCbz (1) of 20 mM. The reaction mixture was incubated at 30° C. in an incubator shaker for 18 h with agitation at 180 rpm. Reaction was monitored as described above for preparation of LNT-IIβNHCbz (2). After the reaction was completed, the reaction mixture was incubated in a boiling water bath for 5 min and then cooled down. In the same reaction tube without workup or purification, _{L}-fucose (0.16 mmol), ATP (0.16 mmol), and GTP (0.16 mmol) were added, and the pH of the reaction was adjusted to 7.5 by adding 4 M NaOH. BfFKP (1.5 mg), Hp3/4FT (1 mg), and PmPpA (0.5 mg) (OPME3c) were then added. The product formation was monitored by TLC with EtOAc:MeOH:H₂O=5:2:1 (by volume) as the developing solvent and by HRMS. The reaction mixture was incubated at 30° C. for 14 h with agitation at 180 rpm. After reaction was completed, the reaction mixture was incubated in a boiling water bath for 5 min and then cooled down. In the same reaction tube without workup or purification, Gal (0.16 mmol), ATP (0.16 mmol), and UTP (0.16 mmol) were added, and the pH of the reaction was adjusted to 7.5 by adding 4 M NaOH. SpGalK (1.5 mg), BLUSP (1.2 mg), NmLgtB (1.8 mg), and PmPpA (0.5 mg) (OPME2a) were then added. The final concentration of the acceptor was kept at around 10 mM by adding Tris-HCl buffer (100 mM, pH 7.5) and MgCl₂ (20 mM) to bring the final volume of the reaction to around 10.5 mL. The reaction mixture was incubated at 30° C. for 16 h with agitation at 180 rpm. The product formation was monitored by TLC with EtOAc:MeOH:H₂O=5:2.4:1 (by volume) as the developing solvent and by HRMS. After the reaction was completed, the reaction mixture was incubated in a boiling water bath for 5 min to denature the enzymes, cooled down, and purified. LNnFP-V βNHCbz (5) was obtained as a white powder (83 mg, 80% yield for three steps from 50 mg LacβNHCbz). ¹H NMR (600 MHz, D₂O, 30° C.) δ 7.48-7.39 (m, 5H), 5.44 (d, J=4.0 Hz, 1H), 5.18 (s, 2H), 4.82 (d, J=5.7 Hz, 1H), 4.80 (s, 1H), 4.70 (d, J=8.3 Hz, 1H), 4.48 (d, J=7.8 Hz, 1H), 4.42 (d, J=7.7 Hz, 1H), 4.09 (d, J=3.5 Hz, 1H), 3.98-3.90 (m, 4H), 3.90-3.42 (m, 23H), 2.03 (s, 3H), 1.16 (d, J=6.6 Hz, 3H). ¹³C NMR (150 MHz, D₂O, 30° C.) δ 174.91, 158.18, 136.09, 128.84, 128.53, 127.85, 127.74, 102.91, 102.74, 101.74, 98.53, 81.88, 81.54, 78.25, 77.67, 76.80, 75.39, 74.57, 74.53, 73.54, 73.15, 72.55, 72.17, 72.07, 71.96, 71.01, 70.68, 69.26, 68.59, 68.31, 68.02, 67.44, 66.57, 61.52, 61.06, 59.91, 59.66, 55.20, 22.20, 15.25. HRMS (ESI-Orbitrap) m/z: [M+Na]⁺ calculated for C₄₀H₆₂N₂NaO₂₆1009.3488; found 1009.3410.

MSOPME preparative-scale synthesis of Galβ4(Fucα3)GlcNAcβ3Galβ4(Fucα3)-GlcβNHCbz (LNnDFH-IIβNHCbz) (6). LNnTβNHCbz (3) was prepared as an intermediate from 50 mg LacβNHCbz (1) (OPME1a and OPME2a) similar to that described above for the preparation of GlcNAc-LNnTβNHCbz (4). After the reaction for the formation of LNnTβNHCbz (3) was completed, the reaction mixture was incubated in a boiling water bath for 5 min and then cooled down. In the same reaction tube without workup or purification, _{L}-fucose (0.3 mmol), ATP (0.32 mmol), and GTP (0.32 mmol) were added. The pH of the reaction was adjusted to 7.5 by adding 4 M NaOH. BfFKP (4 mg), Hp3/4FT (1.5 mg), and PmPpA (1 mg) (OPME3c) were then added. The final concentration of the acceptor was kept at around 10 mM, and the final volume of the reaction was kept at around 10.5 mL by adding Tris-HCl buffer (100 mM, pH 7.5) and MgCl₂ (20 mM). The reaction mixture was incubated at 30° C. in an incubator shaker for 14 h with agitation at 180 rpm. The product formation was monitored by TLC with EtOAc:MeOH:H₂O=5:2.6:1 (by volume) as the developing solvent and by HRMS. After the reaction was completed, the reaction mixture was incubated in a boiling water bath for 5 min to denature the enzymes, cooled down, and purified. LNnDFH-IIβNHCbz (6) was obtained as a white powder (94 mg, 80% yield for three steps from 50 mg LacβNHCbz). ¹H NMR (600 MHz, D₂O, 30° C.) δ 7.48-7.39 (m, 5H), 5.44 (d, J=4.0 Hz, 1H), 5.18 (s, 2H), 5.14 (d, J=4.0 Hz, 1H), 4.84 (d, J=7.2 Hz, 2H), 4.71 (d, J=8.4 Hz, 1H), 4.47 (d, J=7.8 Hz, 1H), 4.42 (d, J=7.8 Hz, 1H), 4.09 (d, J=3.6 Hz, 1H), 4.01-3.55 (m, 29H), 3.50 (ddd, J=10.6, 5.4, 3.3 Hz, 2H), 2.02 (s, 3H), 1.20-1.12 (m, 6H). ¹³C NMR (150 MHz, D₂O, 30° C.) δ 174.64, 158.11, 136.02, 128.78, 128.47, 127.78, 127.68, 102.48, 101.73, 101.67, 98.56, 98.46, 81.82, 81.48, 77.60, 76.73, 75.06, 74.89, 74.73, 74.47, 73.02, 72.46, 71.99, 71.89, 71.03, 70.64, 69.18, 68.33, 68.24, 67.96, 67.68, 67.38, 66.67, 66.50, 61.49, 61.46, 59.62, 55.93, 22.22, 15.29, 15.18. HRMS (ESI-Orbitrap) m/z: [M+Na]⁺ calculated for C₄₆H₇₂N₂NaO₃₀ 1155.4068; found 1155.4071.

MSOPME preparative-scale synthesis of GalβGlcNAcβ3Galβ4GlcβNHCbz (LNTβNHCbz) (7). LNT-IIβNHCbz (2) was formed as an intermediate from 100 mg LacβNHCbz (1) (OPME1a) similar to that described above. After the reaction was completed, the reaction mixture was incubated in a boiling water bath for 5 min and then cooled down. In the same reaction tube without workup or purification, Gal (0.32 mmol), ATP (0.32 mmol), and UTP (0.32 mmol) were added, and the pH of the reaction was adjusted to 7.5 by adding 4 M NaOH. SpGalK (2.5 mg), BLUSP (2 mg), Cvβ3GalT (4 mg), and PmPpA (1 mg) (OPME2b) were then added and the volume of the reaction mixture was kept at around 21 mL. The reaction mixture was incubated at 30° C. in an incubator shaker for 14 h with agitation at 180 rpm. The product formation was monitored by TLC with EtOAc:MeOH:H₂O=5:2:1 (by volume) as the developing solvent and by HRMS. After the reaction was completed, the reaction mixture was incubated in a boiling water bath for 5 min to denature the enzymes, cooled down, and purified. LNTβNHCbz (7) was obtained as a white powder (155 mg, 88% yield for two steps from 100 mg LacβNHCbz). ¹H NMR (800 MHz, D₂O, 30° C.) δ 7.45-7.39 (m, 5H), 5.17 (t, J=9.4 Hz, 2H), 4.81 (d, J=9.1 Hz, 1H), 4.72 (d, J=8.5 Hz, 1H), 4.43 (dt, J=7.8, 1.3 Hz, 2H), 4.14 (d, J=3.3 Hz, 1H), 3.92-3.87 (m, 4H), 3.82-3.61 (m, 14H), 3.60-3.50 (m, 3H), 3.47 (ddd, J=10.0, 5.1, 2.3 Hz, 1H), 3.42-3.33 (m, 1H), 2.01 (s, 3H). ¹³C NMR (200 MHz, D₂O, 30° C.) δ 175.00, 158.15, 136.06, 128.83, 128.52, 127.87, 103.52, 102.88, 102.60, 82.07, 81.95, 81.84, 81.70, 77.76, 77.33, 76.48, 76.18, 75.31, 75.22, 75.06, 74.92, 72.49, 71.44, 70.71, 70.05, 69.27, 68.56, 68.48, 68.38, 67.45, 61.07, 61.01, 60.53, 59.90, 54.73, 22.26. HRMS (ESI-Orbitrap) m/z: [M+Na]⁺ calculated for C₃₄H₅₂N₂NaO₂₂ 863.2909; found 863.2911.

MSOPME gram-scale synthesis of Fucα2Galβ3GlcNAcβ3Galβ4GlcβNHCbz (LNFP-IβNHCbz) (8). LacβNHCbz (1, 1 g, 2.1 mmol), GlcNAc (3.2 mmol), ATP (3.2 mmol), UTP (3.2 mmol) were dissolved in water in a 250 mL wide-mouth bottle. Tris-HCl buffer (100 mM, pH 7.5) and MgCl₂ (20 mM) were then added. After BLNahK (8 mg), PmGlmU (9 mg), NmLgtA (12 mg), and PmPpA (3 mg) (OPME1a) were added, water was added to bring the final concentration of LacβNHCbz (1) to 25 mM. The reaction mixture was incubated at 30° C. in an incubator shaker for 3 days with agitation at 180 rpm. The product formation was monitored by TLC with EtOAc:MeOH:H₂O=5:1.6:1 (by volume) as the developing solvent and by HRMS. After the reaction was completed, Gal (3.2 mmol), ATP (3.2 mmol), UTP (3.2 mmol) were added and the pH was adjusted to 7.5. SpGalK (16 mg), BLUSP (16.5 mg), Cvβ3GalT (14 mg), and PmPpA (3 mg) (OPME2b) were added and the concentration of the acceptor was kept at 25 mM. The reaction mixture was incubated at 30° C. for 2 days with agitation at 180 rpm. The product formation was monitored by TLC with EtOAc:MeOH:H₂O=5:2:1 (by volume) as the developing solvent and by HRMS. After the reaction was completed, Fuc (3.2 mmol), ATP (3.2 mmol), and GTP (3.2 mmol) were added and the pH was adjusted to 7.5. BfFKP (30 mg), Te2FT (34 mg), and PmPpA (3 mg) (OPME3b) were then added and the concentration of the acceptor was kept at 25 mM. The reaction mixture was incubated at 30° C. for 5 days with agitation at 180 rpm. The product formation was monitored by TLC with EtOAc:MeOH:H₂O=5:2.4:1 (by volume) as the developing solvent and by HRMS. After the reaction was completed, the reaction mixture was incubated in a boiling water bath for 5 min to denature the enzymes, cooled down, and then centrifuge at 9016×g for 30 min at 4° C. The supernatant was concentrated and purified by ODS-SM column (140 g, 50 μm, 120 Å, Yamazen) using CombiFlash® Rf 200i system, with a flow rate of 40 mL/min and a gradient elution of 0-100% acetonitrile in water for 60 min. Mobile phase A: water (v/v); Mobile phase B: acetonitrile (v/v); Gradient: 0% B for 15 min, 0% to 100% B over 40 min, 100% B for 2 min, then 100% to 80% B over 3 min. LNFP-IβNHCbz (8) was obtained as a white powder (1.73 g, 84% yield for three steps from 1 g of LacβNHCbz). ¹H NMR (800 MHz, D₂O, 30° C.) δ 7.53-7.48 (m, 5H), 5.25 (m, J=5.7 Hz, 3H), 4.92-4.83 (m, 1H), 4.70 (d, J=7.7 Hz, 1H), 4.69 (d, J=8.4 Hz, 1H), 4.49 (d, J=7.9 Hz, 1H), 4.35 (q, J=6.7 Hz, 1H), 4.21-4.17 (m, 1H), 4.05 (dd, J=10.5, 8.6 Hz, 1H), 4.00-3.68 (m, 21H), 3.66-3.54 (m, 4H), 3.47 (dt, J=10.0, 8.1 Hz, 1H), 2.12 (s, 3H), 1.29 (d, J=6.6 Hz, 3H). ¹³C NMR (200 MHz, D₂O, 30° C.) δ 174.27, 159.29, 136.08, 128.85, 128.54, 128.48, 127.88, 127.75, 103.27, 102.95, 100.30, 99.56, 81.65, 81.59, 78.10, 77.76, 77.35, 77.28, 76.71, 76.51, 76.22, 75.32, 75.13, 75.07, 74.87, 73.56, 71.91, 71.48, 70.26, 69.51, 69.33, 69.19, 68.64, 68.63, 68.55, 68.12, 67.46, 66.54, 62.56, 61.19, 61.01, 60.62, 60.50, 59.96, 55.02, 22.22, 15.32. HRMS (ESI-Orbitrap) m/z: [M+Na]⁺ calculated for C₄₀H₆₂N₂NaO₂₆1009.3488; found 1009.3459.

MSOPME preparative-scale synthesis of GalβGlcNAcβ3Galβ4(Fucα3)Glc-βNHCbz (LNFP-VβNHCbz) (9). LacβNHCbz (1, 50 mg, 0.105 mmol), GlcNAc (0.16 mmol), ATP (0.16 mmol), and UTP (0.16 mmol) were dissolved in water in a 50 mL centrifuge tube containing Tris-HCl buffer (200 mM, pH 7.5) and MgCl₂ (40 mM). BLNahK (1.2 mg), PmGlmU (1 mg), NmLgtA (1.5 mg), and PmPpA (0.5 mg) (OPME1a) were then added and water was added to bring the total volume to 5 mL and the concentration of LacβNHCbz (1) to 20 mM. The reaction mixture was incubated at 30° C. in an incubator shaker for 18 h with agitation at 180 rpm. Reaction was monitored similar to that described for preparation of LNT-IIβNHCbz (2). After the reaction was completed, the reaction mixture was incubated in a boiling water bath for 5 min and then cooled down. In the same reaction tube without workup or purification, _{L}-fucose (0.16 mmol), ATP (0.16 mmol), and GTP (0.16 mmol) were added and the pH was adjusted to 7.5 by adding 4 M NaOH. BfFKP (1.5 mg), Hp3/4FT (1 mg), and PmPpA (0.5 mg) (OPME3c) were then added and the acceptor concentration was around 15 mM. The product formation was monitored by TLC with EtOAc:MeOH:H₂O=5:2:1 (by volume) as the developing solvent and by HRMS. The reaction mixture was incubated at 30° C. for 14 h with agitation at 180 rpm. After the reaction was completed, the reaction mixture was incubated in a boiling water bath for 5 min and then cooled down. In the same reaction tube without workup or purification, Gal (0.16 mmol), ATP (0.16 mmol), and UTP (0.16 mmol) were added and the pH was adjusted to 7.5 by adding 4 M NaOH. SpGalK (1.5 mg), BLUSP (1.2 mg), Cvβ3GalT (2.5 mg), and PmPpA (0.5 mg) (OPME2b) were then added and the final volume of the reaction was kept at around 10.5 mL with the final concentration of the acceptor at around 10 mM. The reaction mixture was incubated at 30° C. for 16 h with agitation at 180 rpm. The product formation was monitored by TLC with EtOAc:MeOH:H₂O=5:2.4:1 (by volume) as the developing solvent and by HRMS. After the reaction was completed, the reaction mixture was incubated in a boiling water bath for 5 min to denature the enzymes, cooled down, and purified. LNFP-VβNHCbz (9) was obtained as a white powder (84 mg, 81% yield for three steps from 50 mg LacβNHCbz). ¹H NMR (800 MHz, D₂O, 30° C.) δ 7.45-7.41 (m, 5H), 5.44 (d, J=4.0 Hz, 1H), 5.18 (d, J=5.5 Hz, 2H), 4.82 (s, 1H), 4.80 (s, 1H), 4.72 (d, J=8.5 Hz, 1H), 4.44 (d, J=7.7 Hz, 1H), 4.42 (d, J=7.8 Hz, 1H), 4.09 (d, J=3.5 Hz, 1H), 3.95-3.68 (m, 19H), 3.65-3.46 (m, 8H), 2.02 (s, 3H), 1.16 (d, J=6.7 Hz, 3H). ¹³C NMR (200 MHz, D₂O, 30° C.) δ 174.98, 158.17, 136.08, 128.84, 128.83, 128.53, 127.84, 127.74, 103.50, 102.56, 101.72, 98.53, 82.01, 81.87, 81.48, 77.67, 76.80, 76.49, 75.32, 75.19, 74.54, 73.14, 72.50, 72.07, 71.95, 70.73, 70.72, 69.28, 69.25, 68.57, 68.48, 68.30, 68.02, 67.44, 66.56, 61.52, 61.08, 60.53, 59.67, 54.73, 29.64, 22.25, 15.25. HRMS (ESI-Orbitrap) m/z: [M+Na]⁺ calculated for C₄₀H₆₂N₂NaO₂₆1009.3488; found 1009.3409.

MSOPME preparative-scale synthesis of Galβ33(Fucα4)GlcNAcβ3Galβ4(Fucα3)-GlcβNHCbz (LNDFH-IIβNHCbz) (10). LNFP-VβNHCbz (9) was prepared as an intermediate from 50 mg LacβNHCbz (1) (OPME1a, OPME3c, and OPME2b) as described above. After the reaction was completed, the reaction mixture was incubated in a boiling water bath for 5 min and then cooled down. In the same reaction tube without workup or purification, _{L}-fucose (0.16 mmol), ATP (0.16 mmol), and GTP (0.16 mmol) were added and the pH of the reaction was adjusted to 7.5 by adding 4 M NaOH. BfFKP (1.5 mg), Hp3/4FT (0.8 mg), and PmPpA (0.5 mg) (OPME3c) were then added and the final volume of the reaction was kept at around 10.5 mL. The reaction mixture was incubated at 30° C. in an incubator shaker for 14 h with agitation at 180 rpm. The product formation was monitored by TLC with EtOAc:MeOH:H₂O=5:2.6:1 (by volume) as the developing solvent and by HRMS. After the reaction was completed, the reaction mixture was incubated in a boiling water bath for 5 min to denature the enzymes, cooled down, and purified. LNDFH-IIβNHCbz (10) was obtained as a white powder (95 mg, 80% yield for four steps from 50 mg LacβNHCbz). ¹H NMR (600 MHz, D₂O, 30° C.) δ 7.48-7.39 (m, 5H), 5.44 (d, J=4.0 Hz, 1H), 5.18 (s, 2H), 5.03 (d, J=4.0 Hz, 1H), 4.88 (q, J=6.7 Hz, 1H), 4.83-4.80 (m, 1H), 4.76 (s, 1H), 4.69 (d, J=8.5 Hz, 1H), 4.51 (d, J=7.7 Hz, 1H), 4.42 (d, J=7.7 Hz, 1H), 4.10-4.05 (m, 2H), 3.98-3.67 (m, 22H), 3.65-3.46 (m, 8H), 2.03 (s, 3H), 1.17 (dd, J=9.1, 6.6 Hz, 6H). ¹³C NMR (150 MHz, D₂O, 30° C.) δ 174.71, 158.11, 136.01, 128.77, 128.46, 127.77, 127.67, 102.81, 102.56, 101.68, 98.46, 97.97, 81.80, 81.50, 77.59, 76.73, 75.89, 75.16, 74.78, 74.47, 73.07, 72.28, 72.09, 72.00, 71.92, 71.89, 70.62, 70.55, 70.48, 69.19, 69.11, 68.33, 68.23, 67.95, 67.77, 67.37, 66.82, 66.50, 61.63, 61.46, 59.58, 55.84, 22.25, 15.34, 15.18. HRMS (ESI-Orbitrap) m/z: [M+Na]⁺ calculated for C₄₆H₇₂N₂NaO₃₀ 1155.4068; found 1155.4080.

MSOPME preparative-scale synthesis of Galβ4GlcNAcβ3Galβ4GlcNAcβ3Gal-β4GlcβNHCbz (pLNnHβNHCbz) (11). GlcNAc-LNnTβNHCbz (4) was prepared as an intermediate from 50 mg LacβNHCbz (1) (OPME1a, OPME2a, and OPME1b) as described above. After the reaction was completed, the reaction mixture was incubated in a boiling water bath for 5 min and then cooled down. In the same reaction tube without workup or purification, Gal (0.16 mmol), ATP (0.16 mmol), and UTP (0.16 mmol) were added, and the pH of the reaction was adjusted to 7.5 by adding 4 M NaOH. SpGalK (1.5 mg), BLUSP (1.2 mg), NmLgtB (1.8 mg), and PmPpA (0.5 mg) (OPME2a) were then added and the final volume of the reaction was kept at around 10.5 mL. The reaction mixture was incubated at 30° C. in an incubator shaker for 16 h with agitation at 180 rpm. The product formation was monitored by TLC with EtOAc:MeOH:H₂O=5:2.8:1 (by volume) as the developing solvent and by HRMS. After the reaction was completed, the reaction mixture was incubated in a boiling water bath for 5 min to denature the enzymes, cooled down, and purified. pLNnHβNHCbz (11) was obtained as a white powder (100 mg, 79% yield for four steps from 50 mg LacβNHCbz). ¹H NMR (600 MHz, D₂O, 30° C.) δ 7.48-7.39 (m, 4H), 5.18 (d, J=3.6 Hz, 2H), 4.82 (d, J=9.5 Hz, 1H), 4.70 (d, J=8.4 Hz, 2H), 4.46 (dt, J=15.5, 7.8 Hz, 3H), 4.15 (dd, J=5.2, 3.3 Hz, 2H), 3.94 (tt, J=10.9, 4.7 Hz, 4H), 3.87-3.63 (m, 24H), 3.61-3.50 (m, 5H), 3.42 (t, J=8.9 Hz, 1H), 2.03 (s, 6H). ¹³C NMR (150 MHz, D₂O, 30° C.) δ 174.87, 158.08, 136.00, 128.78, 128.47, 127.82, 127.68, 102.87, 102.84, 102.73, 102.71, 82.05, 81.95, 81.65, 78.18, 78.15, 78.04, 77.71, 76.12, 75.33, 75.01, 74.85, 74.53, 72.49, 72.16, 72.14, 72.03, 71.40, 70.94, 69.96, 69.94, 68.53, 68.33, 68.29, 67.40, 63.02, 62.46, 61.00, 60.95, 60.93, 59.84, 56.34, 55.17, 55.13, 22.16. HRMS (ESI-Orbitrap) m/z: [M+Na]⁺ calculated for C₄₈H₇₅N₃NaO₃₂ 1228.4231; found 1228.4226.

MSOPME preparative-scale synthesis of GlcNAcβ3Galβ4GlcNAcβ3Gal-β4GlcNAcβ3Galβ4GlcβNHCbz (GlcNAc-pLNnHβNHCbz) (12). pLNnHβNHCbz (11) was prepared as an intermediate from 50 mg LacβNHCbz (1) (OPME1a, OPME2a, OPME1b, and OPME2a) as described above. After the reaction was completed, the reaction mixture was incubated in a boiling water bath for 5 min and then cooled down. In the same reaction tube without workup or purification, GlcNAc (0.16 mmol), ATP (0.16 mmol), and UTP (0.16 mmol) were added, and the pH of the reaction was adjusted to 7.5 by adding 4 M NaOH. BLNahK (1 mg), PmGlmU (0.8 mg), Hpβ3GlcNAcT (1.5 mg), and PmPpA (0.5 mg) (OPME1b) were then added and the final volume of the reaction was kept at around 10.5 mL. The reaction mixture was incubated at 30° C. in an incubator shaker for 18 h with agitation at 180 rpm. The product formation was monitored by TLC with EtOAc:MeOH:H₂O=5:3.2:1 (by volume) as the developing solvent and by HRMS. After the reaction was completed, the reaction mixture was incubated in a boiling water bath for 5 min to denature the enzymes, cooled down, and purified. GlcNAc-pLNnHβNHCbz (12) was obtained as a white powder (114 mg, 77% yield for five steps from 50 mg LacβNHCbz). ¹H NMR (800 MHz, D₂O, 30° C.) δ 7.46-7.39 (m, 5H), 5.18 (d, J=6.1 Hz, 2H), 4.69 (d, J=8.4 Hz, 2H), 4.67 (d, J=8.4 Hz, 1H), 4.46 (d, J=7.9 Hz, 2H), 4.43 (d, J=7.9 Hz, 1H), 4.14 (t, J=3.6 Hz, 3H), 3.92 (ddd, J=43.6, 12.5, 2.2 Hz, 4H), 3.85-3.64 (m, 26H), 3.60-3.54 (m, 6H), 3.48-3.39 (m, 3H), 2.03 (s, 3H), 2.02 (s, 6H). ¹³C NMR (200 MHz, D₂O, 30° C.) δ 174.92, 174.87, 158.07, 136.00, 128.77, 128.46, 128.41, 127.81, 127.67, 102.86, 102.83, 102.72, 102.71, 82.03, 81.96, 81.94, 81.64, 78.14, 77.69, 76.12, 75.63, 75.00, 74.86, 74.84, 74.52, 73.53, 72.14, 71.39, 69.97, 69.95, 69.93, 69.65, 68.33, 68.29, 67.39, 60.94, 60.93, 60.45, 59.83, 55.63, 55.12, 29.57, 22.15. HRMS (ESI-Orbitrap) m/z: [M+Na]⁺ calculated for C₅₆H₈₈N₄NaO₃₇ 1431.5025; found 1431.5033.

MSOPME preparative-scale synthesis of Fucα2Galβ4GlcNAcβ3Galβ4GlcNAc-β3Galβ4GlcβNHCbz (F-pLNnH-IβNHCbz) (13). pLNnHβNHCbz (11) was prepared as an intermediate from 50 mg LacβNHCbz (1) (OPME1a, OPME2a, OPME1b, and OPME2a) as described above. After the reaction was completed, the reaction mixture was incubated in a boiling water bath for 5 min and then cooled down. In the same reaction tube without workup or purification, L-fucose (0.16 mmol), ATP (0.16 mmol), and GTP (0.16 mmol) were added and the pH of the reaction was adjusted to 7.5 by adding 4 M NaOH. BfFKP (1.5 mg), Hm2FT (1.5 mg), and PmPpA (0.5 mg) (OPME3a) were then added and the final volume of the reaction was kept at around 10.5 mL. The reaction mixture was incubated at 30° C. in an incubator shaker for 18 h with agitation at 180 rpm. The product formation was monitored by TLC with EtOAc:MeOH:H₂O=5:3:1 (by volume) as the developing solvent and by HRMS. After the reaction was completed, the reaction mixture was incubated in a boiling water bath for 5 min to denature the enzymes, cooled down, and purified. F-pLNnH-Iβ3NHCbz (13) was obtained as a white powder (108 mg, 76% yield for five steps from 50 mg LacβNHCbz). ¹H NMR (600 MHz, D₂O, 30° C.) δ 7.48-7.38 (m, 5H), 5.31 (d, J=3.0 Hz, 1H), 5.18 (d, J=3.8 Hz, 2H), 4.82 (s, 1H), 4.70 (d, J=8.4 Hz, 2H), 4.55 (d, J=7.7 Hz, 1H), 4.47 (d, J=8.1 Hz, 1H), 4.44 (d, J=7.9 Hz, 1H), 4.22 (d, J=6.7 Hz, 1H), 4.15 (d, J=3.3 Hz, 2H), 4.01-3.62 (m, 33H), 3.62-3.52 (m, 3H), 3.49-3.38 (m, 2H), 2.04 (s, 3H), 2.03 (s, 3H), 1.23 (d, J=6.6 Hz, 3H). ¹³C NMR (150 MHz, D₂O, 30° C.) δ 174.88, 158.07, 135.98, 128.77, 128.47, 128.40, 127.81, 127.67, 102.86, 102.83, 102.75, 102.70, 100.22, 99.39, 81.98, 81.94, 78.16, 77.69, 76.43, 76.12, 75.84, 75.23, 75.07, 75.00, 74.84, 74.82, 74.52, 73.50, 72.14, 72.04, 71.64, 71.37, 69.96, 69.59, 69.09, 68.34, 68.28, 68.18, 67.39, 66.92, 61.10, 60.95, 60.89, 59.84, 55.36, 55.13, 22.18, 22.15, 15.29. HRMS (ESI-Orbitrap) m/z: [M+Na]⁺ calculated for C₅₄H₈₅N₃NaO₃₆ 1374.4810; found 1374.4807.

MSOPME preparative-scale synthesis of Galβ4(Fucα3)GlcNAcβ3Galβ4(Fucα3)-GlcNAcβ3Galβ4(Fucα3)GlcβNHCbz (TF-pLNnHβNHCbz) (14). pLNnHβNHCbz (11) was prepared as an intermediate from 50 mg LacβNHCbz (1) (OPME1a, OPME2a, OPME1b, and OPME2a) as described above. After the reaction was completed, the reaction mixture was incubated in a boiling water bath for 5 min and then cooled down. In the same reaction tube without workup or purification, _{L}-fucose (0.53 mmol), ATP (0.53 mmol), and GTP (0.53 mmol) were added and the pH of the reaction was adjusted to 7.5 by adding 4 M NaOH. BfFKP (5.5 mg), Hp3/4FT (2.5 mg), and PmPpA (1 mg) (OPME3c) were then added and the final volume was kept at around 10.5 mL. The reaction mixture was incubated at 30° C. in an incubator shaker for 18 h with agitation at 180 rpm. The product formation was monitored by HRMS. After the reaction was completed, the reaction mixture was incubated in a boiling water bath for 5 min to denature the enzymes, cooled down, and purified. TF-pLNnHβNHCbz (14) was obtained as a white powder (131 mg, 76% yield for five steps from 50 mg LacβNHCbz). ¹H NMR (600 MHz, D₂O, 30° C.) δ 7.49-7.39 (m, 5H), 5.44 (d, J=4.0 Hz, 1H), 5.18 (s, 2H), 5.13 (dd, J=8.3, 4.0 Hz, 2H), 4.84 (d, J=6.6 Hz, 1H), 4.82 (d, J=8.1 Hz, 2H), 4.81-4.80 (m, 1H), 4.70 (d, J=8.4 Hz, 2H), 4.49-4.39 (m, 3H), 4.09 (dd, J=6.0, 3.4 Hz, 2H), 3.99-3.55 (m, 43H), 3.49 (ddt, J=9.6, 7.9, 4.9 Hz, 3H), 2.07-1.98 (m, 6H), 1.20-1.12 (m, 9H). ¹³C NMR (150 MHz, D₂O, 30° C.) δ 174.64, 174.62, 158.10, 136.01, 128.77, 128.46, 127.77, 127.67, 102.46, 101.72, 101.67, 98.66, 98.56, 98.45, 81.80, 81.59, 81.45, 77.59, 76.72, 75.04, 74.89, 74.73, 74.45, 74.41, 73.08, 73.00, 72.74, 72.45, 71.97, 71.88, 71.83, 71.03, 70.64, 70.50, 69.17, 69.14, 68.33, 68.22, 67.95, 67.68, 67.61, 67.37, 66.66, 66.49, 61.48, 61.44, 59.61, 55.92, 23.24, 22.21, 15.29, 15.25, 15.18. HRMS (ESI-Orbitrap) m/z: [M+Na]⁺ calculated for C₆₆H₁₀₅N₃NaO₄₄ 1666.5969; found 1666.5840.

MSOPME preparative-scale synthesis of Neu5Acα3Galβ4GlcNAcβ3Gal-β4GlcNAcβ3Galβ4GlcβNHCbz (Neu5Acα2-3pLNnHβNHCbz) (15). pLNnHβNHCbz (11) was prepared as an intermediate from 50 mg LacβNHCbz (1) (OPME1a, OPME2a, OPME1b, and OPME2a) as described above. After the reaction was completed, the reaction mixture was incubated in a boiling water bath for 5 min and then cooled down. In the same reaction tube without workup or purification, Neu5Ac (0.16 mmol) and CTP (0.26 mmol) were added and the pH was adjusted to 8.5 by adding 4 M NaOH. NmCSS (0.5 mg) and PmST3 (1.5 mg) (OPME4a) were then added and the final volume of the reaction was kept at around 10.5 mL. The reaction mixture was incubated at 30° C. in an incubator shaker for 14 h with agitation at 180 rpm. The product formation was monitored by HRMS. After the reaction was completed, the reaction mixture was incubated in a boiling water bath for 5 min to denature the enzymes, cooled down, and purified. Neu5Acα2-3pLNnHβNHCbz (15) was obtained as a white powder (123 mg, 78% yield for five steps from 50 mg LacβNHCbz). ¹H NMR (600 MHz, D₂O, 30° C.) δ 7.48-7.39 (m, 5H), 5.18 (d, J=3.7 Hz, 2H), 4.82 (s, 1H), 4.70 (dd, J=8.5, 2.3 Hz, 2H), 4.56 (d, J=7.9 Hz, 1H), 4.45 (dd, J=15.4, 7.8 Hz, 2H), 4.15 (dd, J=5.6, 3.3 Hz, 2H), 4.12 (dd, J=9.9, 3.1 Hz, 1H), 3.99-3.38 (m, 40H), 2.76 (dd, J=12.4, 4.6 Hz, 1H), 2.03 (s, 9H), 1.80 (t, J=12.1 Hz, 1H). ¹³C NMR (150 MHz, D₂O, 30° C.) δ 175.06, 174.94, 174.92, 173.90, 158.15, 136.06, 128.84, 128.54, 127.88, 127.74, 102.92, 102.85, 102.79, 102.60, 99.86, 82.12, 82.02, 81.72, 78.24, 78.05, 77.79, 76.19, 75.54, 75.22, 75.08, 74.94, 74.92, 74.60, 72.94, 72.21, 72.19, 71.82, 71.47, 70.02, 69.43, 68.39, 68.35, 68.14, 67.53, 67.46, 62.63, 61.08, 61.05, 61.02, 59.91, 59.69, 55.23, 55.19, 54.41, 51.73, 42.59, 39.69, 22.23, 22.09, 12.19. HRMS (ESI-Orbitrap) m/z: [M−H]⁻ calculated for C₅₉H₉₁N₄O₄₀ 1495.5215; found 1495.5212.

MSOPME preparative-scale synthesis of Neu5Acα6Galβ4GlcNAcβ3Gal-β4GlcNAcβ3Galβ4GlcβNHCbz (Neu5Acα2-6pLNnHβNHCbz) (16). pLNnHβNHCbz (11) was prepared as an intermediate from 50 mg LacβNHCbz (1) (OPME1a, OPME2a, OPME1b, and OPME2a) as described above. After the reaction was completed, the reaction mixture was incubated in a boiling water bath for 5 min and then cooled down. In the same reaction tube without workup or purification, Neu5Ac (0.13 mmol) and CTP (0.21 mmol) were added and the pH of the reaction was adjusted to 8.5 by adding 4 M NaOH. NmCSS (0.5 mg) and Pd2,6ST_A200Y/S232Y (1.5 mg) (OPME4b) were then added and the final volume of the reaction was kept at around 10.5 mL. The reaction mixture was incubated at 30° C. in an incubator shaker for 14 h with agitation at 180 rpm. The product formation was monitored by HRMS. After the reaction was completed, the reaction mixture was incubated in a boiling water bath for 5 min to denature the enzymes, cooled down, and purified. Neu5Acα2-6pLNnHβNHCbz (16) was obtained as a white powder (118 mg, 75% yield for five steps from 50 mg LacβNHCbz). ¹H NMR (600 MHz, D₂O, 30° C.) δ 7.49-7.38 (m, 5H), 5.23-5.15 (m, 2H), 4.83 (s, 1H), 4.73 (d, J=7.7 Hz, 1H), 4.70 (d, J=8.3 Hz, 1H), 4.50-4.40 (m, 3H), 4.15 (t, J=4.0 Hz, 2H), 4.05-3.36 (m, 40H), 2.72-2.62 (m, 1H), 2.05 (s, 3H), 2.03 (s, 6H), 1.72 (t, J=12.2 Hz, 1H). ¹³C NMR (150 MHz, D₂O, 30° C.) δ 174.89, 173.50, 158.09, 136.00, 128.77, 128.47, 127.81, 103.44, 102.84, 102.73, 102.56, 100.11, 82.00, 81.96, 81.64, 80.46, 78.17, 77.72, 76.12, 75.00, 74.86, 74.53, 74.24, 73.68, 72.52, 72.40, 72.21, 72.15, 71.69, 71.39, 70.71, 69.95, 68.38, 68.34, 68.28, 68.19, 67.39, 63.33, 62.64, 60.95, 60.12, 59.83, 59.29, 55.11, 54.92, 51.86, 40.05, 22.27, 22.15, 22.01. HRMS (ESI-Orbitrap) m/z: [M−H]⁻ calculated for C₅₉H₉₁N₄O₄₀ 1495.5215; found 1495.5140.

MSOPME preparative-scale synthesis of GalβGlcNAcβ3Galβ4GlcNAcβ3Gal-β4GlcβNHCbz (pLNHβNHCbz) (17). GlcNAc-LNnTβNHCbz (4) was prepared as an intermediate from 50 mg LacβNHCbz (1) (OPME1a, OPME2a, and OPME1b) as described above. After the reaction was completed, the reaction mixture was incubated in a boiling water bath for 5 min and then cooled down. In the same reaction tube without workup or purification, Gal (0.16 mmol), ATP (0.16 mmol), and UTP (0.16 mmol) were added, and the pH was adjusted to 7.5 by adding 4 M NaOH. SpGalK (1.5 mg), BLUSP (1.2 mg), Cvβ3GalT (2.5 mg), and PmPpA (0.5 mg) (OPME2b) were then added and the final volume of the reaction was kept at around 10.5 mL. The reaction mixture was incubated at 30° C. in an incubator shaker for 18 h with agitation at 180 rpm. The product formation was monitored by TLC with EtOAc:MeOH:H₂O=5:2.8:1 (by volume) as the developing solvent and by HRMS. After the reaction was completed, the reaction mixture was incubated in a boiling water bath for 5 min to denature the enzymes, cooled down, and purified. pLNHβNHCbz (17) was obtained as a white powder (101 mg, 80% yield for four steps from 50 mg LacβNHCbz). ¹H NMR (600 MHz, D₂O, 30° C.) δ 7.50-7.39 (m, 5H), 5.19 (t, J=3.4 Hz, 2H), 4.82 (d, J=9.8 Hz, 1H), 4.73 (d, J=8.4 Hz, 1H), 4.70 (d, J=8.3 Hz, 1H), 4.47 (d, J=7.8 Hz, 1H), 4.44 (d, J=7.8 Hz, 2H), 4.15 (t, J=3.8 Hz, 2H), 3.98-3.88 (m, 4H), 3.87-3.33 (m, 30H), 1.97-2.08 (m, 6H). ¹³C NMR (150 MHz, D₂O, 30° C.) δ 174.94, 174.88, 158.09, 136.00, 128.78, 128.47, 127.82, 127.68, 103.47, 102.84, 102.73, 102.71, 102.55, 82.05, 82.00, 81.95, 81.65, 78.18, 78.15, 77.71, 76.12, 75.33, 75.26, 75.16, 75.01, 74.85, 74.53, 72.49, 72.45, 72.14, 71.40, 70.94, 70.66, 69.96, 68.51, 68.42, 68.34, 68.28, 67.40, 63.02, 61.01, 60.95, 60.93, 60.47, 59.84, 55.17, 55.13, 54.68, 22.21, 22.15. HRMS (ESI-Orbitrap) m/z: [M+Na]⁺ calculated for C₄₈H₇₅N₃NaO₃₂ 1228.4231; found 1228.4233.

MSOPME preparative-scale synthesis of Fucα2GalβGlcNAcβ3Galβ4GlcNAc-β3Galβ4GlcβNHCbz (F-pLNH-IβNHCbz) (18). pLNHβNHCbz (17) was prepared as an intermediate from 50 mg LacβNHCbz (1) (OPME1a, OPME2a, OPME1b, and OPME2b) as described above. After the reaction was completed, the reaction mixture was incubated in a boiling water bath for 5 min and then cooled down. In the same reaction tube without workup or purification, _{L}-fucose (0.16 mmol), ATP (0.16 mmol), and GTP (0.16 mmol) were added and the pH was adjusted to 7.5 by adding 4 M NaOH. BfFKP (1.5 mg), Hm2FT (1.5 mg), and PmPpA (0.5 mg) (OPME3a) were then added and the final volume of the reaction was kept at around 10.5 mL. The reaction mixture was incubated at 30° C. in an incubator shaker for 18 h with agitation at 180 rpm. The product formation was monitored by TLC with EtOAc:MeOH:H₂O=5:3:1 (by volume) as the developing solvent and by HRMS. After the reaction was completed, the reaction mixture was incubated in a boiling water bath for 5 min to denature the enzymes, cooled down, and purified. F-pLNH-Iβ3NHCbz (18) was obtained as a white powder (105 mg, 74% yield for five steps from 50 mg LacβNHCbz). ¹H NMR (600 MHz, D₂O, 30° C.) δ 7.44 (d, J=4.4 Hz, 5H), 5.18 (dd, J=5.7, 3.7 Hz, 3H), 4.81 (d, J=9.4 Hz, 1H), 4.69 (d, J=8.3 Hz, 1H), 4.64 (d, J=7.7 Hz, 1H), 4.61 (d, J=8.4 Hz, 1H), 4.44 (dd, J=7.9, 6.2 Hz, 2H), 4.29 (q, J=6.7 Hz, 1H), 4.14 (q, J=3.3 Hz, 2H), 4.01-3.86 (m, 5H), 3.85-3.63 (m, 24H), 3.61-3.33 (m, 9H), 1.98-2.07 (m, 6H), 1.23 (d, J=6.6 Hz, 3H). ¹³C NMR (150 MHz, D₂O, 30° C.) δ 174.87, 174.20, 158.09, 135.99, 128.76, 128.46, 127.80, 103.22, 102.88, 102.83, 102.70, 100.21, 99.48, 81.93, 81.76, 81.58, 78.04, 77.67, 77.26, 77.12, 76.63, 76.41, 76.11, 75.21, 75.04, 74.99, 74.83, 74.78, 74.53, 73.45, 72.10, 71.82, 71.70, 71.38, 70.16, 69.94, 69.40, 69.20, 69.09, 68.53, 68.42, 68.33, 68.01, 67.38, 66.46, 61.12, 60.91, 60.50, 60.35, 59.82, 55.11, 54.94, 22.14, 22.11, 15.23. HRMS (ESI-Orbitrap) m/z: [M+Na]⁺ calculated for C₅₄H₈₅N₃NaO₃₆ 1374.4810; found 1374.4860.

MSOPME preparative-scale synthesis of Galβ3(Fucα4)GlcNAcβ3Galβ4(Fucα3)-GlcNAcβ3Galβ4(Fucα3)GlcβNHCbz (TF-pLNH-IIIβNHCbz) (19). pLNHβNHCbz (17) was prepared as an intermediate from 50 mg LacβNHCbz (1) (OPME1a, OPME2a, OPME1b, and OPME2b) as described above. After the reaction was completed, the reaction mixture was incubated in a boiling water bath for 5 min and then cooled down. In the same reaction tube without workup or purification, _{L}-fucose (0.53 mmol), ATP (0.53 mmol), and GTP (0.53 mmol) were added and the pH was adjusted to 7.5 by adding 4 M NaOH. BfFKP (5.5 mg), Hp3/4FT (2.5 mg), and PmPpA (1 mg) (OPME3c) were then added and the final volume of the reaction was kept at around 10.5 mL. The reaction mixture was incubated at 30° C. in an incubator shaker for 18 h with agitation at 180 rpm. The product formation was monitored by HRMS. After the reaction was completed, the reaction mixture was incubated in a boiling water bath for 5 min to denature the enzymes, cooled down, and purified. TF-pLNH-IIIβNHCbz (19) was obtained as a white powder (124 mg, 72% yield for five steps from 50 mg LacβNHCbz). ¹H NMR (600 MHz, D₂O, 30° C.) δ 7.46-7.39 (m, 5H), 5.43 (d, J=4.0 Hz, 1H), 5.18 (d, J=6.4 Hz, 2H), 5.12 (d, J=4.0 Hz, 1H), 5.02 (d, J=4.0 Hz, 1H), 4.87 (q, J=6.7 Hz, 1H), 4.84-4.79 (m, 3H), 4.69 (t, J=7.8 Hz, 2H), 4.51 (d, J=7.6 Hz, 1H), 4.43 (dd, J=16.3, 7.8 Hz, 2H), 4.11-4.04 (m, 3H), 3.98-3.66 (m, 33H), 3.65-3.44 (m, 12H), 2.02 (s, 3H), 2.01 (s, 3H), 1.19-1.12 (m, 9H). ¹³C NMR (150 MHz, D₂O, 30° C.) δ 174.69, 174.64, 158.11, 136.01, 128.76, 128.46, 127.77, 127.67, 102.82, 102.55, 102.45, 101.69, 98.66, 98.45, 97.97, 81.79, 81.63, 81.44, 77.58, 76.72, 75.89, 75.15, 75.03, 74.78, 74.72, 74.45, 74.42, 73.07, 72.76, 72.27, 72.07, 71.96, 71.92, 71.87, 71.83, 70.63, 70.46, 69.18, 69.14, 69.10, 68.32, 68.22, 67.94, 67.75, 67.61, 67.37, 66.82, 66.65, 66.49, 61.63, 61.44, 59.59, 55.92, 55.84, 22.24, 22.20, 15.34, 15.25, 15.17. HRMS (ESI-Orbitrap) m/z: [M+Na]⁺ calculated for C₆₆H₁₀₅N₃NaO₄₄ 1666.5969; found 1666.5909.

MSOPME preparative-scale synthesis of Galβ4GlcNAcβ3Galβ4GlcNAcβ3Gal-β4GlcNAcβ3Galβ4GlcβNHCbz (pLNnOβNHCbz) (20). GlcNAc-pLNnHβNHCbz (12) was prepared as an intermediate from 50 mg LacβNHCbz (1) (OPME1a, OPME2a, OPME1b, OPME2a, and OPME1b) as described above. After the reaction was completed, the reaction mixture was incubated in a boiling water bath for 5 min and then cooled down. In the same reaction tube without workup or purification, Gal (0.16 mmol), ATP (0.16 mmol), and UTP (0.16 mmol) were added, and the pH was adjusted to 7.5 by adding 4 M NaOH. SpGalK (1.5 mg), BLUSP (1.2 mg), NmLgtB (1.8 mg), and PmPpA (0.5 mg) (OPME2a) were then added and the final volume of the reaction was kept at around 10.5 mL. The reaction mixture was incubated at 30° C. in an incubator shaker for 16 h with agitation at 180 rpm. The product formation was monitored by HRMS. After the reaction was completed, the reaction mixture was incubated in a boiling water bath for 5 min to denature the enzymes, cooled down, and purified. pLNnOβNHCbz (20) was obtained as a white powder (119 mg, 72% yield for six steps from 50 mg LacβNHCbz). ¹H NMR (800 MHz, D₂O, 30° C.) δ 7.46-7.38 (m, 5H), 5.18 (dd, J=11.8, 6.8 Hz, 2H), 4.81 (d, J=10.4 Hz, 1H), 4.69 (dd, J=8.4, 2.5 Hz, 3H), 4.48-4.44 (m, 3H), 4.43 (d, J=7.8 Hz, 1H), 4.14 (dt, J=5.3, 2.9 Hz, 3H), 3.95-3.85 (m, 5H), 3.85-3.61 (m, 32H), 3.60-3.50 (m, 7H), 3.36-3.44 (m, 1H), 2.00-2.03 (m, 9H). ¹³C NMR (200 MHz, D₂O, 30° C.) δ 174.86, 135.98, 128.76, 128.46, 128.40, 127.80, 127.66, 102.85, 102.82, 102.73, 102.71, 82.03, 81.94, 81.63, 78.11, 78.01, 77.67, 76.11, 75.32, 74.99, 74.83, 74.51, 72.47, 72.14, 72.13, 71.38, 70.93, 69.94, 69.92, 68.51, 68.32, 68.28, 67.39, 61.00, 60.94, 60.92, 59.81, 55.15, 55.11, 22.14. HRMS (ESI-Orbitrap) m/z: [M+Na]⁺ calculated for C₆₂H₉₈N₄NaO₄₂ 1593.5553; found 1593.5419.

MSOPME preparative-scale synthesis of GalβGlcNAcβ3Galβ4GlcNAcβ3Gal-β4GlcNAcβ3Galβ4GlcβNHCbz (pLNOβNHCbz) (21). GlcNAc-pLNnHβNHCbz (12) was prepared as an intermediate from 50 mg LacβNHCbz (1) (OPME1a, OPME2a, OPME1b, OPME2a, and OPME1b) as described above. After the reaction was completed, the reaction mixture was incubated in a boiling water bath for 5 min and then cooled down. In the same reaction tube without workup or purification, Gal (0.16 mmol), ATP (0.16 mmol), and UTP (0.16 mmol) were added, and the pH was adjusted to 7.5 by adding 4 M NaOH. SpGalK (1.5 mg), BLUSP (1.2 mg), Cvβ3GalT (2.5 mg), and PmPpA (0.5 mg) (OPME2b) were then added and the final volume of the reaction was kept at around 10.5 mL. The reaction mixture was incubated at 30° C. in an incubator shaker for 18 h with agitation at 180 rpm. The product formation was monitored by HRMS. After the reaction was completed, the reaction mixture was incubated in a boiling water bath for 5 min to denature the enzymes, cooled down, and purified. pLNOβNHCbz (21) was obtained as a white powder (117 mg, 71% yield for six steps from 50 mg LacβNHCbz). ¹H NMR (800 MHz, D₂O, 30° C.) δ 7.47-7.38 (m, 5H), 5.17 (d, J=6.2 Hz, 2H), 4.81 (d, J=9.1 Hz, 1H), 4.69 (dd, J=8.4, 2.2 Hz, 3H), 4.46 (dd, J=11.2, 7.9 Hz, 3H), 4.43 (d, J=7.9 Hz, 1H), 4.14 (dt, J=6.0, 2.6 Hz, 3H), 3.95-3.87 (m, 6H), 3.85-3.74 (m, 12H), 3.73-3.68 (m, 16H), 3.66-3.62 (m, 3H), 3.59-3.51 (m, 7H), 3.40 (t, J=9.1 Hz, 1H), 2.00-2.03 (m, 9H). ¹³C NMR (200 MHz, D₂O, 30° C.) δ 174.86, 158.08, 135.98, 128.75, 128.46, 127.80, 103.45, 102.85, 102.82, 102.72, 102.71, 102.54, 82.02, 81.93, 81.62, 78.11, 77.66, 76.11, 75.31, 75.24, 75.14, 74.99, 74.83, 74.51, 72.46, 72.43, 72.12, 71.37, 70.93, 70.64, 69.96, 69.94, 69.92, 68.51, 68.49, 68.40, 68.32, 68.28, 67.39, 60.99, 60.93, 60.92, 60.45, 59.81, 55.15, 55.11, 22.19, 22.14. HRMS (ESI-Orbitrap) m/z: [M+Na]⁺ calculated for C₆₂H₉₈N₄NaO₄₂ 1593.5553; found 1593.5554.

Preparative-scale synthesis of Fucα2Galβ3GlcNAcβ3Galβ4GlcNAcβ3Gal-β4GlcNAcβ3Galβ4GlcβNHCbz (Fucα2pLNOβNHCbzβNHCbz) (22) from 21 by OPME3a. pLNOβNHCbz (21, 20 mg, 0.0127 mmol), _{L}-fucose (0.019 mmol), ATP (0.019 mmol), and GTP (0.019 mmol) were dissolved in a small amount of water. Tris-HCl buffer (100 mM, pH 7.5) and MgCl₂ (20 mM) were added. After the addition of BfFKP (0.8 mg), Hm2FT (0.5 mg), and PmPpA (0.1 mg) (OPME3a), water was added to bring the final concentration of pLNOβNHCbz (21) to 10 mM. The reaction mixture was incubated at 30° C. in an incubator shaker for 12 h with agitation at 180 rpm. The product formation was monitored by TLC with EtOAc:MeOH:H₂O=4:3:1 (by volume) as the developing solvent and by HRMS. After the reaction was completed, the reaction mixture was incubated in a boiling water bath for 5 min to denature the enzymes, cooled down, and purified. Fucα2pLNOβNHCbzβNHCbz (22) was obtained as a white powder (20 mg, 92% yield from 20 mg of pLNOβNHCbz 21). ¹H NMR (600 MHz, D₂O, 30° C.) δ 7.52-7.36 (m, 5H), 5.19 (d, J=4.0 Hz, 3H), 4.84-4.81 (m, 1H), 4.70 (d, J=8.4 Hz, 2H), 4.63 (dd, J=15.8, 8.0 Hz, 2H), 4.48-4.42 (m, 3H), 4.29 (q, J=6.5 Hz, 1H), 4.15 (q, J=3.6 Hz, 3H), 4.01-3.88 (m, 6H), 3.86-3.64 (m, 34H), 3.61-3.48 (m, 8H), 3.41 (q, J=8.2, 7.5 Hz, 1H), 2.06 (s, 3H), 2.03 (s, 3H), 1.23 (d, J=6.6 Hz, 3H). ¹³C NMR (150 MHz, D₂O, 30° C.) δ 174.95, 174.27, 136.07, 128.84, 128.54, 128.48, 127.88, 127.74, 103.29, 102.93, 102.90, 102.82, 102.79, 100.29, 99.56, 99.46, 82.10, 82.03, 78.21, 78.10, 77.77, 77.20, 76.70, 76.49, 76.18, 75.91, 75.29, 75.13, 75.07, 74.91, 74.59, 73.57, 72.21, 72.11, 71.90, 71.71, 71.46, 70.23, 70.02, 70.00, 69.66, 69.48, 69.16, 68.60, 68.51, 68.40, 68.36, 68.25, 68.09, 67.46, 66.99, 66.53, 61.16, 60.99, 60.43, 60.04, 59.90, 55.42, 55.19, 55.02, 22.24, 22.22, 22.19, 15.36, 15.30. HRMS (ESI-Orbitrap) m/z: [M+Na]⁺ calculated for C₆₅H₁₀₈N₄NaO₄₆ 1739.6132; found 1739.6113.

### Example 3. Tag Removal

Hydrogenation procedures. To remove the Cbz tag from the obtained glycosides (2-22), a catalytic amount (10-20%) of palladium on charcoal (Pd/C) (2 mg) was added to a solution containing 20 mg of each compound in H₂O:MeOH=1:1 (by volume) (3 mL). The mixture was stirred at room temperature under a hydrogen atmosphere with a balloon. The reaction was monitored by high-resolution mass spectrometry (“HRMS”). When the reaction was completed (2-12 hours (h)), the mixture was passed through a 0.45 μm syringe filter to remove palladium and charcoal. The solvent was removed in vacuo. The residue obtained was dissolved in 3 mL of H₂O and the mixture was incubated at 37° C. with 120 rpm agitation in an incubator shaker. The reaction was monitored by HRMS until the β-glycosylamine was completely converted to the target HMO with a free reducing end (4-5 days for HMOs containing an _{L}-fucose linked to the reducing end Glc residue or 20-48 hours for other HMOs). The pure HMO was obtained by lyophilization without further purification.

GlcNAcβ3Galβ4Glc (LNT-II, 2a). White powder, 15.4 mg, 95% yield. ¹H NMR (800 MHz, D₂O, 30° C.) δ 5.21 (d, J=3.7 Hz, 0.4H), 4.67 (d, J=8.5 Hz, 1H), 4.65 (d, J=8.0 Hz, 0.6H), 4.42 (dd, J=7.9, 1.4 Hz, 1H), 4.14 (d, J=3.4 Hz, 1H), 3.95-3.68 (m, 9H), 3.68-3.22 (m, 7H), 2.02 (s, 3H). ¹³C NMR (200 MHz, D₂O, 30° C.) δ 174.92, 102.89, 102.85, 102.81, 95.70, 91.77, 81.91, 81.89, 78.32, 78.22, 75.65, 75.62, 74.86, 74.76, 74.31, 73.97, 73.75, 73.52, 71.36, 71.09, 70.08, 70.00, 69.97, 69.66, 69.65, 68.34, 68.31, 60.94, 60.93, 60.44, 60.04, 59.91, 55.68, 55.62, 22.14, 22.12. HRMS (ESI-Orbitrap) m/z: [M+Na]⁺ calculated for C₂₀H₃₅NNaO₁₆ 568.1854; found 568.1889.

Galβ4GlcNAcβ3Galβ4Glc (LNnT, 3a). White powder, 16.5 mg, 95% yield. ¹H NMR (600 MHz, D₂O, 30° C.) δ 5.22 (d, J=3.8 Hz, 0.4H), 4.73-4.71 (d, 1H), 4.67 (d, J=7.9 Hz, 0.6H), 4.48 (d, J=7.8 Hz, 1H), 4.44 (d, J=7.8 Hz, 1H), 4.16 (d, J=3.3 Hz, 1H), 3.99-3.92 (m, 3H), 3.90-3.71 (m, 13H), 3.69-3.53 (m, 7H), 3.28 (t, J=8.5 Hz, 0.6H), 2.04 (s, 3H). ¹³C NMR (150 MHz, D₂O, 30° C.) δ 174.88, 102.91, 102.87, 102.84, 102.72, 95.72, 91.79, 82.01, 78.36, 78.26, 78.16, 75.33, 74.86, 74.78, 74.54, 74.33, 73.77, 72.49, 72.16, 71.38, 71.11, 70.95, 70.10, 69.95, 68.53, 68.32, 61.01, 60.95, 60.06, 59.93, 59.85, 55.18, 22.16. HRMS (ESI-Orbitrap) m/z: [M+Na]⁺ calculated for C₂₆H₄₅NNaO₂₁ 730.2382; found 730.2371.

GlcNAcβ3Galβ4GlcNAcβ3Galβ4Glc (4a). White powder, 17.2 mg, 95% yield. ¹H NMR (800 MHz, D₂O, 30° C.) δ 5.21 (d, J=3.8 Hz, 0.4H), 4.69 (dd, J=8.4, 3.0 Hz, 1H), 4.67 (d, J=8.5 Hz, 1H), 4.65 (d, J=8.0 Hz, 0.6H), 4.45 (d, J=7.9 Hz, 1H), 4.42 (d, J=7.9, 1.4 Hz, 1H), 4.14 (d, J=3.3 Hz, 2H), 3.91 (ddd, J=44.3, 12.1, 2.0 Hz, 3H), 3.84-3.54 (m, 23H), 3.47-3.26 (m, 2H), 2.04-2.00 (m, 6H). ¹³C NMR (200 MHz, D₂O, 30° C.) δ 174.91, 174.86, 102.89, 102.85, 102.83, 102.70, 95.70, 91.77, 81.99, 81.97, 81.96, 78.32, 78.21, 78.13, 75.62, 74.85, 74.83, 74.76, 74.51, 74.31, 73.75, 73.52, 72.13, 71.36, 71.09, 70.08, 69.96, 69.93, 69.63, 68.33, 68.30, 68.28, 60.93, 60.92, 60.43, 60.03, 59.90, 59.82, 55.62, 55.11, 22.14, 22.12. HRMS (ESI-Orbitrap) m/z: [M+Na]⁺ calculated for C₃₄H₅₈N₂NaO₂₆ 933.3175; found 933.3238.

Galβ4GlcNAcβ3Galβ4(Fucα3)Glc (LNnFP-V, 5a). White powder, 17.1 mg, 96% yield. ¹H NMR (800 MHz, D₂O, 30° C.) δ 5.41 (d, J=4.0 Hz, 0.4H), 5.36 (d, J=4.0 Hz, 0.4H), 5.17 (d, J=3.8 Hz, 0.3H), 4.80 (d, J=6.8 Hz, 1H), 4.69 (dd, J=8.4, 2.9 Hz, 1H), 4.64 (d, J=8.0 Hz, 0.4H), 4.47 (d, J=7.8 Hz, 1H), 4.40 (d, J=8.2 Hz, 1H), 4.08 (d, J=3.4 Hz, 1H), 3.93 (tdd, J=12.1, 6.2, 2.7 Hz, 4H), 3.86-3.65 (m, 18H), 3.58-3.43 (m, 5H), 2.01-2.03 (m, 3H), 1.15 (dd, J=6.7, 4.6 Hz, 3H). ¹³C NMR (200 MHz, D₂O, 30° C.) δ 174.83, 102.82, 102.66, 101.69, 98.52, 98.41, 95.80, 92.08, 81.53, 81.46, 78.14, 76.96, 75.50, 75.33, 75.31, 74.65, 74.48, 74.46, 72.64, 72.46, 72.34, 72.24, 72.09, 71.89, 71.88, 70.93, 70.91, 70.63, 70.58, 69.24, 69.18, 68.51, 68.25, 68.22, 67.99, 67.96, 66.46, 66.42, 61.46, 61.43, 60.99, 59.82, 59.75, 59.68, 55.13, 22.12, 15.19, 15.17. HRMS (ESI-Orbitrap) m/z: [M+Na]⁺ calculated for C₃₂H₅₅NNaO₂₅ 876.2961; found 876.2956.

Galβ4(Fucα3)GlcNAcβ3Galβ4(Fucα3)Glc (LNnDFH-II, 6a). White powder, 17.3 mg, 96% yield. ¹H NMR (800 MHz, D₂O, 30° C.) δ 5.41 (d, J=4.0 Hz, 0.4H), 5.36 (d, J=4.0 Hz, 0.4H), 5.17 (d, J=3.8 Hz, 0.4H), 5.12 (d, J=4.1 Hz, 1H), 4.82 (d, J=6.7 Hz, 1H), 4.80 (d, 1H), 4.69 (dd, J=8.5, 2.8 Hz, 1H), 4.64 (d, J=8.0 Hz, 0.4H), 4.45 (d, J=7.8 Hz, 1H), 4.40 (d, J=7.7 Hz, 1H), 4.08 (d, J=3.4 Hz, 1H), 3.97-3.83 (m, 11H), 3.81-3.41 (m, 20H), 1.99-2.02 (m, 3H), 1.18-1.13 (m, 6H). ¹³C NMR (200 MHz, D₂O, 30° C.) δ 174.62, 102.47, 101.71, 101.69, 98.55, 98.51, 98.40, 95.80, 92.09, 81.52, 81.45, 76.95, 75.50, 75.33, 75.04, 74.88, 74.72, 74.64, 74.45, 72.99, 72.88, 72.64, 72.43, 72.31, 72.21, 71.89, 71.87, 71.01, 70.91, 70.66, 70.61, 69.23, 69.18, 69.15, 68.31, 68.24, 68.21, 67.99, 67.96, 67.66, 66.65, 66.45, 66.41, 61.47, 61.43, 59.76, 59.69, 59.59, 55.91, 22.20, 15.27, 15.18, 15.17. HRMS (ESI-Orbitrap) m/z: [M+Na]⁺ calculated for C₃₈H₆₅NNaO₂₉ 1022.3540; found 1022.3510.

Galβ3GlcNAcβ3Galβ4Glc (LNT, 7a). White powder, 15.6 mg, 95%. ¹H NMR (800 MHz, D₂O, 30° C.) δ 5.28 (d, J=3.8 Hz, 0.3H), 4.80 (d, J=4.9 Hz, 1H), 4.72 (d, J=7.9 Hz, 0.5H), 4.50 (dd, J=7.8, 3.0 Hz, 2H), 4.21 (d, J=3.3 Hz, 1H), 4.03-3.75 (m, 15H), 3.72-3.32 (m, 8H), 2.09 (s, 3H). ¹³C NMR (200 MHz, D₂O, 30° C.) δ 174.99, 103.52, 102.97, 102.93, 102.56, 95.79, 91.86, 82.18, 82.03, 82.00, 81.82, 78.70, 78.52, 78.42, 75.33, 75.25, 74.94, 74.85, 74.41, 73.85, 72.54, 71.46, 71.20, 70.74, 70.18, 70.09, 70.06, 68.59, 68.53, 68.39, 68.37, 61.07, 61.01, 60.99, 60.59, 60.18, 60.05, 54.76, 22.30. HRMS (ESI-Orbitrap) m/z: [M+Na]⁺ calculated for C₂₆H₄₅NNaO₂₁ 730.2382; found 730.2361.

Fucα2Galβ3GlcNAcβ3Galβ4Glc (LNFP-I, 8a). White powder, 16.6 mg, 96% yield. ¹H NMR (600 MHz, D₂O, 30° C.) δ 5.22 (d, J=3.6 Hz, 0.4H), 5.19 (d, J=4.1 Hz, 1H), 4.68-4.61 (m, 2.6H), 4.42 (d, J=7.9 Hz, 1H), 4.29 (q, J=6.6 Hz, 1H), 4.14 (d, J=3.3 Hz, 1H), 4.02-3.45 (m, 27H), 3.28 (t, J=8.5 Hz, 0.5H), 2.06 (s, 3H), 1.23 (d, J=6.6 Hz, 3H). 13C NMR (150 MHz, D₂O, 30° C.) δ 174.22, 103.21, 102.93, 102.89, 100.22, 99.49, 95.87, 95.71, 92.05, 91.78, 81.54, 78.21, 78.12, 77.13, 76.64, 75.91, 75.71, 75.22, 75.04, 74.79, 74.30, 74.09, 73.76, 73.46, 72.72, 71.83, 71.34, 71.10, 70.18, 70.12, 69.60, 69.56, 69.40, 69.10, 68.56, 68.45, 68.02, 66.47, 61.12, 60.93, 60.70, 60.54, 60.37, 60.05, 59.92, 54.95, 22.11, 15.24.

Galβ33GlcNAcβ3Galβ4(Fucα3)Glc (LNFP-V, 9a). White powder, 16.8 mg, 95% yield. ¹H NMR (800 MHz, D₂O, 30° C.) δ 5.41 (d, J=4.0 Hz, 0.5H), 5.36 (d, J=4.0 Hz, 0.4H), 5.17 (d, J=3.8 Hz, 0.4H), 4.81 (d, J=1.3 Hz, 1H), 4.71 (dd, J=8.5, 3.2 Hz, 1H), 4.64 (d, J=8.0 Hz, 0.5H), 4.43 (d, J=7.7 Hz, 1H), 4.40 (d, J=7.9 Hz, 1H), 4.08 (d, J=3.4 Hz, 1H), 3.97-3.67 (m, 24H), 3.63-3.37 (m, 9H), 2.00-2.02 (m, 3H), 1.15 (dd, J=6.7, 4.6 Hz, 3H). ¹³C NMR (200 MHz, D₂O, 30° C.) δ 174.91, 103.42, 102.48, 101.68, 98.52, 98.41, 95.85, 95.80, 92.08, 81.93, 81.47, 81.39, 76.96, 75.70, 75.50, 75.33, 75.24, 75.11, 74.65, 74.47, 72.64, 72.41, 72.34, 72.24, 71.89, 71.87, 71.82, 70.91, 70.69, 70.63, 69.54, 69.23, 69.18, 68.49, 68.39, 68.24, 68.20, 67.99, 67.96, 66.45, 66.41, 61.45, 61.43, 61.00, 60.69, 60.44, 59.76, 59.69, 54.66, 22.17, 15.18. HRMS (ESI-Orbitrap) m/z: [M+Na]⁺ calculated for C₃₂H₅₅NNaO₂₅ 876.2961; found 876.2968.

Galβ33(Fucα4)GlcNAcβ3Galβ4(Fucα3)Glc (LNDFH-II, 10a). White powder, 17.5 mg, 97% yield. ¹H NMR (600 MHz, D₂O, 30° C.) δ 5.42 (d, J=4.0 Hz, 0.4H), 5.36 (d, J=4.0 Hz, 0.4H), 5.17 (d, J=3.8 Hz, 0.4H), 5.02 (d, J=4.0 Hz, 1H), 4.87 (q, J=6.8 Hz, 1H), 4.83-4.80 (m, 1H), 4.68 (d, J=8.4 Hz, 1H), 4.64 (d, J=8.0 Hz, 0.5H), 4.51 (d, J=7.7 Hz, 1H), 4.41 (d, J=7.8 Hz, 1H), 4.10-4.03 (m, 2H), 3.99-3.65 (m, 22H), 3.65-3.42 (m, 8H), 2.05-2.00 (m, 3H), 1.16 (dd, J=10.9, 6.8 Hz, 6H). ¹³C NMR (150 MHz, D₂O, 30° C.) δ 174.71, 102.81, 102.55, 101.71, 98.52, 98.41, 97.97, 95.81, 92.09, 81.56, 81.49, 76.96, 75.89, 75.51, 75.34, 75.15, 74.77, 74.65, 74.47, 72.65, 72.35, 72.27, 72.08, 71.91, 70.92, 70.64, 70.59, 70.46, 69.24, 69.18, 69.10, 68.32, 68.20, 68.00, 67.97, 67.75, 66.81, 66.46, 66.42, 61.62, 61.44, 59.76, 59.56, 55.83, 22.24, 15.33, 15.17. HRMS (ESI-Orbitrap) m/z: [M+Na]⁺ calculated for C₃₈H₆₅NNaO₂₉ 1022.3540; found 1022.3551.

Galβ4GlcNAcβ3Galβ4GlcNAcβ3Galβ4Glc (pLNnH, 11a). White powder, 17.4 mg, 96% yield. ¹H NMR (800 MHz, D₂O, 30° C.) δ 5.21 (d, J=3.8 Hz, 0.3H), 4.69 (d, J=8.4 Hz, 2H), 4.65 (d, J=8.0 Hz, 0.5H), 4.46 (dd, J=10.2, 7.9 Hz, 2H), 4.42 (d, J=7.9 Hz, 1H), 4.14 (t, J=3.8 Hz, 2H), 3.95-3.90 (m, 4H), 3.88-3.69 (m, 21H), 3.67-3.51 (m, 9H), 2.02 (s, 6H). ¹³C NMR (200 MHz, D₂O, 30° C.) δ 174.86, 102.89, 102.85, 102.82, 102.72, 102.70, 95.70, 91.77, 82.03, 81.99, 81.97, 78.32, 78.21, 78.12, 78.10, 75.31, 74.83, 74.76, 74.51, 74.31, 73.74, 72.46, 72.14, 72.13, 71.36, 71.09, 70.92, 70.08, 69.95, 69.92, 68.51, 68.30, 68.27, 60.99, 60.93, 60.92, 60.03, 59.81, 55.15, 55.11, 22.14. HRMS (ESI-Orbitrap) m/z: [M+Na]⁺ calculated for C₄₀H₆₈N₂NaO₃₁ 1095.3704; found 1095.3761.

GlcNAcβ3Galβ4GlcNAcβ3Galβ4GlcNAcβ3Galβ4Glc (GlcNAc-pLNnH, 12a). White powder, 17.7 mg, 96% yield. ¹H NMR (800 MHz, D₂O, 30° C.) δ 5.21 (d, J=3.8 Hz, 0.3H), 4.70-4.66 (m, 3H), 4.65 (d, J=7.9 Hz, 0.6H), 4.45 (d, J=7.9 Hz, 2H), 4.42 (dd, J=7.9, 1.4 Hz, 1H), 4.14 (t, J=2.7 Hz, 3H), 3.95-3.68 (m, 29H), 3.64-3.54 (m, 8H), 3.47-3.41 (m, 2H), 3.29-3.24 (m, 0.5H), 2.00-2.05 (m, 9H). ¹³C NMR (200 MHz, D₂O, 30° C.) δ 174.91, 174.86, 102.89, 102.85, 102.83, 102.72, 102.70, 95.70, 91.77, 82.02, 81.99, 81.97, 81.95, 78.32, 78.21, 78.11, 75.61, 74.85, 74.83, 74.76, 74.51, 74.31, 73.75, 73.52, 72.13, 71.36, 71.09, 70.08, 69.96, 69.92, 69.63, 68.30, 68.28, 60.92, 60.43, 60.03, 59.90, 59.81, 55.62, 55.11, 22.14. HRMS (ESI-Orbitrap) m/z: [M+Na]⁺ calculated for C₄₈H₈₁N₃NaO₃₆ 1298.4497; found 1298.4452.

Fucα2Galβ4GlcNAcβ3Galβ4GlcNAcβ3Galβ4Glc (F-pLNnH-I, 13a). White powder, 17.6 mg, 96% yield. ¹H NMR (800 MHz, D₂O, 30° C.) δ 5.29 (d, J=3.4 Hz, 1H), 5.20 (d, J=3.7 Hz, 0.4H), 4.70-4.67 (m, 2H), 4.65 (d, J=8.0 Hz, 0.6H), 4.53 (d, J=7.7 Hz, 1H), 4.45 (d, J=7.8 Hz, 1H), 4.42 (dd, J=7.9, 1.4 Hz, 1H), 4.20 (q, J=6.7 Hz, 1H), 4.13 (t, J=3.5 Hz, 2H), 3.94 (ddd, J=12.5, 8.6, 2.2 Hz, 3H), 3.88-3.43 (m, 35H), 3.26 (dd, J=9.2, 7.9 Hz, 0.6H), 2.05-2.00 (m, 6H), 1.21 (d, J=6.6 Hz, 3H). ¹³C NMR (200 MHz, D₂O, 30° C.) δ 174.88, 174.87, 102.89, 102.85, 102.84, 102.75, 102.70, 100.21, 99.37, 95.70, 91.77, 81.99, 81.97, 78.32, 78.21, 78.12, 76.40, 75.82, 75.22, 75.05, 74.83, 74.81, 74.76, 74.51, 74.31, 73.75, 73.49, 72.13, 72.03, 71.63, 71.36, 71.09, 70.08, 69.95, 69.93, 69.57, 69.08, 68.32, 68.30, 68.27, 68.16, 66.91, 61.09, 60.93, 60.92, 60.89, 60.03, 59.96, 59.90, 59.81, 55.34, 55.12, 22.16, 22.13, 15.28. HRMS (ESI-Orbitrap) m/z: [M+Na]⁺ calculated for C₄₆H₇₈N₂NaO₃₅ 1241.4283; found 1241.4262.

Galβ4(Fucα3)GlcNAcβ3Galβ4(Fucα3)GlcNAcβ3Galβ4(Fucα3)Glc (TF-pLNnH, 14a). White powder, 17.9 mg, 95% yield. ¹H NMR (800 MHz, D₂O, 30° C.) δ 5.41 (d, J=4.0 Hz, 0.4H), 5.36 (d, J=4.0 Hz, 0.3H), 5.17 (d, J=3.8 Hz, 0.3H), 5.12 (d, J=4.0 Hz, 1H), 5.11 (d, J=4.2 Hz, 1H), 4.82 (d, J=6.7 Hz, 1H), 4.81 (d, J=6.8 Hz, 2H), 4.69 (d, J=8.4 Hz, 2H), 4.64 (d, J=8.0 Hz, 0.4H), 4.45 (d, J=7.8 Hz, 1H), 4.43 (d, J=7.8 Hz, 1H), 4.40 (d, J=7.8 Hz, 1H), 4.08 (t, J=4.1 Hz, 2H), 3.98-3.81 (m, 18H), 3.79-3.44 (m, 28H), 2.02-1.98 (m, 6H), 1.17-1.11 (m, 9H). ¹³C NMR (200 MHz, D₂O, 30° C.) δ 174.64, 174.61, 102.46, 101.70, 98.65, 98.55, 81.58, 76.95, 75.51, 75.33, 75.03, 74.88, 74.72, 74.45, 74.41, 72.98, 72.88, 72.72, 72.43, 71.87, 71.82, 71.62, 71.01, 70.49, 69.23, 69.15, 69.13, 68.31, 68.21, 67.99, 67.66, 67.60, 66.66, 66.45, 61.47, 61.43, 59.59, 55.92, 22.20, 15.47, 15.28, 15.24, 15.17. HRMS (ESI-Orbitrap) m/z: [M+Na]⁺ calculated for C₅₈H₉₈N₂NaO₄₃ 1533.5441; found 1533.5476.

Neu5Acα3Galβ4GlcNAcβ3Galβ4GlcNAcβ3Galβ4Glc (Neu5Acα2-3pLNnH, 15a). White powder, 17.1 mg, 94% yield. ¹H NMR (800 MHz, D₂O, 30° C.) δ 5.28 (d, J=3.8 Hz, 0.4H), 4.76 (d, J=4.2 Hz, 2H), 4.72 (d, J=8.0 Hz, 0.7H), 4.61 (d, J=7.8 Hz, 1H), 4.53 (d, J=7.9 Hz, 1H), 4.50 (d, J=7.9 Hz, 1H), 4.21 (t, J=4.1 Hz, 2H), 4.17 (dd, J=9.9, 3.2 Hz, 1H), 4.04-3.99 (m, 4H), 3.96-3.75 (m, 25H), 3.73-3.61 (m, 11H), 3.34 (t, J=8.5 Hz, 0.6H), 2.82 (dd, J=12.5, 4.7 Hz, 1H), 2.12-2.07 (m, 9H), 1.86 (t, J=12.1 Hz, 1H). ¹³C NMR (200 MHz, D₂O, 30° C.) δ 175.08, 174.92, 173.86, 102.98, 102.94, 102.81, 102.74, 102.63, 99.88, 95.79, 91.86, 88.76, 82.12, 82.08, 82.05, 78.51, 78.41, 78.34, 78.15, 75.57, 75.23, 74.95, 74.93, 74.85, 74.62, 74.41, 73.86, 72.95, 72.23, 72.21, 71.81, 71.45, 71.20, 70.18, 70.03, 69.43, 68.41, 68.38, 68.35, 68.18, 67.55, 62.67, 61.08, 61.01, 61.00, 60.17, 60.04, 59.94, 59.63, 55.25, 55.22, 51.76, 39.72, 22.24, 22.10. HRMS (ESI-Orbitrap) m/z: [M−H]⁻ calculated for C₅₁H₈₄N₃O₃₉ 1362.4687; found 1362.4706.

Neu5Acα6Galβ4GlcNAcβ3Galβ4GlcNAcβ3Galβ4Glc (Neu5Acα2-6pLNnH, 16a). White powder, 17.1 mg, 94% yield. ¹H NMR (800 MHz, D₂O, 30° C.) δ 5.33 (d, J=3.8 Hz, 0.4H), 4.85 (d, J=7.6 Hz, 1H), 4.83 (dd, J=8.4, 3.0 Hz, 1H), 4.77 (s, 0.5H), 4.61-4.54 (m, 3H), 4.26 (t, J=3.6 Hz, 2H), 4.12-4.03 (m, 5H), 4.01-3.81 (m, 22H), 3.79-3.64 (m, 14H), 3.39 (dd, J=9.0, 7.9 Hz, 0.6H), 2.79 (dd, J=12.4, 4.7 Hz, 1H), 2.18-2.13 (m, 9H), 1.82 (t, J=12.2 Hz, 1H). ¹³C NMR (200 MHz, D₂O, 30° C.) δ 175.05, 174.99, 174.98, 173.57, 103.53, 103.05, 103.01, 102.78, 102.63, 100.30, 95.86, 91.93, 82.14, 82.12, 80.56, 78.64, 78.54, 78.48, 74.99, 74.91, 74.69, 74.48, 74.43, 73.94, 73.82, 72.67, 72.59, 72.35, 72.31, 71.84, 71.52, 71.28, 70.88, 70.24, 70.13, 70.10, 68.57, 68.52, 68.44, 68.41, 68.32, 63.47, 62.83, 61.06, 60.34, 60.26, 60.05, 55.28, 55.13, 54.51, 52.03, 40.22, 22.43. HRMS (ESI-Orbitrap) m/z: [M−H]⁻ calculated for C₅₁H₈₄N₃O₃₉ 1362.4687; found 1362.4661.

Galβ3GlcNAcβ3Galβ4GlcNAcβ3Galβ4Glc (pLNH, 17a). White powder, 17.4 mg, 96% yield. ¹H NMR (600 MHz, D₂O, 30° C.) δ 5.22 (d, J=3.8 Hz, 0.3H), 4.74-4.68 (m, 2H), 4.47 (d, J=7.9, 2.2 Hz, 0.5H), 4.44 (dd, J=7.8, 2.4 Hz, 2H), 4.15 (t, J=2.6 Hz, 2H), 3.95 (d, J=12.3 Hz, 2H), 3.92-3.87 (m, 3H), 3.85-3.69 (m, 20H), 3.65-3.46 (m, 9H), 3.28 (t, J=8.5 Hz, 0.5H), 2.01-2.04 (m, 6H). ¹³C NMR (150 MHz, D₂O, 30° C.) δ 174.94, 174.88, 103.46, 102.90, 102.86, 102.71, 102.55, 95.71, 91.79, 82.04, 82.00, 78.35, 78.24, 78.16, 75.26, 75.16, 74.85, 74.77, 74.52, 74.32, 73.76, 72.44, 72.15, 71.37, 71.10, 70.65, 70.09, 69.97, 68.50, 68.42, 61.00, 60.93, 60.46, 60.05, 59.83, 55.13, 54.67, 54.33, 22.20, 22.15. HRMS (ESI-Orbitrap) m/z: [M+Na]⁺ calculated for C₄₀H₆₈N₂NaO₃₁ 1095.3704; found 1095.3770.

Fucα2Galβ3GlcNAcβ3Galβ4GlcNAcβ3Galβ4Glc (F-pLNH-I, 18a). White powder, 17.1 mg, 95% yield. ¹H NMR (600 MHz, D₂O, 30° C.) δ 5.22 (d, J=3.6 Hz, 0.4H), 5.19 (d, J=4.1 Hz, 1H), 4.70 (dd, J=8.3, 2.2 Hz, 1H), 4.64 (ddd, J=15.9, 10.7, 8.2 Hz, 2.6H), 4.44 (t, J=8.2 Hz, 2H), 4.29 (q, J=6.5 Hz, 1H), 4.14 (dd, J=6.3, 3.3 Hz, 2H), 4.01-3.87 (m, 6H), 3.85-3.70 (m, 21H), 3.69-3.63 (m, 3H), 3.61-3.38 (m, 8H), 3.28 (dd, J=9.1, 8.0 Hz, 0.6H), 2.09-2.00 (m, 6H), 1.23 (d, J=6.6 Hz, 3H). ¹³C NMR (150 MHz, D20, 30° C.) δ 174.88, 174.21, 103.23, 102.89, 102.70, 100.22, 99.49, 95.87, 95.71, 92.05, 91.78, 82.00, 81.60, 78.34, 78.24, 78.05, 77.13, 76.63, 75.91, 75.71, 75.22, 75.05, 74.84, 74.78, 74.54, 74.32, 74.09, 73.76, 73.46, 72.72, 72.12, 71.83, 71.44, 71.37, 71.10, 70.16, 70.09, 69.94, 69.56, 69.41, 69.10, 68.54, 68.44, 68.32, 68.02, 66.47, 61.12, 60.92, 60.70, 60.36, 60.05, 59.83, 55.13, 54.95, 22.15, 22.12, 15.23. HRMS (ESI-Orbitrap) m/z: [M+Na]⁺ calculated for C₄₆H₇₈N2NaO₃₅ 1241.4283; found 1241.4254.

Galβ33(Fucα4)GlcNAcβ3Galβ4(Fucα3)GlcNAcβ3Galβ4(Fucα3)Glc (TF-pLNH-III, 19a). White powder, 17.8 mg, 97% yield. ¹H NMR (600 MHz, D₂O, 30° C.) δ 5.43 (d, J=4.0 Hz, 0.5H), 5.37 (d, J=4.0 Hz, 0.4H), 5.18 (d, J=3.8 Hz, 0.4H), 5.12 (d, J=4.0 Hz, 1H), 5.03 (d, J=4.0 Hz, 1H), 4.88 (q, J=6.7 Hz, 1H), 4.81 (d, J=6.6 Hz, 2H), 4.70 (t, J=8.0 Hz, 2H), 4.65 (d, J=8.0 Hz, 0.5H), 4.51 (d, J=7.7 Hz, 1H), 4.45 (d, J=7.9 Hz, 1H), 4.41 (d, J=7.8 Hz, 1H), 4.10 (t, J=4.0 Hz, 2H), 4.06 (d, J=9.7 Hz, 1H), 3.98-3.83 (m, 16H), 3.82-3.67 (m, 18H), 3.64-3.44 (m, 11H), 2.03 (s, 3H), 2.02 (s, 3H), 1.21-1.13 (m, 9H). ¹³C NMR (150 MHz, D₂O, 30° C.) δ 174.69, 174.64, 102.82, 102.55, 102.45, 101.70, 98.66, 97.97, 95.81, 92.10, 81.64, 81.52, 76.97, 75.90, 75.52, 75.34, 75.16, 75.04, 74.79, 74.73, 74.43, 72.77, 72.66, 72.34, 72.29, 72.08, 71.92, 71.84, 70.93, 70.63, 70.47, 69.55, 69.20, 69.15, 69.11, 68.33, 68.21, 67.97, 67.76, 67.62, 66.82, 66.66, 66.46, 61.63, 61.44, 59.58, 55.93, 55.84, 22.25, 22.21, 15.34, 15.25, 15.18. HRMS (ESI-Orbitrap) m/z: [M+Na]⁺ calculated for C₅₈H₉₈N2NaO₄₃ 1533.5441; found 1553.5425.

Galβ4GlcNAcβ3Galβ4GlcNAcβ3Galβ4GlcNAcβ3Galβ4Glc (pLNnO, 20a). White powder, 17.6 mg, 96% yield. ¹H NMR (800 MHz, D₂O, 30° C.) δ 5.21 (d, J=3.8 Hz, 0.3H), 4.68 (dd, J=8.3, 2.3 Hz, 3H), 4.65 (d, J=8.0 Hz, 0.7H), 4.46 (dd, J=10.8, 7.8 Hz, 3H), 4.42 (d, J=7.9 Hz, 1H), 4.14 (q, J=3.2, 2.4 Hz, 3H), 3.93 (td, J=13.1, 12.7, 2.8 Hz, 5H), 3.85-3.69 (m, 29H), 3.68-3.61 (m, 3H), 3.59-3.51 (m, 8H), 3.29-3.24 (m, 0.5H), 2.02 (s, 9H). ¹³C NMR (200 MHz, D₂O, 30° C.) δ 174.86, 102.89, 102.85, 102.82, 102.72, 102.70, 95.70, 91.77, 82.03, 81.99, 81.97, 78.32, 78.21, 78.11, 75.31, 74.83, 74.76, 74.51, 74.31, 73.75, 72.46, 72.14, 72.13, 71.36, 71.09, 70.93, 70.08, 69.95, 69.92, 68.51, 68.30, 68.28, 60.99, 60.93, 60.92, 60.03, 59.90, 59.81, 55.15, 55.11, 22.14. HRMS (ESI-Orbitrap) m/z: [M+Na]⁺ calculated for C₅₄H₉₁N3NaO₄₁ 1460.5026; found 1460.5006.

Galβ3GlcNAcβ3Galβ4GlcNAcβ3Galβ4GlcNAcβ3Galβ4Glc (pLNO, 21a). White powder, 17.8 mg, 96% yield. ¹H NMR (800 MHz, D₂O, 30° C.) δ 5.28 (d, J=3.8 Hz, 0.3H), 4.80 (s, 1H), 4.76 (d, J=6.4 Hz, 2H), 4.72 (d, J=8.0 Hz, 0.7H), 4.53 (dd, J=9.7, 7.8 Hz, 3H), 4.50 (dd, J=7.8, 1.7 Hz, 1H), 4.21 (d, J=3.1 Hz, 3H), 4.02-3.76 (m, 34H), 3.73-3.53 (m, 11H), 3.34 (t, J=8.5 Hz, 0.6H), 2.07-2.11 (m, 9H). ¹³C NMR (200 MHz, D₂O, 30° C.) δ 174.93, 103.52, 102.98, 102.95, 102.93, 102.75, 102.74, 102.58, 95.79, 91.86, 85.43, 82.18, 82.11, 82.07, 78.50, 78.40, 78.32, 75.40, 75.33, 75.25, 74.92, 74.85, 74.61, 74.41, 73.86, 72.58, 72.54, 72.23, 71.45, 71.20, 71.02, 70.74, 70.18, 70.05, 70.02, 68.61, 68.52, 68.38, 68.36, 61.07, 60.99, 60.17, 59.96, 55.26, 55.22, 54.75, 22.29, 22.24. HRMS (ESI-Orbitrap) m/z: [M+Na]⁺ calculated for C₅₄H₉₁N3NaO₄₁ 1460.5026; found 1460.4964.

Fucα2Galβ3GlcNAcβ3Galβ4GlcNAcβ3Galβ4GlcNAcβ3Galβ4Glc (Fucα1-2pLNO, 22a). White powder, 17.5 mg, 95% yield. ¹H NMR (600 MHz, D₂O, 30° C.) δ 5.21 (d, J=3.8 Hz, 0.3H), 5.18 (d, J=4.1 Hz, 1H), 4.69 (d, J=8.3 Hz, 2H), 4.65 (dd, J=9.9, 7.8 Hz, 1.5H), 4.61 (d, J=8.4 Hz, 1H), 4.47-4.42 (m, 3H), 4.28 (q, J=6.7 Hz, 1H), 4.14 (dd, J=7.4, 3.2 Hz, 3H), 4.01-3.86 (m, 7H), 3.85-3.69 (m, 32H), 3.68-3.62 (m, 4H), 3.61-3.46 (m, 10H), 3.27 (t, J=8.5 Hz, 0.5H), 2.05 (s, 3H), 2.02 (s, 6H), 1.23 (d, J=6.6 Hz, 3H). ¹³C NMR (150 MHz, D₂O, 30° C.) δ 174.87, 174.20, 103.22, 102.88, 102.86, 102.71, 100.22, 99.48, 98.39, 82.03, 81.58, 78.02, 77.12, 76.63, 75.21, 75.04, 74.83, 74.78, 74.76, 74.52, 74.32, 73.75, 73.45, 72.14, 71.37, 71.10, 69.93, 69.40, 69.12, 69.09, 68.53, 68.43, 68.31, 68.01, 66.85, 66.46, 62.44, 61.12, 60.92, 60.35, 59.81, 55.12, 54.94, 22.14, 22.11, 15.23. HRMS (ESI-Orbitrap) m/z: [M+Na]⁺ calculated for C₆₀H₁₀₁N₃NaO₄₅ 1606.5605; found 1606.5542.

### Example 4. Results and Discussion

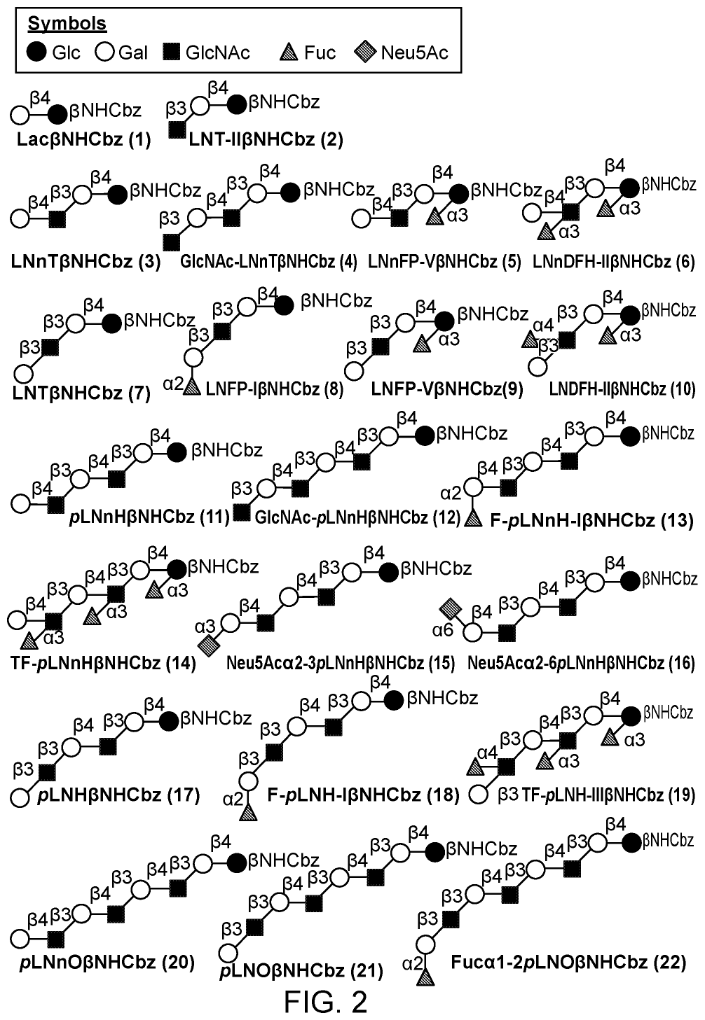

The efficiency of the MSOPME strategy presented herein demonstrated high-yield (71-91%) preparative-scale (83-155 mg) synthesis of more than twenty tagged HMOs (2-21) including fucosylated or sialylated long-chain oligosaccharides up to nonaoses (FIG. 2). Compound 21 was observed to be well-suited acceptor substrate for an OPME α1-2-fucosylation system for the preparation of Cbz-tagged fucosylated nonasaccharide 22 in an excellent 92% yield. Gram-scale synthesis of lacto-N-fucopentaose-I (LNFP-I) was achieved in 1.74 grams with 84% yield without the need for intermediate enzyme inactivation processes. Tag removal was achieved in high efficiency (94-97%) without the need for column purification to produce the desired natural HMOs with a free reducing end. The method provided herein can be readily adapted for automation where the process is controlled by computer program carried out using proper instrumentation.

### 1. Glycosyltransferase Acceptor Substrate Engineering by Cbz Tagging

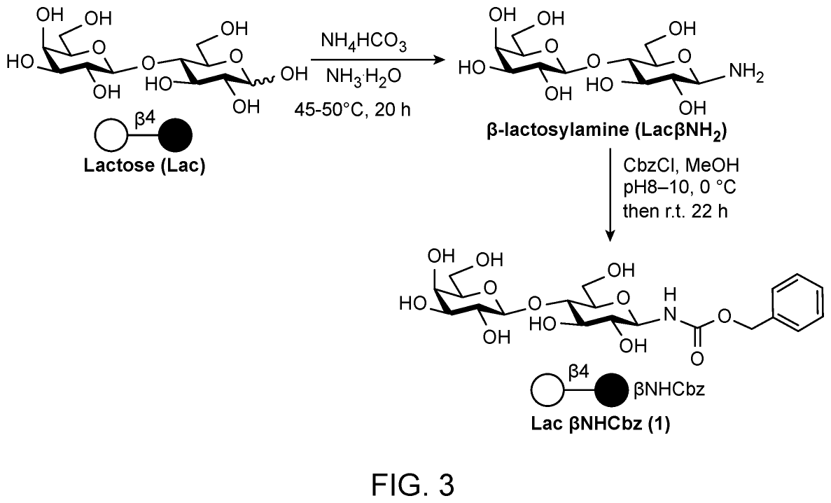

HMOs are extended from lactose, which is a low-cost starting material that can be used directly for enzymatic synthesis of HMOs by reactions catalyzed by glycosyltransferases or mutant glycosidases. Nevertheless, lactose is a disaccharide with a free reducing terminus which forms a mixture of α and β-anomers as well as a small percentage of open-chain structure on its glucose unit in aqueous solutions. This property leads to complications in product characterization and purification. To simplify the reaction monitoring and product purification processes, carboxybenzyl (Cbz) protected β-lactosylamine (LacβNHCbz, 1) was designed herein. The Cbz tag is an ultraviolet (UV)-active hydrophobic tag that can be easily installed using commercially available materials. The Cbz tag can also be easily removed by simple catalytic hydrogenation and hydrolysis without the need for a chromatography purification process. As shown in FIG. 3, LacβNHCbz was synthesized from lactose in two steps. See, Li, Ghosh, Bai, Santra, Xiao, and X. Chen, et al. Carbohydr Res 2019, 479, 41-47, which is incorporated herein by reference in its entirety. Lactose was incubated with ammonium bicarbonate in ammonium hydroxide to form lactosylamine (LacβNH₂). The solvent was removed and the dried residue was dissolved in anhydrous methanol and coupled with benzyl chloroformate (CbzCl) to obtain LacβNHCbz (1) which was readily purified by a C18 cartridge. The procedure was robust and LacβNHCbz (1) was consistently produced in high yields (75-80%) in multi-gram (3-5 g)-scale syntheses.

### 2. One-Pot Multienzyme (OPME) Systems and Selection of Biocatalysts

HMOs are complex glycans that are extended from lactose with N-acetyl-D-glucosamine (GlcNAc), D-galactose (Gal), L-fucose (Fuc), and/or N-acetyl-D-neuraminic acid (Neu5Ac) units. One-pot multienzyme (OPME) systems, each containing a suitable glycosyltransferase and a monosaccharide sugar activation (SA) enzymes and reagents, are designed to form the target HMOs with in situ generation of sugar nucleotide donor of the glycosyltransferase from inexpensive monosaccharide and nucleoside triphosphates such as adenosine 5′-triphosphate (ATP), uridine 5′-triphosphate (UTP), and/or cytidine 5′-triphosphate (CTP). As shown in FIG. 4, four SA components are needed for the activation of GlcNAc (SA1), Gal (SA2), Fuc (SA3), and Neu5Ac (SA4), respectively.

Three enzymes including Bifidobacterium longum strain ATCC55813 N-acetylhexosamine-1-kinase (BLNahK), Pasteurella multocida N-acetylglucosamine uridylyltransferase (PmGlmU), and Pasteurella multocida inorganic pyrophosphatase (PmPpA) were chosen for GlcNAc activation SA1. BLNahK was found to be highly active in catalyzing the direct phosphorylation of GlcNAc (k_{cat}/Kₘ=18.3 s⁻¹ mM⁻¹) using ATP for the formation of GlcNAc-1-phosphate. It had a high expression level in E. coli (185 mg/L culture). PmGlmU, with an expression level of 170 mg/L culture, catalyzed highly efficient synthesis of UDP-GlcNAc from GlcNAc-1-P and UTP. PmPpA, with an expression level of 488 mg/L culture, was used to break down the pyrophosphate formed in the PmGlmU reaction to inorganic phosphate to shift the coupled enzymatic reactions towards the formation of UDP-GlcNAc. Two β1-3-N-acetylglucosaminyltransferases (β3GlcNAcTs) from Neisseria meningitidis (NmLgtA) and Helicobacter pylori (Hpβ3GlcNAcT), respectively, were selected. NmLgtA (10 mg/L) with a better expression level than Hpβ3GlcNAcT (1 mg/L), was used to add β1-3-linked GlcNAc to short acceptor substrates such as LacβNHCbz (1) while Hpβ3GlcNAcT was used to add β1-3-linked GlcNAc to longer acceptor substrates which were less efficient for NmLgtA-catalyzed reactions.

Three enzymes including Streptococcus pneumoniae TIGR4 galactokinase (SpGalK), Bifidobacterium longum UDP-sugar pyrophosphorylase (BLUSP), and PmPpA were chosen for Gal activation SA2. SpGalK has an expression level of 100 mg/L culture and BLUSP has an expression level of 170 mg/L culture. Two bacterial galactosyltransferases Chromobacterium violaceum β1-3-galactosyltransferase (Cvβ3GalT) and β1-4-galactosyltransferase (NmLgtB) were chosen for the addition of a Gal with β1-3- and β1-4-linkages, respectively, to the GlcNAc-terminated glycans. Cvβ3GalT has an expression level of 40 mg/L culture and NmLgtB has an expression level of 12.5 mg/L culture. Cvβ3GalT is a highly efficient β1-3-galactosyltransferase which was used previously to synthesize LNT successfully in multi-gram scale. NmLgtB is an efficient 1-4-galactosyltransferase for synthesizing LacNAc-containing structures.

Two enzymes including a bifunctional L-fucokinase/GDP-fucose pyrophosphorylase Bacteroides fragilis NCTC9343 (BfFKP) and PmPpA were chosen for L-fucose activation SA3. BfFKP has an expression level of 20 mg/L culture. The bifunctional BfFKP contains a C-terminal fucokinase domain and an N-terminal GDP-fucose pyrophosphorylase domain. Three bacterial fucosyltransferases, including Helicobacter mustelae α1-2-fucosyltransferase (Hm2FT), Thermosynechococcus elongatus α1-2-fucosyltransferase (Te2FT), and Helicobacter pylori α11-3/4 fucosyltransferase (Hp3/4FT), were chosen for the addition of an α1-2-, or one or more α11-3/4-linked L-fucose residues. Hm2FT, with an expression level of 10 mg/L culture, catalyzed the transfer of L-fucose to both β1-3 and β1-4-linked galactosides while Te2FT, with an expression level of 15 mg/L culture, showed high selectivity towards β1-3-linked galactosides. Te2FT was chosen for the synthesis of LNFP-I. Hp3/4FT, with an expression level of 30 mg/L culture, catalyzed the transfer of L-fucose to form α1-3 and α1-4-fucosylated products. Hp3/4FT was shown herein to be a powerful enzyme for α1-3/4-fucosylation of the GlcNAc and Glc residues in HMOs.

Sialic acid was shown herein to be activated by Neisseria meningitidis CMP-sialic acid synthetase (NmCSS) (SA4) and transferred by Pasteurella multocida α2-3-sialyltransferase 3 (PmST3) or Photobacterium damselae α2-6-sialyltransferase (Pd2,6ST_A200Y/S232Y) (OPME4). NmCSS showedan expression level of 100 mg/L culture. PmST3, with an expression level of 29 mg/L culture, showed a preference for β1-4-over β1-3-linked galactoside and was used for sialylating β1-4-linked terminal galactose. Pd2,6ST_A200Y/S232Y, with an expression level of 32 mg/L culture, showed high selectivity in sialylating the terminal galactose in HMOs with an α2-6-linkage.

### 3. The Multistep One-Pot Multienzyme (MSOPME) Strategy

To assemble desired oligosaccharides, a previous common practice was to perform a product purification process after every glycosyltransferase-catalyzed reaction. Numerous methods have been used to facilitate the product purification of these enzyme-catalyzed glycosylation reactions, including solid phase immobilization, tagging with a thermosensitive polymer, a hydrophobic or an anionic tail, etc. Precipitation or solid phase extraction was used to purify every oligosaccharide intermediate before it was used as the acceptor substrate for the next glycosylation reaction.

As provided herein, the isolation and purification of oligosaccharide intermediates was not necessary and can be bypassed by using the methods provided herein. This bypassing of isolating and purifying intermediates allowed one-pot access to the desired compound from LacβNHCbz (1) with a single purification process for the final product despite its length and complexity. Such a process can be readily adaptable for automation and is a step forward towards industrial biocatalytic production of HMOs using purified or partially purified enzymes.

To avoid uncontrolled polymerization when both OPME2a containing a 1-4-galactosyltransferase, and OPME1a or OPME1b containing a β1-3-GlcNAc transferase, are present, the reaction mixture was incubated in a boiling water bath for 5 minutes to deactivate the enzymes after each OPME glycosylation is completed as indicated by the disappearance of the acceptor substrate by mass spectrometry analyses and/or thin-layer chromatography (TLC) assays. The reaction mixture was then cooled down and used as the acceptor substrate for the glycosyltransferase in the next OPME reaction without any workup or purification processes. This process is referred to herein as the multistep sequential one-pot multienzyme (MSOPME) strategy.

As shown in Table 1, LNT-IIβNHCbz (2) was synthesized from 100 mg LacβNHCbz (1) using OPME1a and purified with a 91% yield. Two-step sequential OPME reactions followed by a single C18-cartridge purification produced 152 mg LNnTβNHCbz (3) (OPME1a and OPME2a) or 155 mg LNTβNHCbz (7) (OPME1a and OPME2b) from 100 mg of LacβNHCbz (1) in 86-88% yield. Three-step sequential OPME reactions with a single C18-cartridge purification produced 83-91 mg of each of the three pure pentasaccharides GlcNAc-LNnTβ3NHCbz (4) (OPME1a, OPME2a, and OPME1b), LNnFP-Vβ3NHCbz (5) ((OPME1a, OPME3c, and OPME2a), and LNFP-VβNHCbz (9) (OPME1a, OPME3c, and OPME2b) as well as 94 mg pure hexasaccharide LNnDFH-IIβNHCbz (6) (OPME1a, OPME2a, and OPME3c) in 80% yield from 50 mg LacβNHCbz (1). Four-step sequential OPME reactions with a single C18-cartridge purification produced 95-101 mg of each of the three pure hexasaccharides LNDFH-IIβNHCbz (10) (OPME1a, OPME3c, OPME2b, and OPME3c), pLNnHβNHCbz (11) (OPME1a, OPME2a, OPME1b, and OPME2a), and pLNHβNHCbz (17) (OPME1a, OPME2a, OPME1b, and OPME2b) in 79-80% yield from 50 mg LacβNHCbz (1).

TABLE 1
Reaction processes and yields for the synthesis of target glycanβNHCbz (2-22).

| | | Yield | | | Yield |
|---|---|---|---|---|---|
| Product | Process | (amount) | Product | Process | (amount) |
| LNT-IIβNHCbz | OPME1a | 91% | F-pLNnH- | OPME1a | 76% |
| (2) | | (130 mg) ^{[b]} | IβNHCbz (13) | OPME2a | (108 mg) ^{[a]} |
| | | | | OPME1b | |
| | | | | OPME2a | |
| | | | | OPME3a | |
| LNnTβNHCbz | OPME1a | 86% | TF- | OPME1a | 76% |
| (3) | OPME2a | (152 mg) ^{[b]} | pLNnHβNHCbz | OPME2a | (131 mg) ^{[a]} |
| | | | (14) | OPME1b | |
| | | | | OPME2a | |
| | | | | OPME3c | |
| GlcNAc- | OPME1a | 83% | Neu5Acα2- | OPME1a | 78% |
| LNnTβNHCbz | OPME2a | (91 mg) ^{[a]} | 3pLNnHβNHCbz | OPME2a | (123 mg) ^{[a]} |
| (4) | OPME1b | | (15) | OPME1b | |
| | | | | OPME2a | |
| | | | | OPME4a | |
| LNnFP- | OPME1a | 80% | Neu5Acα2- | OPME1a | 75% |
| VβNHCbz (5) | OPME3c | (83 mg) | 6pLNnHβNHCbz | OPME2a | (118 mg) ^{[a]} |
| | OPME2a | | (16) | OPME1b | |
| | | | | OPME2a | |
| | | | | OPME4b | |
| LNnDFH- | OPME1a | 80% | pLNHβNHCbz | OPME1a | 80% |
| IIβNHCbz (6) | OPME2a | (94 mg) ^{[a]} | (17) | OPME2a | (101 mg) ^{[a]} |
| | OPME3c | | | OPME1b | |
| | | | | OPME2b | |
| LNTβNHCbz (7) | OPME1a | 88% | F-pLNH- | OPME1a | 74% |
| | OPME2b | (155 mg) ^{[b]} | IβNHCbz (18) | OPME2a | (105 mg) ^{[a]} |
| | | | | OPME1b | |
| | | | | OPME2b | |
| | | | | OPME3a | |
| LNFP-I (8) | OPME1a | 84% | TF-pLNH- | OPME1a | 72% |
| | OPME2b | (1.73 g)^{[c]} | IIIβNHCbz (19) | OPME2a | (124 mg) ^{[a]} |
| | OPME3b | | | OPME1b | |
| | | | | OPME2bOPME3c | |
| LNFP-VβNHCbz | OPME1a | 81% | pLNnOβNHCbz | OPME1a | 72% |
| (9) | OPME3c | (84 mg) ^{[a]} | (20) | OPME2a | (119 mg) ^{[a]} |
| | OPME2b | | | OPME1b | |
| | | | | OPME2a | |
| | | | | OPME1b | |
| | | | | OPME2a | |
| LNDFH- | OPME1a | 80% | pLNOβNHCbz | OPME1a | 71% |
| IIβNHCbz (10) | OPME3c | (95 mg) ^{[a]} | (21) | OPME2a | (117 mg) ^{[a]} |
| | OPME2b | | | OPME1b | |
| | OPME3c | | | OPME2a | |
| | | | | OPME1b | |
| | | | | OPME2b | |
| pLNnHβNHCbz | OPME1a | 79% | Fucα1- | Formed from | 92% |
| (11) | OPME2a | (100 mg) ^{[a]} | 2pLNOβNHCbz | pLNOβNHCbz | (20 mg) |
| | OPME1b | | (22) | (21) (20 mg) | |
| | OPME2a | | | with OPME3a | |
| GlcNAc- | OPME1a | 77% | | | |
| pLNnHβNHCbz | OPME2a | (114 mg) ^{[a]} | | | |
| (12) | OPME1b | | | | |
| | OPME2a | | | | |
| | OPME1b | | | | |

Reactions were carried out using ^{[a]} 50 mg, ^{[b]} 100 mg, or ^{[c]}1 gram of LacβNHCbz (1).

Similar processes also worked well for more complex longer chain glycans. Five-step sequential OPME reactions with a single C18-cartridge purification produced 105-123 mg of each of the pure heptasaccharides including GlcNAc-pLNnHβNHCbz (12) (OPME1a, OPME2a, OPME1b, OPME2a, and OPME1b), F-pLNnH-IβNHCbz (13) (OPME1a, OPME2a, OPME1b, OPME2a, and OPME3a), Neu5Acα2-3pLNnHβNHCbz (15) (OPME1a, OPME2a, OPME1b, OPME2a, and OPME4a), Neu5Acα2-6pLNnHβNHCbz (16) (OPME1a, OPME2a, OPME1b, OPME2a, and OPME4b), and F-pLNH-IβNHCbz (18) (OPME1a, OPME2a, OPME1b, OPME2b, and OPME3a) in 74-78% yield, as well as 124-131 mg of each of the two pure nonasaccharides TF-pLNnHβNHCbz (14) (OPME1a, OPME2a, OPME1b, OPME2a, and OPME3c) and TF-pLNH-IIIβNHCbz (19) (OPME1a, OPME2a, OPME1b, OPME2b, and OPME3c) in 72-76% yield from 50 mg of LacβNHCbz (1). Six-step sequential OPME reactions with a single C18-cartridge purification produced 117-119 mg of each of the two pure octasaccharides pLNnOβNHCbz (20) (OPME1a, OPME2a, OPME1b, OPME2a, OPME1b, and OPME2a) and pLNOβNHCbz (21) (OPME1a, OPME2a, OPME1b, OPME2a, OPME1b, and OPME2b) in 71-72% yield from 50 mg of LacβNHCbz (1).

Purified octasaccharide pLNOβNHCbz (21) was used as the acceptor substrate for Hm2FT in OPME3a for the synthesis of Fucα1-2pLNOβNHCbz (22) which was obtained as a pure compound in 92% yield after a C18-cartridge purification.

Mono-fucosylated pentasaccharide LNnFP-VβNHCbz (5) was formed from LacβNHCbz (1) by adding a β1-3-linked GlcNAc using OPME1a, followed by α1-3-fucosylation with OPME3c and 1-4-galactosylation with OPME2a. Altering the order of the latter two OPME steps led to the formation of difucosylated hexasaccharide LNnDFH-IIβNHCbz (6) (OPME1a, OPME2a, and OPME3c) when 2.86 equivalents of _{L}-fucose and 3.05 equivalents of ATP and UTP were used in OPME3c. See, Yu, X. Chen, et al. Chem. Commun. 2017, 53, 11012-11015, which is incorporated herein by reference in its entirety. Mono-fucosylated pentasaccharide LNFP-VβNHCbz (9) was formed similarly by extending LacβNHCbz (1) with a β1-3-linked GlcNAc using OPME1a, followed by α1-3-fucosylation with OPME3c and 1-3-galactosylation with OPME2b. On the other hand, to ensure the installation of two _{L}-fucose residues to form the difucosylated hexasaccharide LNDFH-IIβNHCbz (10), a four-step OPME process was used by adding an additional α1-3/4-fucosylation OPME3c to the three-step OPME preparation of LNFP-VβNHCbz (9). Installation of all three _{L}-fucose residues in trifucosylated nonasaccharides TF-pLNnHβNHCbz (14) and TF-pLNH-IIIβNHCbz (19) was achieved in one-step OPME3c al-3/4-fucosylation process from pLNnHβNHCbz (11) and pLNHβNHCbz (17) intermediates, respectively, formed from four-step OPME glycosylation of LacβNHCbz (1).

As provided herein, if the glycosyltransferases used for the MSOPME synthesis of the target glycans are selective for the acceptor substrates without the complication of polymerization, the MSOPME can be carried out without the heat inactivation of the enzymes in the intermediate OPME steps. Such an example was demonstrated for gram-scale synthesis of LNFP-I (8). The LNFP-I (8) was successfully synthesized in 1.74 grams in 84% yield after three OPME steps.

### 4. Tag Removal and Hydrolysis of Glycosylamines to Convert HMOβNHCbz Compounds to HMOs

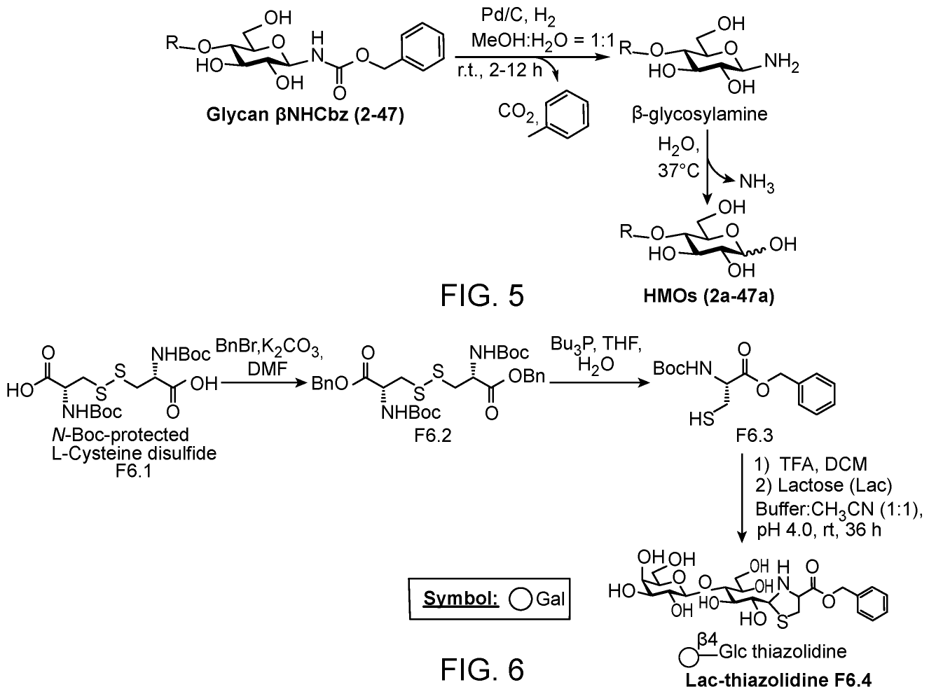

The Cbz tag in the obtained glycosides (2-47) was readily removed by catalytic hydrogenation using palladium and charcoal in methanol and H₂O. The glycosylamine formed was partially converted to the target HMO with a free reducing end during the hydrogenation process. After the palladium and charcoal were removed by filtering the mixture using a 0.45 μm syringe filter, the solvent was removed by rotavap and the complete conversion to the desired HMO with a free reducing end was achieved by dissolving the residue in water and incubating the mixture at 37° C. As shown in FIG. 5, the NHCbz group on the glycosides (2-47) was converted to toluene, CO₂, and NH₃ which were removed by rotary evaporation and lyophilization without purification. Excellent yields (94-97%) were achieved.

Thus, described herein is an efficient process engineering strategy that can be combined with the substrate engineering strategy and OPME systems for multistep one-pot multienzyme (MSOPME) synthesis of target tagged glycans from a simple lactoside without the purification of intermediate oligosaccharides. A single C18-cartridge-based purification of the final tagged product followed by catalytic hydrogenation and spontaneous hydrolysis led to the formation of the desired HMO with a naturally occurring free reducing end. The strategy has been demonstrated for facile synthesis of twenty-one HMOs including those with or without sialic acid or _{L}-fucose and a size up to nonasaccharide with high yields using only a single C18-cartridge purification process for any given target. Gram-scale synthesis has also been demonstrated for an important HMO, lacto-N-fucopentaose-I (LNFP-I). The method can be readily adapted for automation to allow quick access of HMOs as well as other glycans and glycoconjugates.

### Example 5. Development of an Improved Fucosylation Strategy

About 60-77% of HMOs are fucosylated and many of these fucosylated HMOs have more than one fucosyl residues on the structure. While the Fucα1-3Glc linkage on the Lac core is common for some short length HMOs, it is usually missing from the longer or branched-chain HMOs. Both Helicobacter pylori α1-3-fucosyltransferase (Hp3FT) and Helicobacter pylori α1-3/4-fucosyltransferase (Hp3/4FT) showed preference towards LacNAc or Galβ1-3GlcNAc type acceptors and selective fucosylation of GlcNAc over Glc by these fucosyltransferases can be achieved for some short chain simple acceptors (e.g., tetrasaccharides LNT or LNnT) by controlling the donor and acceptor ratios, the amount of the fucosyltransferase used, and the time of the reaction. However, the selective fucosylation cannot be achieved for some longer chain or more complex substrates, is not precise, and will be extremely challenging for large-scale synthesis.

In order to achieve a precise control of fucosylating the GlcNAc, but not the core Glc residue, in the HMOs, provided herein is benzyl protected L-cysteine (F6.3) (FIG. 6) which can be used to form a lactose derivative (Lac-thiazolidine) with a thiazolidine protection of the aldehyde group in the open-chain Glc structure (FIG. 6). The thiazolidine can be easily installed and easily removed with high yields. Benzyl protected L-cysteine (F6.3) can be readily synthesized from L-cysteine (FIG. 6). Briefly, by reacting L-cysteine with di-tert-butyl dicarbonate (Boc₂O), then protecting the carboxyl group with benzyl bromide, followed by reduction with tri-n-butylphosphine (Bu₃P) and removing the Boc protecting group using trifluoroacetic acid (TFA), provided the desired benzyl protected cysteine (F6.3). Coupling Lac with benzyl protected L-cysteine (F6.3) in a water-acetonitrile mixture (pH 4.0) produced the thiazolidine-derivatized lactoside (Lac-thiazolidine, F6.4) where the aldehyde in the open-chain Glc is protected. The open chain structure of the Glc in the lactoside prevents its fucosylation by fucosyltransferases. After MSOPME reactions including fucosylation, removal of the thiazolidine by treating the glycan-thiazolidine with CuSO₄ in the presence of ascorbate will produce the desired HMO with a free reducing end (FIG. 6).

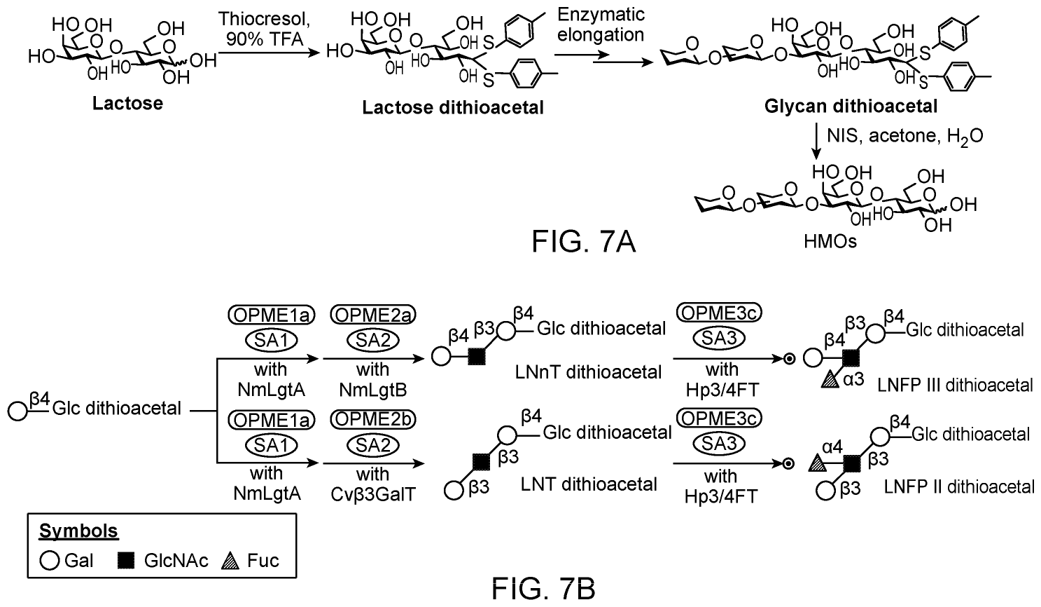

Similarly, as shown in FIG. 7A, a lactose dithioacetal can be synthesized from lactose and thiocresol in 90% trifluoroacetic acid (TFA). It can then be used as the acceptor substrate for OPME glycosylation for the formation of longer glycosides. The dithioacetal-protection of the open-chain Glc prevents it from fucosylation, leading to selective fucosylation of GlcNAc residues by α1-3/4-fucosyltransferases. Afterwards, the resulting glycan dithioacetal with the selective fucosylation pattern is treated with N-iodo-succinimide (NIS) in aqueous acetone to form the desired HMO with a free reducing end.

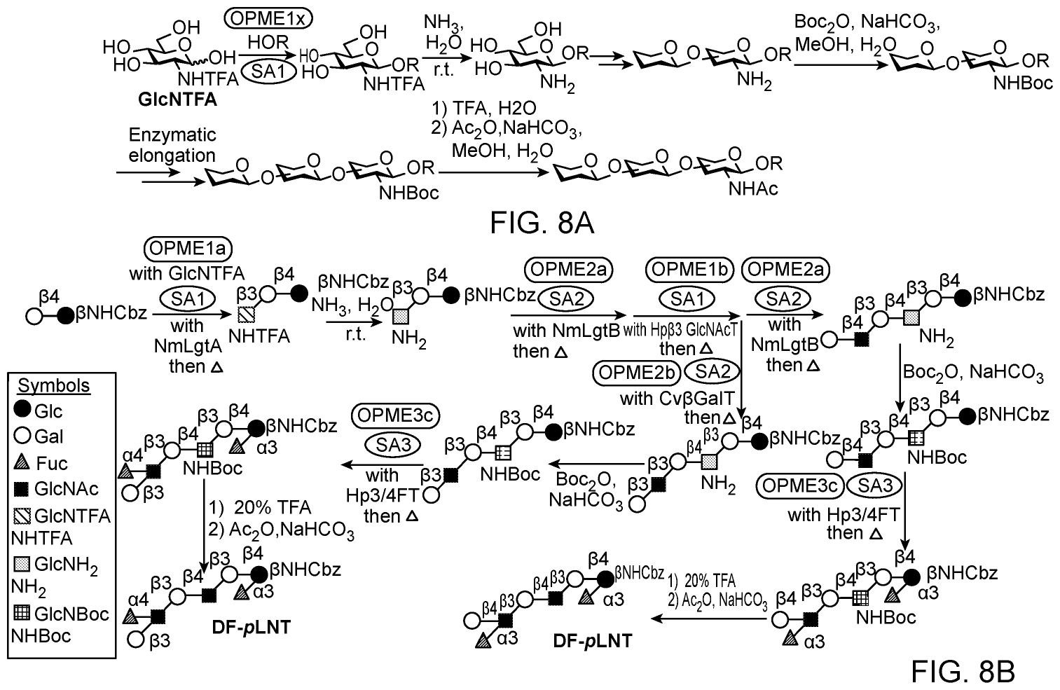

To precisely control selective fucosylation of internal GlcNAc residues in long-chain HMO backbones, GlcNTFA was designed herein as a chemoenzymatic synthon for the GlcNAc that is not intended for enzymatic fucosylation (FIG. 8A). GlcNTFA was readily formed from commercially available inexpensive D-glucosamine hydrochloride by dissolving it in dry methanol (anhydrous methanol), and treating with sodium carbonate (Na₂CO₃) and ethyl trifluoroacetate (EtOTFA). The OPME1a/2a GlcNAc activation and transfer systems readily tolerated GlcNTFA to form the desired GlcNTFAβ1-3Gal linkage. The TFA was then removed from GlcNTFA in the glycan by treating with ammonium hydroxide at room temperature to form GlcNH₂. This step can also be achieved by dissolving the GlcNTFA-containing glycan in methanol and water (1:1 by volume) and incubating at pH 8-10 (adjusting pH using K₂CO₃). The resulting GlcNH₂β1-3Gal-containing glycan is a suitable acceptor for NmLgtB and Hpβ3GalT in OPME2a or OPME2b for the formation of Galβ1-4GlcNH₂β1-3Gal-glycans which can be used for further enzymatic glycosylation to form HMO backbones by MSOPME reactions. Without being limited by theory, the GlcNH₂ in the HMOs prevents fucosylation by either Hp3FT or Hp3/4FT. After fucosylation at the desired GlcNAc sites, GlcNH₂ was easily converted to GlcNAc using Ac₂O and NaHCO₃(FIG. 8A). Alternatively, TFA in GlcNTFA can be removed after elongating the GlcNTFA by NmLgtB or Hpβ3GalT in OPME2a or OPME2b (FIG. 8B).

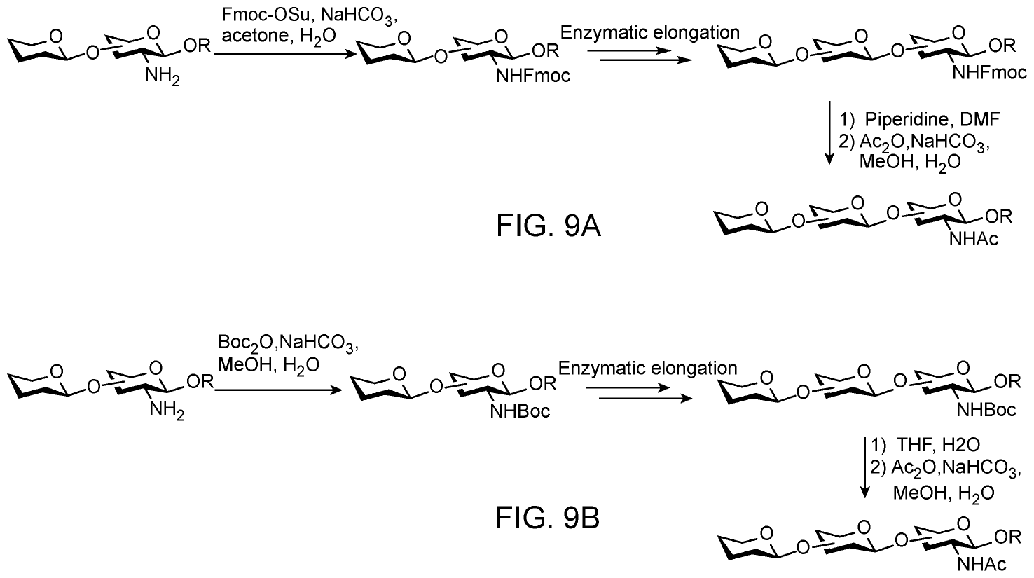

To further improve the protection of the selective internal GlcNAc residue from enzymatic fucosylation, the GlcNH₂-containing glycans obtained from the GlcNTFA/GlcNH₂ synthon strategies illustrated in FIG. 8A and FIG. 8B can be protected with Fmoc or Boc as provided herein and shown in FIG. 9A and FIG. 9B, respectively. The Fmoc can be removed by treating with 20% piperidine in DMF and the Boc can be removed by treating with an aqueous TFA solution. The resulting GlcNH₂ in the glycans can be converted to GlcNAc by acetylation with acetic anhydride and NaHCO₃ in MeOH and water.

Synthesis of (2R, 2′R)-dibenzyl 3,3′-disulfanediylbis(2-((tert-butyloxycarbonyl)amino)propanoate) (F6.2). To a solution of (2R, 2′R)-3,3′-disulfanediylbis(2-((tert-butyloxycarbonyl)amino)propanoic acid) (F6.1) (1.0 g, 2.27 mmol) in DMF (12 mL), benzyl bromide (0.59 mL, 4.99 mmol, 2.2 equiv) and K₂CO₃ (0.721 g, 5.22 mmol, 2.3 equiv) were added. The reaction mixture was then stirred for 12 h. After the reaction was completed as monitored by TLC, the crude mixture was poured in an ice-water (100 mL) solution and the precipitate was filtered. The filtrate was washed with water and was dried to obtain the crude. The crude was subjected to silica gel column purification and eluted with a mixed solvent of 10% ethyl acetate/n-hexane to obtain the desired compound F6.2 with 85% yield. ¹H NMR (300 MHz, CDCl₃) δ 7.48-7.30 (s, 10H), 5.61-5.27 (s, 2H), 5.25-5.14 (s, 4H), 4.78-4.50 (d, J=6.8, 2H), 3.26-3.02 (d, J=5.3, 4H), 1.53-1.40 (s, 18H); ¹³C NMR (100 MHz, CDCl₃) δ 170.56, 155.05, 135.05, 128.64, 80.31, 67.57, 53.02, 41.19, 28.31 ppm.

Synthesis of R-benzyl 2-((tert-butyloxycarbonyl)amino)-3-mercaptopropanoate F6.3. Compound F6.2 (0.5 g, 0.8 mmol) was dissolved in THF (10 mL), tri-n-butylphosphine (0.2 mL, 0.9 mmol) was added and the reaction mixture was stirred at room temperature for 30 min. Then H₂O (0.1 mL, 4.0 mmol) was added and the reaction mixture was stirred at room temperature for 1 h. After the reaction was completed as monitored by TLC, the reaction mixture was extracted with ethyl acetate (150 mL) followed by water (2×50 mL) wash. The organic layer was dried over Na₂SO₄, evaporated under vacuum to produce the crude. The crude was subjected to silica gel column purification and eluted with a mixed solvent of 5-10% ethyl acetate/n-hexane to produce targeted compound F6.3 with 94% yield as a transparent liquid. ¹H NMR (400 MHz, CDCl₃) δ=7.63-7.33 (s, 5H), 5.56-5.39 (d, J=8.0, 1H), 5.43-5.03 (m, 2H), 4.80-4.52 (m, 1H), 3.13-2.69 (m, 2H), 1.85-1.50 (m, 1H), 1.38-1.64 (m, 9H).

Synthesis of Lac-thiazolidine (F6.4). (R)-Benzyl-2-((tert-butyloxycarbonyl)amino)-3-mercaptopropanoate 3 (0.2 g, 0.6 mmol) was dissolved in a mixed solvent of TFA:DCM (1:1, by volume) (10 mL) and stirred at room temperature for 2 h. After the reaction was completed as monitored by TLC, solvent was evaporated at room temperature under vacuum, the resulting residue was dried for 1 h under high vacuum. The obtained crude was dissolved in water (10 mL), neutralized with K₂CO₃ until neutral pH. The solution was extracted with ethyl acetate (3×20 mL) and the combined extracts were dried over Na₂SO₄ and concentrated to obtain the crude. The crude was directly used for the next step reaction without further purification. The crude (0.12 g, 0.56 mmol, 1.0 equiv.) was dissolved in 15 mL of acetonitrile:phosphate buffer (pH=4)=1:1 (by volume). Then D-lactose (0.19 g, 0.56 mmol, 1.0 equiv.) was added. The clear reaction mixture at pH 4 was stirred at room temperature for 36 h. The reaction was monitored (Rf=0.4) by TLC analysis using ACN:MeOH:H₂O=6:1:1 (by volume) as a developing solvent. Once the reaction was completed, solvent of the reaction mixture was completely evaporated at room temperature to produce the crude thiazolidine derivative. Then, C18 column (water/CH₃CN) purification was performed to obtain the pure Lac-thiazolidine (F6.4, 76 mg, 25%, white solid) as two diastereomers (R and S configurations). ¹H NMR (400 MHz, D₂O) R & S mixture δ 7.42-7.29 (m, 5H), 5.22-5.06 (m, 2H), 4.80-4.74 (dd, J=7.0, 4.0, 1H), 4.50-4.37 (m, 1H), 4.29-4.18 (d, J=7.5, 1H), 4.16-4.04 (dd, J=6.9, 3.5, 1H), 3.99-3.54 (m, 9H), 3.54-3.38 (m, 2H), 3.38-3.30 (m, 1H), 3.24-3.09 (dd, J=11.0, 3.3, 1H), 3.07-2.81 (m, 1H). ¹³C NMR (100 MHz, D₂O) δ 172.6, 172.0, 135.35, 135.35, 135.1, 128.88, 128.85, 128.7, 128.3, 128.3, 103.0, 102.9, 78.73, 78.73, 75.2, 75.0, 72.5, 72.4, 71.7, 71.6, 71.1, 71.0, 70.98, 70.93, 69.7, 69.3, 68.8, 68.5, 67.7, 67.6, 65.0, 64.4, 62.2, 61.9, 61.1, 60.9, 36.0, 35.7. HRMS calculated for C₂₂H₃₃NO₁₂S ([M+H]⁺) 536.1797, found 536.1781.

### Example 6. Materials and Methods for HMO Compounds Prepared Using StOPMe Strategy

Materials. All chemicals were obtained from commercial suppliers and used without further purification. ¹H NMR and ¹³C NMR spectra were recorded on a Bruker 400 MHz, a 600 MHz or a 800 MHz Bruker Avance III Spectrometer at the University of California, Davis (UC Davis) NMR Facility. High-resolution electrospray ionization (ESI-Orbitrap) mass spectra were obtained from a Thermo Scientific Q Exactive HF Orbitrap Mass Spectrometer at the Mass Spectrometry Facilities in the University of California, Davis. Thin-layer chromatography (TLC, Sorbent Technologies) was performed on silica gel plates using anisaldehyde sugar stain for detection.

Bacterial strains, plasmids, materials and general methods. E. coli chemically competent BL21 (DE3) and Origami B (DE3) cells were purchased from Invitrogen (Carlsbad, CA) and Takara Bio USA Inc. (San Jose, CA), respectively. Synthetic gene for hST6GALNAC V with codon optimized for E. coli system was synthesized by Genewiz, LLC (South Plainfield, NJ, USA). Vector plasmid pMAL-c4X and Q5 mutagenesis kit were purchased from New England Biolabs, Inc. (Beverly, MA, USA). GeneJET Plasmid Miniprep kit was from Thermo scientific (San Diego, CA, USA). NmLgtA, Hpβ3GlcNAcT, NmLgtB, Cvβ3GalT, PmST3, Pd2,6ST_A200Y/S232Y, Pd2,6ST, PmST1_M144D, SpNanA, Hm2FT, Hp3/4FT, Te2FT, SpGalK, BLUSP, BLNahK, PmGlmU, NmCSS, BfFKP, and PmPpA have been cloned in other reports.

Cloning of MBP-Δ50hST6GALNAC V-His₆. Δ50hST6GALNAC V-His₆ with an N-terminal truncation was cloned as a C-terminal His₆-tagged and N-terminal MBP fusion protein in pMAL-c4X vector using a synthetic gene with codon optimization for E. coli expression. Plasmids containing MBP-Δ50hST6GALNAC V-His₆ were transformed to the E. coli BL21 (DE3) and Origami B (DE3) cells.

Cloning of V99M, ext20, and the combination mutants of MBP-Δ50hST6GALNAC V-His₆. The V99M and ext20 mutants were cloned using the plasmid containing MBP-Δ50hST6GALNAC V-His₆ in the pMAL-c4X vector as the template for polymer chain reactions (PCR). The primers used for the V99M mutant were forward: 5′-TTGCGCGCTGGGCACCAGTAGCG-3′ (SEQ ID NO: 15) and reverse: 5′-TCACGGCAGTGCATTTTCAGC-3′ (SEQ ID NO: 16). The primers used for the ext20 mutant were forward: 5′-CGATACAGCTTGCACCAGACTCCGAGACATCATCATCATCATCATCACTGATC-3′ (SEQ ID NO: 17) and reverse: 5′-AGGGTTACAGTCGGGCATTGACATTCCTTGGGATCCGAAAACCGGTTTG-3′(SEQ ID NO: 18). To clone the combination mutant, the plasmid for the V99M mutant was used as the template for PCR using the same primers to clone the ext20 mutant. Cloning was performed by following the procedures described in the manual of Q5® Site-Directed Mutagenesis Kit. Briefly, PCR was carried out in a total volume of 25 μL containing 12.5 μL of Q5 Hot Start High-Fidelity 2× Master Mix, 10 ng (1 μL) of plasmid pMAL-c4X-MBP-Δ50hST6GALNAC V-His₆ as the template, 0.5 M (1.25 μL) of each primer, and 9 μL of nuclease-free water. Thirty cycles of PCR were completed (30 seconds of annealing with an annealing temperature of 69° C. for the V99M mutant and 61° C. for the ext20 mutant, 3.5 minutes of extension at 72° C.) followed by two more minutes of extension at 72° C. and incubation at 4° C. After a standard KLD reaction by preparing a solution of 10 μL each containing 1 μL of PCR product, 5 μL of 2×KLD Reaction Buffer, 1 μL of 10×KLD Enzyme Mix, and 3 μL of Nuclease-free Water, and incubation at room temperature for 5 minutes, the mixture was transformed to DH5α competent cell prepared by Mix and Go!™ Competent Cells protocol (Zymo Research). The plasmids with target mutants were confirmed by DNA sequencing.

Protein expression in E. coli BL21(DE3) cells. E. coli BL21 (DE3) cells harboring the recombinant plasmid containing the target gene were cultured in 50 mL Luria-Bertani (LB) media (10 g L⁻¹ tryptone, 5 g L⁻¹ yeast extract, and 10 g L⁻¹ NaCl) containing 0.1 mg mL⁻¹ ampicillin and incubated at 37° C. for overnight with shaking at 220 rpm in a C25KC incubator shaker (New Brunswick Scientific, Edison, NJ). Then 15 mL of the overnight cell culture was transferred to 1 L of LB media containing 0.1 mg mL⁻¹ ampicillin and the resulting culture was incubated at 37° C. When the OD₆₀₀ₙₘ of the cell culture reached 0.6-0.8 (usually reach in 2-3 h), isopropyl-1-thio-β-D-galactopyranoside (IPTG, 0.1 mM) was added to induce the expression of the recombinant enzyme. The culture was then incubated at 20° C. for 20 h with shaking at 220 rpm.

Protein expression in E. coli Origami B(DE3) cells. E. coli Origami B(DE3) cells with pGro7 harboring the recombinant plasmid containing the target gene were cultured at 37° C. in 50 mL LB media containing 0.05 mg mL-1 ampicillin, 0.025 mg mL⁻¹ kanamycin, 0.0175 mg mL⁻¹ chloramphenicol, 0.01 mg mL⁻¹ tetracycline with rapid shaking at 220 rpm. Then 15 mL of the overnight cell culture was transferred to 1 L of LB media containing 0.05 mg mL-1 ampicillin, 0.025 mg mL-1 kanamycin, 0.0175 mg mL⁻¹ chloramphenicol, 0.01 mg mL⁻¹ tetracycline, and L-arabinose 0.5 mg mL⁻¹ and incubated at 37° C. When the OD600 nm of the cell culture reached 0.5-0.6, IPTG (0.1 mM) was added to induce the expression of the recombinant enzyme followed by incubation at 16° C. for 48 h with shaking at 220 rpm in a C25KC incubator shaker (New Brunswick Scientific, Edison, NJ).

Protein purification. Bacterial cells were harvested by centrifugation at 4° C. in a Thermo Lynx 6000 centrifuge with Rotor Lynx F9-6×1000 at 4,392×g for 30 min. The cell pellet was re-suspended with lysis buffer (Tris-HCl buffer, 100 mM, pH 8.0 containing 0.1% Triton X-100) (20 mL for cells collected from 1 L cell culture). The cells were lysed by homogenization using an Avestin EmulsiFlex-C3 homogenizer or sonication using a Sonics Vibra-Cell system with the following protocol: 10 s (sonication)/30 s (rest) for a total of 6 min on ice. Cell lysate was obtained by centrifugation at 4° C. with Sorvall ST16R, HIGHConic II 6×94 mL fixed angle rotor at 9,016×g for 1 h and the supernatant (lysate) was collected. Protein purification was carried out using 5 mL Bio-Scale Mini Profinity IMAC Cartridges in a Bio-Rad NGC 100 Medium-Pressure Chromatography System with a flow rate of 5 mL min⁻¹. The supernatant was loaded to the Ni²⁺-NTA column pre-equilibrated with 10 column volumes of a binding buffer (5 mM imidazole, 0.5 M NaCl, 50 mM Tris-HCl buffer, pH 7.5). The column was washed with 10 column volumes of the binding buffer followed by 10 column volumes of a washing buffer (10 mM imidazole, 0.5 M NaCl, 50 mM Tris-HCl buffer, pH 7.5). The target protein was eluted with 10 column volumes of a elution buffer (200 mM imidazole, 0.5 M NaCl, 50 mM Tris-HCl buffer, pH 7.5). Fractions containing the target protein were collected and stored at 4° C. The flow rate was 5 mL min⁻¹ for the supernatant loading and all other steps during the purification. PmST3, SpGalK, BLUSP, BLNahK, PmGlmU, NmCSS, and PmPpA were dialyzed against 20 mM Tris-HCl buffer, pH 7.5, lyophilized, and stored at −20° C. NmLgtA, NmLgtB, PmST3, PmST1_M144D Pd2,6ST_A200Y/S232Y, Pd2,6ST, Hp3/4FT, BfFKP, SpNanA, SpGalK, BLUSP, BLNahK, PmGlmU, NmCSS, and PmPpA were dialyzed against a dialysis buffer (10% glycerol, 20 mM Tris-HCl buffer, pH 7.5) and stored at 4° C. The purified Hpβ3GlcNAcT, Cvβ3GalT, Hm2FT, and Te2FT without dialysis were added with 10% glycerol and stored at −20° C. Mutants of MBP-Δ50hST6GALNAC V-His₆ were dialyzed in a dialysis buffer (10% glycerol, 50 mM Tris-HCl buffer, pH 7.5, 250 mM NaCl) and stored at −20° C.

Acceptor substrate specificity of MBP-Δ50hST6GALNAC V-His₆ and mutants. Reactions were performed in a reaction mixture (10 L) containing pH 7.5 Tris-HCl (100 mM), CMP-Neu5Ac (2 mM), one acceptor (1 mM), MgCl₂ (20 mM), and MBP-Δ50hST6GALNAC V-His₆ (0.22 mU or 2.2 mU). The reactions were allowed to proceed at 37° C. for 30 min and 20 h, respectively. The reactions were stopped by adding 10 μL of cold ethanol to each reaction mixture, followed by incubation on ice for 30 min and centrifugation at 11,337 g for 10 min. Chromatographic separation and detection were achieved with an Infinity 1290 II HPLC (UHPLC) equipped with 1260 Infinity II Diode Array Detector WR (monitored at 215 nm, Agilent Technologies, CA) with a ZORBAX Bonus-RP (1.8 m particle, 2.1×150 mm, Agilent Technologies, CA) and an gradient flow (0.25 mL/min) of a mixed solvent (5% to 30% acetonitrile, and 95% to 70% of water with 0.1% TFA).

Ten gram-scale synthesis of LacβNHCbz (1) with an improved procedure. D-Lactose monohydrate (10.0 g, 27.8 mmol) was dissolved in 60 mL NH₄₀H in a 500 mL round bottom flask, NH₄HCO₃ (3 g, 38 mmol) was then added. The reaction was heated to 45-47° C. in an oil bath using water condenser for 20 h to form LacβNH₂. The solvent was removed by rotary evaporation, and LacβNH₂ was re-dissolved in 20% Na₂CO₃ solution (65 mL) in a 1 L round bottom flask. The solution was incubated in an ice bath. Benzyl chloroformate (9.86 mL, 69.4 mmol, 2.5 equiv.) was dissolved in ethanol (“EtOH”) (15 mL) in a 50 mL centrifuge tube, and the mixture was added drop-wisely to the LacβNH₂ in aqueous Na₂CO₃ solution. Another 70 mL of EtOH was added to the reaction. The reaction pH was adjusted by adding 20% Na₂CO₃ (−35 mL) solution and was kept at pH 8-9. The reaction was then removed from the ice bath and kept at room temperature for 4 hours. Furthermore, another batch of benzyl chloroformate (11.8 mL, 83.2 mmol, 3.0 equiv.) was dissolved in EtOH (20 mL) and added to the reaction mixture, followed by addition of EtOH (−100 mL) to prevent precipitation. Again, to maintain the pH, 20% sodium carbonate (−50 mL) was added drop wisely. Once the pH is adjusted, the reaction was stirred for 20 h. After the reaction was completed as monitored by TLC with EA:MeOH:H₂O=7:2:1 (by volume) as the developing solvent, EtOH was removed from the reaction by rotary evaporation. To the resulting residue in a 1 L round bottom flask, 600 mL of a mixed solvent H₂O and ethyl acetate (1:1 by volume) was added. The mixture was transferred to a separation funnel to extract the crude product in the aqueous layer. The ethyl acetate (referred to herein as “EA” or EtOAc) layer was washed with 60 mL of water each time for three times. The aqueous layer samples were combined, the solvent was removed by rotary evaporation, and the crude product (55 g) was collected. For crystallization, 275 mL of a mixed solvent (5 mL for each gram of crude) of H₂O and 1-butanol (1:1 by volume) was transferred to a 1 μL Erlenmeyer flask. It was heated to almost boiling. To the solvent, 55 g crude was added and completely dissolved. After cooling the mixture to room temperature, a small amount of pure compound was added for seeding. The mixture was incubated at 4° C. for 24 h and the crystals were filtered and collected. The flow-through was collected, dried and used for recrystallization by following a procedure similar to that described above. A total of 9.6 g (73% yield) pure crystals were obtained from two rounds of crystallization procedures.

Single C18-cartridge purification process for StOPMe-synthesized βNHCbz-tagged HMOs. After the reaction was completed, the reaction mixture was incubated in a boiling water bath for 5 min to denature the enzymes. The mixture was then cooled down to room temperature and centrifuged at 9016×g at 4° C. for 30 min. The supernatant was collected. The precipitate was washed twice, each time with H₂O (3 mL), and the supernatants were combined. The combined supernatant was concentrated by rotavap to reduce the volume to about 3-5 mL which was purified by passing through a ODS-SM column (51 g, 50 μm, 120 Å, Yamazen) pre-equilibrated with three column volumes of mobile phase A (water) on a CombiFlash® Rf 200i system and monitored at 214 nm. The product was eluted with a mixed solvent of acetonitrile and water with a flow rate of 20 mL min¹. The eluting program used was the following: Mobile phase A: water (v/v); Mobile phase B: acetonitrile (v/v); 0% B for 8 min followed by gradient 0% to 40% B over 25 min, gradient 40% to 100% B over 3 min, 100% B for 2 min, then 100% to 80% B over 2 min.

### Example 7. Preparative-Scale Synthesis and Purification of βNHCbz-Tagged Human Milk Oligosaccharides Prepared Using the StOPMe Strategy

GalβGlcNAcβ3Galβ4GlcβNHCbz (LNTβNHCbz, 7). GlcNAc (0.32 mmol), Gal (0.32 mmol), ATP (0.55 mmol), UTP (0.55 mmol) were dissolved in water in a 50 mL centrifuge tube containing Tris-HCl buffer (100 mM, pH 7.5), MgCl₂ (20 mM), BLNahK (2 mg), PmGlmU (1.5 mg), SpGalK (2 mg), BLUSP (2 mg), and PmPpA (2 mg) to generate the glycosyltransferase donors UDP-GlcNAc and UDP-Gal. The reaction mixture was incubated at 30° C. for 14 h with agitation at 180 rpm. LacβNHCbz (100 mg, 0.21 mmol) and NmLgtA (3 mg) were then added to the reaction mixture to bring the concentration of LacβNHCbz to around 25 mM. The reaction mixture was incubated at 30° C. for 14 h with agitation at 180 rpm. The product formation was monitored by thin-layer chromatography (TLC) with ethyl acetate (EtOAc): Methanol (MeOH): H₂O=5:1.6:1 (by volume) as the developing solvent and by high-resolution mass spectrometry (HRMS). After the trisaccharide formation process went to completion, the reaction mixture was incubated in a boiling water bath for 5 min and cooled down to room temperature. In the same reaction tube, Cvβ3GalT (4.5 mg) was added and the reaction volume was kept at around 20 mM. The reaction mixture was incubated at 30° C. for 10 h with agitation at 180 rpm. The product formation was monitored with TLC EtOAc:MeOH:H₂O=5:2:1 (by volume) as the developing solvent and by HRMS. After the tetrasaccharide formation process was completed, the reaction mixture was incubated in a boiling water bath for 5 min to denature the enzymes, cooled down to room temperature, and then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (162 mg, 90%). NMR data were consistent with those described herein using the MSOPME strategy described herein.

Fucα2Galβ3GlcNAcβ3Galβ4GlcβNHCbz (LNFP-IβNHCbz, 8). GlcNAc (0.32 mmol), Gal (0.32 mmol), L-fucose (0.32 mmol), ATP (0.82 mmol), UTP (0.55 mmol) and GTP (0.32 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2 mg), BLUSP (2 mg), BfFKP (2 mg), and PmPpA (3 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were then added and the process was the same as for the preparation of LNTβNHCbz (7) described above without the final centrifugation and purification steps. After the reaction was completed, the reaction mixture was incubated in a boiling water bath for 5 min and cooled down to room temperature, Te2FT (4 mg) was added to the same reaction tube. The reaction mixture was incubated at 30° C. for 14 h with agitation at 180 rpm. The product formation was monitored by TLC with EtOAc: MeOH: H₂O=5:2.2:1 (by volume) as the developing solvent and by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (176 mg, 85%). NMR data were consistent with those described herein using the MSOPME strategy described herein.

Fucα2Galβ3(Fucα4)GlcNAcβ3Galβ4(Fucα3)GlcβNHCbz (LNTFHepβNHCbz, 23). GlcNAc (0.32 mmol), Gal (0.32 mmol), L-fucose (0.82 mmol), ATP (1.37 mmol), UTP (0.55 mmol), and GTP (0.88 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (4 mg), and PmPpA (3.5 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were then added, and the process was the same as for the preparation of LNFP-IβNHCbz (8) described above without the final centrifugation and purification steps. After the reaction was completed, the reaction mixture was incubated in a boiling water bath for 5 min to denature the enzymes and cooled down to room temperature, Hp3/4FT (3 mg) was added to the same reaction tube. The reaction mixture was incubated at 30° C. for 8 h with agitation at 180 rpm. The product formation was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (224 mg, 84%). ¹H NMR (600 MHz, D₂O) δ 7.49-7.39 (m, 5H), 5.43 (d, J=4.0 Hz, 1H), 5.18 (s, 2H), 5.15 (d, J=4.1 Hz, 1H), 5.03 (d, J=3.9 Hz, 1H), 4.88 (t, J=6.7 Hz, 1H), 4.82 (s, 1H), 4.66 (d, J=7.7 Hz, 1H), 4.60 (d, J=8.5 Hz, 1H), 4.41 (d, J=7.8 Hz, 1H), 4.35 (q, J=6.7 Hz, 1H), 4.13 (dd, J=10.6, 9.2 Hz, 1H), 4.07 (d, J=3.4 Hz, 1H), 3.93 (dq, J=10.7, 3.5 Hz, 4H), 3.89-3.67 (m, 20H), 3.64-3.56 (m, 5H), 3.51 (dt, J=9.8, 2.9 Hz, 1H), 3.46 (dd, J=9.8, 7.8 Hz, 1H), 2.06 (s, 3H), 1.27 (dd, J=10.3, 6.6 Hz, 6H), 1.16 (d, J=6.6 Hz, 3H). ¹³C NMR (150 MHz, D₂O) δ 174.16, 158.11, 136.01, 128.77, 128.46, 127.77, 103.19, 101.64, 100.58, 99.52, 98.55, 97.74, 81.83, 81.10, 77.72, 76.76, 76.44, 75.08, 74.72, 74.44, 74.38, 73.59, 73.05, 72.01, 71.97, 71.91, 71.80, 71.73, 70.85, 69.38, 69.20, 69.08, 68.71, 68.53, 68.12, 67.93, 67.78, 67.38, 67.00, 66.49, 66.21, 61.58, 61.48, 59.61, 59.42, 55.79, 22.15, 15.35, 15.33, 15.26. HRMS (ESI-Orbitrap) m/z: [M+H]⁺ calculated for C₅₂H₈₃N₂O₃₄ 1279.4827; found 1279.4829.

Galβ3GlcNAcβ3Galβ4(Fucα3)GlcβNHCbz (LNFP-VβNHCbz, 9). GlcNAc (0.32 mmol), Gal (0.32 mmol), L-fucose (0.32 mmol), ATP (0.82 mmol), UTP (0.55 mmol), and GTP (0.32 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (2 mg), and PmPpA (2 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were added into the reaction mixture to bring the concentration of LacβNHCbz to around 20 mM. The reaction mixture was incubated at 30° C. for 14 h with agitation at 180 rpm. The product formation was monitored by TLC with EtOAc:MeOH:H₂O=5:1.6:1 (by volume) as the developing solvent and by HRMS. After the formation of the trisaccharide was completed, the reaction mixture was incubated in a boiling water bath for 5 min and cooled down to room temperature, Hp3/4FT (1.5 mg) was added to the same reaction tube and the reaction concentration was kept around 20 mM. The reaction mixture was incubated at 30° C. for 5 h with agitation at 180 rpm. The product formation was monitored by TLC with EtOAc:MeOH:H₂O=5:2:1 (by volume) as the developing solvent and by HRMS. After the reaction was completed, the reaction mixture was incubated in a boiling water bath for 5 min and cooled down to room temperature. In the same reaction tube, Cvβ3GalT (4 mg) was added, and the reaction concentration was kept around 15 mM. The reaction mixture was incubated at 30° C. for 10 h with agitation at 180 rpm. The product formation was monitored by TLC with EtOAc:MeOH:H₂O=5:2.4:1 (by volume) as the developing solvent and by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (183 mg, 88%). NMR data were consistent with those described herein using the MSOPME strategy described herein.

Galβ33(Fucα4)GlcNAcβ3Galβ4(Fucα3)GlcβNHCbz (LNDFH-IIβNHCbz, 10). GlcNAc (0.32 mmol), Gal (0.32 mmol), L-fucose (0.55 mmol), ATP (1.1 mmol), UTP (0.55 mmol), and GTP (0.59 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (3 mg), and PmPpA (3 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were then added and the process was the same as for the preparation of LNFP-VβNHCbz (9) described above without the final centrifugation and purification steps. After the reaction was completed, the reaction mixture was incubated in a boiling water bath for 5 min and cooled down to room temperature, Hp3/4FT (1.5 mg) was added to the same reaction tube. After the reaction was completed, the reaction mixture was incubated in a boiling water bath for 5 min to denature the enzymes, cooled down to room temperature, then centrifuged at 8000 rpm for min at 4° C. The supernatant was concentrated and purified by a C18 cartridge (51 g, 50 μm, 120 Å, Yamazen) on a CombiFlash system to obtain the pure product as a white powder (210 mg, 88%). NMR data were consistent with those described herein using the MSOPME strategy described herein.

Fucα2Galβ3GlcNAcβ3Galβ4(Fucα3)GlcβNHCbz (LNDFH-IIIβNHCbz, 24). GlcNAc (0.32 mmol), Gal (0.32 mmol), L-fucose (0.59 mmol), ATP (1.1 mmol), UTP (0.55 mmol), and GTP (0.59 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (3 mg), and PmPpA (3 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were then added and the process was the same as for the preparation of LNFP-VβNHCbz (9) described above without the final centrifugation and purification steps. After the reaction was completed, the reaction mixture was incubated in a boiling water bath for 5 min and cooled down to room temperature. Te2FT (5 mg) was added to the same reaction tube. The reaction mixture was incubated at 30° C. for 16 h with agitation at 180 rpm. The product formation was monitored by HRMS. After the reaction was completed, the reaction was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (220 mg, 92%). ¹H NMR (600 MHz, D₂O) δ 7.48-7.38 (m, 5H), 5.43 (d, J=4.0 Hz, 1H), 5.25-5.15 (m, 3H), 4.81 (d, J=6.7 Hz, 1H), 4.63 (dd, J=17.3, 8.0 Hz, 2H), 4.41 (d, J=7.8 Hz, 1H), 4.29 (q, J=6.6 Hz, 1H), 4.08 (d, J=3.4 Hz, 1H), 3.99 (dd, J=10.5, 8.5 Hz, 1H), 3.95-3.64 (m, 22H), 3.61-3.45 (m, 6H), 2.05 (s, 3H), 1.24 (d, J=6.6 Hz, 3H), 1.16 (d, J=6.6 Hz, 3H). ¹³C NMR (150 MHz, D₂O) δ 174.19, 158.12, 136.01, 128.77, 128.46, 127.77, 103.20, 101.64, 100.22, 99.49, 98.54, 81.82, 81.11, 77.72, 77.13, 76.75, 76.65, 75.16, 75.03, 74.39, 73.46, 73.05, 71.90, 71.86, 71.81, 70.91, 69.38, 69.19, 69.10, 68.50, 68.45, 67.93, 67.37, 66.49, 66.44, 61.46, 61.13, 60.36, 59.61, 55.01, 22.11, 15.28, 15.25. HRMS (ESI-Orbitrap) m/z: [M+H]⁺ calculated for C₄₆H₇₃N₂O₃₀ 1133.4248; found 1133.4214.

Galβ33(Fucα4)GlcNAcβ3Galβ4GlcβNHCbz (LNFP-IIβNHCbz, 25). GlcNAc (0.32 mmol), Gal (0.32 mmol), L-fucose (0.32 mmol), ATP (0.82 mmol), UTP (0.55 mmol), and GTP (0.32 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (2 mg), and PmPpA (2 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were added into the reaction mixture containing Tris-HCl buffer (100 mM, pH 7.5), MgCl₂ (20 mM), and water was added to bring the concentration of LacβNHCbz (1) to around 20 mM. The reaction mixture was incubated at 30° C. for 14 h with agitation at 180 rpm. The product formation was monitored by TLC with EtOAc:MeOH:H₂O=5:1.6:1 (by volume) as the developing solvent and by HRMS. After getting the trisaccharide the reaction was incubated in a boiling water bath for 5 min and cooled down to room temperature. In the same reaction tube, CTP (0.53 mmol) and Neu5Ac (0.32 mmol) were added. The pH was adjusted to pH 7.5 by adding 4 M NaOH. NmCSS (1 mg) and Pd2,6ST (1.2 mg) were then added to the reaction tube. The reaction mixture was incubated at 30° C. for 4 h with agitation at 180 rpm. The product formation was monitored by TLC with EtOAc:MeOH:H₂O=5:2:1 (by volume) as the developing solvent and by HRMS. After the trisaccharide formation was completed, the reaction mixture was incubated in a boiling water bath for 5 min and cooled down to room temperature. In the same reaction tube, Cvβ3GalT (4 mg) was added and the reaction concentration was kept at around 15 mM. The reaction mixture was incubated at 30° C. for 20 h with agitation at 180 rpm. The product formation was monitored by TLC with EtOAc:MeOH:H₂O=5:2.2:1 (by volume) as the developing solvent and by HRMS. After the pentasaccharide formation was completed, the reaction mixture was incubated in a boiling water bath for 5 min and cooled down to room temperature. In the same reaction tube, Hp3/4FT (1.5 mg) was added. The reaction mixture was incubated at 30° C. for 8 h with agitation at 180 rpm. The product formation was monitored by TLC with EtOAc:MeOH:H₂O=5:2.4:1 (by volume) as the developing solvent and by HRMS. After the reaction was completed, the reaction mixture was incubated in a boiling water bath for 5 min to denature the enzymes and cooled down to room temperature. Then the sialidase SpNanA (0.3 mg) was added to catalyze the cleavage of Neu5Ac. The reaction was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (203 mg, 85%). ¹H NMR (600 MHz, D₂O) δ 7.50-7.39 (m, 5H), 5.23-5.16 (m, 2H), 5.03 (d, J=4.0 Hz, 1H), 4.88 (d, J=6.7 Hz, 1H), 4.83 (s, 1H), 4.70 (d, J=8.4 Hz, 1H), 4.51 (d, J=7.7 Hz, 1H), 4.44 (d, J=7.9 Hz, 1H), 4.15 (d, J=3.3 Hz, 1H), 4.08 (t, J=9.7 Hz, 1H), 3.98-3.85 (m, 6H), 3.82-3.52 (m, 18H), 3.49 (dd, J=9.8, 7.7 Hz, 1H), 3.42 (t, J=8.9 Hz, 1H), 2.03 (s, 3H), 1.18 (d, J=6.6 Hz, 3H). ¹³C NMR (150 MHz, D₂O) δ 174.74, 158.09, 136.00, 128.77, 128.47, 127.82, 102.84, 102.83, 102.60, 98.00, 81.98, 81.64, 77.71, 76.12, 75.88, 75.20, 75.00, 74.85, 74.79, 72.29, 72.11, 71.92, 71.39, 70.48, 69.95, 69.11, 68.33, 68.31, 67.77, 67.40, 66.83, 61.63, 60.96, 59.84, 59.58, 55.85, 22.26, 15.35. HRMS (ESI-Orbitrap) m/z: [M+H]⁺ calculated for C₄₀H₆₃N₂O₂₆ 987.3669; found 987.3668.

Fucα2Galβ3(Fucα4)GlcNAcβ3Galβ4GlcβNHCbz (LNDFH-IβNHCbz, 26). GlcNAc (0.32 mmol), Gal (0.32 mmol), L-fucose (0.55 mmol), ATP (1.1 mmol), UTP (0.55 mmol), and GTP (0.59 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (3 mg), and PmPpA (3 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were then added and the process was the same as for the formation of the Neu5Ac-protected LNTβNHCbz pentasaccharide intermediate during the preparation of LNFP-IIβNHCbz (25) described above. The reaction mixture containing the Neu5Ac-protected LNTβNHCbz pentasaccharide intermediate was incubated in a boiling water bath for 5 min and cooled down to room temperature. In the same reaction tube, Te2FT (4 mg) was added and the reaction mixture was incubated at 30° C. for 14 h with agitation at 180 rpm. The product formation was monitored by HRMS. After the reaction was completed, the reaction was incubated in a boiling water bath for 5 min to denature the enzymes and cooled down to room temperature. In the same reaction tube, Hp3/4FT (1.5 mg) was added. The reaction mixture was incubated at 30° C. for 8 h with agitation at 180 rpm. The product formation was monitored by HRMS. After the reaction was completed, the reaction mixture was incubated in a boiling water bath for 5 min to denature the enzymes and cooled down to room temperature. Then the sialidase SpNanA (0.3 mg) was added to catalyze the cleavage of Neu5Ac and the reaction was monitored by HRMS. Once the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (193 mg, 80%). ¹H NMR (600 MHz, D₂O) δ 7.47-7.39 (m, 5H), 5.25-5.17 (m, 2H), 5.15 (d, J=4.1 Hz, 1H), 5.02 (d, J=3.9 Hz, 1H), 4.87 (t, J=6.7 Hz, 1H), 4.82 (d, J=9.5 Hz, 1H), 4.66 (d, J=7.7 Hz, 1H), 4.60 (d, J=8.4 Hz, 1H), 4.42 (d, J=7.9 Hz, 1H), 4.34 (q, J=6.7 Hz, 1H), 4.17-4.10 (m, 2H), 3.92 (tt, J=6.8, 3.7 Hz, 3H), 3.88-3.50 (m, 25H), 3.41 (t, J=8.9 Hz, 1H), 2.09-2.04 (m, 3H), 1.27 (dd, J=9.3, 6.6 Hz, 6H). ¹³C NMR (150 MHz, D₂O) δ 174.16, 158.09, 135.99, 128.77, 128.47, 127.81, 103.21, 102.87, 100.61, 99.54, 97.75, 81.64, 81.45, 77.57, 76.46, 76.14, 75.15, 74.98, 74.78, 74.73, 74.45, 73.60, 71.97, 71.95, 71.75, 71.39, 70.15, 69.42, 69.08, 68.72, 68.62, 68.24, 67.78, 67.40, 67.01, 66.22, 61.57, 60.95, 59.83, 59.44, 55.73, 22.15, 15.34, 15.30. HRMS (ESI-Orbitrap) m/z: [M+H]⁺ calculated for C₄₆H₇₃N₂O₃₀ 1133.4248; found 1133.4211.

Galβ4GlcNAcβ3Galβ4GlcβNHCbz (LNnTβNHCbz, 3). GlcNAc (0.32 mmol), Gal (0.32 mmol), ATP (0.55 mmol), UTP (0.55 mmol) were dissolved in water in a 50 mL centrifuge tube containing Tris-HCl buffer (100 mM, pH 7.5), MgCl₂ (20 mM), BLNahK (2 mg), PmGlmU (1.5 mg), SpGalK (2 mg), BLUSP (2 mg), and PmPpA (2 mg) to generate UDP-GlcNAc and UDP-Gal. The reaction mixture was incubated at 30° C. for 14 h with agitation at 180 rpm. LacβNHCbz (100 mg, 0.21 mmol) and NmLgtA (3 mg) were added and the concentration of LacβNHCbz was around 25 mM. The reaction mixture was incubated at 30° C. for 14 h with agitation at 180 rpm. The product formation was monitored by TLC with EtOAc:MeOH:H₂O=5:1.6:1 (by volume) as the developing solvent and by HRMS. After the trisaccharide formation was completed, the reaction mixture was incubated in a boiling water bath for 5 min and cooled down to room temperature. In the same reaction tube, NmLgtB (0.5 mg) was added and the reaction concentration was kept at around 20 mM. The reaction mixture was incubated at 30° C. for 4 h with agitation at 180 rpm. The product formation was monitored by TLC with EtOAc:MeOH:H₂O=5:2:1 (by volume) as the developing solvent and by HRMS. After the tetrasaccharide formation was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (161 mg, 91%). NMR data were consistent with those described herein using the MSOPME strategy described herein.

Fucα2Galβ4GlcNAcβ3Galβ4GlcβNHCbz (LNnFP-IβNHCbz, 27). GlcNAc (0.32 mmol), Gal (0.32 mmol), L-fucose (0.32 mmol), ATP (0.82 mmol), UTP (0.55 mmol), and GTP (0.32 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2 mg), BLUSP (2 mg), BfFKP (2 mg), and PmPpA (3 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were then added and the process was the same as for the preparation of LNnTβNHCbz (3) described above without the final centrifugation and purification steps. After the tetrasaccharide formation was completed, the reaction mixture was incubated in a boiling water bath for 5 min and cooled down to room temperature. In the same reaction tube, Hm2FT (3 mg) was added. The reaction mixture was incubated at 30° C. for 14 h with agitation at 180 rpm. The product formation was monitored by TLC with EtOAc:MeOH:H₂O=5:2.2:1 (by volume) as the developing solvent and by HRMS. After the reaction was completed, the reaction was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (191 mg, 91%). ¹H NMR (600 MHz, D₂O) δ 7.48-7.39 (m, 5H), 5.31 (d, J=3.2 Hz, 1H), 5.18 (d, J=3.7 Hz, 2H), 4.70 (d, J=8.4 Hz, 1H), 4.54 (d, J=7.8 Hz, 1H), 4.44 (d, J=7.8 Hz, 1H), 4.22 (q, J=6.7 Hz, 1H), 4.14 (d, J=3.4 Hz, 1H), 3.98-3.86 (m, 4H), 3.79 (ddt, J=12.5, 8.3, 5.1 Hz, 9H), 3.70 (dddd, J=27.1, 13.8, 6.8, 3.9 Hz, 10H), 3.61-3.57 (m, 1H), 3.48-3.39 (m, 2H), 2.04 (s, 3H), 1.23 (d, J=6.6 Hz, 3H). ¹³C NMR (150 MHz, D₂O) δ 174.89, 158.09, 136.00, 128.77, 128.47, 127.82, 102.82, 102.73, 100.23, 99.39, 81.88, 81.64, 77.70, 76.42, 76.12, 75.87, 75.23, 75.07, 75.00, 74.83, 73.51, 72.03, 71.65, 71.40, 69.98, 69.59, 69.09, 68.32, 68.18, 67.40, 66.92, 61.09, 60.92, 59.99, 59.84, 55.36, 22.17, 15.29. HRMS (ESI-Orbitrap) m/z: [M+H]⁺ calculated for C₄₀H₆₃N₂O₂₆ 987.3669; found 987.3652.

Fucα2Galβ4(Fucα3)GlcNAcβ3Galβ4(Fucα3)GlcβNHCbz (LNnTFHepβNHCbz, 28). GlcNAc (0.32 mmol), Gal (0.32 mmol), L-fucose (0.82 mmol), ATP (1.37 mmol), UTP (0.55 mmol), and GTP (0.88 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (4 mg), and PmPpA (3.5 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were then added and the process was the same as for the preparation of LNnFP-IβNHCbz (27) described above without the final centrifugation and purification steps. After the reaction was completed, the reaction mixture was incubated in a boiling water bath for 5 min to denature the enzymes and cooled down to room temperature. In the same reaction tube, Hp3/4FT (4 mg) was added. The reaction mixture was incubated at 30° C. for 10 h with agitation at 180 rpm. The product formation was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (223 mg, 83%). ¹H NMR (600 MHz, D₂O) δ 7.49-7.38 (m, 5H), 5.44 (d, J=4.0 Hz, 1H), 5.28 (d, J=3.5 Hz, 1H), 5.18 (s, 2H), 5.12 (d, J=4.0 Hz, 1H), 4.88 (t, J=6.7 Hz, 1H), 4.71 (d, J=8.5 Hz, 1H), 4.51 (d, J=7.8 Hz, 1H), 4.42 (d, J=7.8 Hz, 1H), 4.25 (q, J=6.7 Hz, 1H), 4.08 (d, J=3.4 Hz, 1H), 4.01-3.90 (m, 6H), 3.89-3.56 (m, 25H), 3.52-3.43 (m, 2H), 2.02 (s, 3H), 1.25 (dd, J=16.6, 6.6 Hz, 6H), 1.16 (d, J=6.6 Hz, 3H). ¹³C NMR (150 MHz, D₂O) δ 174.65, 158.11, 136.02, 128.77, 128.46, 127.77, 102.42, 101.66, 100.17, 99.39, 98.57, 98.46, 81.81, 81.37, 77.59, 76.73, 76.33, 75.30, 74.84, 74.78, 74.44, 73.54, 73.03, 71.99, 71.92, 71.88, 71.68, 70.69, 69.71, 69.19, 69.15, 68.73, 68.25, 68.22, 67.95, 67.67, 67.38, 66.90, 66.76, 66.50, 61.46, 61.42, 59.76, 59.61, 56.04, 22.24, 15.44, 15.41, 15.18. HRMS (ESI-Orbitrap) m/z: [M+H]⁺ calculated for C₅₂H₈₃N₂O₃₄ 1279.4827; found 1279.4783.

Galβ4GlcNAcβ3Galβ4(Fucα3)GlcβNHCbz (LNnFP-VβNHCbz, 5). GlcNAc (0.32 mmol), Gal (0.32 mmol), L-fucose (0.32 mmol), ATP (0.82 mmol), UTP (0.55 mmol), and GTP (0.32 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (2 mg), and PmPpA (2 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were added into the reaction mixture to bring the concentration of LacβNHCbz to around 20 mM. The reaction mixture was incubated at 30° C. for 14 h with agitation at 180 rpm. The product formation was monitored by TLC with EtOAc:MeOH:H₂O=5:1.6:1 (by volume) as the developing solvent and by HRMS. After the trisaccharide formation was completed, the reaction mixture was incubated in a boiling water bath for 5 min and cooled down to room temperature. In the same reaction tube, Hp3/4FT (1.5 mg) was added and the reaction volume was kept to maintain the acceptor substrate concentration to around 20 mM. The reaction mixture was incubated at 30° C. for 5 h with agitation at 180 rpm. The product formation was monitored by TLC with EtOAc:MeOH:H₂O=5:2:1 (by volume) as the developing solvent and by HRMS. After the reaction was completed, the reaction mixture was incubated in a boiling water bath for 5 min and cooled down to room temperature. In the same reaction tube, NmLgtB (0.5 mg) was added and the reaction acceptor concentration was kept to around 15 mM. The reaction mixture was incubated at 30° C. for 4 h with agitation at 180 rpm. The product formation was monitored by TLC with EtOAc:MeOH:H₂O=5:2.4:1 (by volume) as the developing solvent and by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (183 mg, 88%). NMR data were consistent with those described herein using the MSOPME strategy described herein.

Galβ4(Fucα3)GlcNAcβ3Galβ4(Fucα3)GlcβNHCbz (LNnDFH-IIβNHCbz, 6). GlcNAc (0.32 mmol), Gal (0.32 mmol), L-fucose (0.55 mmol), ATP (1.1 mmol), UTP (0.55 mmol), and GTP (0.59 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (3 mg), and PmPpA (3 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were then added and the process was the same as for the preparation of LNnFP-VβNHCbz (5) described above without the final centrifugation and purification steps. After the reaction was completed, the reaction mixture was incubated in a boiling water bath for 5 min and cooled down to room temperature. In the same reaction tube, Hp3/4FT (1.5 mg) was added and the reaction was monitored by HRMS. After the reaction was completed, the reaction mixture was incubated in a boiling water bath for 5 min to denature the enzymes, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (207 mg, 87%). NMR data were consistent with those described herein using the MSOPME strategy described herein.

Fucα2Galβ4GlcNAcβ3Galβ4(Fucα3)GlcβNHCbz (LNnDFH-IIIβNHCbz, 29). GlcNAc (0.32 mmol), Gal (0.32 mmol), L-fucose (0.59 mmol), ATP (1.1 mmol), UTP (0.55 mmol), and GTP (0.59 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (3 mg), and PmPpA (3 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were then added and the process was the same as for the preparation of LNnFP-VβNHCbz (5) described above without the final centrifugation and purification steps. After the reaction was completed, the reaction mixture was incubated in a boiling water bath for 5 min and cooled down to room temperature. In the same reaction tube, Hm2FT (3 mg) was added to the reaction. The reaction mixture was incubated at 30° C. for 16 h with agitation at 180 rpm. The product formation was monitored by HRMS. After the reaction was completed, the reaction was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (202 mg, 85%). ¹H NMR (600 MHz, D₂O) δ 7.48-7.39 (m, 5H), 5.44 (d, J=3.9 Hz, 1H), 5.31 (d, J=3.2 Hz, 1H), 5.18 (s, 2H), 4.82 (d, J=7.0 Hz, 2H), 4.70 (d, J=8.4 Hz, 1H), 4.55 (d, J=7.7 Hz, 1H), 4.43 (d, J=7.8 Hz, 1H), 4.22 (q, J=6.6 Hz, 1H), 4.08 (d, J=3.4 Hz, 1H), 3.99-3.91 (m, 3H), 3.90-3.56 (m, 25H), 3.54-3.44 (m, 2H), 2.04 (s, 3H), 1.23 (d, J=6.6 Hz, 3H), 1.17 (d, J=6.6 Hz, 3H). ¹³C NMR (150 MHz, D₂O) δ 174.85, 158.11, 136.02, 128.78, 128.47, 127.78, 102.67, 101.67, 100.23, 99.38, 98.46, 81.81, 81.37, 77.60, 76.74, 76.41, 75.88, 75.23, 75.04, 74.45, 73.52, 73.08, 72.03, 72.00, 71.89, 71.65, 70.65, 69.59, 69.19, 69.10, 68.23, 68.18, 67.96, 67.38, 66.92, 66.50, 61.42, 61.10, 59.97, 59.60, 55.32, 22.16, 15.29, 15.19. HRMS (ESI-Orbitrap) m/z: [M+H]⁺ calculated for C₄₆H₇₃N₂O₃₀ 1133.4248; found 1133.4250.

Galβ4(Fucα3)GlcNAcβ3Galβ4GlcβNHCbz (LNFP-IIIβNHCbz, 30). GlcNAc (0.32 mmol), Gal (0.32 mmol), L-fucose (0.32 mmol), ATP (0.82 mmol), UTP (0.55 mmol), and GTP (0.32 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (2 mg), and PmPpA (2 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were added to the reaction mixture containing Tris-HCl buffer (100 mM, pH 7.5) and MgCl₂ (20 mM). Water was added to bring the concentration of LacβNHCbz to around 20 mM. The reaction mixture was incubated at 30° C. for 14 h with agitation at 180 rpm. The product formation was monitored by TLC with EtOAc:MeOH:H₂O=5:1.6:1 (by volume) as the developing solvent and by HRMS. After the trisaccharide formation was completed, the reaction mixture was incubated in a boiling water bath for 5 min and cooled down to room temperature. In the same reaction tube, CTP (0.53 mmol) and Neu5Ac (0.32 mmol) were added. The pH was adjusted to pH 7.5 by adding 4 M NaOH. NmCSS (1 mg) and Pd2,6ST (1.2 mg) were then added. The reaction mixture was incubated at 30° C. for 4 h with agitation at 180 rpm. The product formation was monitored by TLC with EtOAc:MeOH:H₂O=5:2:1 (by volume) as the developing solvent and by HRMS. After the tetrasaccharide formation was completed, the reaction mixture was incubated in a boiling water bath for 5 min and cooled down to room temperature. In the same reaction tube, NmLgtB (0.5 mg) was added and the acceptor concentration was kept around 15 mM. The reaction mixture was incubated at 30° C. for 4 h with agitation at 180 rpm. The product formation was monitored by TLC EtOAc:MeOH:H₂O=5:2.2:1 (by volume) and HRMS. After the pentasaccharide formation was completed, the reaction mixture was incubated in a boiling water bath for 5 min and cooled down to room temperature. In the same reaction tube, Hp3/4FT (1.5 mg) was added. The reaction mixture was incubated at 30° C. for 8 h with agitation at 180 rpm. The product formation was monitored by TLC with EtOAc:MeOH:H₂O=5:2.4:1 (by volume) as the developing solvent and by HRMS. After the reaction was completed, the reaction mixture was incubated in a boiling water bath for 5 min to denature the enzymes and cooled down to room temperature. Then the sialidase SpNanA (0.3 mg) was added to cleave Neu5Ac and the reaction was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (195 mg, 82%). ¹H NMR (600 MHz, D₂O) δ 7.47-7.41 (m, 5H), 5.22-5.16 (m, 2H), 5.13 (d, J=4.0 Hz, 1H), 4.84 (d, J=13.6 Hz, 1H), 4.72 (d, J=8.4 Hz, 1H), 4.45 (dd, J=14.5, 7.8 Hz, 2H), 4.15 (d, J=3.3 Hz, 1H), 3.99-3.85 (m, 8H), 3.81-3.56 (m, 17H), 3.50 (dd, J=9.8, 7.8 Hz, 1H), 3.42 (t, J=9.0 Hz, 1H), 2.03 (s, 3H), 1.18 (d, J=6.6 Hz, 3H). ¹³C NMR (150 MHz, D₂O) δ 174.68, 158.08, 136.00, 128.77, 128.47, 127.81, 102.84, 102.52, 101.75, 98.58, 81.98, 81.65, 77.69, 76.12, 75.10, 75.01, 74.89, 74.84, 74.72, 73.04, 72.45, 71.89, 71.40, 71.03, 69.96, 69.18, 68.33, 67.68, 67.40, 66.67, 61.48, 60.96, 59.85, 59.62, 55.94, 22.23, 15.29. HRMS (ESI-Orbitrap) m/z: [M+H]⁺ calculated for C₄₀H₆₃N₂O₂₆ 987.3669; found 987.3663.

Fucα2Galβ4(Fucα3)GlcNAcβ3Galβ4GlcβNHCbz (LNnDFH-IβNHCbz, 31). GlcNAc (0.32 mmol), Gal (0.32 mmol), L-fucose (0.59 mmol), ATP (1.1 mmol), UTP (0.55 mmol), and GTP (0.59 mmol) were dissolved in water in a 50 mL centrifuge tube containing Tris-HCl buffer (100 mM, pH 7.5), MgCl₂ (20 mM), BLNahK (2.5 mg), PmGlmU (2 mg), SpGalK (2.5 mg), BLUSP (2.5 mg), BfFKP (3 mg), and PmPpA (3 mg) to generate UDP-GlcNAc, UDP-Gal, and GDP-Fuc. The reaction mixture was incubated at 30° C. for 14 h with agitation at 180 rpm. LacβNHCbz (100 mg, 0.21 mmol) and NmLgtA (3 mg) were then added and the process was the same as for the formation of the Neu5Ac-protected LNnTβNHCbz pentasaccharide intermediate during the preparation of LNFP-IIIβNHCbz (30) described above. The reaction mixture containing the Neu5Ac-protected LNnTβNHCbz pentasaccharide intermediate was incubated in a boiling water bath for 5 min and cooled down to room temperature. In the same reaction tube, Hm2FT (3 mg) was added. The reaction mixture was incubated at 30° C. for 16 h with agitation at 180 rpm. The product formation was monitored by HRMS. After the reaction was completed, the reaction mixture was incubated in a boiling water bath for 5 min to denature the enzymes and cooled down to room temperature. In the same reaction tube, Hp3/4FT (1.5 mg) was added. The reaction mixture was incubated at 30° C. for 8 h with agitation at 180 rpm. The product formation was monitored by HRMS. After the reaction was completed, the reaction mixture was incubated in a boiling water bath for 5 min to denature the enzymes and cooled down to room temperature. Then the sialidase SpNanA (0.3 mg) was added to cleave Neu5Ac and the reaction was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (197 mg, 83%). ¹H NMR (600 MHz, D₂O) δ 7.51-7.36 (m, 5H), 5.28 (d, J=3.5 Hz, 1H), 5.18 (d, J=3.6 Hz, 2H), 5.12 (d, J=3.9 Hz, 1H), 4.88 (d, J=20.2 Hz, 1H), 4.83 (s, 1H), 4.72 (d, J=8.6 Hz, 1H), 4.51 (d, J=7.8 Hz, 1H), 4.44 (d, J=7.8 Hz, 1H), 4.25 (q, J=6.6 Hz, 1H), 4.14 (d, J=3.3 Hz, 1H), 4.05-3.90 (m, 5H), 3.87-3.58 (m, 22H), 3.48-3.38 (m, 2H), 2.03 (s, 3H), 1.25 (dd, J=16.9, 6.6 Hz, 6H). ¹³C NMR (150 MHz, D₂O) δ 174.70, 158.08, 135.99, 128.77, 128.47, 127.81, 102.82, 102.45, 100.19, 99.40, 98.57, 81.90, 81.65, 77.68, 76.35, 76.12, 75.35, 75.00, 74.84, 74.82, 74.73, 73.54, 73.05, 71.92, 71.68, 71.39, 69.99, 69.71, 69.15, 68.73, 68.30, 68.26, 67.67, 67.40, 66.90, 66.77, 61.45, 60.92, 59.85, 59.77, 56.09, 22.25, 15.44, 15.42. HRMS (ESI-Orbitrap) m/z: [M+H]⁺ calculated for C₄₆H₇₃N₂O₃₀ 1133.4248; found 1133.4210.

Neu5Acα3Galβ3GlcNAcβ3Galβ4GlcβNHCbz (LSTaβNHCbz, 32). The same procedure described above for the formation of LNTβNHCbz (7) from LacβNHCbz (100 mg, 0.21 mmol) was followed without the final centrifugation and purification steps. After the tetrasaccharide formation was completed, the reaction mixture was incubated in a boiling water bath for 5 min and cooled down to room temperature. In the reaction mixture in the same reaction tube, Neu5Ac (0.32 mmol), CTP (0.42 mmol), PmST1_M144D (2 mg), and NmCSS (1.2 mg) were added. The pH of the reaction mixture was adjusted to pH 7.5 by adding 4 M NaOH. The reaction mixture was incubated at 30° C. for 6 h with agitation at 180 rpm. The product formation was monitored by TLC with EtOAc:MeOH:H₂O=5:2.4:1 (by volume) as the developing solvent and by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (211 mg, 89%). ¹H NMR (600 MHz, D₂O) δ 7.51-7.36 (m, 5H), 5.18 (d, J=3.7 Hz, 2H), 4.82 (d, J=9.4 Hz, 1H), 4.74 (d, J=8.5 Hz, 1H), 4.51 (d, J=7.8 Hz, 1H), 4.44 (dd, J=7.9, 3.8 Hz, 1H), 4.15 (d, J=3.3 Hz, 1H), 4.09 (dd, J=9.8, 3.2 Hz, 1H), 3.94-3.40 (m, 29H), 2.76 (dd, J=12.4, 4.7 Hz, 1H), 2.03 (s, 6H), 1.79 (t, J=12.2 Hz, 1H). ¹³C NMR (150 MHz, D₂O) δ 174.95, 174.89, 173.87, 136.00, 128.77, 128.47, 128.41, 127.82, 127.67, 103.35, 102.84, 102.47, 99.62, 82.13, 81.83, 81.65, 78.08, 77.76, 76.12, 75.59, 75.19, 75.07, 75.01, 74.87, 72.78, 71.82, 71.40, 70.00, 69.07, 68.46, 68.36, 68.32, 68.03, 67.41, 67.23, 62.45, 61.01, 60.96, 60.50, 59.85, 59.64, 54.56, 51.64, 39.76, 23.67, 22.28, 22.02. HRMS (ESI-Orbitrap) m/z: [M−H]⁻ calculated for C₄₅H₆₈N₃O₃₀ 1130.3893; found 1130.3874.

Neu5Acα3Galβ4GlcNAcβ3Galβ4GlcβNHCbz (38). The same procedure described above for the formation of LNnTβNHCbz (3) from LacβNHCbz (100 mg, 0.21 mmol) was followed without the final centrifugation and purification steps. After the tetrasaccharide formation was completed, the reaction mixture was incubated in a boiling water bath for 5 min and cooled down to room temperature. In the same reaction tube, Neu5Ac (0.32 mmol), CTP (0.42 mmol), PmST3 (2 mg), and NmCSS (1.2 mg) were added. The pH of the reaction mixture was adjusted to pH 7.5 by adding 4 M NaOH. The reaction mixture was incubated at 30° C. for 6 h with agitation at 180 rpm. The product formation was monitored by TLC with EtOAc:MeOH:H₂O=5:2.4:1 (by volume) as the developing solvent and by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (219 mg, 92%). HRMS (ESI-Orbitrap) m/z: [M−H]⁻ calculated for C₄₅H₆₉N₃O₃₀ 1130.3893; found 1130.3874. ¹H NMR (600 MHz, D₂O) δ 7.49-7.38 (m, 5H), 5.18 (d, J=3.7 Hz, 2H), 4.70 (d, J=8.3 Hz, 1H), 4.56 (d, J=7.9 Hz, 1H), 4.44 (d, J=7.9 Hz, 1H), 4.15 (d, J=3.3 Hz, 1H), 4.12 (dd, J=9.8, 3.2 Hz, 1H), 3.99-3.62 (m, 26H), 3.58 (qd, J=9.9, 9.3, 4.7 Hz, 4H), 3.42 (t, J=8.9 Hz, 1H), 2.76 (dd, J=12.4, 4.6 Hz, 1H), 2.03 (s, 6H), 1.80 (t, J=12.1 Hz, 1H). ¹³C NMR (150 MHz, D₂O) δ 174.99, 174.87, 173.76, 158.09, 136.00, 128.77, 128.47, 127.82, 102.85, 102.78, 102.52, 99.75, 81.97, 81.65, 77.99, 77.74, 76.13, 75.47, 75.15, 75.01, 74.87, 74.54, 72.88, 72.12, 71.72, 71.40, 69.96, 69.36, 68.33, 68.30, 68.07, 67.46, 62.57, 61.01, 60.97, 59.84, 59.30, 55.16, 51.66, 46.65, 39.60, 22.16, 22.02, 19.13, 12.80, 8.21. HRMS (ESI-Orbitrap) m/z: [M−H]⁻ calculated for C₄₅H₆₈N₃O₃₀ 1130.3893; found 1130.3873.

Neu5Acα3Galβ3(Neu5Acα6)GlcNAcβ3Galβ4GlcβNHCbz (46). GlcNAc (35 mg, 0.16 mmol), Gal (29 mg, 0.16 mmol), ATP (185 mg, 0.34 mmol), and UTP (178 mg, 0.34 mmol) were dissolved in water in a 50 mL centrifuge tube containing Tris-HCl buffer (100 mM, pH 7.5), MgCl₂ (100 mM), BLNahK (1.5 mg), PmGlmU (1.2 mg), SpGalK (1.6 mg), BLUSP (1.6 mg), and PmPpA (1.6 mg) to generate UDP-GlcNAc and UDP-Gal. The reaction mixture in a 3 mL volume was incubated at 30° C. for 14 h with agitation at 180 rpm. LacβNHCbz (1) (50 mg, 0.11 mmol) and NmLgtA (2.5 mg) were added. Then water was added to bring the concentration of LacβNHCbz to around 20 mM. The reaction mixture was incubated at 30° C. for 16 h with agitation at 180 rpm. The product formation was monitored by HRMS. When all LacβNHCbz was consumed, the reaction mixture was incubated in a boiling water bath for 5 min and cooled down to room temperature. To the same reaction tube, Cvβ3GalT (2.5 mg) was added and the reaction volume was around 7 mL. The reaction mixture was incubated at 30° C. for 16 h with agitation at 180 rpm. The product formation was monitored by HRMS. When the tetrasaccharide formation was completed, the reaction mixture was incubated in a boiling water bath for 5 min and cooled down to room temperature. CTP (94 mg, 0.18 mmol) and Neu5Ac (49 mg, 0.16 mmol) were added and the pH of the solution was adjusted to pH 8.0 by adding 4 M NaOH. NmCSS (0.6 mg) and CjCst-I (2.0 mg) were added and the reaction mixture was incubated at 30° C. for 24 h with agitation at 180 rpm. The product formation was monitored by HRMS. After the reaction was completed, CTP (94 mg, 0.18 mmol) and Neu5Ac (49 mg, 0.16 mmol) were added and the pH of the solution was adjusted to 8.0 using 4 M NaOH. NmCSS (0.6 mg) and MBP-Δ50hST6GALNAC V_Design7_ext20-His₆ (6.0 mg) were added and the reaction mixture with a 10 mL volume was incubated at 30° C. with agitation at 180 rpm. The product formation was monitored by HRMS. After 20 h, additional 5 mg of MBP-Δ50hST6GALNAC V_Design7_ext20-His₆ was added and the reaction was incubated at 30° C. for another 24 h with agitation at 180 rpm. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (131 mg, 85%). ¹H NMR (800 MHz, D₂O) δ 7.46-7.37 (m, 5H), 5.17 (t, J=9.3 Hz, 2H), 4.68 (d, J=8.5 Hz, 1H), 4.48 (d, J=7.8 Hz, 1H), 4.42 (d, J=7.9 Hz, 1H), 4.15 (d, J=3.3 Hz, 1H), 4.06 (dd, J=9.8, 3.1 Hz, 1H), 3.95 (dd, J=11.0, 5.3 Hz, 1H), 3.40 (t, J=9.1 Hz, 1H), 2.73 (td, J=13.0, 4.7 Hz, 2H), 2.04-1.96 (m, 9H), 1.77 (t, J=12.2 Hz, 1H), 1.67 (t, J=12.1 Hz, 1H). ¹³C NMR (200 MHz, D₂O) δ 174.97, 174.90, 174.80, 173.87, 173.39, 135.98, 128.75, 128.44, 127.79, 103.38, 102.82, 102.52, 100.16, 99.58, 82.15, 81.77, 77.79, 76.11, 75.56, 75.04, 75.00, 74.95, 73.69, 72.74, 72.46, 71.79, 71.66, 71.36, 69.94, 69.06, 68.38, 68.30, 68.19, 68.14, 68.01, 67.37, 67.23, 62.83, 62.53, 62.40, 61.12, 61.09, 61.01, 59.83, 59.48, 54.58, 51.81, 51.61, 40.05, 39.75, 22.27, 22.01, 21.98. HRMS (ESI-Orbitrap) m/z: [M−H]⁻ calculated for C₅₆H₈₅N₄O₃₈ 1421.4847; found 1421.4830.

Neu5Acα3Galβ3(Neu5Acα6)GlcNAcβ3Galβ4(Fucα3)GlcβNHCbz (FDS-LNT-IIβNHCbz, 47). The same procedure described above for the formation of LNFPVβNHCbz (5) from LacβNHCbz (100 mg, 0.21 mmol) was followed without the final centrifugation and purification steps. After the pentasaccharide formation was completed, the reaction mixture was incubated in a boiling water bath for 5 min and cooled down to room temperature, CTP (0.42 mmol) and Neu5Ac (0.32 mmol) were added. NmCSS (1.5 mg) and PmST1_M144D (3 mg) were then added and the pH of the reaction was kept at pH 7.5. The reaction mixture was incubated at 30° C. for 6 h with agitation at 180 rpm. The product formation was monitored by HRMS. After the reaction was completed, the reaction mixture was incubated in a boiling water bath for 5 min to denature the enzymes and cooled down to room temperature. CTP (0.42 mmol) and Neu5Ac (0.32 mmol) were added. NmCSS (1.5 mg) and MBP-Δ50hST6GALNAC V-His₆ purified from 4 L culture were added. The reaction mixture was incubated at 30° C. for 30 h with agitation at 180 rpm. The product formation was monitored by TLC with EtOAc:MeOH:H₂O=5:2.2:1 (by volume) as the developing solvent and by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (132 mg, 40%). ¹H NMR (800 MHz, D₂O) δ 7.46-7.24 (m, 5H), 5.43 (d, J=4.0 Hz, 1H), 5.17 (s, 2H), 4.82-4.80 (m, 1H), 4.69 (d, J=8.8 Hz, 1H), 4.50 (d, J=8.0 Hz, 1H), 4.42 (d, J=8.0 Hz, 1H), 4.11-3.46 (m, 41H), 2.77-2.71 (m, 2H), 2.02 (s, 6H), 2.01 (s, 3H), 1.77 (t, J=12.0 Hz, 1H), 1.68 (t, J=12.0 Hz, 1H), 1.15 (d, J=6.4 Hz, 3H). ¹³C NMR (200 MHz, D₂O) δ 175.00, 174.92, 174.79, 173.88, 173.39, 158.10, 136.00, 128.75, 128.44, 127.75, 120.35, 103.37, 102.38, 101.64, 100.14, 99.59, 98.45, 81.75, 77.60, 76.75, 75.57, 75.05, 74.56, 73.68, 73.09, 72.75, 72.45, 72.02, 71.87, 71.81, 71.66, 70.59, 69.15, 69.07, 68.42, 68.38, 68.32, 68.21, 68.06, 68.02, 67.95, 67.35, 67.24, 66.51, 62.91, 62.55, 62.41, 61.62, 61.02, 60.04, 59.55, 54.59, 51.84, 51.63, 40.08, 39.76, 22.27, 22.02, 22.00, 15.20. HRMS (ESI-Orbitrap) m/z: [M−2H]²⁻ calculated for C₆₂H₉₆N₄O₄₂ 783.2677; found 783.2671.

### Example 8. The Synthesis of Sialylated HMO βNHCbz (33-37, 39-46) by One-Pot Two-Enzyme (OP2E) Sialylation Processes

Neu5Acα3Galβ3GlcNAcβ3Galβ4(Fucα3)GlcβNHCbz (34). LNFP-VβNHCbz (9) (50 mg, 0.051 mmol), CTP (0.13 mmol), and Neu5Ac (0.076 mmol) were dissolved in water in a 50 mL centrifuge tube containing Tris-HCl buffer (100 mM, pH 7.5) and MgCl₂ (20 mM). NmCSS (0.4 mg) and PmST1 M144D (0.4 mg) were added and the final concentration of LNFP-V βNHCbz (9) was about 20 mM. The reaction mixture was incubated at 30° C. for 6 h with agitation at 180 rpm. The product formation was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (59 mg, 91%). ¹H NMR (600 MHz, D₂O) δ 7.49-7.38 (m, 5H), 5.44 (d, J=4.0 Hz, 1H), 5.18 (s, 2H), 4.81 (s, 2H), 4.74 (d, J=8.5 Hz, 1H), 4.52 (d, J=7.8 Hz, 1H), 4.43 (d, J=7.8 Hz, 1H), 4.09 (dt, J=7.4, 3.4 Hz, 2H), 3.96-3.77 (m, 15H), 3.75-3.46 (m, 20H), 2.76 (dd, J=12.4, 4.6 Hz, 1H), 2.03 (d, J=3.3 Hz, 6H), 1.79 (t, J=12.1 Hz, 1H), 1.16 (d, J=6.6 Hz, 3H). ¹³C NMR (150 MHz, D₂O) δ 174.94, 174.85, 173.87, 158.10, 136.02, 128.77, 128.46, 127.78, 103.31, 102.39, 101.64, 99.61, 98.46, 82.05, 81.81, 81.32, 77.60, 76.74, 75.58, 75.16, 75.06, 74.48, 73.10, 72.78, 72.00, 71.88, 71.82, 70.69, 69.18, 69.06, 68.44, 68.36, 68.22, 68.03, 67.96, 67.38, 67.22, 66.51, 62.45, 61.46, 61.28, 61.01, 60.49, 59.60, 59.40, 54.56, 51.64, 39.77, 22.26, 22.02, 15.18. HRMS (ESI-Orbitrap) m/z: [M−H]⁻ calculated for C₅₁H₇₈N₃O₃₄ 1276.4472; found 1276.4448.

Neu5Acα3Galβ3(Fucα4)GlcNAcβ3Galβ4(Fucα3)GlcβNHCbz (36). LNDFH-IIβNHCbz (10) (50 mg, 0.044 mmol), CTP (0.11 mmol), and Neu5Ac (0.066 mmol) were dissolved in water in a 50 mL centrifuge tube containing Tris-HCl buffer (100 mM, pH 7.5) and MgCl₂ (20 mM). NmCSS (0.3 mg) and PmST1 M144D (0.4 mg) were added and the final concentration of LNDFH-IIβNHCbz (10) was about 20 mM. The reaction mixture was incubated at 30° C. for 16 h with agitation at 180 rpm. The product formation was monitored by HRMS. After the reaction was completed, the reaction was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (41 mg, 65%). ¹H NMR (400 MHz, D₂O) δ 7.56-7.31 (m, 5H), 5.44 (d, J=4.0 Hz, 1H), 5.18 (s, 2H), 5.01 (t, J=3.7 Hz, 1H), 4.93-4.79 (m, 3H), 4.83 (d, J=1.9 Hz, OH), 4.69 (dd, J=11.8, 8.5 Hz, 2H), 4.53 (dd, J=21.5, 7.7 Hz, 1H), 4.42 (d, J=7.8 Hz, 1H), 4.10-4.03 (m, 2H), 3.97-3.76 (m, 17H), 3.71-3.47 (m, 17H), 2.76 (dt, J=12.6, 5.2 Hz, 1H), 2.03 (dd, J=2.8, 1.0 Hz, 6H), 1.74 (dt, J=21.7, 12.2 Hz, 1H), 1.17 (td, J=3.7, 1.9 Hz, 6H). 13C NMR (100 MHz, D₂O) δ 174.92, 173.92, 173.46, 136.02, 135.30, 128.78, 128.47, 128.03, 127.78, 102.80, 102.43, 101.68, 100.79, 99.36, 98.46, 77.59, 76.74, 75.59, 75.22, 75.16, 74.48, 73.53, 73.10, 72.72, 72.17, 72.02, 71.88, 69.19, 69.06, 68.26, 68.15, 67.96, 67.85, 67.79, 67.40, 66.95, 66.80, 66.73, 66.51, 65.26, 62.64, 61.64, 61.48, 60.93, 59.66, 59.59, 59.50, 55.79, 22.00, 15.18.

Neu5Acα3Galβ3(Fucα4)GlcNAcβ3Galβ4GlcβNHCbz (37). LNFP-IIβNHCbz (25) (50 mg, 0.051 mmol), CTP (0.13 mmol), and Neu5Ac (0.076 mmol) were dissolved in water in a 50 mL centrifuge tube containing Tris-HCl buffer (100 mM, pH 7.5) and MgCl₂ (20 mM). NmCSS (0.4 mg) and PmST1 M144D (0.4 mg) were added into the reaction and the final concentration of LNFP-IIβNHCbz (25) was about 20 mM. The reaction mixture was incubated at 30° C. for 6 h with agitation at 180 rpm. The product formation was monitored by HRMS. After the reaction was completed, the reaction was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (56 mg, 86%). ¹H NMR (600 MHz, D₂O) δ 7.54-7.26 (m, 5H), 5.19 (d, J=3.7 Hz, 2H), 5.01 (d, J=4.0 Hz, 1H), 4.87 (d, J=6.8 Hz, 1H), 4.71 (d, J=8.5 Hz, 1H), 4.55 (d, J=7.8 Hz, 1H), 4.44 (d, J=7.9 Hz, 1H), 4.15 (d, J=3.3 Hz, 1H), 4.12-4.01 (m, 2H), 3.97-3.58 (m, 28H), 3.55-3.49 (m, 3H), 3.42 (t, J=9.0 Hz, 1H), 2.77 (dd, J=12.4, 4.7 Hz, 1H), 2.03 (d, J=5.2 Hz, 6H), 1.77 (t, J=12.2 Hz, 1H), 1.18 (d, J=6.6 Hz, 3H). ¹³C NMR (150 MHz, D₂O) δ 174.92, 174.59, 173.91, 158.09, 136.01, 128.77, 128.47, 127.82, 102.86, 102.73, 102.52, 99.37, 97.98, 81.94, 81.65, 77.76, 76.12, 75.87, 75.60, 75.25, 75.01, 74.86, 74.73, 72.73, 72.03, 71.92, 71.85, 71.39, 69.95, 69.09, 68.78, 68.40, 68.31, 68.01, 67.78, 67.40, 66.92, 66.82, 62.31, 61.64, 60.97, 59.83, 59.60, 55.80, 51.66, 39.98, 22.38, 22.03, 15.32. HRMS (ESI-Orbitrap) m/z: [M−H]⁻ calculated for C₅₁H₇₈N₃O₃₄ 1276.4472; found 1276.4432.

Neu5Acα3Galβ4GlcNAcβ3Galβ4(Fucα3)GlcβNHCbz (40). LNnFP-VβNHCbz (5) (50 mg, 0.051 mmol), CTP (0.13 mmol), and Neu5Ac (0.076 mmol) were dissolved in water in a 50 mL centrifuge tube containing Tris-HCl buffer (100 mM, pH 7.5) and MgCl₂ (20 mM). NmCSS (0.4 mg) and PmST3 (0.4 mg) were added and the final concentration of LNnFP-VβNHCbz (5) was about 20 mM. The reaction mixture was incubated at 30° C. for 6 h with agitation at 180 rpm. The product formation was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (60 mg, 92%). ¹H NMR (600 MHz, D₂O) δ 7.48-7.39 (m, 5H), 5.44 (d, J=4.0 Hz, 1H), 5.18 (s, 2H), 4.82 (d, J=6.9 Hz, 2H), 4.69 (d, J=8.3 Hz, 1H), 4.56 (d, J=7.9 Hz, 1H), 4.42 (d, J=7.8 Hz, 1H), 4.13-4.08 (m, 2H), 3.99-3.68 (m, 25H), 3.66-3.55 (m, 8H), 3.52-3.48 (m, 1H), 2.76 (dd, J=12.4, 4.6 Hz, 1H), 2.07-2.00 (m, 6H), 1.80 (t, J=12.2 Hz, 1H), 1.17 (d, J=6.6 Hz, 3H). ¹³C NMR (150 MHz, D₂O) δ 174.99, 174.83, 173.83, 136.02, 128.77, 128.46, 127.78, 102.72, 102.53, 101.67, 99.78, 98.46, 81.81, 81.51, 78.02, 77.60, 76.74, 75.47, 75.15, 74.50, 74.48, 73.09, 72.87, 72.06, 72.01, 71.89, 71.74, 70.61, 69.36, 69.18, 68.33, 68.24, 68.07, 67.97, 67.45, 67.37, 66.51, 62.56, 61.48, 61.32, 61.01, 59.83, 59.60, 59.37, 55.13, 51.66, 39.62, 22.14, 22.02, 15.19. HRMS (ESI-Orbitrap) m/z: [M−H]⁻ calculated for C₅₁H₇₈N₃O₃₄ 1276.4472; found 1276.4436.

Neu5Acα3Galβ3(Fucα4)GlcNAcβ3Galβ4(Fucα3)GlcβNHCbz (42). LNnDFH-IIβNHCbz (6) (50 mg, 0.044 mmol), CTP (0.11 mmol), and Neu5Ac (0.066 mmol) were dissolved in water in a 50 mL centrifuge tube containing Tris-HCl buffer (100 mM, pH 7.5) and MgCl₂ (20 mM). NmCSS (0.3 mg) and PmST1 M144D (0.4 mg) were added and the final concentration of LNnDFH-IIβNHCbz (6) was about 20 mM. The reaction mixture was incubated at 30° C. for 10 h with agitation at 180 rpm. The product formation was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (57 mg, 91%). ¹H NMR (400 MHz, D₂O) δ 7.49-7.39 (m, 5H), 5.44 (d, J=4.0 Hz, 1H), 5.18 (s, 2H), 5.13 (d, J=4.0 Hz, 1H), 4.84-4.81 (m, 2H), 4.70 (d, J=8.4 Hz, 1H), 4.53 (d, J=7.9 Hz, 1H), 4.42 (d, J=7.8 Hz, 1H), 4.13-4.06 (m, 2H), 3.99-3.77 (m, 18H), 3.74-3.47 (m, 19H), 2.82-2.70 (m, 1H), 2.11-1.98 (m, 6H), 1.79 (t, J=12.1 Hz, 1H), 1.16 (dd, J=6.7, 3.2 Hz, 6H). ¹³C NMR (100 MHz, D₂O) δ 175.01, 174.63, 174.37, 173.84, 128.78, 128.47, 127.78, 102.52, 101.68, 101.55, 99.64, 98.54, 98.47, 81.82, 81.54, 77.60, 76.73, 75.66, 74.98, 74.90, 74.63, 74.48, 73.10, 73.02, 72.90, 71.98, 71.89, 71.84, 70.63, 69.25, 69.17, 68.29, 68.24, 68.08, 67.96, 67.69, 67.40, 67.37, 67.29, 66.64, 66.51, 62.58, 61.48, 61.01, 59.63, 55.93, 51.68, 22.22, 22.01, 15.26, 15.18.

Neu5Acα3Gal β4(Fucα3)GlcNAcβ3Galβ4GlcβNHCbz (43). LNFP-IIIβNHCbz (30) (50 mg, 0.051 mmol), CTP (0.13 mmol), and Neu5Ac (0.076 mmol) were dissolved in water in a 50 mL centrifuge tube containing Tris-HCl buffer (100 mM, pH 7.5) and MgCl₂ (20 mM). NmCSS (0.4 mg) and PmST1 M144D (0.4 mg) were added and the final concentration of LNFP-IIIβNHCbz (30) was about 20 mM. The reaction mixture was incubated at 30° C. for 6 h with agitation at 180 rpm. The product formation was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (59 mg, 90%). ¹H NMR (400 MHz, D₂O) δ 7.56-7.35 (m, 5H), 5.19 (s, 2H), 5.12 (d, J=4.0 Hz, 1H), 4.83 (s, 2H), 4.71 (d, J=8.3 Hz, 1H), 4.53 (d, J=7.8 Hz, 1H), 4.44 (d, J=7.7 Hz, 1H), 4.16 (d, J=3.2 Hz, 1H), 4.09 (dd, J=9.8, 3.2 Hz, 1H), 3.99-3.85 (m, 11H), 3.83-3.75 (m, 5H), 3.73-3.64 (m, 14H), 3.62-3.49 (m, 5H), 3.41 (t, J=8.8 Hz, 1H), 2.76 (dt, J=12.6, 4.5 Hz, 1H), 2.10-1.98 (m, 6H), 1.75 (dt, J=37.4, 12.1 Hz, 1H), 1.17 (d, J=6.4 Hz, 3H). ¹³C NMR (100 MHz, D₂O) δ 175.01, 174.66, 173.83, 128.77, 128.47, 127.81, 102.85, 102.56, 101.54, 99.64, 98.55, 82.02, 81.67, 77.73, 76.12, 75.65, 75.01, 74.87, 73.03, 71.84, 71.39, 69.95, 69.25, 69.16, 68.29, 68.09, 67.69, 67.40, 67.30, 66.65, 66.60, 62.58, 61.47, 60.98, 59.85, 59.56, 55.94, 51.68, 22.23, 22.01, 15.26.

Neu5Acα6Galβ3GlcNAcβ3Galβ4GlcβNHCbz (LSTcβNHCbz, 33). LNTβNHCbz (7) (50 mg, 0.06 mmol), CTP (0.15 mmol), and Neu5Ac (0.09 mm ol) were dissolved in water in a 50 mL centrifuge tube containing Tris-HCl buffer (100 mM, pH 7.5) and MgCl₂ (20 mM). NmCSS (0.5 mg) and Pd2,6ST A200Y/S292Y (0.4 mg) were added and the final concentration of LNTβNHCbz (7) was about 20 mM. The reaction mixture was incubated at 30° C. for 6 h with agitation at 180 rpm. The product formation was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (62 mg, 92%). ¹H NMR (600 MHz, D₂O) δ 7.49-7.39 (m, 5H), 5.18 (d, J=3.8 Hz, 2H), 4.82 (d, J=9.7 Hz, 1H), 4.74 (d, J=8.5 Hz, 1H), 4.45 (d, J=7.9 Hz, 1H), 4.39 (d, J=7.8 Hz, 1H), 4.16 (d, J=3.3 Hz, 1H), 4.00-3.70 (m, 19H), 3.69-3.49 (m, 12H), 3.42 (t, J=8.9 Hz, 1H), 2.70 (dd, J=12.4, 4.7 Hz, 1H), 2.03 (d, J=4.5 Hz, 6H), 1.70 (t, J=12.2 Hz, 1H). ¹³C NMR (150 MHz, D₂O) δ 174.95, 174.88, 173.47, 158.08, 136.00, 128.77, 128.47, 127.81, 103.86, 102.84, 102.52, 100.11, 83.63, 81.89, 81.64, 77.76, 76.13, 75.28, 75.02, 74.90, 73.58, 72.43, 72.36, 71.80, 71.39, 70.51, 70.01, 68.64, 68.44, 68.39, 68.31, 68.28, 67.40, 63.51, 62.61, 61.17, 60.98, 60.63, 59.85, 59.48, 54.43, 51.78, 40.13, 22.19, 22.03. HRMS (ESI-Orbitrap) m/z: [M−H]⁻ calculated for C₄₅H₆₈N₃O₃₀ 1130.3893; found 1130.3860.

Neu5Acα6Galβ3GlcNAcβ3Galβ4(Fucα3)GlcβNHCbz (35) LNFP-VβNHCbz (9) (50 mg, 0.051 mmol), CTP (0.13 mmol), and Neu5Ac (0.076 mmol) were dissolved in water in a 50 mL centrifuge tube containing Tris-HCl buffer (100 mM, pH 7.5) and MgCl₂ (20 mM). NmCSS (0.4 mg) and Pd2,6ST A200Y/S292Y (0.4 mg) were added and the final concentration of LNFP-VβNHCbz (9) was about 20 mM. The reaction mixture was incubated at 30° C. for 6 h with agitation at 180 rpm. The product formation was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (60 mg, 93%). ¹H NMR (600 MHz, D₂O) δ 7.51-7.37 (m, 5H), 5.44 (d, J=4.0 Hz, 1H), 5.18 (s, 2H), 4.82 (d, J=7.0 Hz, 2H), 4.74 (d, J=8.5 Hz, 1H), 4.43 (d, J=7.8 Hz, 1H), 4.39 (d, J=7.8 Hz, 1H), 4.10 (d, J=3.4 Hz, 1H), 4.00-3.69 (m, 23H), 3.68-3.49 (m, 12H), 2.71 (dd, J=12.4, 4.7 Hz, 1H), 2.03 (d, J=6.9 Hz, 6H), 1.70 (t, J=12.2 Hz, 1H), 1.16 (d, J=6.6 Hz, 3H). ¹³C NMR (150 MHz, D₂O) δ 174.93, 174.88, 173.47, 158.11, 136.02, 128.78, 128.46, 127.78, 103.85, 102.46, 101.65, 100.12, 98.46, 83.54, 81.81, 81.42, 77.60, 76.74, 75.25, 74.50, 73.57, 73.09, 72.44, 72.35, 72.01, 71.89, 71.80, 70.68, 70.51, 69.18, 68.63, 68.44, 68.39, 68.28, 68.22, 67.97, 67.38, 66.51, 63.51, 62.61, 61.48, 61.06, 60.62, 59.56, 54.43, 51.78, 40.13, 22.18, 22.03, 15.19. HRMS (ESI-Orbitrap) m/z: [M−H]⁻ calculated for C₅₁H₇₈N₃O₃₄ 1276.4472; found 1276.4432.

Neu5Acα6Galβ4GlcNAcβ3Galβ4GlcβNHCbz (39). LNnTβNHCbz (3) (50 mg, 0.06 mmol), CTP (0.15 mmol), and Neu5Ac (0.09 mmol) were dissolved in water in a 50 mL centrifuge tube containing Tris-HCl buffer (100 mM, pH 7.5) and MgCl₂ (20 mM). NmCSS (0.5 mg) and Pd2,6ST A200Y/S292Y (0.4 mg) were added and the final concentration of LNnTβNHCbz (3) was about 20 mM. The reaction mixture was incubated at 30° C. for 6 h with agitation at 180 rpm. The product formation was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (63 mg, 94%). ¹H NMR (600 MHz, D₂O) δ 7.48-7.39 (m, 5H), 5.18 (d, J=3.6 Hz, 2H), 4.83 (s, 1H), 4.73 (d, J=7.6 Hz, 1H), 4.45 (dd, J=7.9, 5.1 Hz, 2H), 4.15 (d, J=3.3 Hz, 1H), 4.02-3.51 (m, 32H), 3.42 (t, J=9.0 Hz, 1H), 2.67 (dd, J=12.4, 4.6 Hz, 1H), 2.04 (d, J=14.9 Hz, 6H), 1.72 (t, J=12.2 Hz, 1H). ¹³C NMR (150 MHz, D₂O) δ 174.90, 173.52, 158.09, 136.01, 128.77, 128.47, 127.82, 103.43, 102.85, 102.55, 100.12, 81.92, 81.65, 80.46, 77.75, 76.12, 75.01, 74.87, 74.25, 73.68, 72.52, 72.41, 72.21, 71.69, 71.40, 70.72, 69.96, 68.38, 68.34, 68.32, 68.19, 67.40, 63.32, 62.64, 61.08, 60.97, 60.13, 59.83, 59.55, 54.92, 51.87, 40.06, 22.26, 22.01. HRMS (ESI-Orbitrap) m/z: [M−H]⁻ calculated for C₄₅H₆₈N₃O₃₀ 1130.3893; found 1130.3865.

Neu5Acα6Galβ4GlcNAcβ3Galβ4(Fucα3)GlcβNHCbz (41). LNnFP-VβNHCbz (5) (50 mg, 0.051 mmol), CTP (0.13 mmol), and Neu5Ac (0.076 mmol) were dissolved in water in a 50 mL centrifuge tube containing Tris-HCl buffer (100 mM, pH 7.5) and MgCl₂ (20 mM). NmCSS (0.4 mg) and Pd2,6ST_A200Y/S292Y (0.4 mg) were added and the final concentration of LNnFP-VβNHCbz (5) was about 20 mM. The reaction mixture was incubated at 30° C. for 6 h with agitation at 180 rpm. The product formation was monitored by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (60 mg, 92%). ¹H NMR (600 MHz, D₂O) δ 7.51-7.38 (m, 5H), 5.44 (d, J=4.0 Hz, 1H), 5.18 (s, 2H), 4.84-4.80 (m, 2H), 4.73 (d, J=7.6 Hz, 1H), 4.44 (dd, J=16.8, 7.9 Hz, 2H), 4.10 (d, J=3.4 Hz, 1H), 4.02-3.77 (m, 17H), 3.76-3.49 (m, 18H), 2.67 (dd, J=12.4, 4.7 Hz, 1H), 2.04 (d, J=11.5 Hz, 6H), 1.72 (t, J=12.2 Hz, 1H), 1.17 (d, J=6.6 Hz, 3H). ¹³C NMR (150 MHz, D₂O) δ 174.89, 174.86, 173.52, 158.10, 136.02, 128.78, 128.46, 127.78, 103.45, 102.51, 101.68, 100.12, 98.46, 81.81, 81.48, 80.47, 77.59, 76.74, 74.48, 74.22, 73.68, 73.10, 72.52, 72.40, 72.19, 72.01, 71.88, 71.69, 70.72, 70.60, 69.18, 68.38, 68.34, 68.23, 68.18, 67.97, 67.37, 66.52, 63.32, 62.63, 61.48, 61.13, 60.13, 59.59, 59.51, 54.88, 51.87, 40.06, 22.24, 22.01, 15.19. HRMS (ESI-Orbitrap) m/z: [M−H]⁻ calculated for C₅₁H₇₈N₃O₃₄ 1276.4472; found 1276.4433.

Galβ33(Neu5Acα6)GlcNAcβ3Galβ4GlcβNHCbz (LSTbβNHCbz, 44). LNTβNHCbz (7) (15 mg, 0.018 mmol), CTP (0.045 mmol), and Neu5Ac (0.027 mmol) were dissolved in water in a 15 mL centrifuge tube containing Tris-HCl buffer (100 mM, pH 7.5) and MgCl₂ (20 mM). NmCSS (0.3 mg) and MBP-Δ50hST6GALNAC V-His₆ (1/3 of the purified enzyme from 2 L culture) were added and the concentration of LNTβNHCbz (7) was about 10 mM. The reaction mixture was incubated at 30° C. for 42 h with agitation at 180 rpm. The product formation was monitored by TLC with EtOAc:MeOH:H₂O=5:2.2:1 (by volume) as the developing solvent and by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (18 mg, 89%). ¹H NMR (800 MHz, D₂O) δ 7.48-7.37 (m, 5H), 5.16 (d, J=6.3 Hz, 2H), 4.68 (d, J=8.5 Hz, 1H), 4.42 (dd, J=7.8, 2.7 Hz, 2H), 4.16 (d, J=3.3 Hz, 1H), 3.96 (dd, J=10.9, 5.2 Hz, 1H), 3.93-3.48 (m, 29H), 3.40 (t, J=9.1 Hz, 1H), 2.73 (dd J=12.4, 4.7 Hz, 1H), 2.01 (dd, J=9.0, 0.8 Hz, 6H), 1.67 (t, J=12.2 Hz, 1H). ¹³C NMR (200 MHz, D₂O) δ 174.99, 174.86, 173.38, 158.08, 135.99, 128.75, 128.45, 127.80, 103.38, 102.81, 102.60, 100.15, 82.19, 81.58, 77.75, 76.11, 75.23, 75.00, 74.95, 73.67, 72.47, 72.44, 71.67, 71.35, 70.65, 69.94, 68.54, 68.31, 68.28, 68.19, 68.14, 67.37, 62.79, 62.55, 61.08, 61.00, 59.83, 54.68, 51.81, 40.05, 22.18, 21.98. HRMS (ESI-Orbitrap) m/z: [M−H]⁻ calculated for C₄₅H₆₈N₃O₃₀ 1130.3893; found 1130.3869.

Fucα2Galβ3(Neu5Acα6)GlcNAcβ3Galβ4GlcβNHCbz ((F-LSTbβNHCbz, 45). LSTbβNHCbz (44) (30 mg, 0.027 mmol), L-fucose (0.041 mmol), GTP (0.041 mmol) were dissolved in water in a 15 mL centrifuge tube containing Tris-HCl buffer (100 mM, pH 7.5) and MgCl₂ (20 mM). BfFKP (1 mg), Hm2FT (2 mg), and PmPpA (0.5 mg) were added and the concentration of LSTbβ3NHCbz (44) was about 10 mM. The reaction mixture was incubated at 30° C. for 3 days with agitation at 180 rpm. The product formation was monitored by TLC with EtOAc:MeOH:H₂O=5:2.2:1 (by volume) as the developing solvent and by HRMS. After the reaction was completed, the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (33 mg, 98%). ¹H NMR (800 MHz, D₂O) δ 7.47-7.24 (m, 5H), 5.20-5.15 (m, 3H), 4.82-4.80 (m, 1H), 4.63 (d, J=8.8 Hz, 1H), 4.58 (d, J=8.0 Hz, 1H), 4.42 (d, J=8.0 Hz, 1H), 4.33-3.39 (m, 41H), 2.77-2.71 (m, 2H), 2.02 (s, 6H), 2.01 (s, 3H), 1.77 (t, J=12.0 Hz, 1H), 1.68 (t, J=12.0 Hz, 1H), 1.15 (d, J=6.4 Hz, 3H). 2.73 (dd, J=12.4, 4.8 Hz, 1H), 2.04 (s, 3H), 2.02 (s, 3H), 1.65 (t, J=12.0 Hz, 1H), 1.24 (d, J=6.4, 2H). ¹³C NMR (200 MHz, D₂O) δ 175.03, 174.16, 173.40, 173.36, 158.09, 135.99, 128.76, 128.45, 127.79, 103.29, 103.27, 102.87, 100.29, 100.21, 100.14, 99.50, 81.84, 81.62, 81.54, 77.65, 77.01, 76.68, 76.34, 76.14, 75.37, 75.09, 75.00, 74.90, 73.80, 73.52, 72.50, 71.82, 71.68, 71.38, 70.19, 70.13, 69.42, 69.39, 69.13, 69.01, 68.45, 68.39, 68.33, 68.27, 68.20, 68.05, 67.39, 66.50, 62.72, 62.56, 61.09, 61.07, 60.97, 60.77, 59.84, 59.76, 54.98, 51.86, 51.76, 40.08, 22.12, 21.99, 15.29. HRMS (ESI-Orbitrap) m/z: [M−H]⁻ calculated for C₅₁H₇₈N₃O₃₄ 1276.4472; found 1276.4471.

Neu5Acα3Galβ3(Neu5Acα6)GlcNAcβ3Galβ4GlcβNHCbz (46). Neu5Acα2-3LNTβNHCbz (32) (100 mg, 0.087 mmol), CTP (77 mg, 0.15 mmol), and Neu5Ac (40 mg, 0.13 mmol) were dissolved in water in a 50 mL centrifuge tube containing Tris-HCl buffer (100 mM, pH 7.5) and MgCl₂ (20 mM). NmCSS (0.8 mg) and MBP-Δ50hST6GALNAC V-His₆_PROSS V7 (4.5 mg) were then added. The reaction mixture (8.6 mL) was incubated at 30° C. with agitation at 180 rpm. The product formation was monitored by HRMS. After the reaction went completion (30 h), the reaction mixture was incubated in a boiling water bath for 5 min to denature the enzymes, cooled down to room temperature, then centrifuged at 8000 rpm for 30 min at 4° C. The supernatant was concentrated and purified by a C18 cartridge (51 g, 50 μm, 120 Å, Yamazen) on a CombiFlash system to obtain the pure product as a white powder (124 mg, 98%). NMR data were consistent with those obtained for DSLNTβNHCbz (46) obtained by the StOPMe process described above.

### Example 9. Production of HMOs (Containing a Free Reducing End) by Hydrogenation of Neutral (3, 5-10, 23-31), Terminal α2-3-Sialylated (32, 34, 36, 37, 38, 40, 42, 43), Terminal α2-6-Sialylated (33, 35, 39, 41), and Neu5Acα2-6GlcNAc-Containing (44-47) HMOβNHCbz

Hydrogenation procedures. To remove the Cbz tag from the obtained glycosides, a catalytic amount (10-20%) of palladium on charcoal (Pd/C) (1.5 mg) was added to a solution containing 15 mg of each compound with a 20 mM final concentration in H₂O:MeOH=1:1 (by volume). The mixture was stirred at room temperature under a hydrogen atmosphere with a balloon for 2-3 hours. The reaction was monitored by HRMS analyses. When the reaction was completed, the mixture was passed through a 0.45 m syringe filter to remove palladium and charcoal. The solvent was removed in vacuo. The residue obtained was dissolved in H₂O and the concentration was kept at 20 mM. The mixture was incubated at 37° C. with 160 rpm agitation in an incubator shaker. The reaction was monitored by HRMS analyses until the β-glycosylamine was completely converted to the target HMO with a free reducing end (4-5 days for HMOs containing an L-fucose linked to the reducing end Glc residue or 20-48 hours for other HMOs). The pure HMO was obtained by lyophilization without further purification. NMR data of 3a and 5a-10a were consistent with those described herein prepared by the MSOPME strategy described herein.

Fucα2Galβ3(Fucα4)GlcNAcβ3Galβ4(Fucα3)Glc (23a) (LNTFHep, 12.2 mg, 91%). ¹H NMR (400 MHz, D₂O) δ 5.42 (d, J=4.1 Hz, 0.4H), 5.36 (d, J=4.0 Hz, 0.3H), 5.18 (d, J=3.8 Hz, 0.4H), 5.15 (d, J=4.0 Hz, 1H), 5.03 (d, J=3.9 Hz, 1H), 4.87 (d, J=6.8 Hz, 1H), 4.65 (dd, J=7.9, 4.8 Hz, 1.4H), 4.60 (dt, J=8.4, 1.9 Hz, 1H), 4.40 (d, J=7.8 Hz, 1H), 4.35 (q, J=6.6 Hz, 1H), 4.16-4.11 (m, 1H), 4.07 (d, J=3.5 Hz, 1H), 3.93 (tdd, J=8.5, 4.7, 1.5 Hz, 4H), 3.90-3.67 (m, 20H), 3.65-3.43 (m, 6H), 2.06 (d, J=4.5 Hz, 3H), 1.33-1.10 (m, 9H). ¹³C NMR (100 MHz, D₂O) δ 174.16, 103.19, 101.68, 100.59, 99.52, 97.74, 96.20, 92.12, 81.13, 77.09, 76.43, 75.49, 75.09, 74.71, 74.44, 73.59, 72.89, 72.62, 71.97, 71.73, 70.84, 69.37, 69.21, 69.08, 68.71, 68.54, 68.11, 67.78, 67.00, 66.42, 66.21, 62.45, 61.57, 61.49, 59.43, 55.79, 22.15, 15.33. HRMS (ESI-Orbitrap) m/z: [M+H]⁺ calculated for C₄₄H₇₆NO₃₃ 1146.4300; found 1146.4315.

Fucα2Galβ3GlcNAcβ3Galβ4(Fucα3)Glc (24a) (LNDFH-III, 12 mg, 90%). ¹H NMR (400 MHz, D₂O) δ 5.42 (d, J=4.0 Hz, 0.4H), 5.36 (d, J=4.0 Hz, 0.3H), 5.18 (dd, J=3.9, 2.2 Hz, 1.4H), 4.67-4.61 (m, 2.4H), 4.40 (d, J=7.8 Hz, 1H), 4.29 (q, J=6.5 Hz, 1H), 4.08 (d, J=3.5 Hz, 1H), 4.02-3.66 (m, 21H), 3.61 (ddt, J=17.3, 7.7, 3.5 Hz, 3H), 3.5-3.44 (m, 3H), 2.05 (d, J=4.7 Hz, 3H), 1.33-1.08 (m, 6H). ¹³C NMR (100 MHz, D₂O) δ 174.20, 103.19, 101.68, 100.21, 99.49, 98.50, 96.20, 81.87, 81.45, 81.16, 77.14, 76.65, 75.17, 75.04, 74.64, 74.40, 73.46, 72.90, 72.62, 72.17, 72.07, 71.86, 71.64, 71.42, 71.11, 70.96, 70.90, 69.39, 69.26, 69.22, 69.11, 68.67, 68.46, 67.94, 66.45, 61.45, 61.13, 61.05, 60.36, 59.79, 55.01, 22.11, 15.48, 15.26. HRMS (ESI-Orbitrap) m/z: [M+H]⁺ calculated for C₃₈H₆₆NO₂₉ 1000.3721; found 1000.3728.

Galβ3(Fucα4)GlcNAcβ3Galβ4Glc (25a) (LNFP-II, 12.1 mg, 94%). ¹H NMR (600 MHz, D₂O) δ 5.22 (d, J=3.8 Hz, 0.3H), 5.03 (d, J=4.0 Hz, 1H), 4.88 (t, J=6.7 Hz, 1H), 4.70 (dd, J=8.5, 2.7 Hz, 1H), 4.66 (d, J=8.0 Hz, 0.5H), 4.50 (d, J=7.6 Hz, 1H), 4.43 (d, J=7.8 Hz, 1H), 4.15 (d, J=3.3 Hz, 1H), 4.08 (t, J=9.7 Hz, 1H), 3.95 (ddd, J=10.0, 5.1, 2.2 Hz, 3H), 3.90-3.70 (m, 15H), 3.66-3.52 (m, 7H), 3.48 (dd, J=9.8, 7.7 Hz, 1H), 3.31-3.24 (m, 0.5H), 2.03 (s, 3H), 1.18 (d, J=6.6 Hz, 3H). ¹³C NMR (150 MHz, D₂O) δ 174.74, 102.91, 102.87, 102.83, 102.60, 97.99, 95.71, 91.78, 82.05, 78.35, 78.25, 75.89, 75.20, 74.86, 74.79, 74.77, 74.32, 73.76, 72.29, 72.10, 71.92, 71.37, 71.10, 70.47, 70.10, 69.94, 69.11, 68.33, 68.29, 67.76, 66.82, 61.63, 60.94, 60.05, 59.92, 59.58, 55.85, 22.26, 15.34. HRMS (ESI-Orbitrap) m/z: [M+H]⁺ calculated for C₃₂H₅₆NO₂₅854.3141; found 854.3152.

Fucα2Galβ3(Fucα4)GlcNAcβ3Galβ4Glc (26a) (LNDFH-I, 12.1 mg, 90%). ¹H NMR (400 MHz, D₂O) δ 5.24-5.19 (m, 0.3H), 5.15 (d, J=3.9 Hz, 1H), 5.02 (d, J=3.9 Hz, 1H), 4.87 (d, J=6.7 Hz, 1H), 4.66 (dd, J=7.9, 1.5 Hz, 1.5H), 4.60 (dd, J=8.4, 1.5 Hz, 1H), 4.42 (d, J=7.9 Hz, 1H), 4.34 (q, J=6.7 Hz, 1H), 4.18-4.08 (m, 2H), 3.99-3.88 (m, 3H), 3.91-3.67 (m, 19H), 3.68-3.48 (m, 7H), 3.34-3.23 (m, 0.5H), 2.06 (s, 3H), 1.26 (t, J=6.5 Hz, 6H). ¹³C NMR (100 MHz, D₂O) δ 174.16, 112.71, 103.21, 102.95, 100.61, 99.54, 97.76, 95.71, 81.52, 78.13, 76.47, 75.15, 74.79, 74.73, 74.46, 74.30, 73.77, 73.61, 71.97, 71.75, 70.13, 69.41, 69.08, 68.72, 68.25, 67.78, 67.00, 66.22, 61.57, 60.94, 59.44, 55.73, 22.15, 15.34, 15.30. HRMS (ESI-Orbitrap) m/z: [M+H]⁺ calculated for C₃₈H₆₆NO₂₉ 1000.3721; found 1000.3730.

Fucα2Galβ4GlcNAcβ3Galβ4Glc (27a) (LNnFP-I, 12.3 mg, 95% yield). ¹H NMR (600 MHz, D₂O) δ 5.31 (t, J=2.5 Hz, 1H), 5.22 (t, J=2.8 Hz, 0.3H), 4.70 (dt, J=8.4, 2.5 Hz, 1H), 4.66 (dd, J=8.0, 2.1 Hz, 0.6H), 4.55 (dd, J=7.8, 2.2 Hz, 1H), 4.46-4.41 (m, 1H), 4.25-4.19 (m, 1H), 4.14 (t, J=2.7 Hz, 1H), 3.95 (tq, J=8.0, 3.2, 2.7 Hz, 2H), 3.88 (dq, J=12.0, 2.7 Hz, 2H), 3.84-3.76 (m, 9H), 3.75-3.63 (m, 9H), 3.62-3.56 (m, 2H), 3.46 (ddd, J=10.1, 5.1, 2.4 Hz, 1H), 3.30-3.24 (m, 0.5H), 2.04 (d, J=2.1 Hz, 3H), 1.23 (dd, J=6.7, 2.1 Hz, 3H). ¹³C NMR (150 MHz, D₂O) δ 174.89, 102.89, 102.73, 100.22, 99.38, 95.71, 91.78, 81.94, 78.35, 78.24, 76.41, 75.86, 75.23, 75.07, 74.83, 74.77, 74.32, 73.76, 73.51, 72.04, 71.64, 71.37, 71.10, 70.10, 69.97, 69.59, 69.09, 68.30, 68.17, 66.92, 61.09, 60.90, 60.05, 59.98, 59.92, 55.36, 22.17, 15.29. HRMS (ESI-Orbitrap) m/z: [M+H]⁺ calculated for C₃₂H₅₆NO₂₅854.3141; found 854.3126.

Fucα2Galβ4(Fucα3)GlcNAcβ3Galβ4(Fucα3)Glc (28a) (LNnTFHep, 12.4 mg, 93%). ¹H NMR (400 MHz, D₂O) δ 5.35 (d, J=4.1 Hz, 0.4H), 5.29 (d, J=4.1 Hz, 0.3H), 5.20 (d, J=3.1 Hz, 1H), 5.10 (d, J=3.7 Hz, 0.4H), 5.04 (t, J=3.5 Hz, 1H), 4.80 (d, J=6.8 Hz, 1H), 4.64 (s, 1H), 4.57 (d, J=8.0 Hz, 0.6H), 4.43 (d, J=7.7 Hz, 1H), 4.34 (d, J=7.8 Hz, 1H), 4.17 (q, J=6.6 Hz, 1H), 4.00 (d, J=3.4 Hz, 1H), 3.93-3.51 (m, 30H), 3.45-3.34 (m, 2H), 1.94 (d, J=2.5 Hz, 3H), 1.20-1.07 (m, 9H). ¹³C NMR (100 MHz, D₂O) δ 174.65, 102.41, 101.70, 100.18, 99.39, 98.57, 96.20, 95.81, 76.97, 76.33, 75.52, 75.30, 74.83, 74.79, 74.65, 74.45, 73.54, 73.03, 72.90, 72.34, 72.26, 71.92, 71.67, 71.64, 71.42, 70.93, 70.84, 69.71, 69.25, 69.24, 69.16, 68.73, 68.25, 68.01, 67.98, 67.67, 66.90, 66.76, 66.46, 66.42, 61.45, 59.76, 59.72, 22.24, 15.41, 15.18. HRMS (ESI-Orbitrap) m/z: [M+H]⁺ calculated for C₄₄H₇₆NO₃₃ 1146.4300; found 1146.4318.

Fucα2Galβ4GlcNAcβ3Galβ4(Fucα3)Glc (29a) (LNnDFH-III, 12.1 mg, 90%). ¹H NMR (400 MHz, D₂O) δ 5.43 (d, J=4.0 Hz, 0.5H), 5.37 (d, J=4.1 Hz, 0.4H), 5.31 (d, J=2.8 Hz, 1H), 5.18 (d, J=3.8 Hz, 0.4H), 4.73-4.69 (m, 1H), 4.65 (d, J=8.0 Hz, 0.4H), 4.55 (dd, J=7.8, 1.1 Hz, 1H), 4.42 (d, J=7.8 Hz, 1H), 4.22 (q, J=6.6 Hz, 1H), 4.09 (d, J=3.3 Hz, 1H), 3.99-3.67 (m, 26H), 3.60 (dt, J=6.2, 2.9 Hz, 1H), 3.53-3.45 (m, 2H), 2.04 (d, J=3.0 Hz, 3H), 1.24-1.15 (m, 6H). ¹³C NMR (100 MHz, D₂O) δ 174.86, 102.70, 101.72, 100.23, 99.38, 95.82, 76.99, 76.41, 75.88, 75.35, 75.24, 75.05, 74.47, 73.53, 72.90, 72.67, 72.40, 72.02, 71.90, 71.65, 70.93, 70.85, 70.69, 69.60, 69.25, 69.10, 68.18, 68.02, 66.93, 66.47, 61.11, 60.01, 55.34, 22.16, 15.29, 15.20. HRMS (ESI-Orbitrap) m/z: [M+H]⁺ calculated for C₃₈H₆₆NO₂₉ 1000.3721; found 1000.3732.

Galβ4(Fucα3)GlcNAcβ3Galβ4Glc (30a) (LNFP-III, 12.3 mg, 95%). ¹H NMR (600 MHz, D₂O) δ 5.22 (d, J=3.8 Hz, 0.3H), 5.13 (d, J=4.0 Hz, 1H), 4.84 (d, J=6.8 Hz, 1H), 4.72 (dd, J=8.5, 2.5 Hz, 1H), 4.66 (d, J=8.0 Hz, 0.5H), 4.45 (dd, J=16.2, 7.8 Hz, 2H), 4.15 (d, J=3.3 Hz, 1H), 4.00-3.93 (m, 4H), 3.92-3.56 (m, 21H), 3.50 (dd, J=9.8, 7.8 Hz, 1H), 3.32-3.24 (m, 0.5H), 2.02 (s, 3H), 1.17 (d, J=6.6 Hz, 3H). ¹³C NMR (150 MHz, D₂O) δ 174.67, 102.90, 102.87, 102.52, 101.74, 98.58, 95.71, 91.78, 82.04, 78.33, 78.23, 75.10, 74.89, 74.85, 74.77, 74.72, 74.32, 73.76, 73.04, 72.45, 71.89, 71.37, 71.10, 71.03, 70.09, 69.95, 69.17, 68.32, 68.29, 67.68, 66.67, 61.48, 60.94, 60.05, 59.92, 59.61, 55.94, 22.23, 15.29. HRMS (ESI-Orbitrap) m/z: [M+H]⁺ calculated for C₃₂H₅₆NO₂₅854.3141; found 854.3127.

Fucα2Galβ4(Fucα3)GlcNAcβ3Galβ4Glc (31a) (LNnDFH-I, 12.2 mg, 92% yield). ¹H NMR (600 MHz, D₂O) δ 5.27 (d, J=3.5 Hz, 1H), 5.22 (d, J=3.8 Hz, 0.3H), 5.11 (d, J=4.0 Hz, 1H), 4.88 (d, J=6.7 Hz, 1H), 4.72 (d, J=10.4 Hz, 1H), 4.68-4.63 (m, 0.6H), 4.51 (d, J=7.8 Hz, 1H), 4.44 (d, J=7.8 Hz, 1H), 4.25 (q, J=6.7 Hz, 1H), 4.16-4.12 (m, 1H), 4.03-3.89 (m, 5H), 3.88-3.55 (m, 23H), 3.48-3.40 (m, 1H), 3.31-3.23 (m, 0.5H), 2.03 (s, 3H), 1.25 (dd, J=17.6, 6.6 Hz, 6H). ¹³C NMR (150 MHz, D₂O) δ 174.70, 102.88, 102.46, 100.18, 99.40, 98.57, 95.71, 91.78, 81.96, 78.33, 78.23, 76.34, 75.35, 74.83, 74.77, 74.32, 73.76, 73.53, 73.05, 71.92, 71.67, 71.37, 71.10, 70.10, 70.01, 69.98, 69.70, 69.15, 68.73, 68.25, 67.67, 66.90, 66.77, 61.45, 60.91, 60.05, 59.76, 56.09, 22.25, 15.44, 15.41. HRMS (ESI-Orbitrap) m/z: [M+H]⁺ calculated for C₃₈H₆₆NO₂₉ 1000.3721; found 1000.3731.

Neu5Acα3Galβ3GlcNAcβ3Galβ4Glc (32a) (LSTa, 12.4 mg, 94% yield). ¹H NMR (600 MHz, D₂O) δ 5.22 (d, J=3.8 Hz, 0.3H), 4.74 (dd, J=8.4, 2.4 Hz, 1H), 4.67 (d, J=8.0 Hz, 0.6H), 4.51 (d, J=7.8 Hz, 1H), 4.44 (dd, J=7.9, 1.2 Hz, 1H), 4.15 (d, J=3.4 Hz, 1H), 4.09 (dd, J=9.8, 3.2 Hz, 1H), 4.00-3.52 (m, 28H), 3.48 (ddd, J=9.9, 5.1, 2.4 Hz, 1H), 3.28 (dd, J=9.3, 8.1 Hz, 0.5H), 2.76 (dd, J=12.4, 4.7 Hz, 1H), 2.03 (s, 6H), 1.79 (t, J=12.2 Hz, 1H). ¹³C NMR (150 MHz, D₂O) δ 174.94, 174.88, 173.87, 103.35, 102.90, 102.47, 99.62, 95.71, 91.79, 82.13, 81.89, 78.41, 78.29, 75.58, 75.19, 75.07, 74.88, 74.78, 74.34, 73.76, 72.78, 71.82, 71.38, 71.10, 70.09, 69.99, 69.06, 68.45, 68.36, 68.29, 68.02, 67.23, 62.44, 61.01, 60.95, 60.50, 60.05, 59.88, 54.56, 51.64, 39.76, 22.28, 22.02. HRMS (ESI-Orbitrap) m/z: [M−H]⁻ calculated for C₃₇H₆₁N₂O₂₉997.3365; found 997.3340.

Neu5Acα3Galβ3GlcNAcβ3Galβ4(Fucα4)Glc (34a) (12.6 mg, 94%). ¹H NMR (600 MHz, D₂O) δ 5.44 (dd, J=13.0, 4.0 Hz, 1H), 5.37 (d, J=4.0 Hz, 0.4H), 5.18 (d, J=3.8 Hz, 0.4H), 4.75-4.72 (m, 1H), 4.65 (d, J=8.0 Hz, 0.5H), 4.52 (d, J=7.8 Hz, 1H), 4.42 (d, J=7.8 Hz, 1H), 4.12-4.07 (m, 2H), 3.99-3.93 (m, 3H), 3.92-3.46 (m, 32H), 2.76 (dd, J=12.4, 4.7 Hz, 1H), 2.03 (d, J=3.1 Hz, 6H), 1.78 (t, J=12.1 Hz, 1H), 1.16 (dd, J=6.7, 3.4 Hz, 3H). ¹³C NMR (150 MHz, D₂O) δ 174.94, 174.85, 173.86, 103.31, 102.39, 101.68, 99.61, 98.41, 95.81, 82.05, 81.38, 76.98, 75.57, 75.53, 75.35, 75.16, 75.06, 74.48, 72.77, 72.35, 71.88, 71.82, 70.93, 70.68, 69.19, 69.06, 68.43, 68.36, 68.20, 68.02, 67.22, 66.48, 62.44, 61.56, 61.45, 61.00, 60.49, 54.56, 51.63, 39.76, 22.26, 22.02, 15.18. HRMS (ESI-Orbitrap) m/z: [M−H]⁻ calculated for C₄₃H₇₁N₂O₃₃1143.3945; found 1143.3901.

Neu5Acα3Galβ3(Fucα4)GlcNAcβ3Galβ4(Fucα3)Glc (36a) (12.4 mg, 91%). ¹H NMR (800 MHz, D₂O) δ 5.42 (d, J=4.0 Hz, 0.6H), 5.37 (d, J=4.0 Hz, 0.4H), 5.17 (d, J=4.0 Hz, 0.4H), 5.01-5.00 (m, 1H), 4.89-4.80 (m, 2H), 4.70 (d, J=8.0, 0.6H), 4.67 (d, J=8.0, 0.6H), 4.65 (d, J=8.0, 0.4H), 4.55 (d, J=8.0 Hz, 1H), 4.50 (d, J=8.0 Hz, 1H), 4.41 (d, J=8.0 Hz, 1H), 4.13-3.40 (m, 36H), 2.78-2.73 (m, 1H), 2.03-2.01 (m, 6H), 1.77-1.68 (m, 1H), 1.17-1.14 (m, 6H). ¹³C NMR (200 MHz, D₂O) δ 175.04, 174.91, 174.72, 174.55, 173.92, 173.45, 102.79, 102.72, 102.59, 102.43, 101.71, 100.79, 99.35, 98.52, 98.41, 98.10, 97.96, 95.81, 92.10, 81.56, 81.46, 76.96, 76.00, 75.90, 75.58, 75.52, 75.35, 75.21, 75.13, 74.71, 74.65, 74.48, 72.72, 72.69, 72.67, 72.38, 72.34, 72.25, 72.16, 72.04, 72.01, 71.92, 71.89, 71.87, 71.85, 71.80, 70.93, 70.84, 70.68, 70.64, 70.61, 70.39, 69.24, 69.19, 69.08, 69.05, 68.77, 68.40, 68.25, 68.15, 68.01, 67.98, 67.92, 67.83, 67.77, 66.90, 66.81, 66.72, 66.48, 66.44, 63.40, 62.62, 62.30, 61.64, 61.49, 61.46, 60.52, 59.76, 59.68, 59.60, 59.51, 55.79, 51.80, 51.64, 40.14, 39.97, 22.36, 22.27, 22.02, 21.99, 15.41, 15.31, 15.18. HRMS (ESI-Orbitrap) m/z: [M−H]⁻ calculated for C₄₉H₈₁N₂O₃₇1289.4524; found 1289.4521.

Neu5Acα3Galβ3(Fucα4)GlcNAcβ3Galβ4Glc (37a) (12.4 mg, 92% yield). ¹H NMR (600 MHz, D₂O) δ 5.22 (d, J=3.8 Hz, 0.3H), 5.02 (t, J=4.4 Hz, 1H), 4.88 (t, J=6.7 Hz, 1H), 4.68 (dd, J=17.6, 8.5 Hz, 2H), 4.55 (d, J=7.7 Hz, 0.5H), 4.43 (dd, J=13.8, 7.9 Hz, 1H), 4.17 (dd, J=16.3, 3.4 Hz, 1H), 4.13-4.03 (m, 2H), 3.99-3.92 (m, 4H), 3.91-3.47 (m, 29H), 3.29 (dt, J=13.4, 8.3 Hz, 0.5H), 2.74 (ddd, J=36.3, 12.4, 4.7 Hz, 1H), 2.03 (d, J=6.1 Hz, 6H), 1.75 (dt, J=22.8, 12.2 Hz, 1H), 1.18 (dd, J=6.7, 2.8 Hz, 3H). ¹³C NMR (150 MHz, D₂O) δ 174.89, 174.70, 174.59, 173.91, 173.46, 102.84, 102.73, 102.52, 100.26, 99.37, 98.00, 95.71, 95.61, 91.79, 82.21, 82.00, 79.62, 75.60, 75.20, 74.87, 74.79, 74.73, 74.64, 74.60, 73.76, 73.25, 72.73, 72.51, 72.29, 72.14, 72.03, 71.93, 71.85, 71.75, 71.60, 71.38, 71.10, 70.48, 69.95, 69.11, 68.78, 68.40, 68.34, 68.15, 68.01, 67.78, 66.92, 66.81, 62.62, 62.31, 61.63, 60.96, 59.60, 55.84, 51.77, 51.66, 40.08, 22.39, 22.27, 22.03, 15.35, 15.32. HRMS (ESI-Orbitrap) m/z: [M−H]⁻ calculated for C₄₃H₇₁N₂O₃₃1143.3945; found 1143.3897.

Neu5Acα3Galβ4GlcNAcβ3Galβ4Glc (38a) (12.6 mg, 95%). ¹H NMR (600 MHz, D₂O) δ 5.22 (d, J=3.8 Hz, 0.3H), 4.70 (dd, J=8.3, 2.3 Hz, 1H), 4.66 (d, J=7.9 Hz, 0.6H), 4.56 (d, J=7.9 Hz, 1H), 4.44 (d, J=7.9 Hz, 1H), 4.16 (d, J=3.3 Hz, 1H), 4.12 (dd, J=9.9, 3.1 Hz, 1H), 3.95 (tq, J=5.6, 3.5, 2.9 Hz, 3H), 3.92-3.67 (m, 18H), 3.69-3.54 (m, 10H), 3.32-3.25 (m, 0.6H), 2.76 (dd, J=12.4, 4.6 Hz, 1H), 2.03 (s, 6H), 1.80 (t, J=12.1 Hz, 1H). 13C NMR (150 MHz, D₂O) δ 174.99, 174.87, 173.83, 102.90, 102.77, 102.52, 99.78, 95.71, 91.78, 82.02, 78.37, 78.27, 77.98, 75.47, 75.15, 74.87, 74.78, 74.53, 74.33, 73.76, 72.86, 72.12, 71.74, 71.37, 71.10, 70.10, 69.95, 69.36, 68.32, 68.06, 67.45, 62.56, 61.01, 60.96, 60.05, 59.92, 59.82, 55.16, 51.66, 39.61, 22.15, 22.02. HRMS (ESI-Orbitrap) m/z: [M−H]⁻ calculated for C₃₇H₆₁N₂O₂₉997.3365; found 997.3329.

Neu5Acα3Galβ4GlcNAcβ3Galβ4(Fucα3)Glc (40a) (12.6 mg, 94%). ¹H NMR (600 MHz, D₂O) δ 5.44 (dd, J=12.9, 4.0 Hz, 0.6H), 5.37 (d, J=4.0 Hz, 0.3H), 5.18 (d, J=3.8 Hz, 0.3H), 4.81 (s, 1H), 4.70 (dd, J=8.3, 2.0 Hz, 1H), 4.65 (d, J=8.0 Hz, 0.4H), 4.56 (d, J=7.8 Hz, 1H), 4.41 (dd, J=7.8, 5.2 Hz, 1H), 4.14-4.08 (m, 2H), 4.00-3.44 (m, 33H), 2.76 (dd, J=12.4, 4.6 Hz, 1H), 2.03 (d, J=3.5 Hz, 6H), 1.80 (t, J=12.1 Hz, 1H), 1.25-1.12 (m, 3H). ¹³C NMR (150 MHz, D₂O) δ 174.99, 174.83, 173.83, 102.71, 102.52, 101.70, 99.78, 98.42, 95.81, 81.57, 78.01, 76.98, 75.46, 75.35, 75.15, 74.67, 74.50, 72.87, 72.67, 72.36, 72.07, 71.90, 71.74, 70.93, 69.36, 69.19, 68.33, 68.07, 67.99, 67.45, 66.47, 62.56, 61.49, 61.01, 60.51, 59.82, 55.14, 51.66, 39.62, 22.14, 22.01, 15.20. HRMS (ESI-Orbitrap) m/z: [M−H]⁻ calculated for C₄₃H₇₁N₂O₃₃1143.3945; found 1143.3905.

Neu5Acα3Galβ4(Fucα3)GlcNAcβ3Galβ4(Fucα3)Glc (42a) (12.5 mg, 92%). ¹H NMR (800 MHz, D₂O) δ 5.42 (d, J=4.0 Hz, 0.6H), 5.36 (d, J=4.0 Hz, 0.4H), 5.17 (d, J=4.0 Hz, 0.4H), 5.11-5.10 (m, 1H), 4.83-4.79 (m, 2H), 4.69 (d, J=8.0, 1H), 4.64 (d, J=8.0, 0.4H), 4.52 (d, J=8.0, 1.0H), 4.40 (d, J=8.0 Hz, 1H), 4.10-3.44 (m, 37H), 2.76 (dd, J=12.0, 4.8 Hz, 1H), 2.03-2.01 (m, 6H), 1.78 (t, J=12.0 Hz, 1H), 1.17-1.14 (m, 6H). ¹³C NMR (200 MHz, D₂O) δ 175.00, 174.62, 173.84, 102.51, 101.70, 101.52, 100.68, 99.63, 98.53, 98.41, 95.81, 92.10, 81.58, 81.51, 76.97, 75.64, 75.52, 75.35, 74.97, 74.89, 74.66, 74.62, 74.48, 73.00, 72.89, 72.66, 72.33, 72.23, 71.88, 71.83, 70.92, 70.66, 70.61, 69.24, 69.18, 69.14, 68.28, 68.21, 68.08, 68.01, 67.98, 67.77, 67.69, 67.29, 66.63, 66.47, 66.43, 62.56, 61.49, 61.47, 60.42, 59.77, 59.50, 55.92, 51.67, 39.76, 22.21, 22.01, 21.99, 15.37, 15.25, 15.19. HRMS (ESI-Orbitrap) m/z: [M−H]⁻ calculated for C₄₉H₈₁N₂O₃₇1289.4524; found 1289.4523.

Neu5Acα3Galβ4(Fucα3)GlcNAcβ3Galβ4Glc (43a) (12.5 mg, 93%). ¹H NMR (800 MHz, D₂O) δ 5.21 (d, J=4.0 Hz, 0.4H), 5.12-5.08 (m, 1H), 4.83-4.81 (m, 1H), 4.71-4.69 (m, 1H), 4.65 (d, J=8.0, 0.6H), 4.52 (d, J=8.0, 0.6H), 4.42 (d, J=8.0, 1H), 4.16-3.24 (m, 36H), 2.77-2.72 (m, 1H), 2.03-2.01 (m, 6H), 1.80-1.67 (m, 1H), 1.17-1.14 (m, 6H). ¹³C NMR (200 MHz, D₂O) δ 175.00, 174.65, 173.83, 102.93, 102.90, 102.87, 102.55, 101.76, 101.54, 100.68, 99.64, 99.63, 98.55, 95.71, 91.78, 82.07, 82.04, 78.35, 78.24, 75.64, 75.15, 75.01, 74.89, 74.87, 74.86, 74.85, 74.77, 74.62, 74.61, 74.33, 73.75, 73.01, 72.89, 71.87, 71.83, 71.81, 71.72, 71.37, 71.09, 70.95, 70.09, 69.96, 69.93, 69.24, 69.15, 68.33, 68.31, 68.29, 68.28, 68.26, 68.23, 68.15, 68.07, 67.77, 67.69, 67.28, 66.64, 62.60, 62.56, 61.48, 61.47, 60.97, 60.95, 60.83, 60.81, 60.79, 60.77, 60.76, 60.05, 59.91, 59.49, 55.93, 51.81, 51.67, 39.76, 39.74, 22.24, 22.22, 22.01, 21.99, 15.36, 15.25, 15.24, 15.22. HRMS (ESI-Orbitrap) m/z: [M−H]⁻ calculated for C₄₃H₇₁N₂O₃₃1143.3945; found 1143.3951.

Neu5Acα6Galβ3GlcNAcβ3Galβ4Glc (33a) (LSTc, 12.2 mg, 93%). ¹H NMR (600 MHz, D₂O) δ 5.22 (d, J=3.6 Hz, 0.3H), 4.74 (d, J=8.5 Hz, 1H), 4.66 (d, J=8.0 Hz, 0.6H), 4.45 (d, J=7.9 Hz, 1H), 4.39 (d, J=8.0 Hz, 1H), 4.16 (d, J=3.3 Hz, 1H), 4.01-3.70 (m, 18H), 3.69-3.49 (m, 13H), 3.28 (t, J=8.5 Hz, 0.5H), 2.70 (dd, J=12.4, 4.7 Hz, 1H), 2.08-1.98 (m, 6H), 1.70 (t, J=12.1 Hz, 1H). ¹³C NMR (150 MHz, D₂O) δ 174.95, 174.88, 173.47, 103.85, 102.89, 102.52, 100.11, 95.71, 91.78, 83.62, 81.94, 78.40, 78.29, 75.27, 74.90, 74.78, 74.34, 73.76, 73.58, 72.43, 72.36, 71.80, 71.38, 71.10, 70.51, 70.10, 70.00, 68.63, 68.43, 68.39, 68.28, 63.51, 62.61, 61.11, 60.96, 60.63, 60.06, 59.92, 54.43, 51.78, 40.12, 22.19, 22.03. HRMS (ESI-Orbitrap) m/z: [M−H]⁻ calculated for C₃₇H₆₁N₂O₂₉ 997.3365; found 997.3322.

Neu5Acα6Galβ3GlcNAcβ3Galβ4(Fucα3)Glc (35a) (12.2 mg, 91%). ¹H NMR (600 MHz, D₂O) δ 5.36 (dd, J=13.1, 4.0 Hz, 0.6H), 5.29 (d, J=4.0 Hz, 0.3H), 5.10 (d, J=3.8 Hz, 0.3H), 4.74 (d, J=6.3 Hz, 1H), 4.66 (d, J=8.5 Hz, 1H), 4.57 (d, J=8.0 Hz, 0.5H), 4.35 (d, J=7.8 Hz, 1H), 4.31 (d, J=7.9 Hz, 1H), 4.02 (s, 1H), 3.92-3.37 (m, 37H), 2.62 (dd, J=12.4, 4.7 Hz, 1H), 1.95 (d, J=7.2 Hz, 6H), 1.62 (t, J=12.1 Hz, 1H), 1.08 (dd, J=6.8, 3.3 Hz, 3H). ¹³C NMR (150 MHz, D₂O) δ 174.93, 174.88, 173.47, 103.84, 102.45, 101.68, 100.12, 98.42, 95.82, 92.10, 83.53, 81.46, 81.40, 76.99, 75.53, 75.35, 75.25, 74.67, 74.51, 73.57, 72.67, 72.43, 72.35, 72.27, 71.90, 71.80, 70.67, 70.50, 69.24, 69.19, 68.63, 68.44, 68.38, 68.28, 67.99, 66.47, 63.51, 62.61, 61.49, 61.38, 60.62, 54.43, 51.78, 40.12, 22.17, 22.03, 15.19. HRMS (ESI-Orbitrap) m/z: [M−H]⁻ calculated for C₄₃H₇₁N₂O₃₃1143.3945; found 1143.3903.

Neu5Acα6Galβ4GlcNAcβ3Galβ4Glc (39a) (12.2 mg, 92% yield). ¹H NMR (600 MHz, D₂O) δ 5.22 (d, J=3.8 Hz, 0.3H), 4.75-4.72 (m, 1H), 4.67 (d, J=7.9 Hz, 0.6H), 4.45 (t, J=7.8 Hz, 2H), 4.16 (d, J=3.3 Hz, 1H), 4.02-3.92 (m, 4H), 3.91-3.52 (m, 28H), 3.31-3.25 (m, 0.6H), 2.67 (dd, J=12.4, 4.6 Hz, 1H), 2.04 (d, J=15.3 Hz, 6H), 1.72 (t, J=12.2 Hz, 1H). ¹³C NMR (150 MHz, D₂O) δ 174.89, 173.52, 103.44, 102.91, 102.55, 100.11, 95.71, 91.78, 81.97, 80.45, 78.40, 78.28, 74.87, 74.78, 74.33, 74.25, 73.76, 73.67, 72.52, 72.40, 72.22, 71.69, 71.38, 71.10, 70.71, 70.09, 69.95, 68.38, 68.34, 68.30, 68.19, 63.32, 62.63, 61.36, 60.95, 60.13, 60.04, 59.91, 54.92, 51.87, 40.06, 22.26, 22.01. HRMS (ESI-Orbitrap) m/z: [M−H]⁻ calculated for C₄₃H₇₁N₂O₃₃1143.3945; found 1143.3907.

Neu5Acα6Galβ4GlcNAcβ3Galβ4(Fucα3)Glc (41a) (12.6 mg, 94%). ¹H NMR (600 MHz, D₂O) δ 5.44 (dd, J=12.9, 4.0 Hz, 0.6H), 5.37 (d, J=4.0 Hz, 0.3H), 5.18 (d, J=3.8 Hz, 0.3H), 4.82 (s, 1H), 4.65 (d, J=8.0, 0.5H), 4.44 (dd, J=21.4, 7.8 Hz, 2H), 4.10 (d, J=3.4 Hz, 1H), 4.02-3.92 (m, 5H), 3.91-3.44 (m, 30H), 2.67 (dd, J=12.4, 4.7 Hz, 1H), 2.09-1.99 (m, 6H), 1.72 (t, J=12.1 Hz, 1H), 1.17 (dd, J=6.7, 3.4 Hz, 3H). ¹³C NMR (150 MHz, D₂O) δ 174.89, 174.86, 173.52, 103.45, 102.50, 101.71, 100.12, 98.41, 95.81, 92.10, 81.53, 80.48, 76.97, 75.53, 75.36, 74.48, 74.22, 73.68, 72.68, 72.52, 72.40, 72.28, 72.19, 71.89, 71.69, 70.93, 70.72, 70.59, 69.19, 68.38, 68.34, 68.17, 67.99, 66.48, 63.32, 62.63, 61.48, 60.77, 60.13, 54.88, 51.87, 40.05, 22.24, 22.01, 15.20. HRMS (ESI-Orbitrap) m/z: [M−H]⁻ calculated for C₄₃H₇₁N₂O₃₃1143.3945; found 1143.3912.

Galβ(Neu5Acα6)GlcNAcβ3Galβ4Glc (44a) (LSTb, 12.5 mg, 95% yield). ¹H NMR (600 MHz, D₂O) δ 5.22 (d, J=3.8 Hz, 0.3H), 4.70 (dd, J=8.5, 2.7 Hz, 1H), 4.66 (d, J=7.9 Hz, 0.5H), 4.44 (d, J=7.8 Hz, 2H), 4.18 (d, J=3.2 Hz, 1H), 4.02-3.50 (m, 30H), 3.33-3.25 (m, 0.5H), 2.75 (dd, J=12.4, 4.7 Hz, 1H), 2.03 (d, J=7.0 Hz, 6H), 1.69 (t, J=12.2 Hz, 1H). 13C NMR (150 MHz, D₂O) δ 175.02, 174.88, 173.40, 103.40, 102.89, 102.61, 100.18, 95.72, 82.28, 82.24, 81.63, 78.43, 78.34, 75.25, 74.97, 74.78, 74.34, 73.74, 73.69, 72.49, 72.46, 71.69, 71.38, 71.09, 70.67, 70.10, 69.98, 69.95, 68.55, 68.35, 68.30, 68.21, 68.14, 62.82, 62.57, 61.09, 61.01, 60.08, 54.70, 51.84, 40.07, 22.21, 22.00. HRMS (ESI-Orbitrap) m/z: [M−H]⁻ calculated for C₃₇H₆₁N₂O₂₉997.3365; found 997.3325.

Fucα2Galβ3(Neu5Acα6)GlcNAcβ3Galβ4Glc (45a) (F-LSTb, 12 mg, 96% yield). 25 ¹H NMR (800 MHz, D₂O) δ 5.21 (d, J=4.0 Hz, 0.4H), 5.18 (d, J=4.0 Hz, 0.4H), 5.17 (d, J=4.0 Hz, 1H), 4.83-4.80 (m, 1H), 4.67-4.40 (m, 4H), 4.30-3.28 (m, 33H), 2.75-2.70 (m, 1H), 2.06-2.01 (m, 6H), 1.71-1.63 (m, 1H), 1.26-1.21 (m, 3H). ¹³C NMR (201 MHz, D₂O) δ 175.03, 174.15, 173.36, 103.29, 102.93, 102.89, 100.33, 100.28, 100.21, 100.14, 99.50, 99.43, 95.70, 91.78, 87.37, 81.91, 81.86, 78.28, 78.20, 77.01, 76.68, 75.09, 74.91, 74.90, 74.80, 74.32, 73.81, 73.74, 73.57, 73.53, 72.50, 71.82, 71.68, 71.35, 71.09, 70.20, 70.16, 70.12, 69.39, 69.13, 68.45, 68.40, 68.36, 68.33, 68.27, 68.20, 68.05, 68.03, 66.50, 62.72, 62.61, 62.56, 61.09, 61.06, 60.35, 60.06, 59.92, 54.99, 51.86, 51.76, 40.13, 40.08, 22.15, 22.12, 22.03, 21.99, 15.29, 15.22. HRMS (ESI-Orbitrap) m/z: [M−H]⁻ calculated for C₄₃H₇₁N₂O₃₃1143.3945; found 1143.3957.

Neu5Acα3Galβ3(Neu5Acα6)GlcNAcβ3Galβ4Glc (46a) (DSLNT, 12.5 mg, 92% yield). ¹H NMR (600 MHz, D₂O) δ 5.22 (d, J=3.7 Hz, 0.3H), 4.72-4.69 (m, 1H), 4.66 (dd, J=8.0, 1.3 Hz, 0.7H), 4.50 (d, J=7.8 Hz, 1H), 4.46-4.43 (m, 1H), 4.17 (d, J=3.3 Hz, 1H), 4.11-4.06 (m, 1H), 4.00-3.51 (m, 36H), 3.32-3.24 (m, 0.5H), 2.75 (ddd, J=12.1, 9.8, 4.6 Hz, 2H), 2.14-1.94 (m, 8H), 1.78 (t, J=12.1 Hz, 1H), 1.69 (t, J=12.1 Hz, 1H). ¹³C NMR (150 MHz, D₂O) δ 174.99, 173.88, 173.41, 103.41, 102.89, 102.53, 100.18, 99.60, 95.71, 82.23, 81.81, 78.37, 75.58, 75.06, 74.97, 74.78, 74.35, 73.74, 72.76, 72.48, 71.81, 71.67, 69.95, 69.08, 68.39, 68.32, 68.22, 68.03, 67.25, 62.86, 62.56, 62.42, 61.10, 61.03, 60.75, 54.60, 51.83, 51.63, 40.06, 39.76, 22.29, 22.02, 22.00. HRMS (ESI-Orbitrap) m/z: [M−H]⁻ calculated for C₄₈H₇₈N₃O₃₇ 1288.4320; found 1288.4264.

Neu5Acα3Galβ3(Neu5Acα6)GlcNAcβ3Galβ4(Fucα3)Glc (47a) (FDS-LNT-II, 12.0 mg, 96%). ¹H NMR (800 MHz, D₂O) δ 5.42 (d, J=4.0 Hz, 0.6H), 5.36 (d, J=4.0 Hz, 0.4H), 5.17 (d, J=4.0 Hz, 1H), 4.83-4.81 (m, 1H), 4.70 (d, J=8.0, 1H), 4.64 (d, J=8.0, 0.6H), 4.50 (d, J=8.0, 0.6H), 4.41 (d, J=8.0, 0.6H), 4.11-3.44 (m, 40H), 2.77-2.71 (m, 2H), 2.02-2.00 (m, 9H), 1.78 (t, J=12.0 Hz, 1H), 1.68 (t, J=12.0 Hz, 1H), 1.16-1.14 (m, 3H). ¹³C NMR (200 MHz, D₂O) δ 175.00, 174.92, 174.79, 173.88, 173.39, 103.37, 102.37, 101.68, 101.66, 100.15, 99.59, 98.52, 98.41, 95.81, 92.10, 81.80, 81.73, 76.99, 75.57, 75.53, 75.36, 75.05, 74.67, 74.57, 73.69, 72.75, 72.68, 72.46, 72.38, 72.29, 71.88, 71.81, 71.66, 70.93, 70.63, 70.59, 69.22, 69.17, 69.07, 68.42, 68.39, 68.33, 68.22, 68.09, 68.05, 68.02, 68.01, 67.98, 67.24, 66.48, 66.44, 62.91, 62.55, 62.41, 61.61, 61.02, 59.76, 54.60, 51.84, 51.63, 40.08, 39.77, 22.28, 22.02, 22.00, 15.20. HRMS (ESI-Orbitrap) m/z: [M−2H]²-calculated for C₅₄H₈₉N₃O₄₁ 716.7413; found 716.7424.

### Example 10. Sugar Activation (SA) Systems and Glycosyltransferases for Synthesizing Target HMOs

Provided herein is an efficient strategy to access target HMOs shown in FIG. 10 by glycosyltransferase (GT)-catalyzed extension of commercially available inexpensive lactose or its derivatives to allow easy purification of products. Glycosyltransferases use activated sugar nucleotides as donor substrates which can be generated in situ from simple monosaccharides and enzymes involved in its activation. Four sugar activation (SA) systems shown in FIG. 4 can be used, as provided herein, for in situ-generation of the corresponding sugar nucleotides which can be combined with suitable GTs for the formation of the desired glycosidic linkages. UDP-GlcNAc was obtained from GlcNAc, ATP, and UTP via SA1 containing three enzymes including Bifidobacterium longum N-acetylhexosamine-1-kinase (BLNahK), Pasteurella multocida N-acetylglucosamine 1-phosphate uridylyltransferase (PmGlmU), and Pasteurella multocida inorganic pyrophosphatase (PmPpA). UDP-Gal was obtained from Gal, ATP, and UTP via SA2 containing three enzymes including Streptococcus pneumoniae galactokinase (SpGalK), Bifidobacterium longum UDP-sugar synthase (BLUSP), and PmPpA. GDP-Fuc was formed from Fuc, ATP, and GTP via SA3 containing two enzymes including a bifunctional enzyme from Bacteroides fragilis that has both L-fucokinase and GDP-fucose pyrophosphorylase activities (BfFKP), and PmPpA.

CMP-Neu5Ac is formed from Neu5Ac and CTP via SA4 containing Neisseria meningitidis CMP-sialic acid synthetase (NmCSS). As provided herein, these SA systems were combined with suitable GTs for highly efficient synthesis of target HMOs. The GTs provided herein include a β1-3-N-acetylglucosaminyltransferase (03GlcNAcT) GT1a; a β1-3-galactosyltransferase (β3GalT) GT2a and a β1-4-galactosyltransferase (β4GalT) GT2b; an α1-2-fucosyltransferase (α2FucT) GT3a and an α1-3/4-fucosyltransferase (α3/4FucT) GT3b; as well as an α2-3-sialyltransferase (α3SiaT) GT4a and several α2-6-sialyltransferases (α6SiaTs) GT4b-GT4e.

### Example 11. HMO EASyMaps and StOPMe Synthesis of Neutral HMOs with a Tetraose Core

HMOs were produced efficiently in a target-oriented chemoenzymatic synthetic process from LacβNHCbz (1) using a Stepwise One-Pot Multienzyme (StOPMe) strategy with a donor pre-generated from the corresponding monosaccharides and nucleoside triphosphates using related sugar activation (SA) enzymes. To guide target-oriented synthesis, enzymatic assembly synthetic maps (EASyMaps) were created herein to access the sixteen possible neutral HMOs containing the LNT (FIG. 11A) or the LNnT (FIG. 11B) tetraose core with 0-3 fucose residues (FIG. 10c) as shown in FIG. 4. Except for SA4 which was designed to be performed together with a suitable sialyltransferse in the same reaction step due to the relative instability of CMP-Neu5Ac, all other sugar activation processes (SA1-SA3) were carried out before the first glycosylation step in the process to reduce overall reaction time. As provided herein, the sugar nucleotides generated such as UDP-GlcNAc, UDP-Gal, and/or GDP-Fuc are stable throughout the whole StOPMe reaction progress to obtain the target HMOβNHCbz.

Every NHCbz-tagged HMO target was synthesized from LacβNHCbz (1) in one-pot in a stepwise procedure. Enzymes were deactivated after each glycosylation step by incubating the reaction mixture in a boiling water bath for 5 min and cooling down. The reaction mixture was then used for the next step without intermediate product purification. An α2-6-sialylation protection strategy was used to achieve regioselective α1-3/4-fucosylation of the internal GlcNAc but not the Glc at the reducing end. In this strategy, trisaccharide was used as the acceptor substrate for Pd2,6ST-catalyzed OPME sialylation to add an Neu5Ac α2-6-linked to the internal Gal on the trisaccharide which protected the Glc at the reducing end from α1-3/4-fucosylation. The resulting tetrasaccharide can be extended with a β1-3 or a β1-4-linked Gal. After regioselective α1-3/4-fucosylation was completed, the undesired α2-6-linked Neu5Ac was removed using a sialidase-catalyzed reaction.

HMOs with a tetraose core all share a common trisaccharide GlcNAcβ3Lac. Therefore, the fist glycosylation step for the synthesis of target HMOβNHCbz was the extension of LacβNHCbz (1) to GlcNAcβ3LacβNHCbz by an NmLgtA (GT1a)-catalyzed glycosylation reaction with the in situ generation of UDP-GlcNAc from GlcNAc using SA1. Before this glycosylation step, except for CMP-Neu5Ac (generated by SA4), all other sugar nucleotides needed for future glycosylation steps were generated by adding all components needed for the corresponding sugar activation systems (SA1-SA3), shown in FIG. 4. Once the sugar nucleotides were generated, LacβNHCbz (1) and NmLgtA (GT1a) were added. After the reaction went to completon, the enzymes were deactivated by heating the reaction mixture in a water bath for 5 min. The mixture was cooled down and used directly for the next step reaction.

By following the bottom route in the map FIG. 11A, extension of the trisaccharide GlcNAcβ3LacβNHCbz using a β1-3-linked Gal by Cvβ3GalT (GT2a)-catalyzed reaction with UDP-Gal produced in the first step via SA2 led to the formation LNTβNHCbz (7). After the reaction went to completon, the enzymes were deactivated by heating the reaction mixture in a water bath for 5 min. The mixture was cooled down. For target LNTβNHCbz (7), the product was purified by a single C18-cartridge. For target LNFP-IβNHCbz (8), the cooled down reaction mixture without purification was used directly for extension using Hm2FT (GT3a)-catalyzed α1-2-fucosylation with GDP-Fuc generated from L-Fuc via SA3 in the first step. After the reaction went to completon, the enzymes were deactivated by heating the reaction mixture in a water bath for 5 min. The mixture was cooled down. The cooled down mixture was used for purification to obtain pure LNFP-IβNHCbz (8) or used without purification for extension using an Hp3/4FT (GT3b)-catalyzed reaction with GDP-Fuc generated from L-Fuc via SA3 in the first step to obtain the pure target LNTFHepβNHCbz (23) after purification. Therefore, tetrasaccharide LNTβNHCbz (2), pentasaccharide LNFP-IβNHCbz (8), and heptasaccharide LNTFHepβNHCbz (23), obtained in 2, 3, and 4 steps, respectively, with overall yields ranging from 84-92%. Each compound was obtained from 100 mg LacβNHCbz (1) with a single C18-cartridge purification process.

By following the top route in the map FIG. 11A, extension of the trisaccharide GlcNAcβ3LacβNHCbz using an Hp3/4FT (GT3b)-catalyzed reaction with GDP-Fuc generated from L-Fuc via SA3 in the first step, deactivating of the enzymes, followed by extension of the product with a β1-3-linked Gal using a Cvβ3GalT (GT2a)-catalyzed reaction with UDP-Gal generated from Gal via SA2 in the first step led to the formation LNFP-VβNHCbz (9). The enzymes are deactivated and the product can be purified to obtain pure LNFP-VβNHCbz (9). Alternatively, the cooled down reaction mixture without purification was used for α1-4-fucosylation using Hp3/4FT, or for α1-2-fucosylation using Hm2FT, for the formation of LNDFH-IIβNHCbz (10) and LNDFH-IIIβNHCbz (24) respectively, with GDP-Fuc generated from L-Fuc via SA3 in the first step. Overall, each of targets LNFP-VβNHCbz (9), LNDFH-IIβNHCbz (10), and LNDFH-IIIβNHCbz (24), was obtained from 100 mg LacβNHCbz (1) in 3 or 4 steps with a yield of 88% or 92%.

By following the middle in the map FIG. 11A, Pd2,6ST-catalyzed α2-6-sialylation with in situ generation of CMP-Neu5Ac from Neu5Ac with SA4 was used to protect the Glc from fucosylation by Hp3/4FT. Followed by enzyme deactivation and extension of the terminal GlcNAc using a β1-3-linked Gal by Cvβ3GalT (GT2a)-catalyzed reaction with UDP-Gal generated from Gal via SA2 in the first step, two different processes were carried out for reaching to two different targets. For one of the processes, after enzyme deactivation, the reaction mixture went through Hp3/4FT (GT3b)-catalyzed reaction with GDP-Fuc generated from L-Fuc via SA3 in the first step, enzyme deactivation, and sialidase SpNanA-catalyzed reaction to remove the α2-6-linked Neu5Ac which served as a protecting group for Glc from Hp3/4FT-catalyzed α1-3-fucosylation, enzyme deactivation, and C18 cartridge purification, pure LNFP-IIβNHCbz (25) was obtained. For the other process, after enzyme deactivation, the reaction mixture went though Hp2FT (GT3a)-catalyzed reaction with GDP-Fuc generated from L-Fuc via SA3 in the first step, enzyme deactivation, Hp3/4FT (GT3b)-catalyzed reaction with GDP-Fuc generated from L-Fuc via SA3 in the first step, sialidase SpNanA-catalyzed reaction, enzyme deactivation, and C18 cartridge purification, pure LNDFH-IβNHCbz (26) was obtained. Overall, LNFP-IIβNHCbz (25) and LNDFH-IβNHCbz (26) were each obtained from 100 mg LacβNHCbz (1) in 85% and 80% yields and 5 or 6 steps, respectively, with a single C18-cartridge purification process. The synthesis of neutral HMOs with the LNnT core followed a similar HMO EASyMap, which is shown in FIG. 11B. The only difference from the map FIG. 11B designed for those containing the LNT core was to replace Cvβ3GalT (GT2a) in FIG. 11B by NmLgtB (GT2b) in FIG. 11B as highlighted in squares.

### Example 12. StOPme or OPME Synthesis of Sialylated HMOs with a Tetraose Core

With the access to all sixteen neutral HMOs containing a tetraose core, sialylation was carried out for those do not contain an α1-2-linked fucose at the terminal Gal (eight HMOβNHCbz targets marked with a four-point star in FIGS. 11A and 11B including compounds 7, 9, 10, 25, 3, 5, 6, and 30). This was achieved by using a single one-pot two-enzyme (OP2E) sialylation reaction from NHCbz-tagged fucosylated HMO product obtained in FIGS. 11A and 11B. By using the OP2E salic acid activation and synthesis system, α2-3-sialyltransferase PmST1_M144D was able to transfer Neu5Ac to the terminal galactose in LNTβNHCbz (7), LNFP-VβNHCbz (9), LNDFH-IIβNHCbz (10), LNFP-IIβNHCbz (25), LNnDFH-IIβNHCbz (6), and LNFP-IIIβNHCbz (30). α2-3-Sialyltransferase PmST3³³ was used to transfer Neu5Ac to the terminal 01-4-linked galactose on LNnTβNHCbz (3) and LNnFP-VβNHCbz (5). For synthesizing α2-6-sialosides, the α2-6-sialyltransferase Pd2,6ST_A200Y/S232Y was used for sialylating LNTβNHCbz (7), LNFP-VβNHCbz (9), LNnTβNHCbz (3), and LNnFP-VβNHCbz (5). However, Pd2,6ST_A200Y/S232Y was not able to transfer Neu5Ac to the acceptor containing a Fucα1-3/4GlcNAc component. StOPMe procedure was also successfully applied to the syntheses of Neu5Acα3LNTβNHCbz (LSTaβNHCbz) (32) and Neu5Acα3LNnTβNHCbz (LSTcβNHCbz) (38) directly from LacβNHCbz (1) (100 mg) by following the EASyMaps shown in FIGS. 11A and 11B for the synthesis of LNTβNHCbz (7) and LNnTβNHCbz (3), respectively, with an additional one-pot two-enzyme (OP2E) sialylation step.

### Example 13. Recombinant hST6GALNAC V and Mutants and their Applications in Synthesizing Neu5Acα2-6GlcNAc-Containing HMOs

The library of HMOs with an LNT core also include five compounds containing a quite unique Neu5Acα2-6GlcNAc linkage that has been found in several colon cancer cell lines. For example, a complex disialyl Lewis a-containing lacto-series ganglioside was shown to be most likely contributed by the combined functions of FUT-3 and ST6GALNAC VI, but not ST6GALNAC V. On the other hand, human ST6GALNAC V (hST6GALNAC V) was shown to be responsible mainly for the production of ganglio-series GD1a ganglioside by sialylating GM1b for the formation of Neu5Acα2-6GalNAc linkage. The sialyltransferase that is responsible for forming the Neu5Acα2-6GlcNAc linkage in HMOs is not clear. Nevertheless, a recombinant hST6GALNAC V expressed in HEK293 cells has been used for catalyze the addition of a Neu5Ac α2-6-linked to the GlcNAc or the GalNAc in the acceptor substrate for the synthesis of a DSLNT derivative and a disialyl Gb5 (DSGb5) ganglioside glycan, respectively. Its expression in E. coli, as provided herein, had not been reported previously.

In order to synthesize target Neu5Acα2-6GlcNAc-containing HMOs, an N-terminal truncated recombinant hST6GALNAC V fused with an N-terminal maltose binding protein (MBP) and a C-terminal His₆-tag was cloned using a codon optimized synthetic gene. The resulting MBP-Δ50hST6GALNAC V-His₆ was successfully expressed as a soluble and active fusion protein in E. coli Origami B (DE3) cells containing pGro7. Its expression level was 440 mU per liter culture when Neu5Acα2-3LNTβNHCbz was used as the acceptor, 1 U=1 μmol min¹ at 37° C., pH 7.5. Acceptor substrate specificity was carried out and the results showed that the recombinant MBP-Δ50hST6GALNAC V-His₆ is promiscuous and is capable of using different HMOβNHCbz as acceptor substrates.

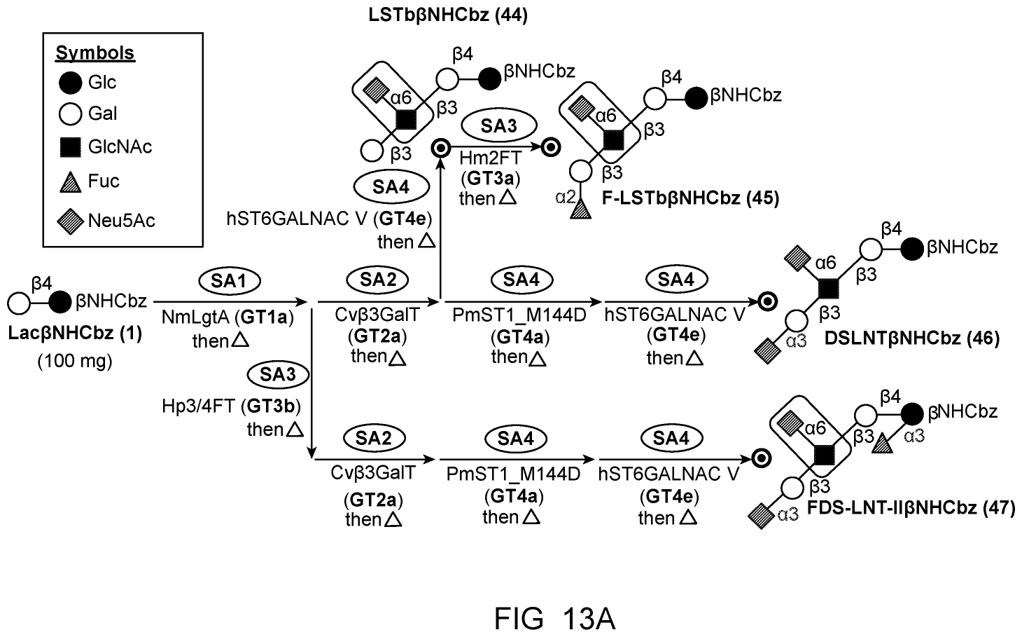

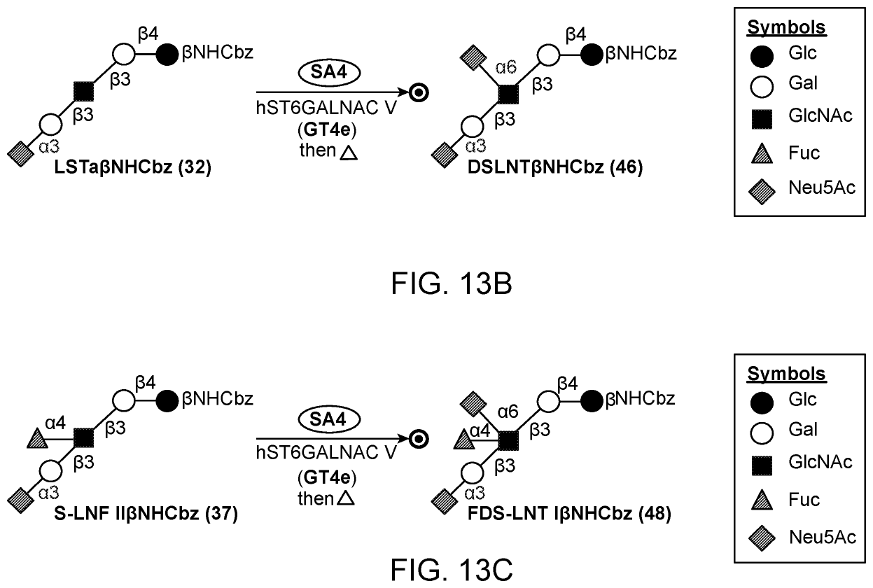

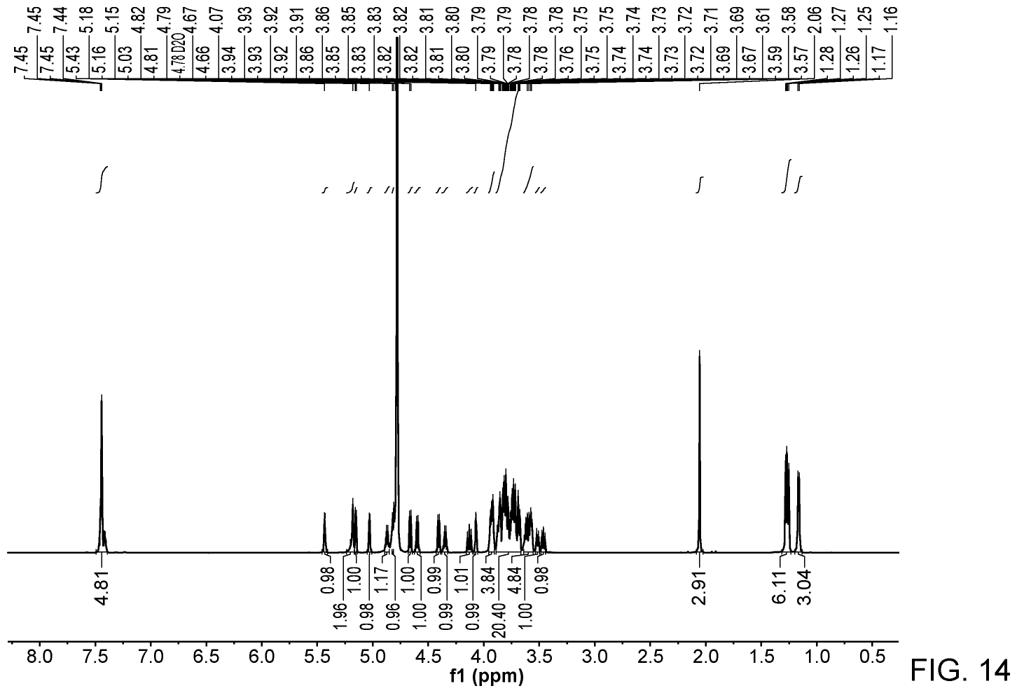

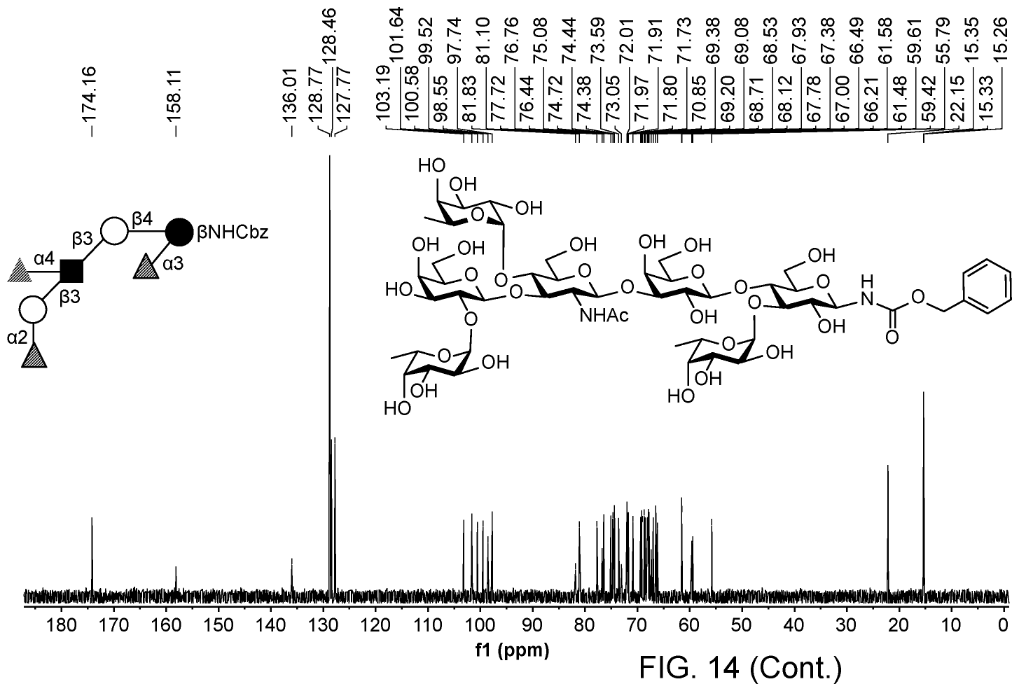

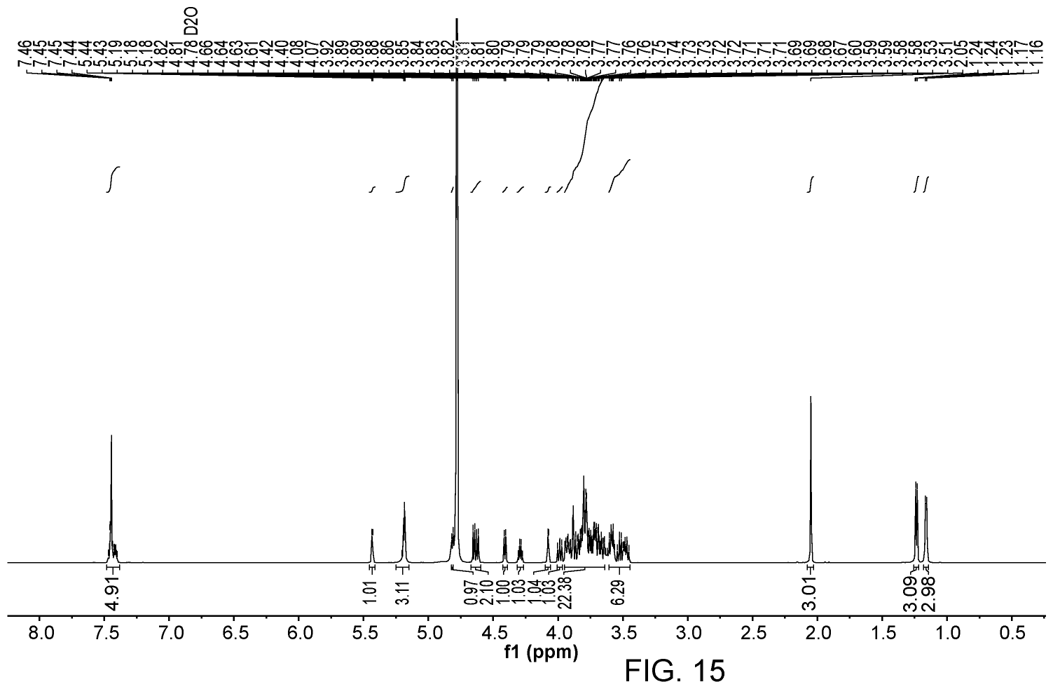

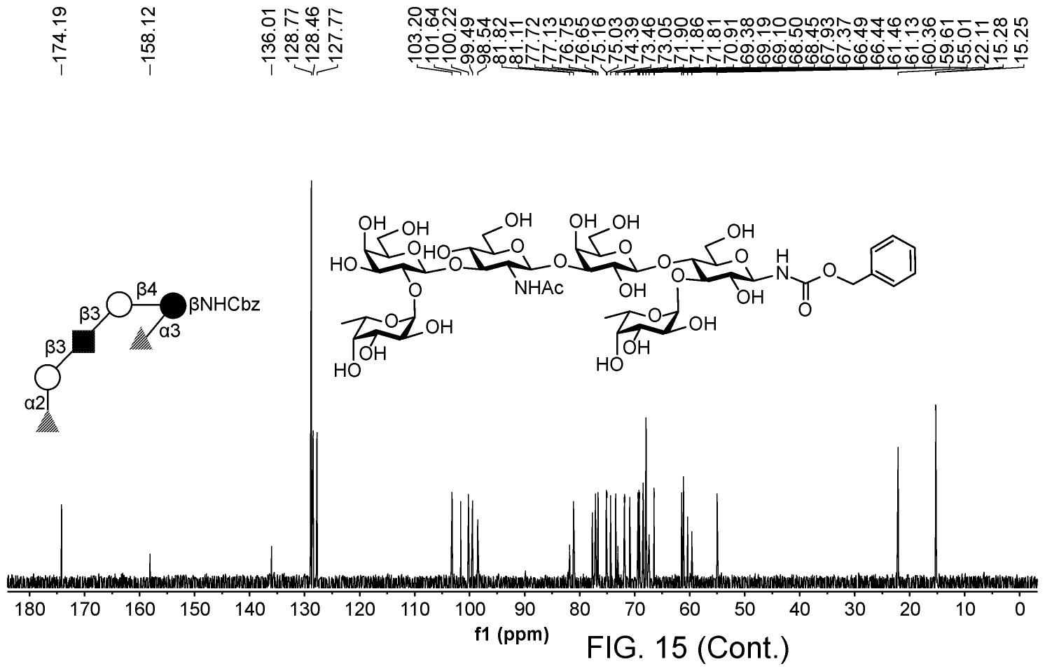

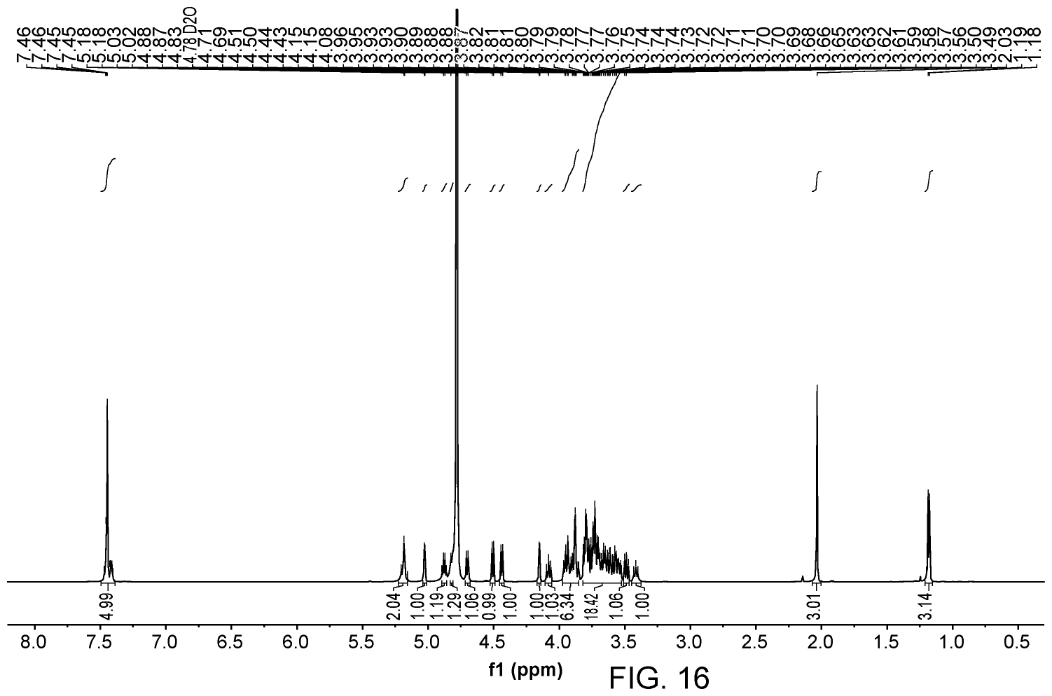

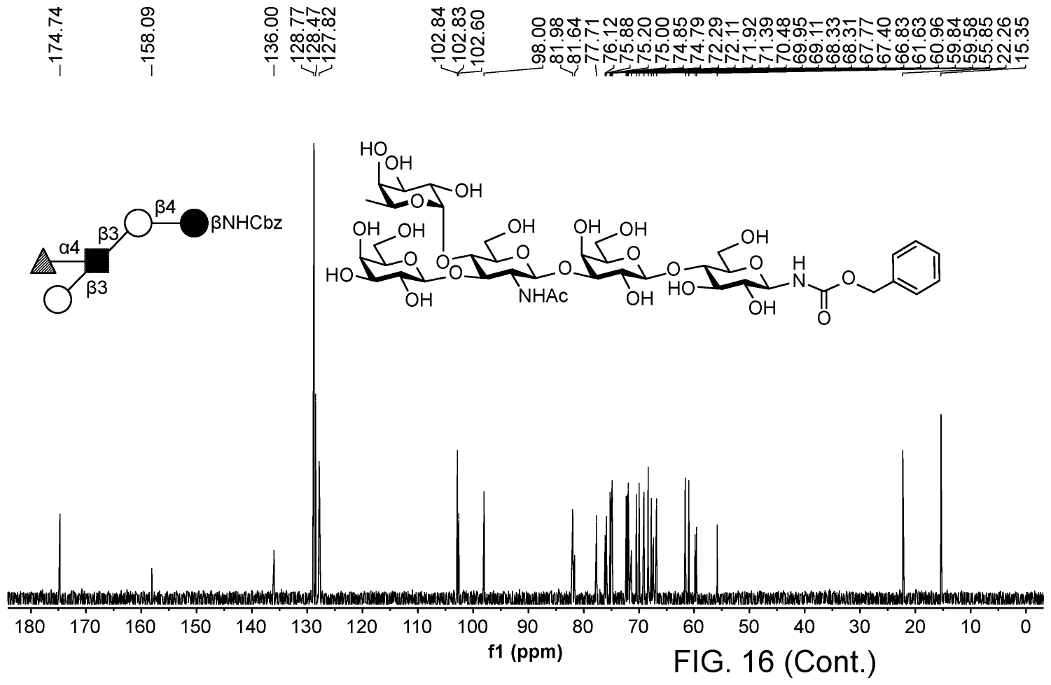

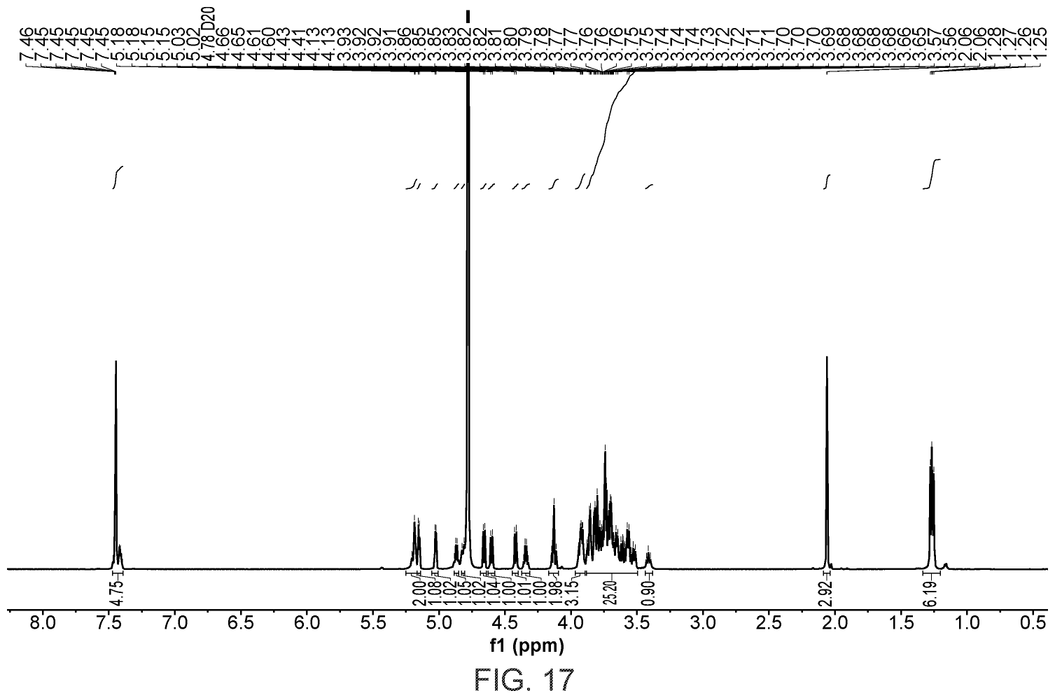

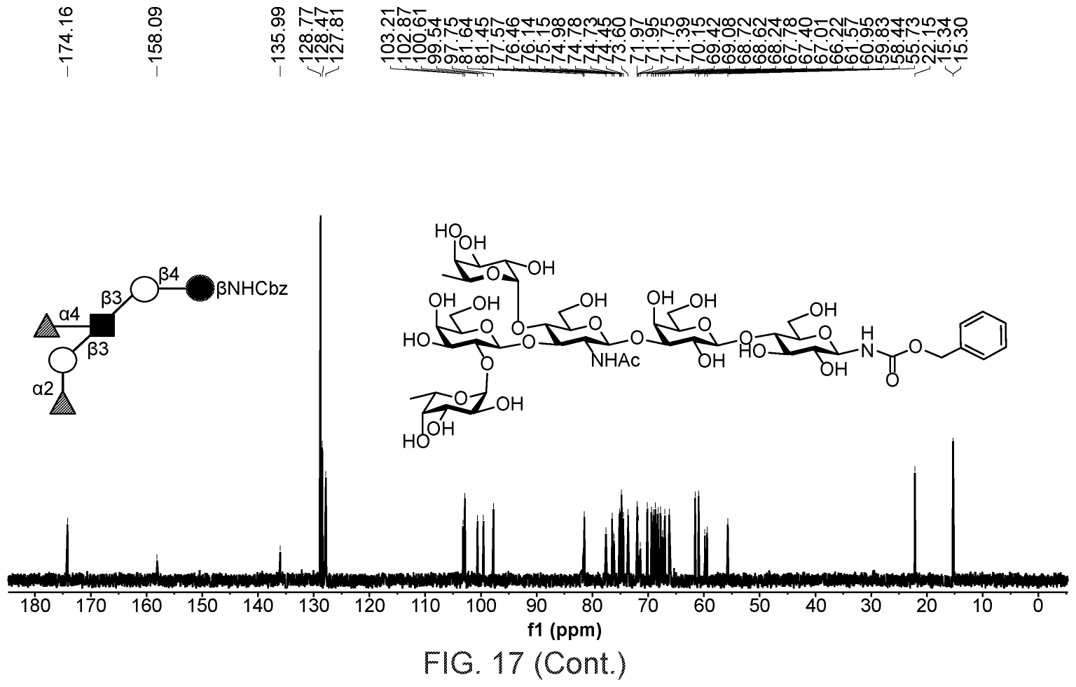

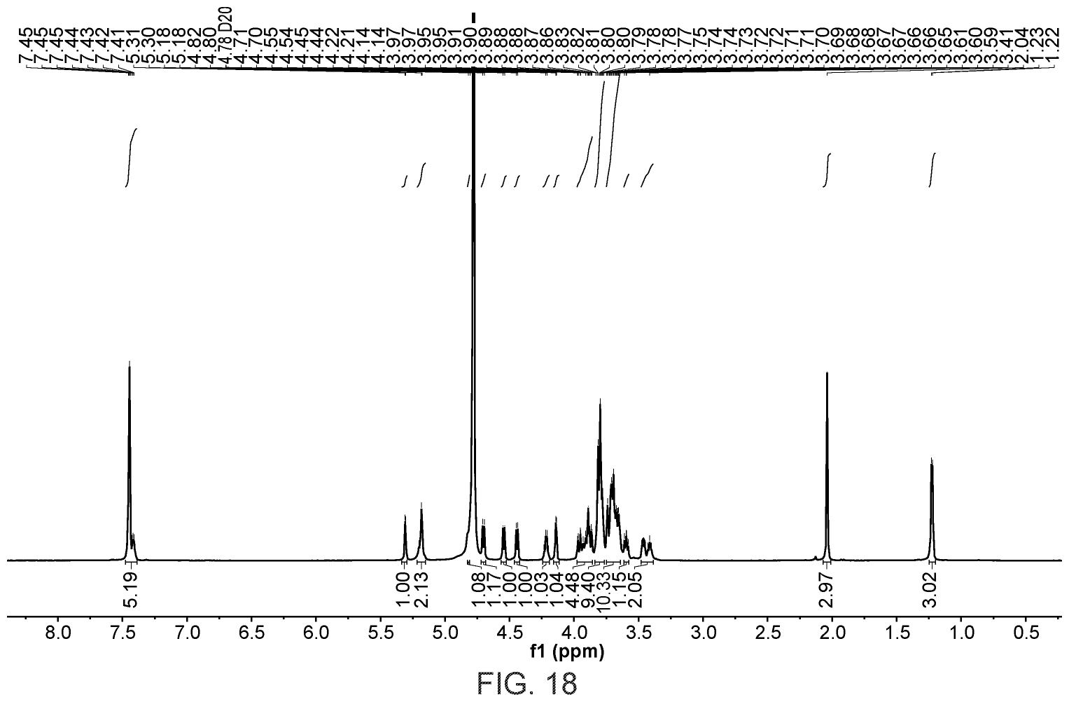

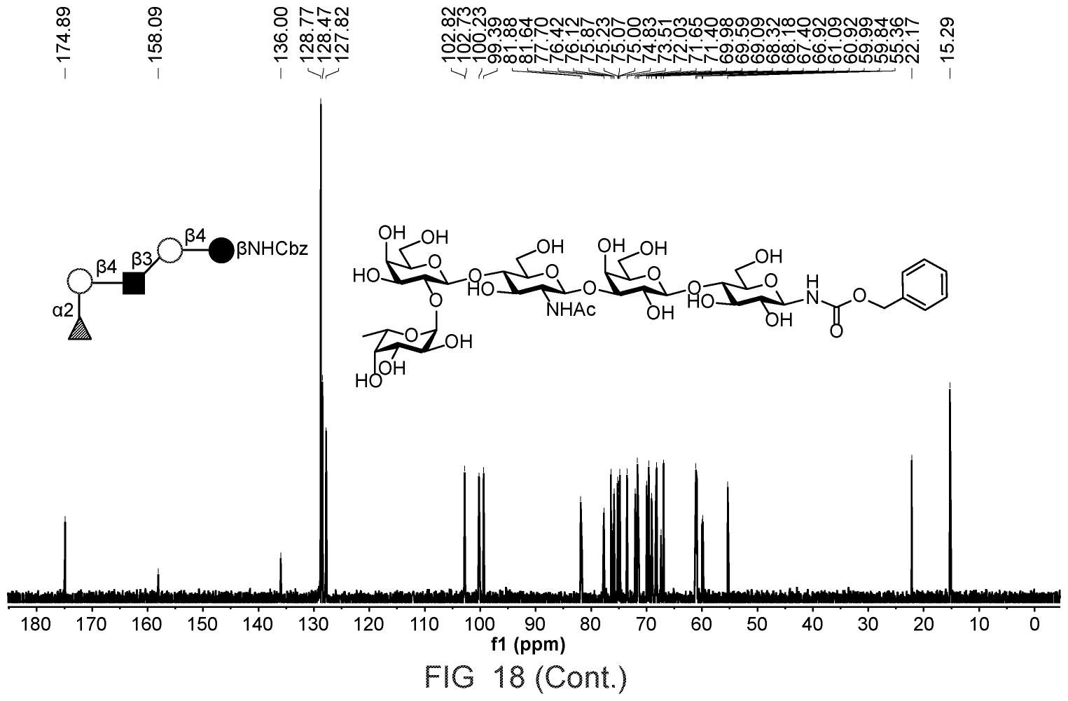

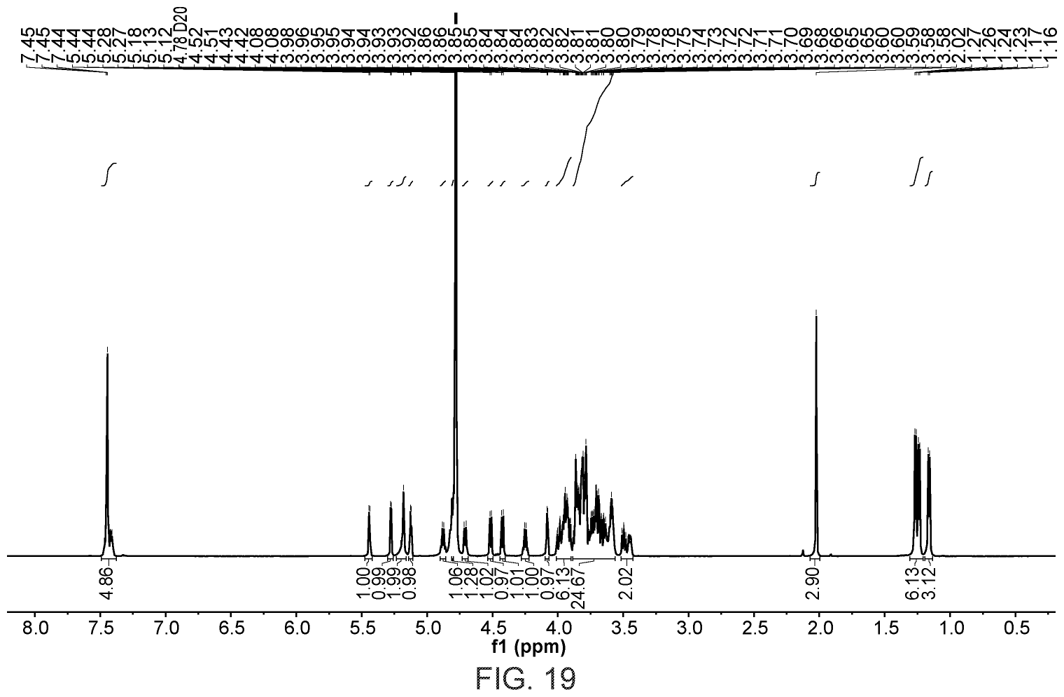

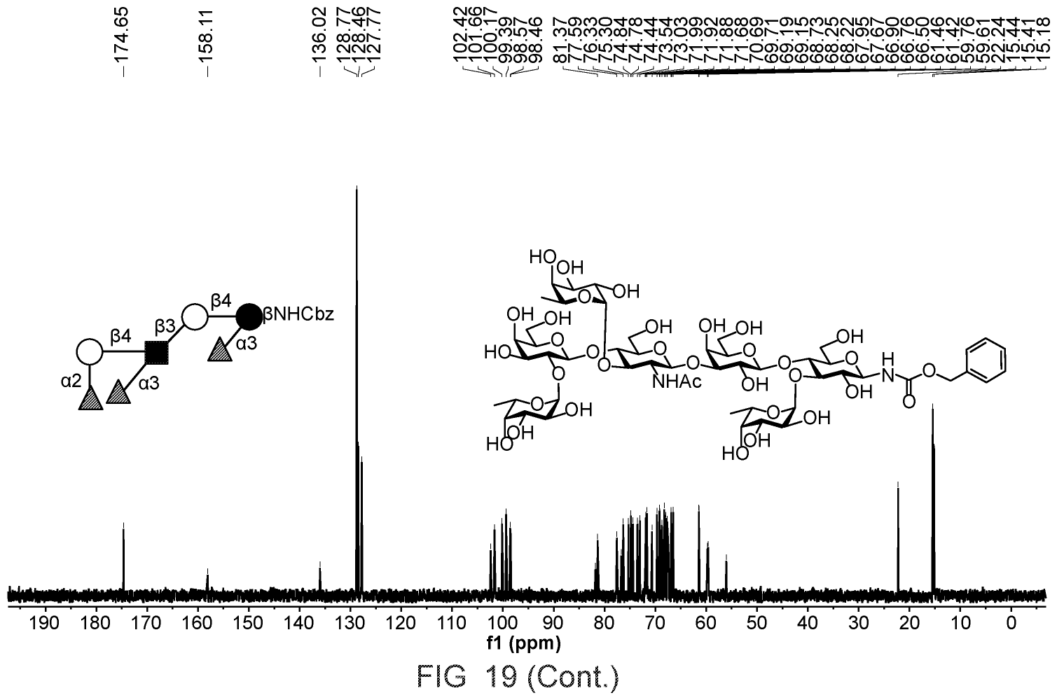

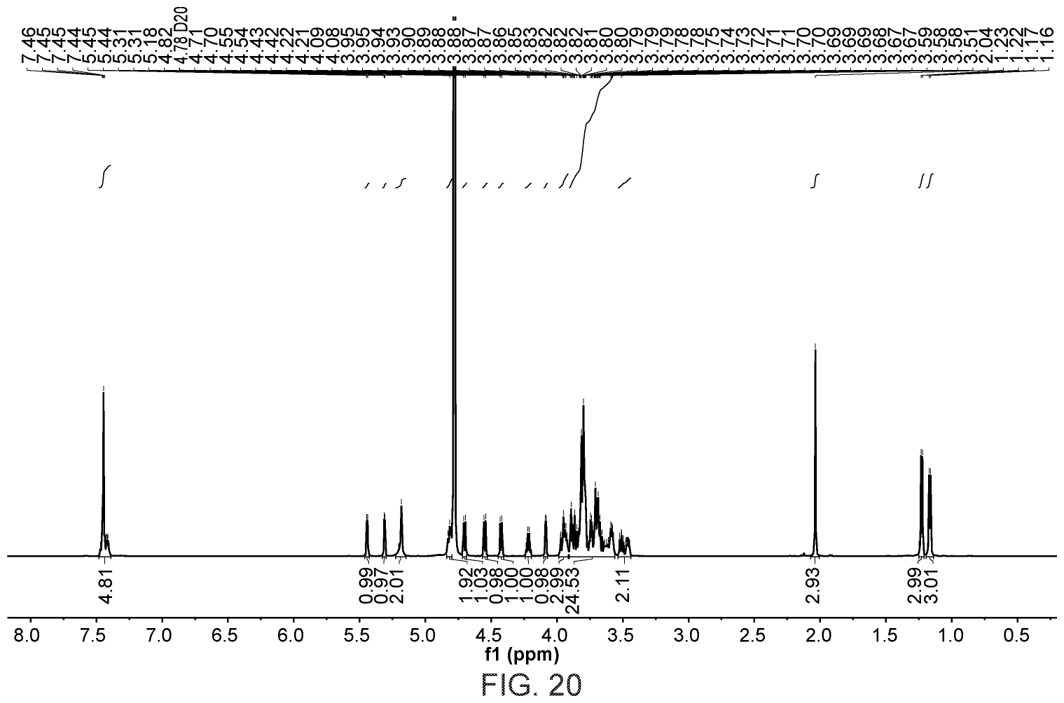

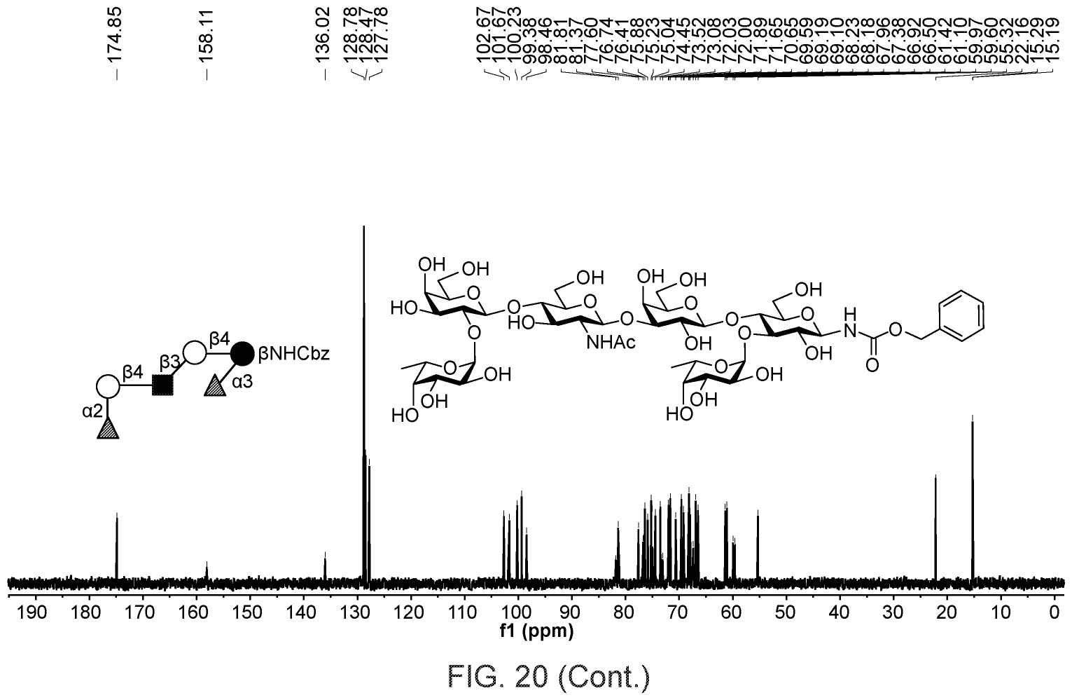

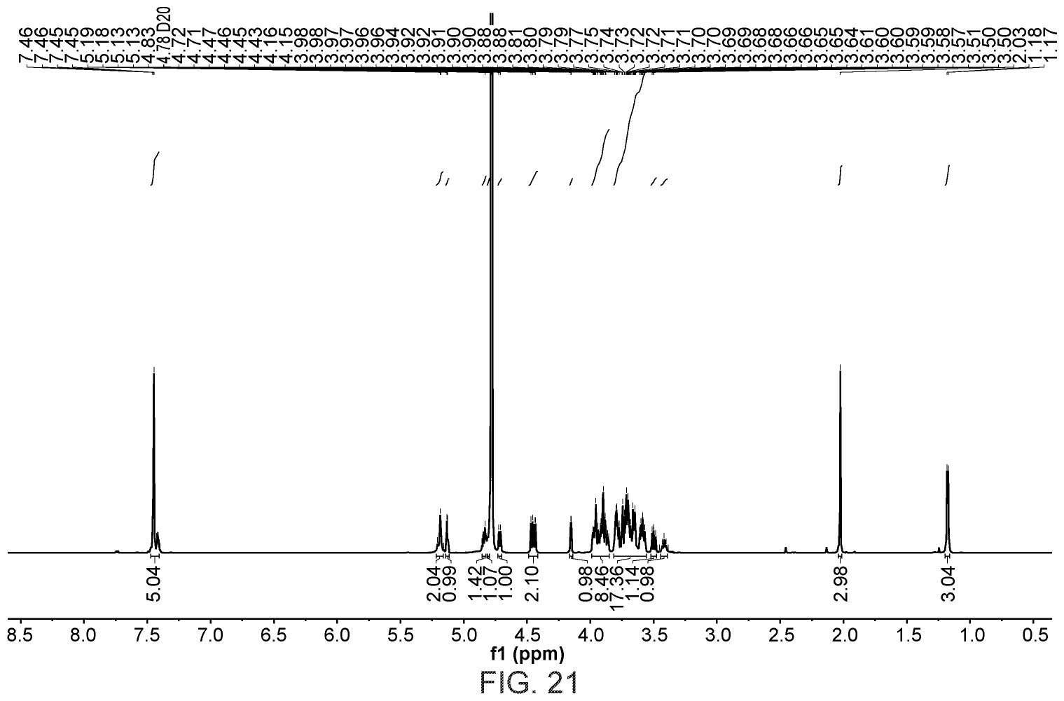

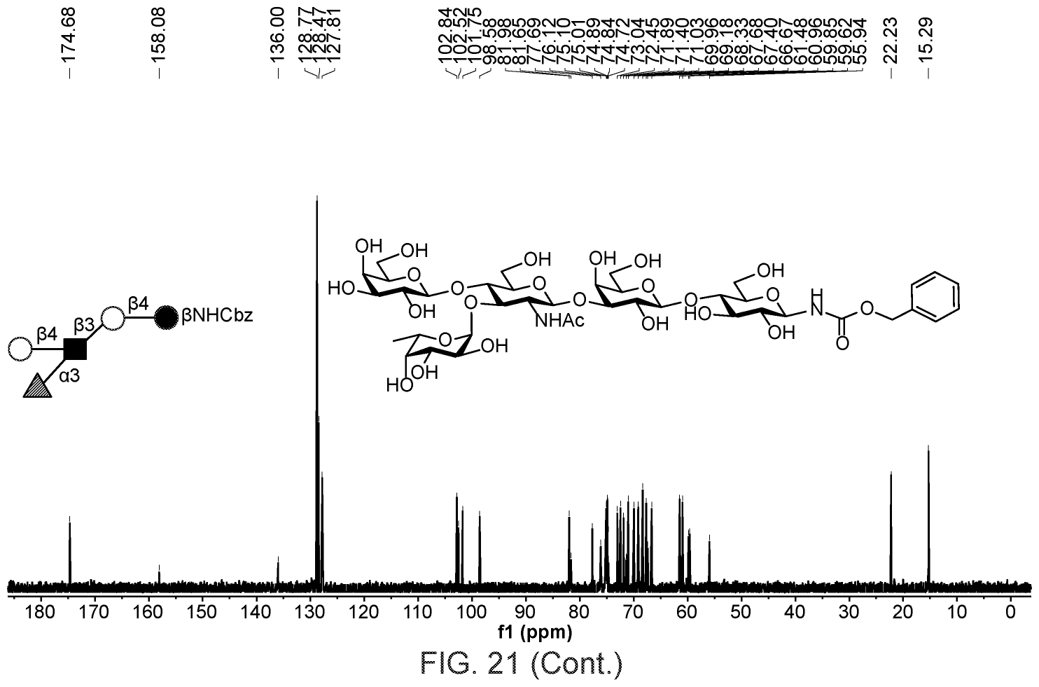

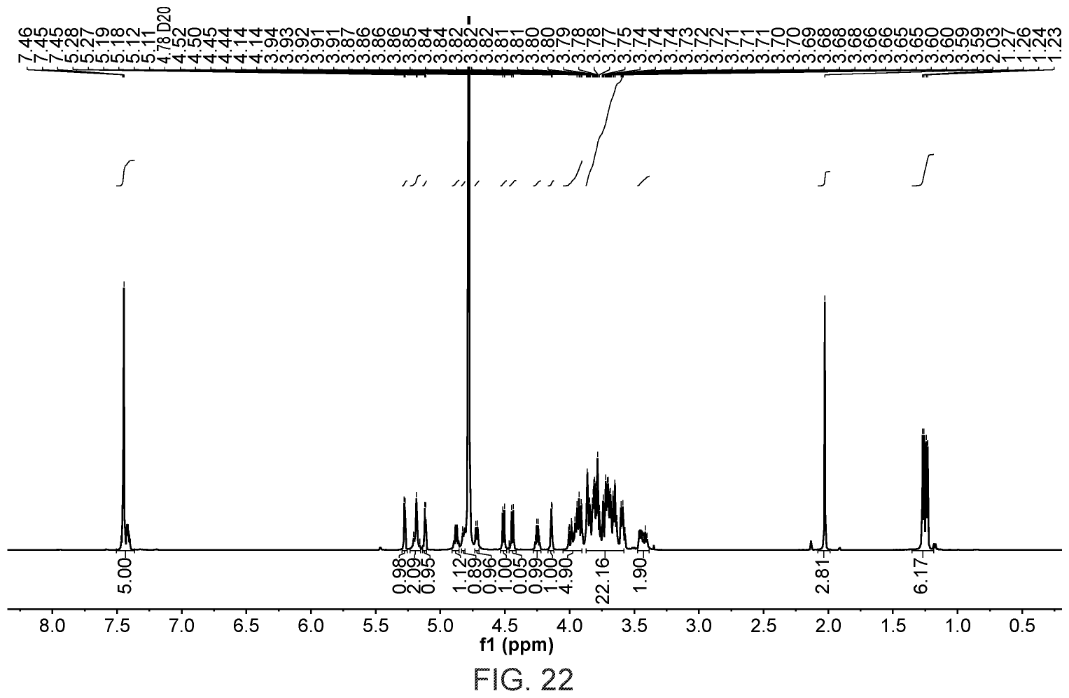

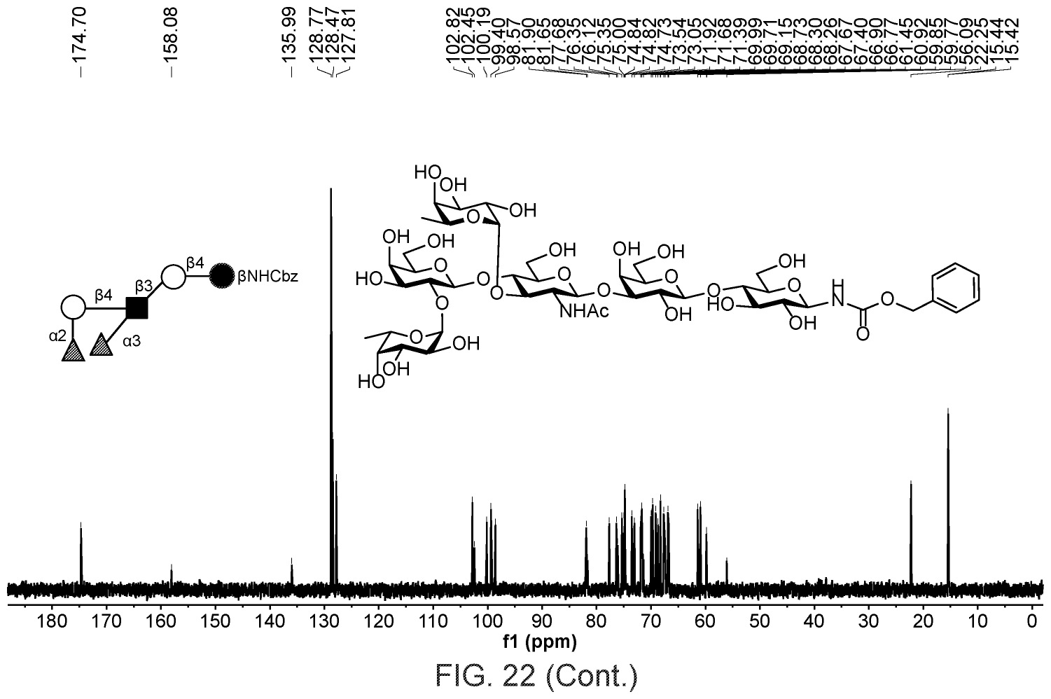

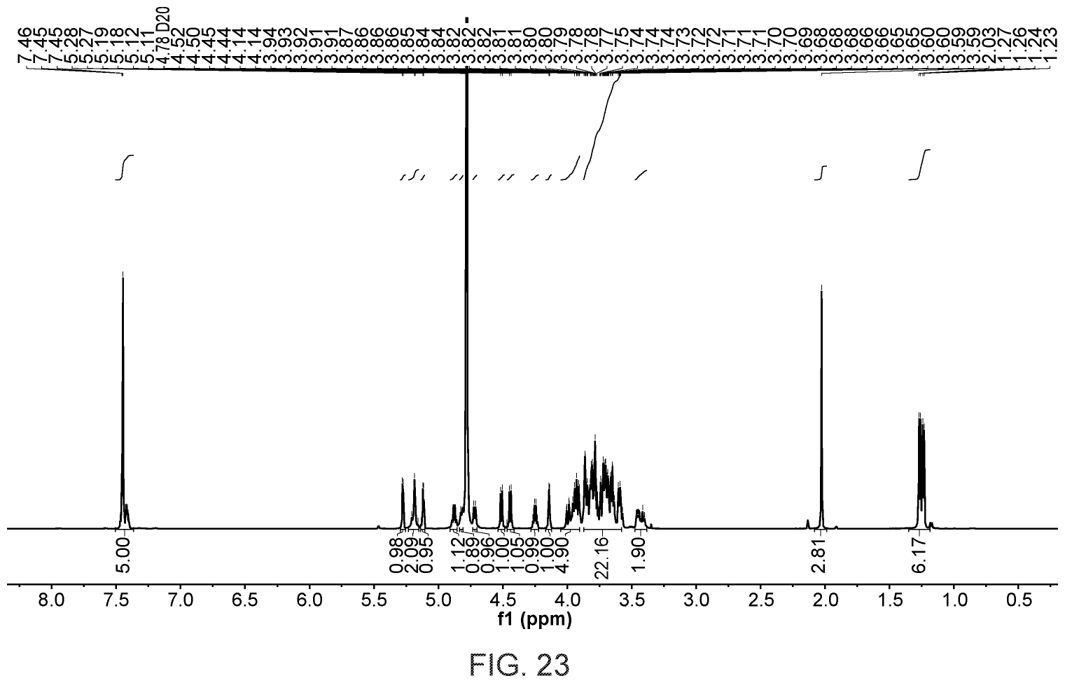

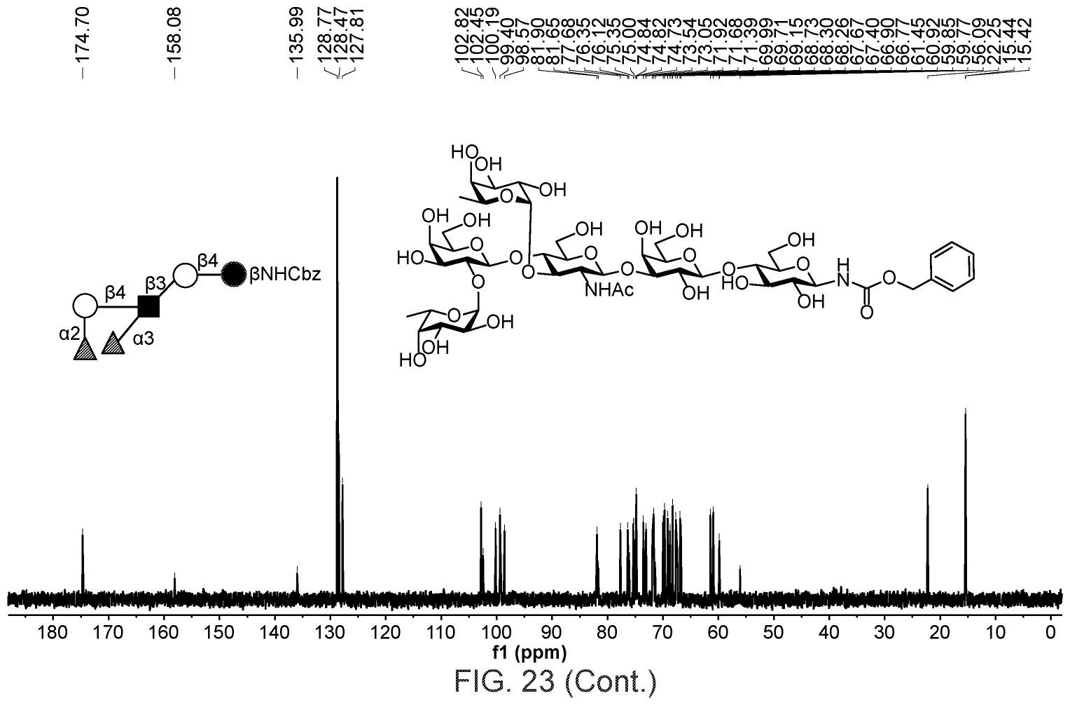

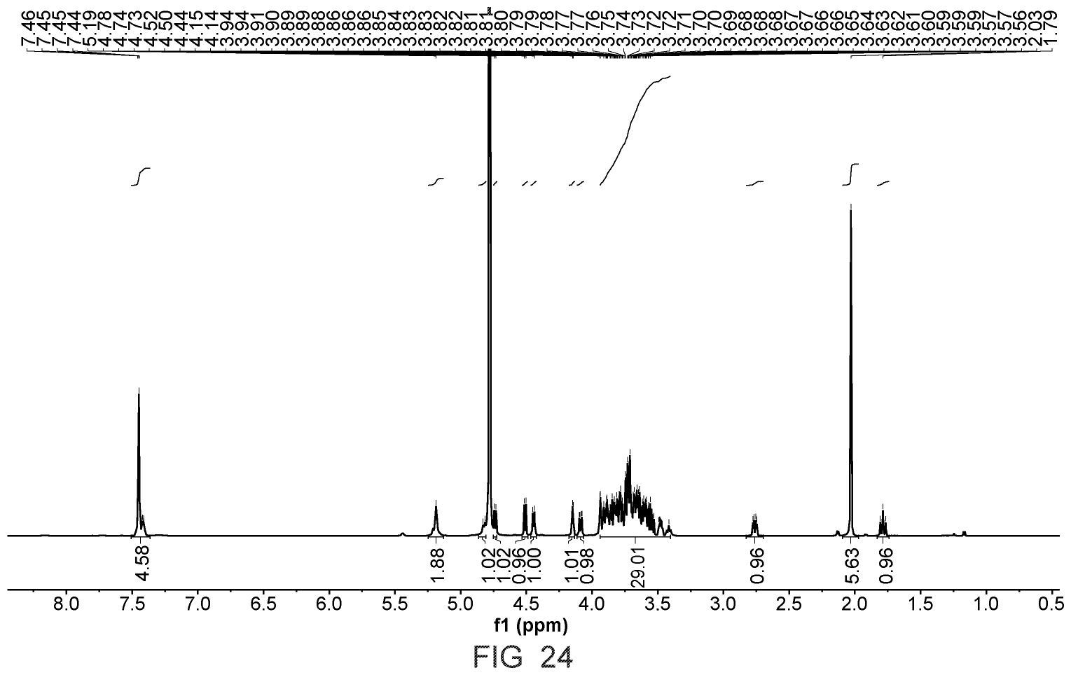

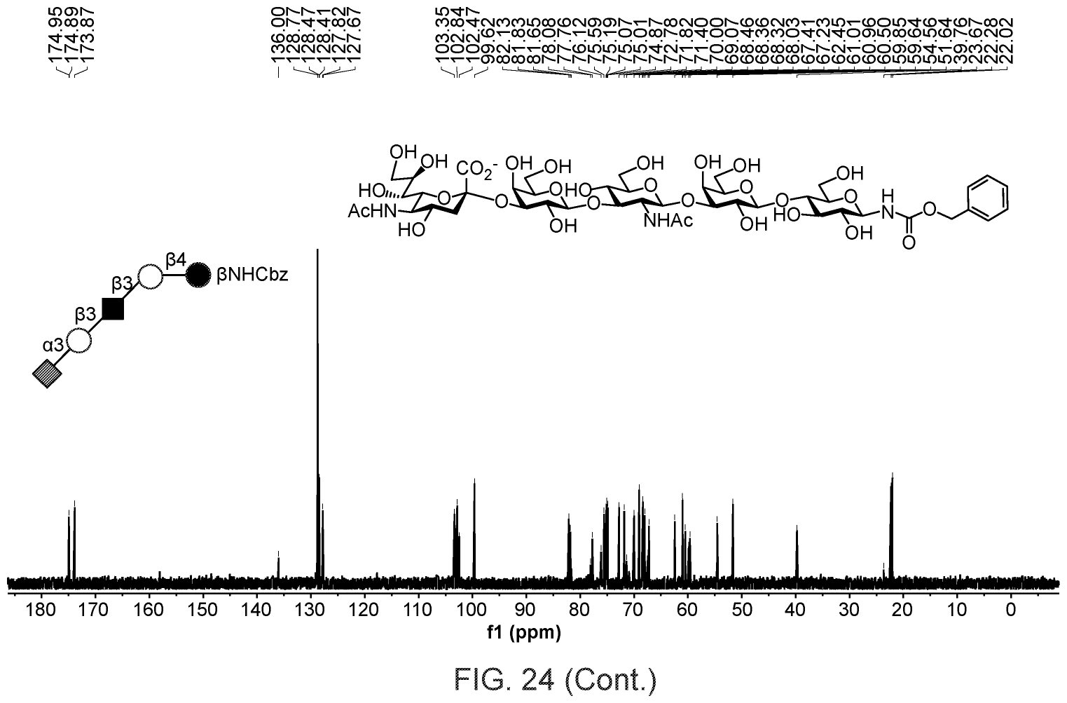

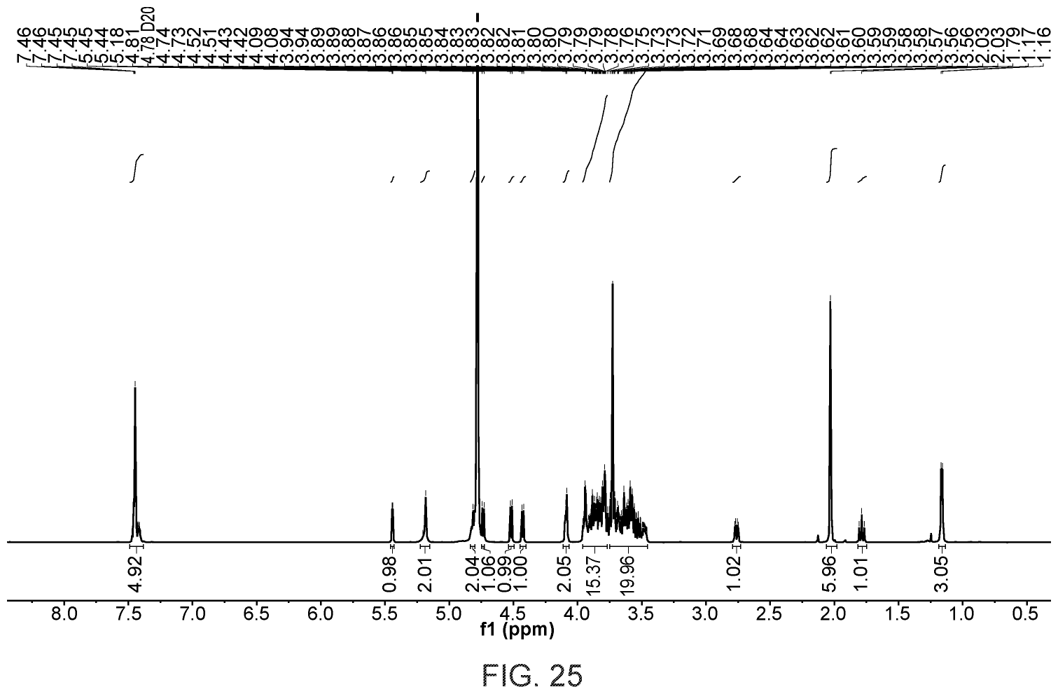

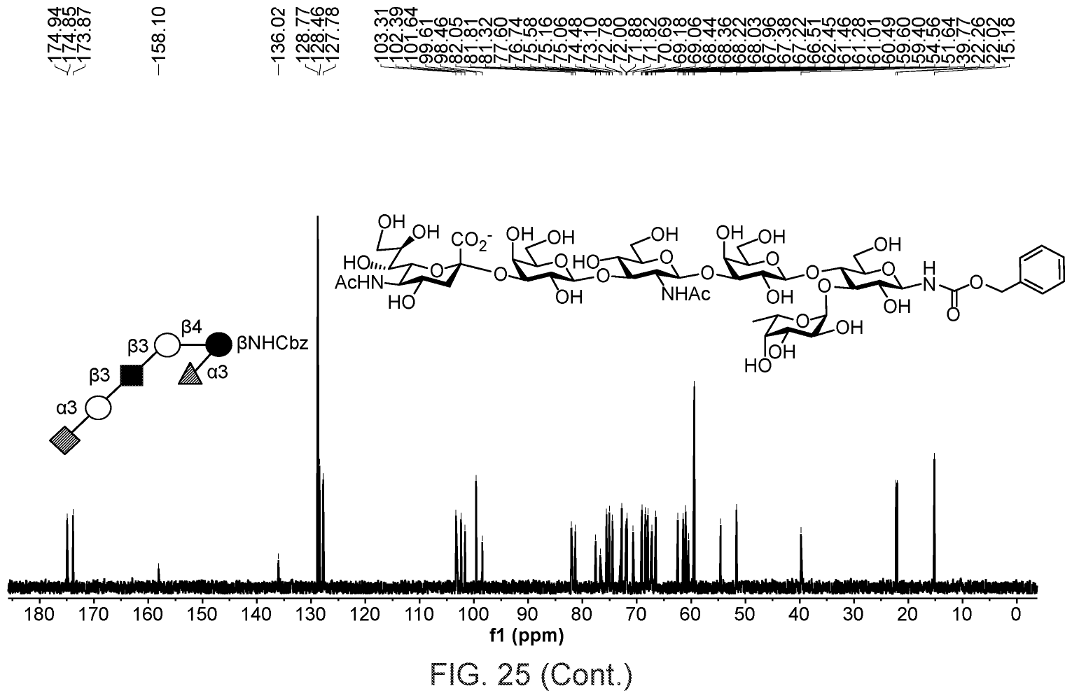

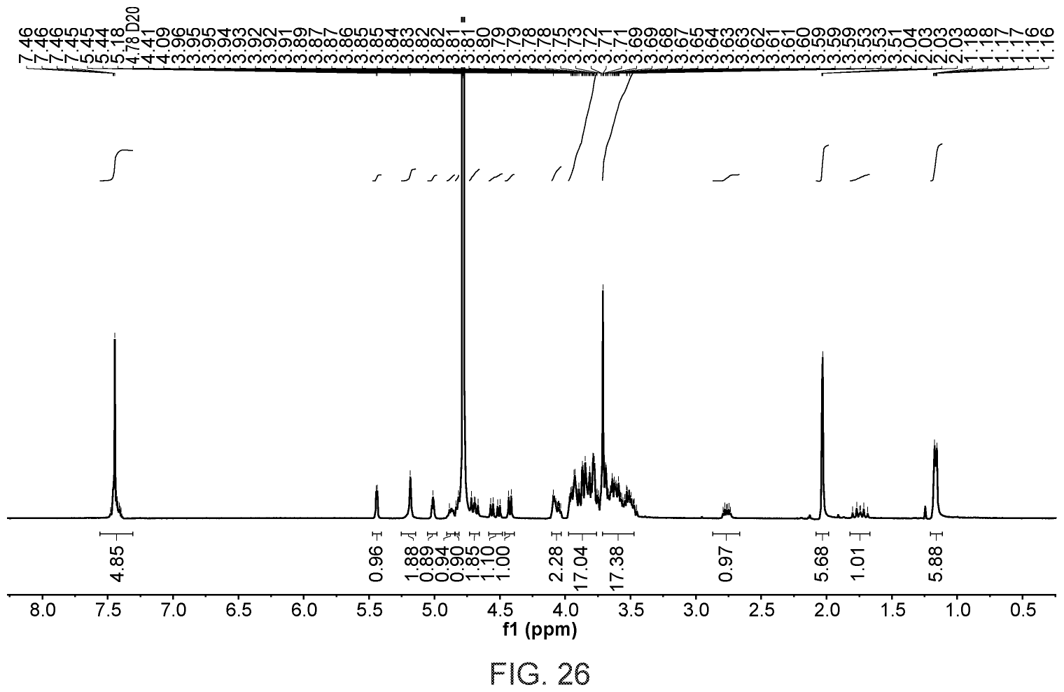

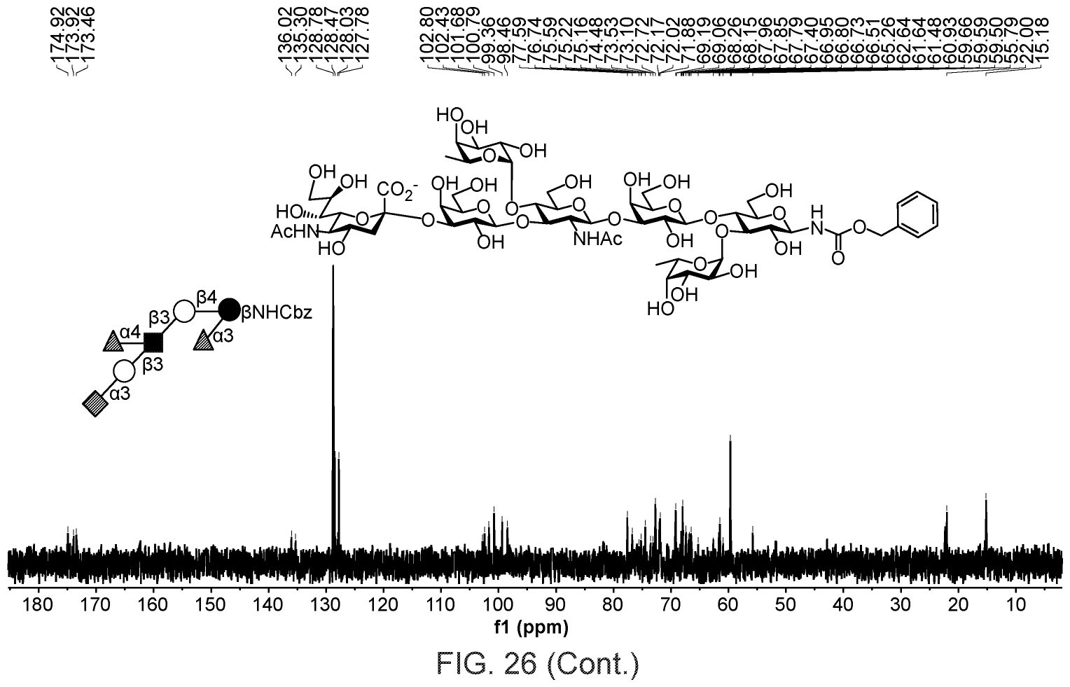

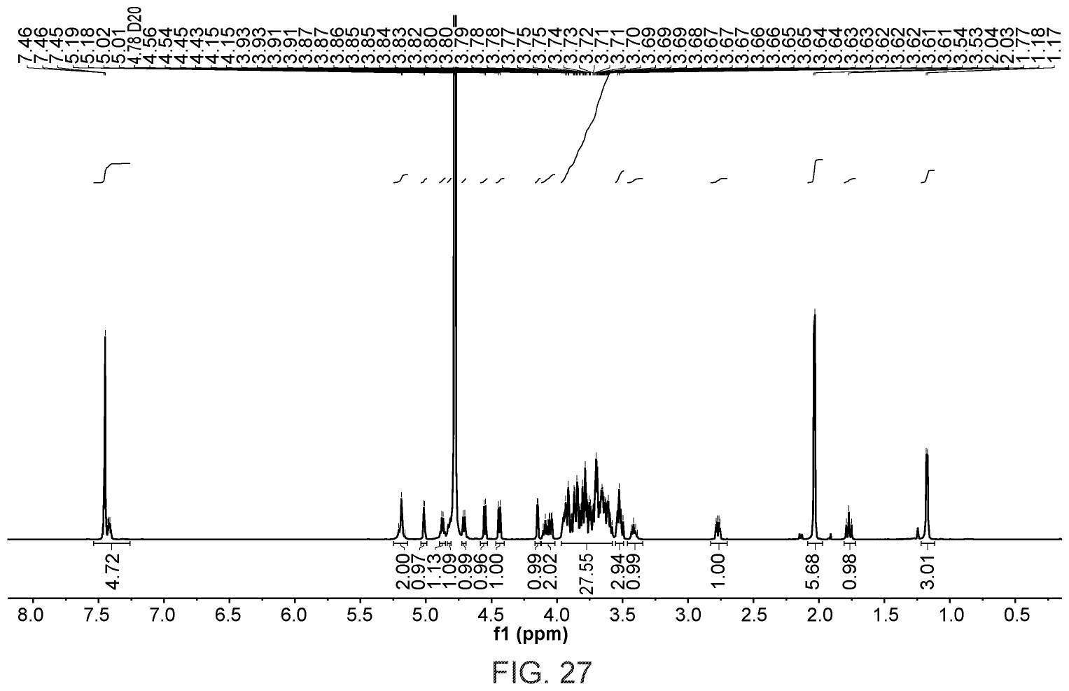

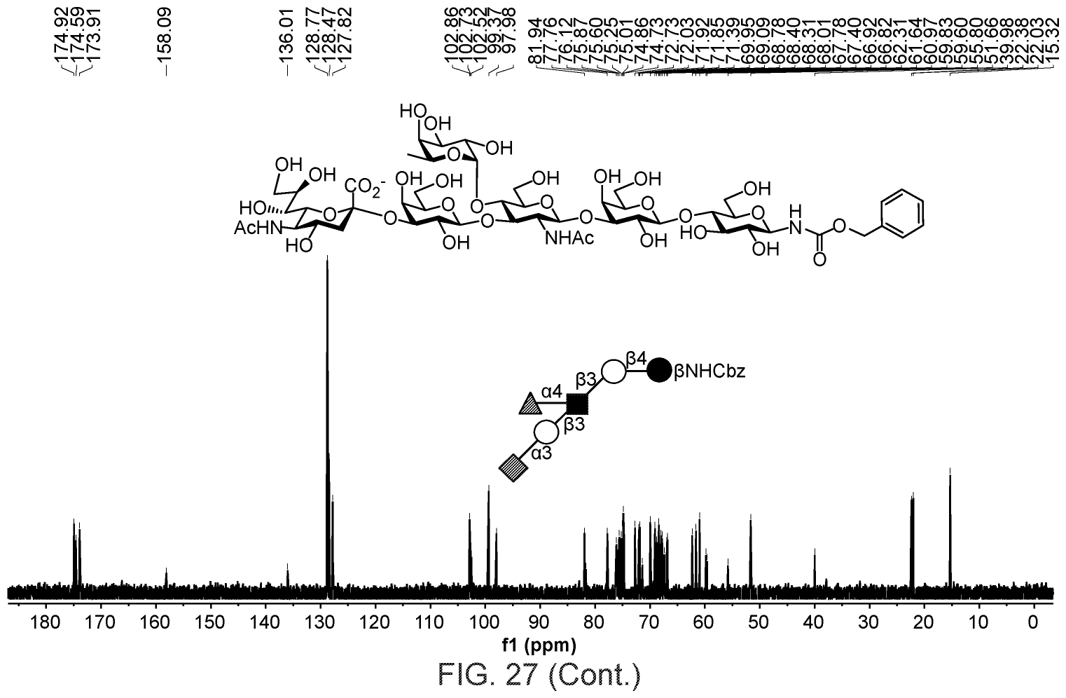

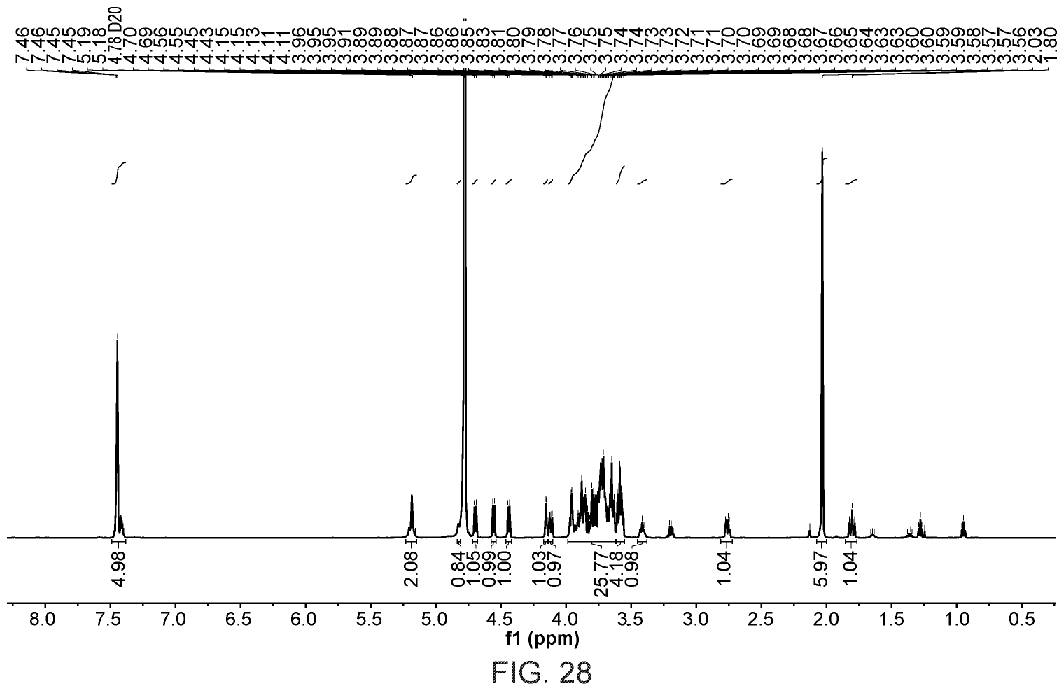

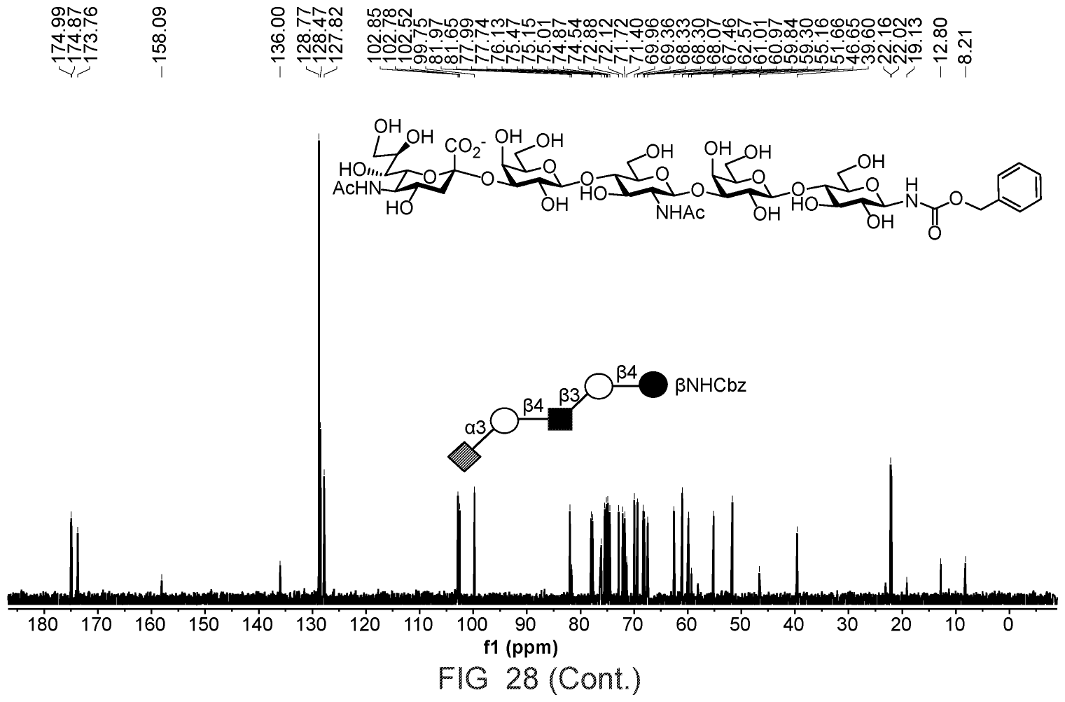

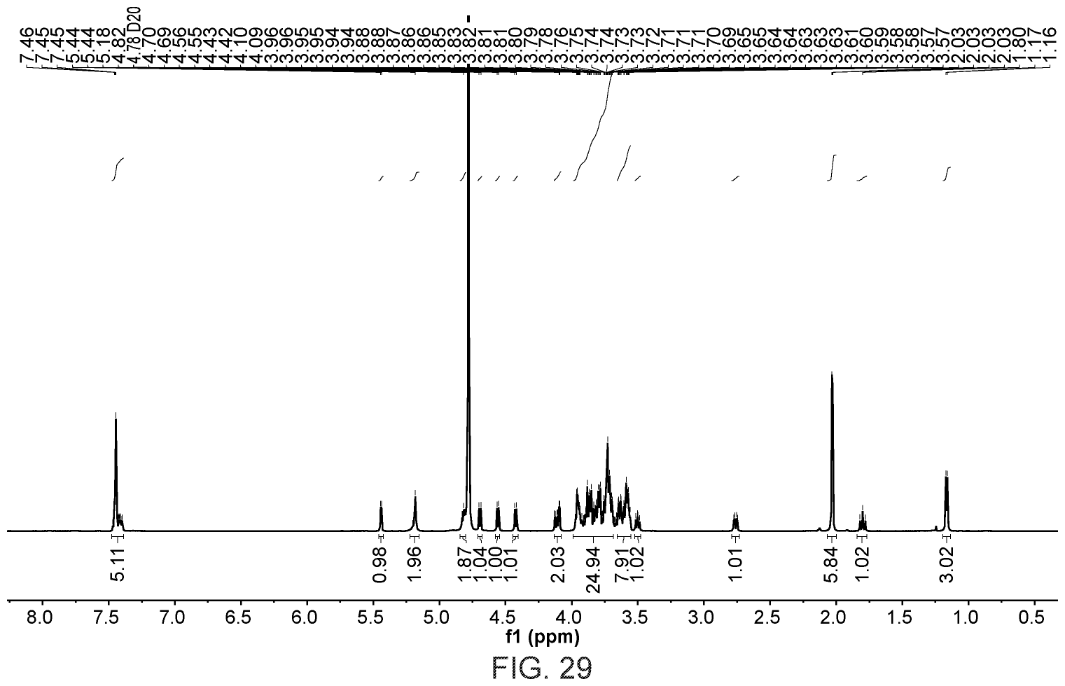

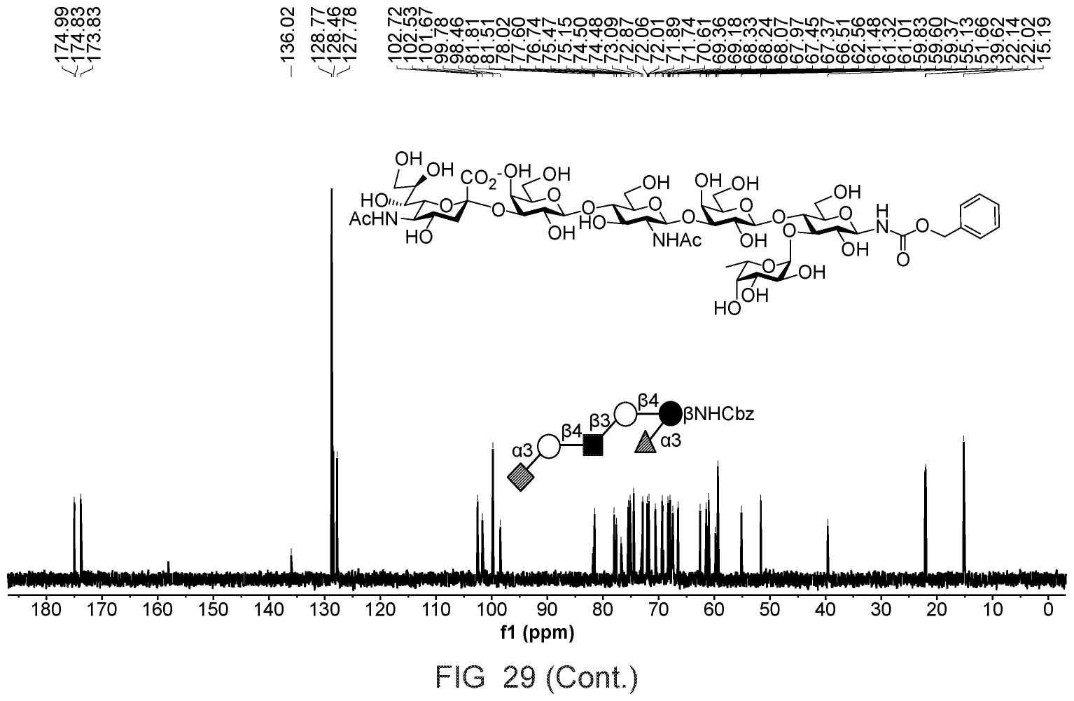

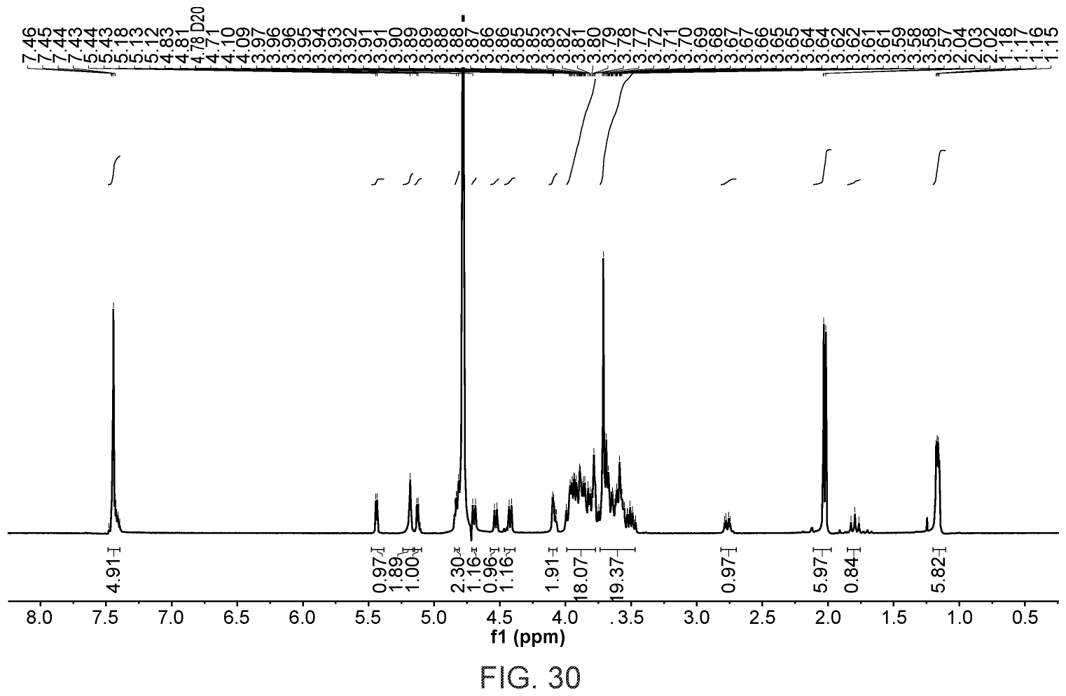

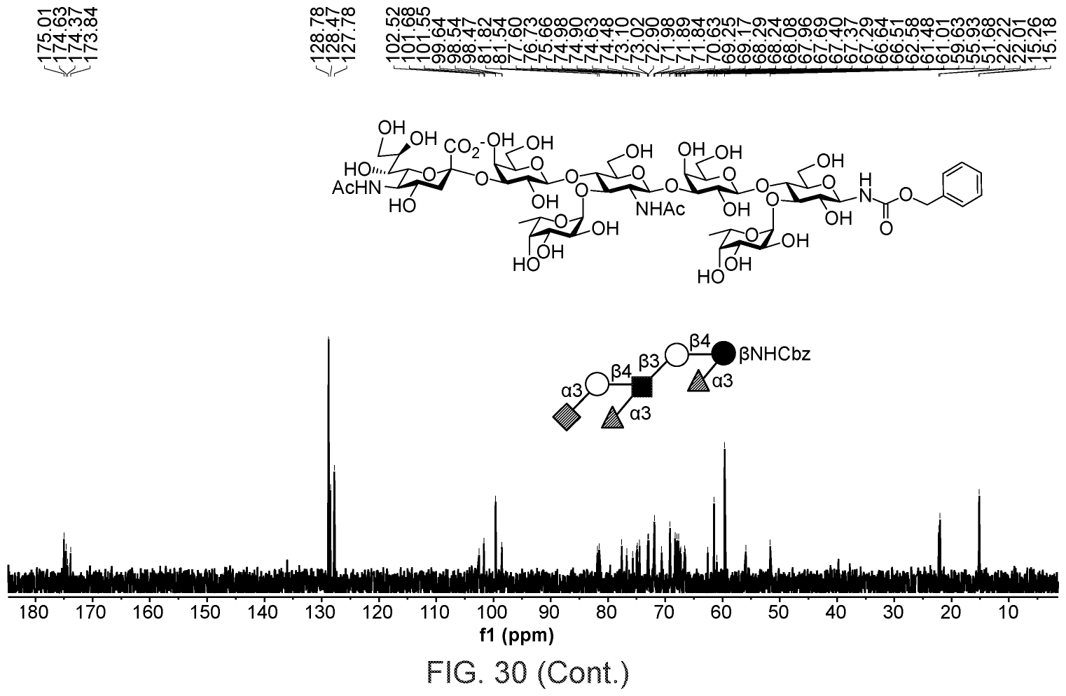

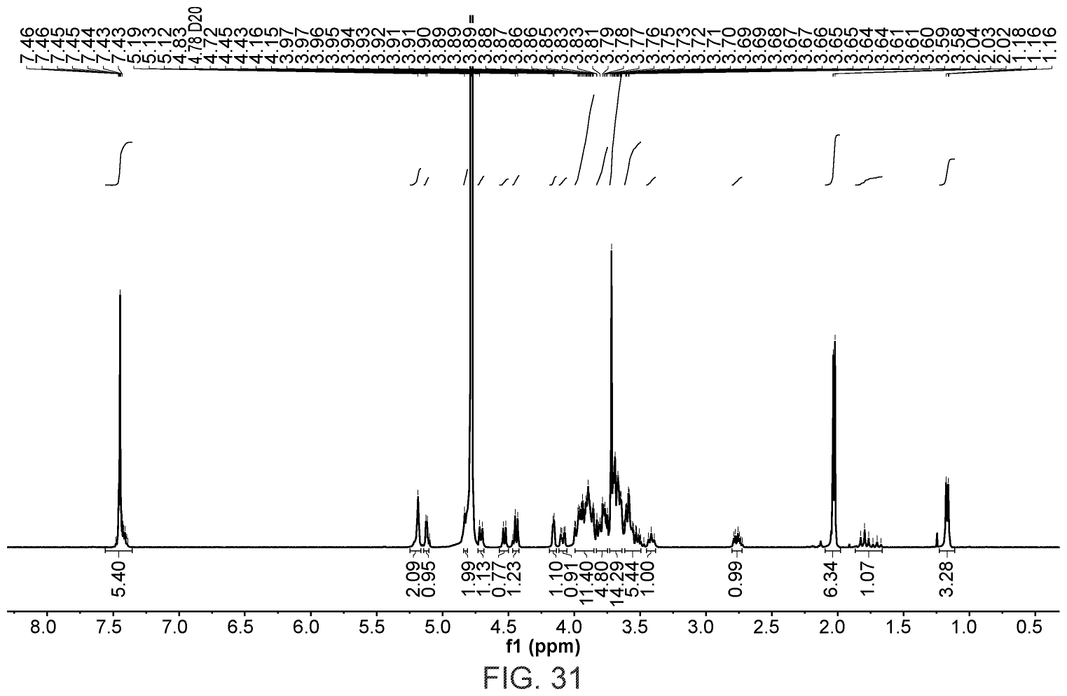

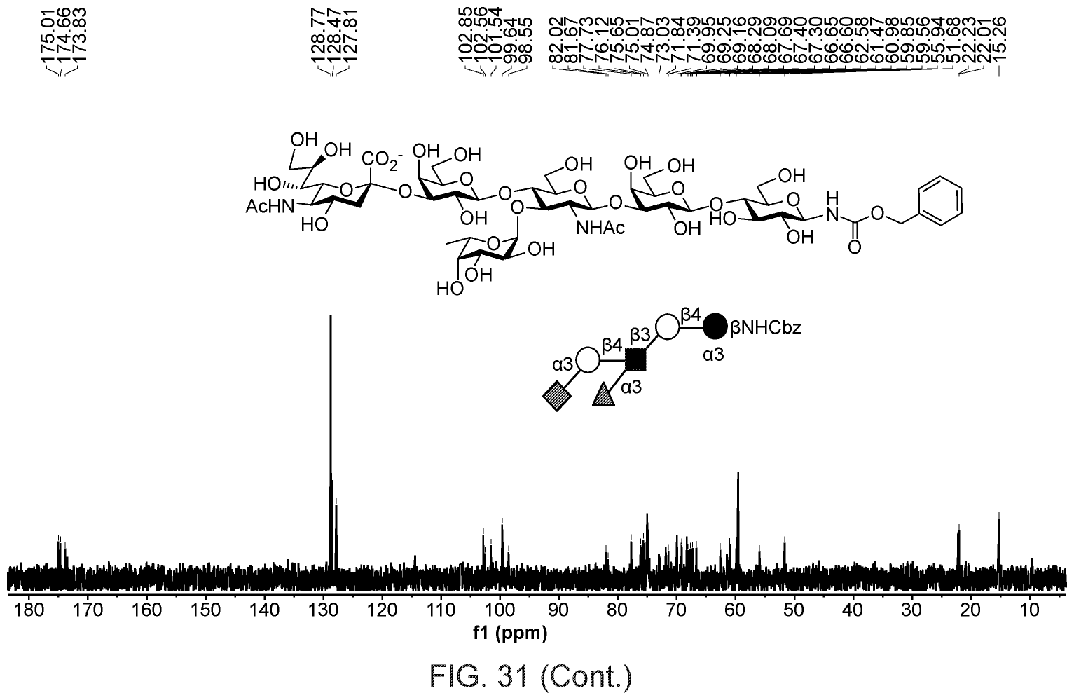

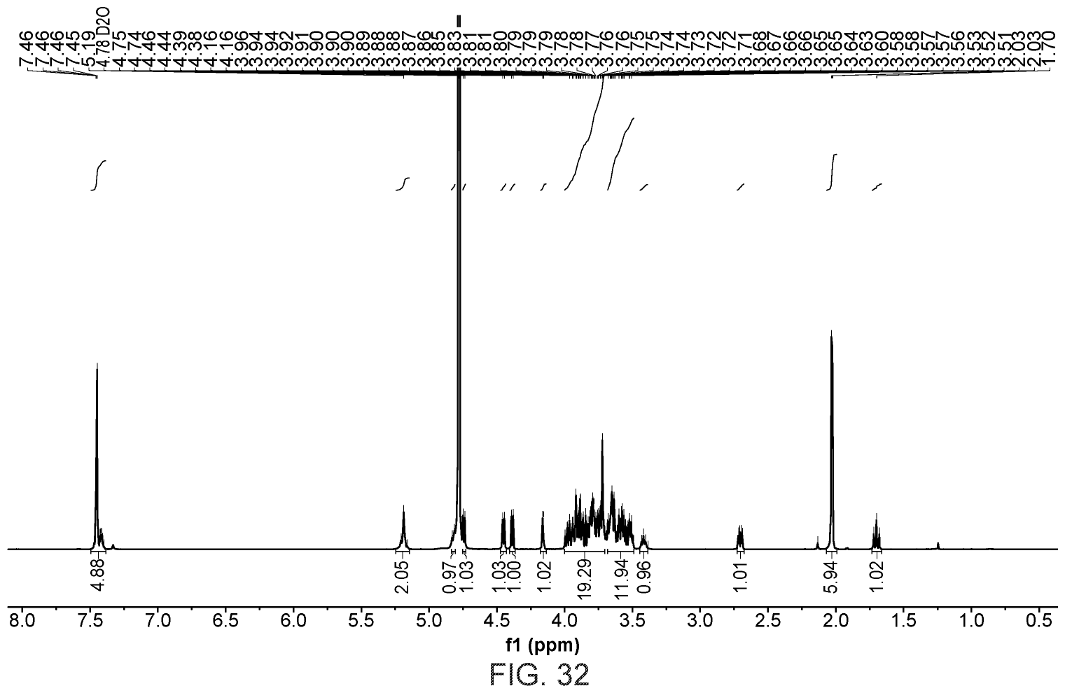

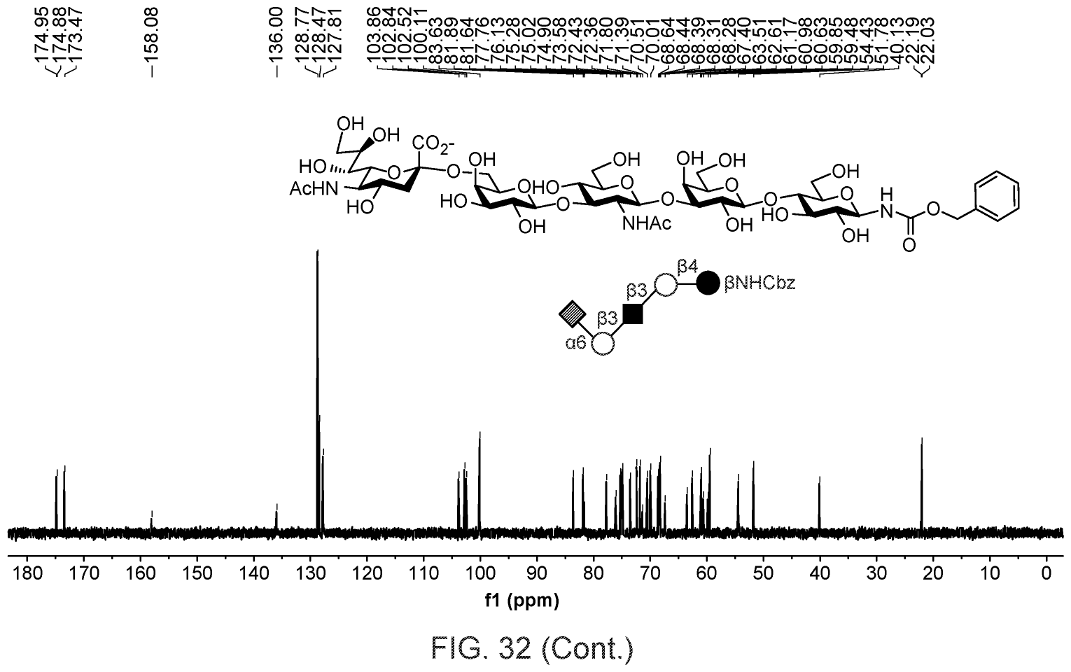

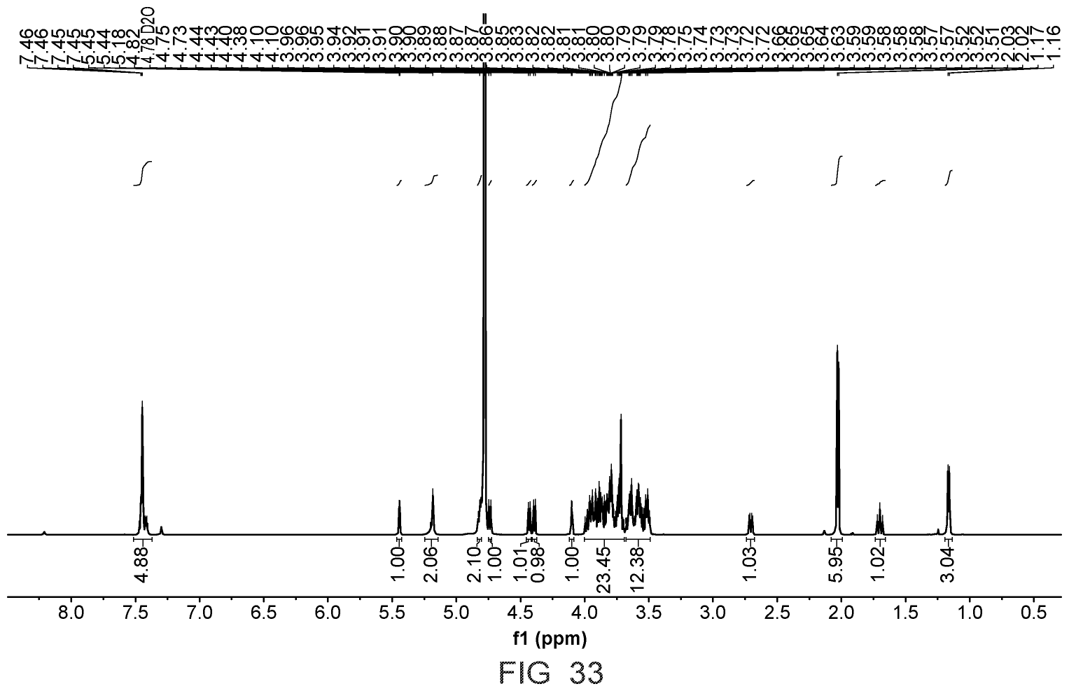

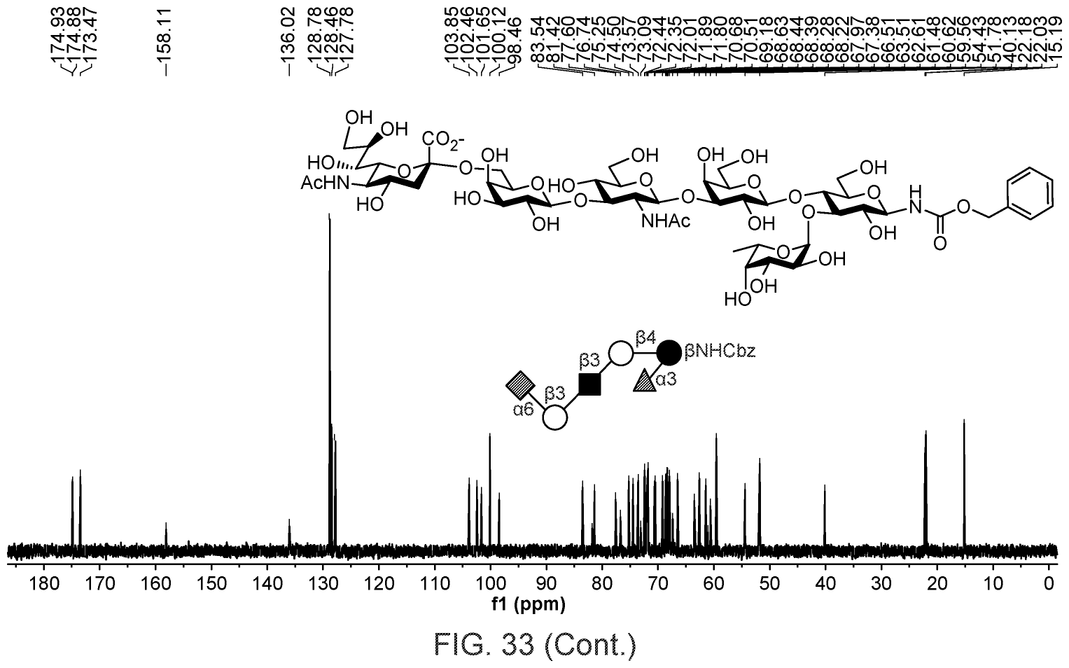

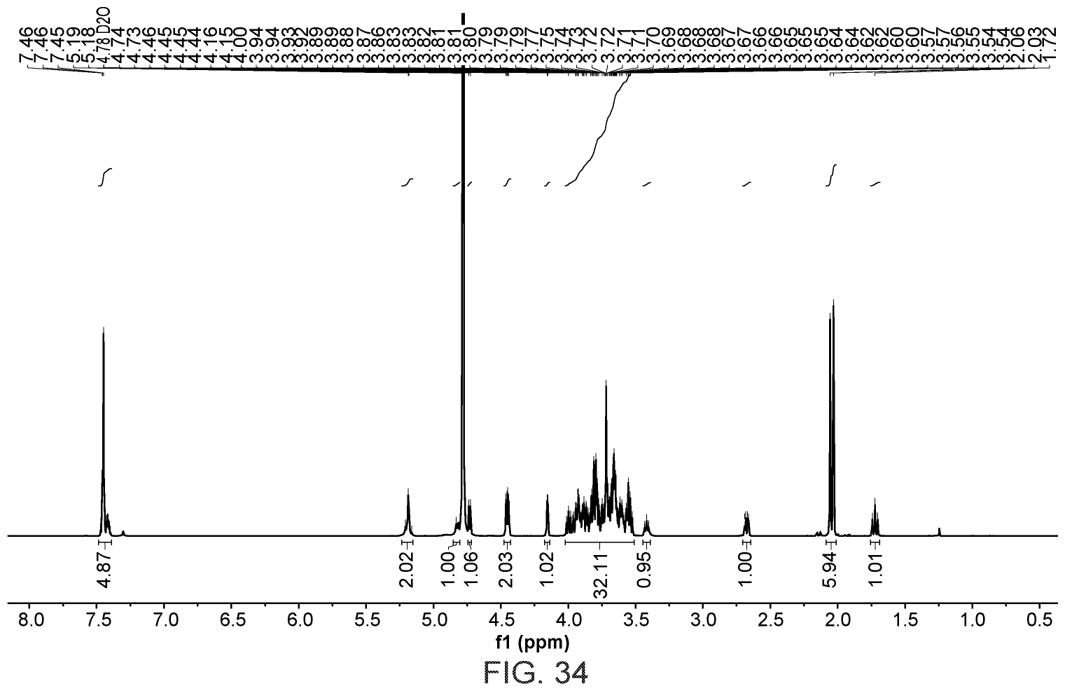

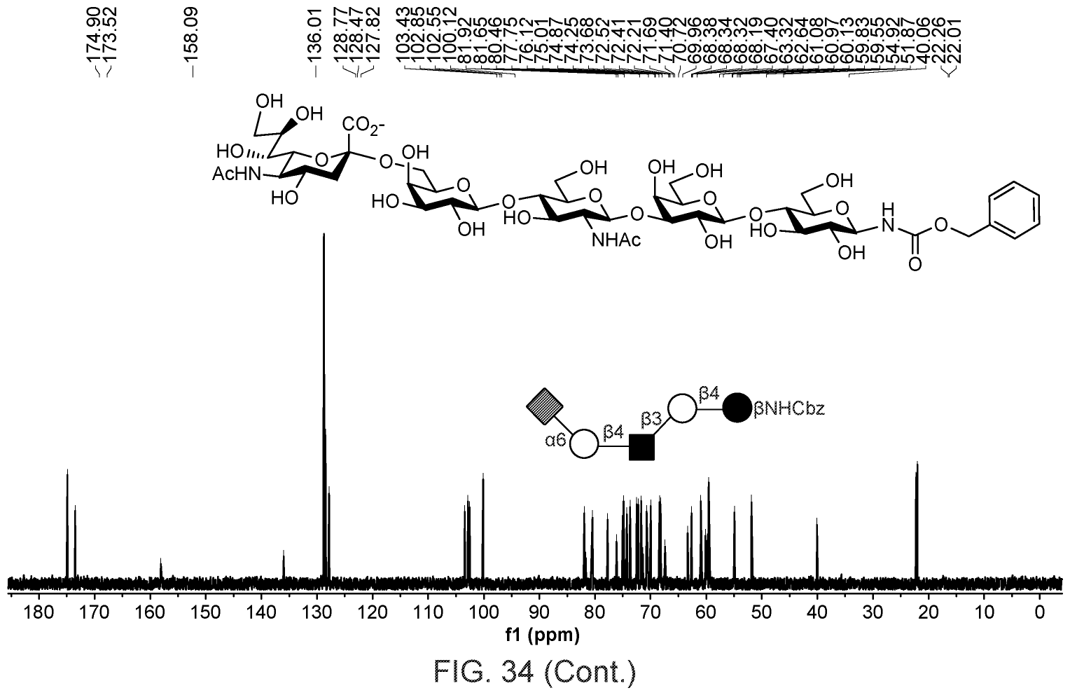

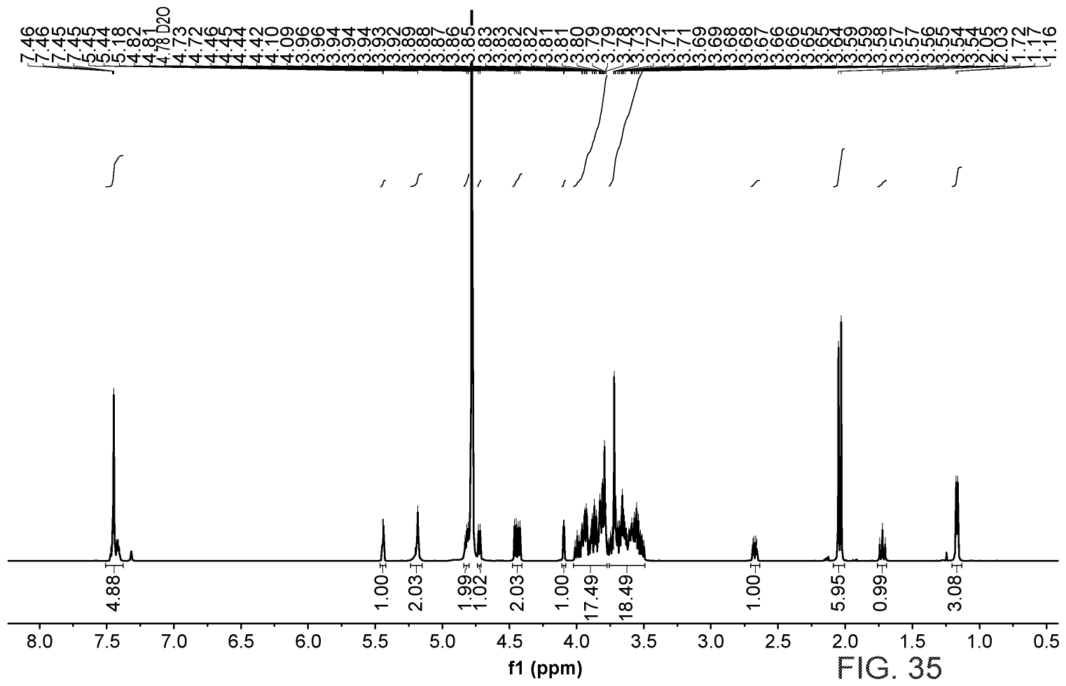

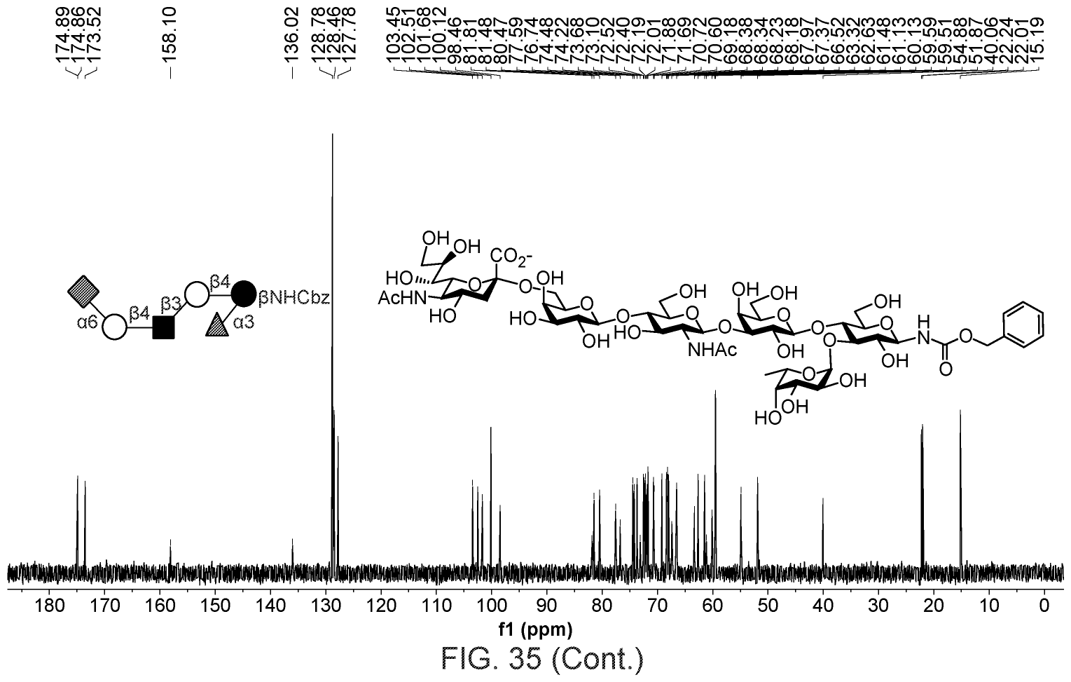

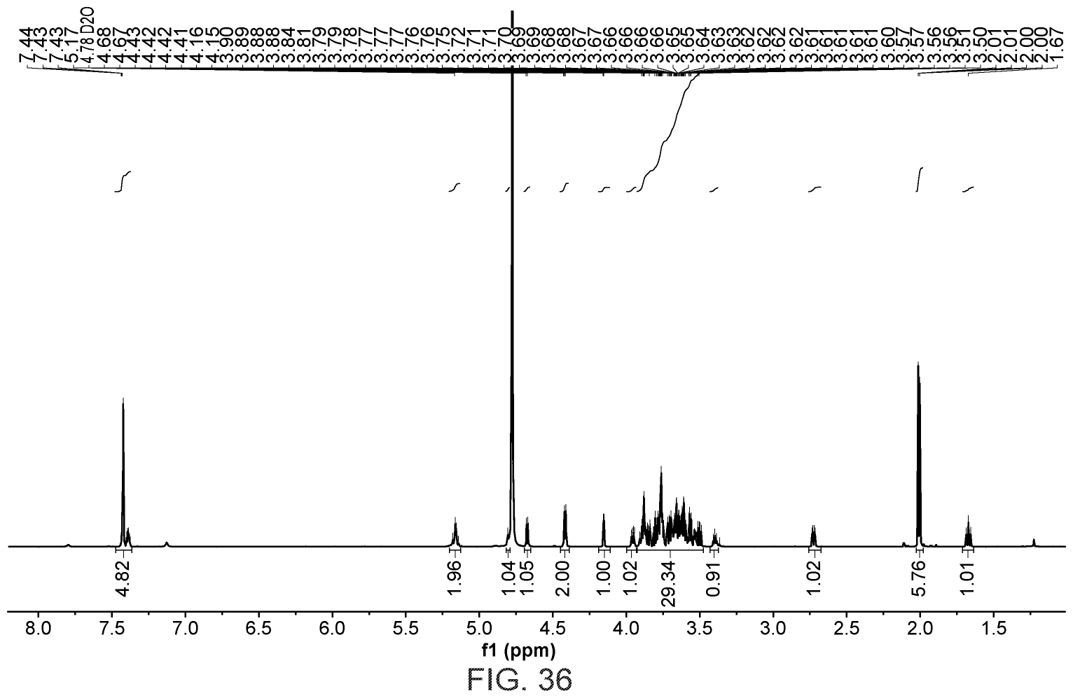

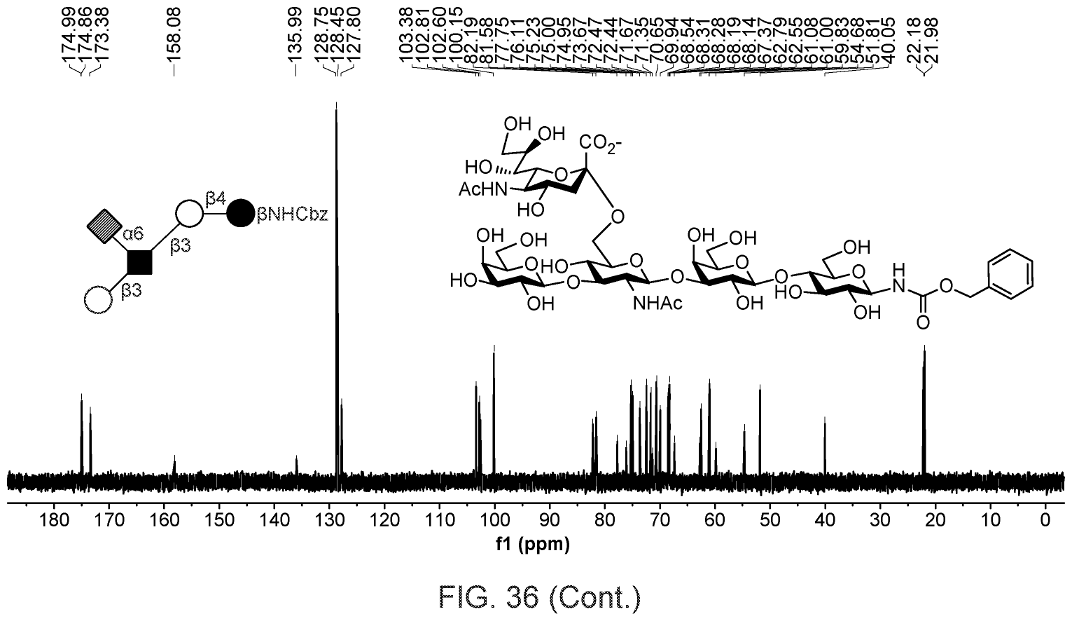

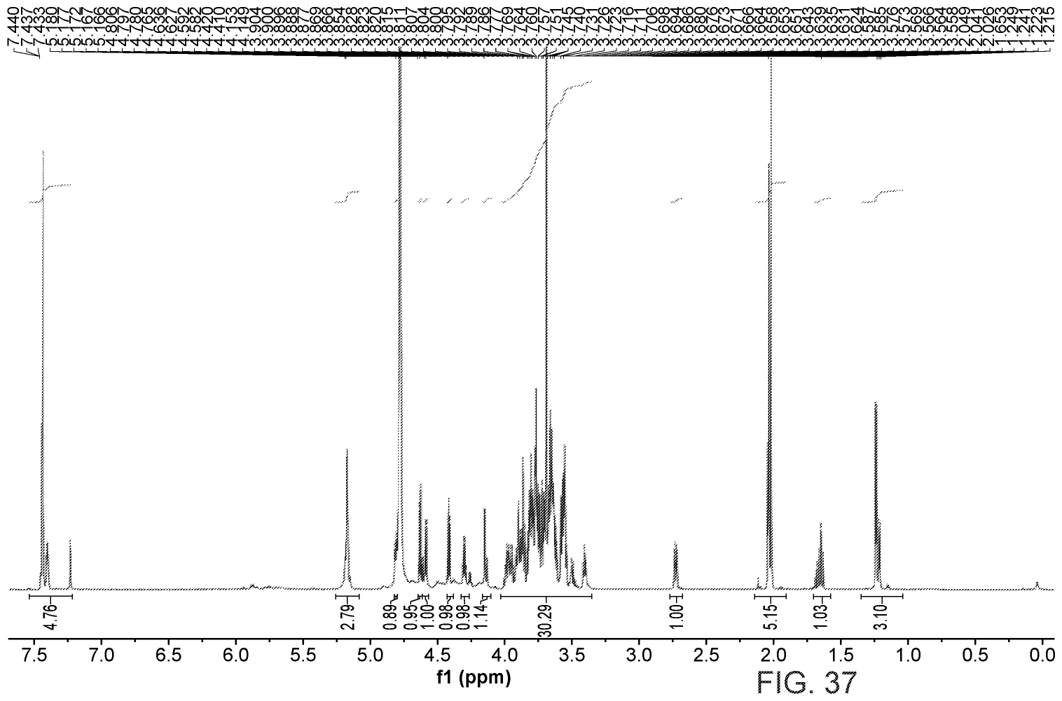

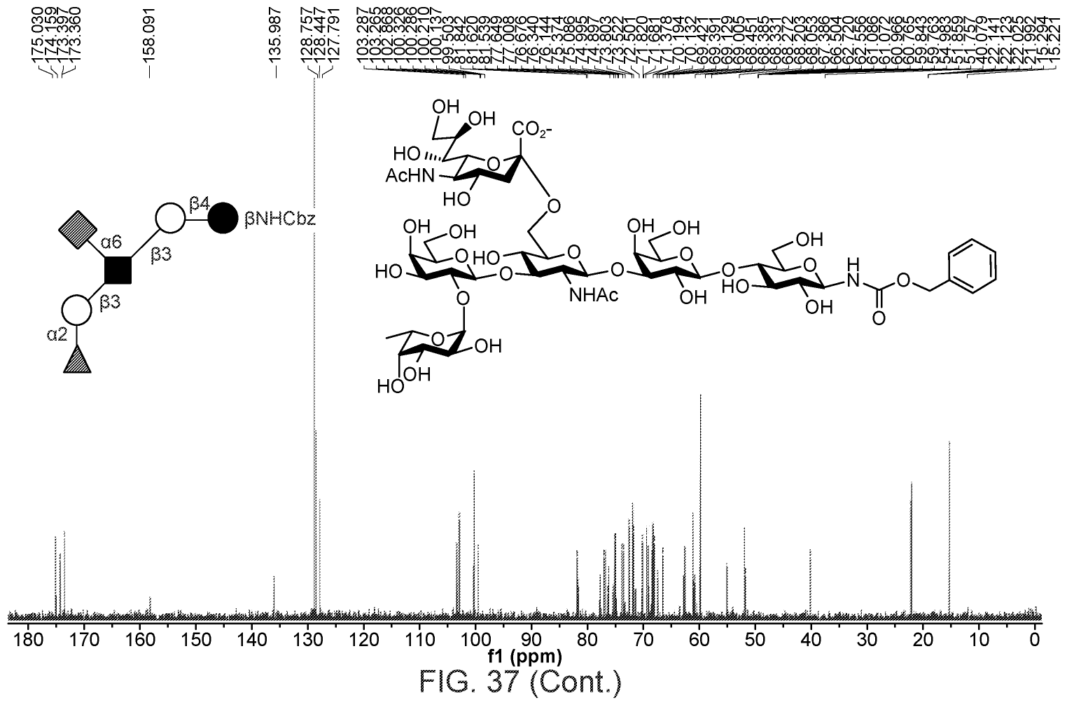

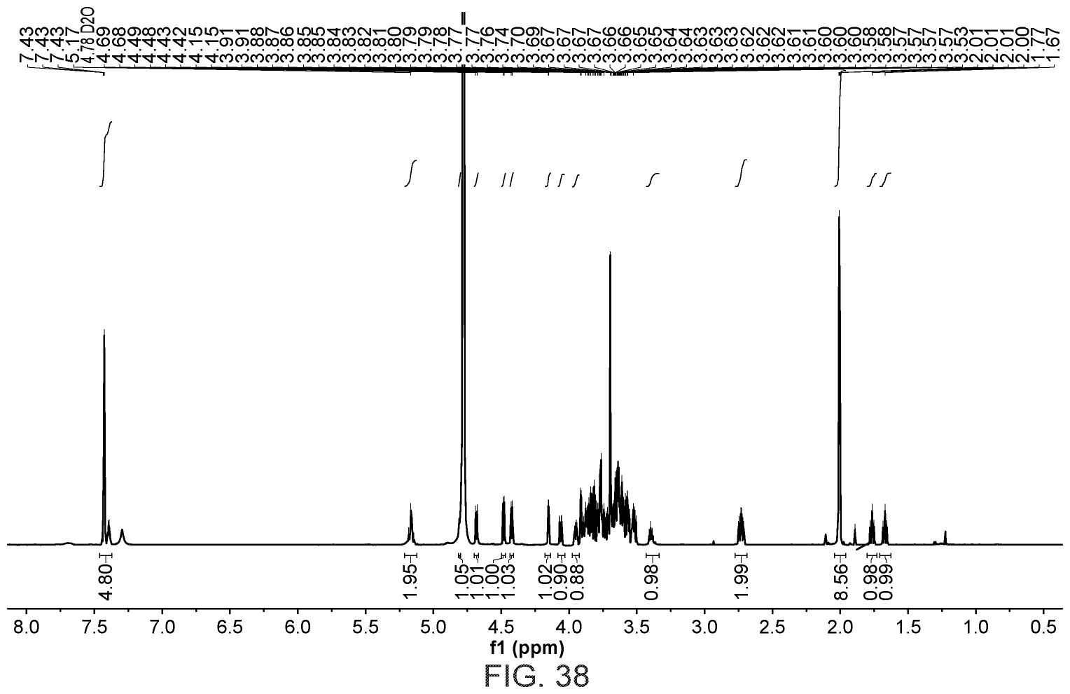

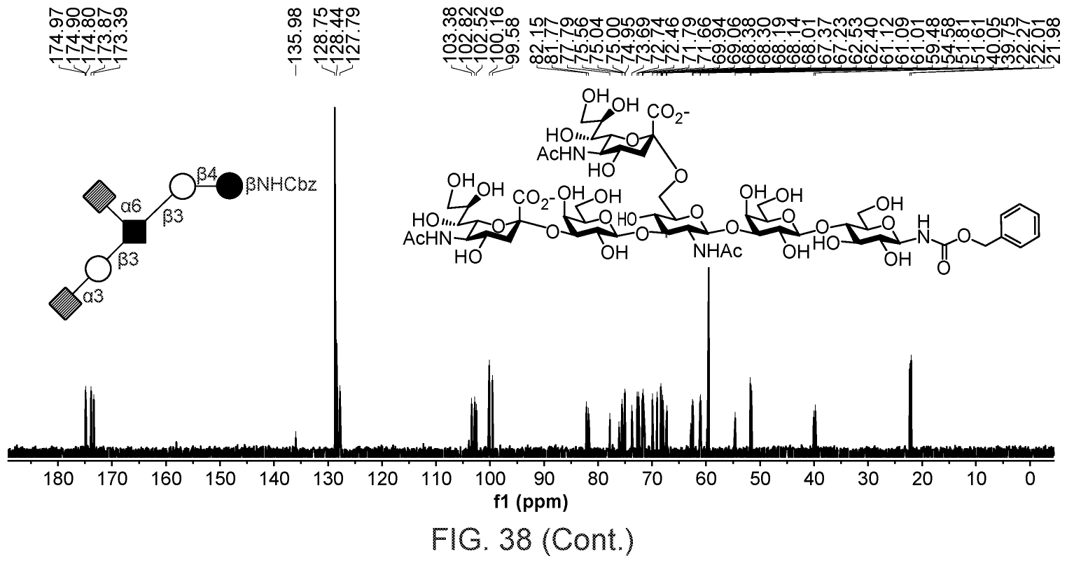

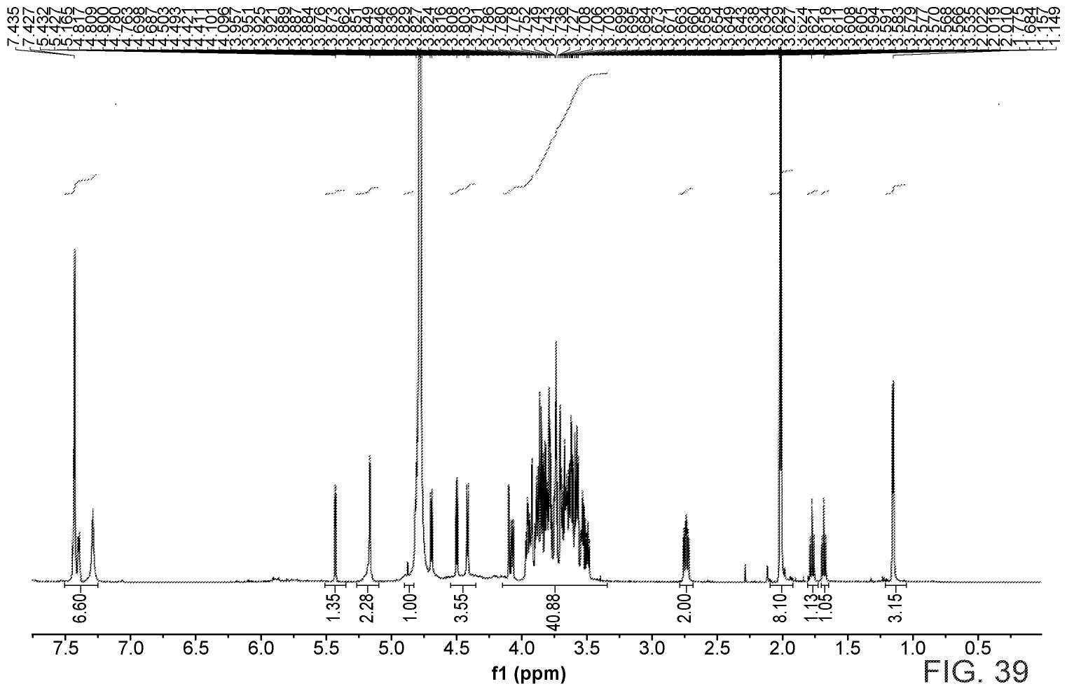

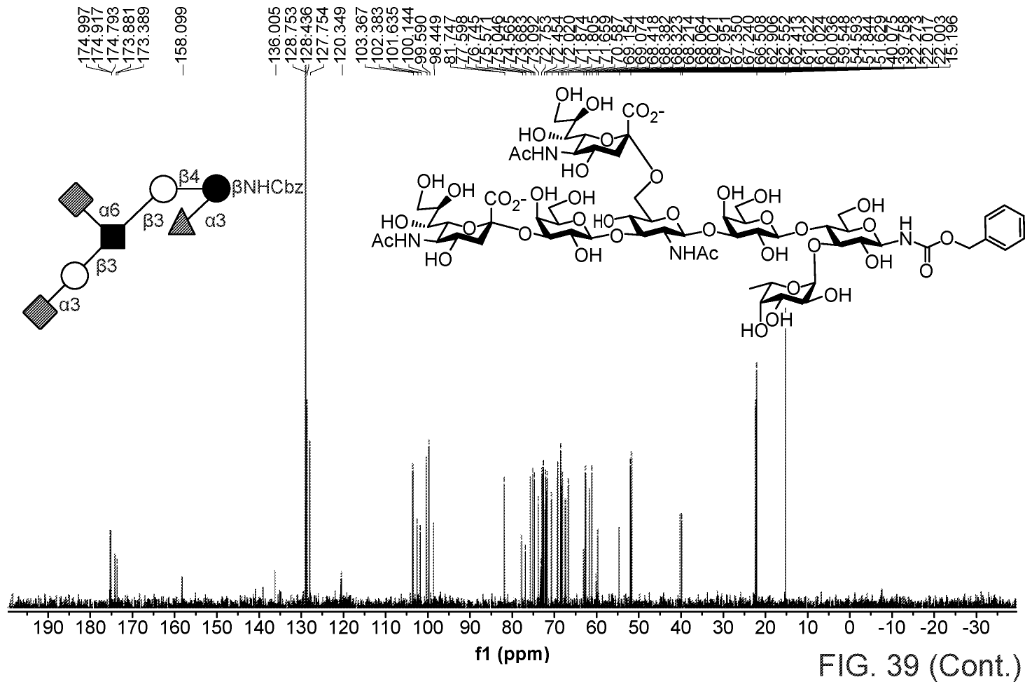

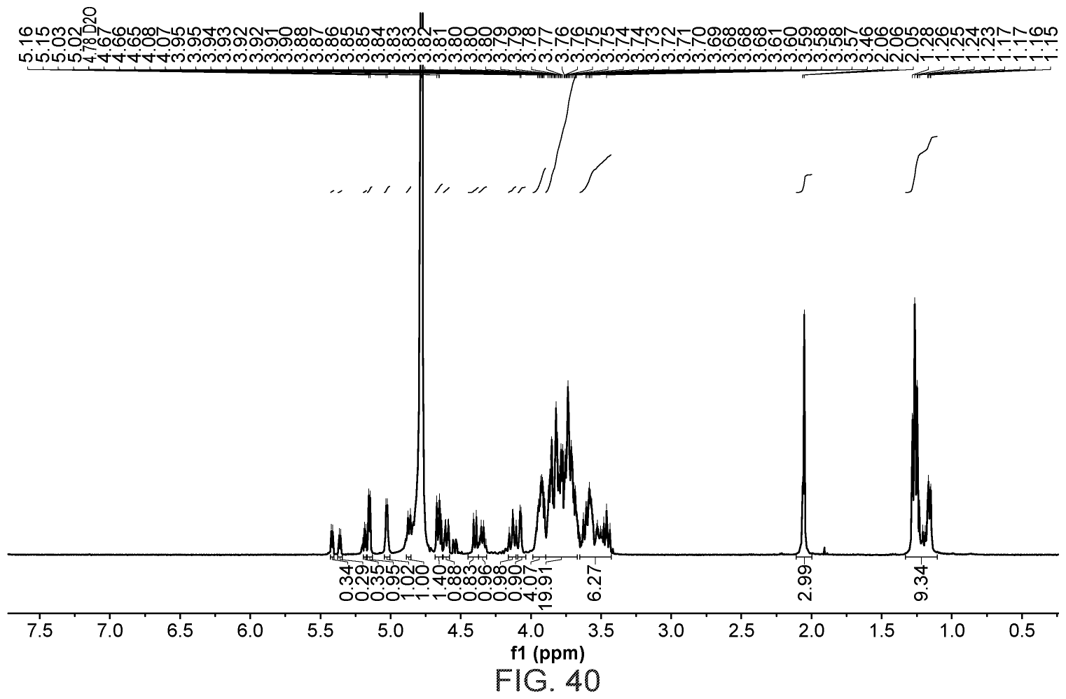

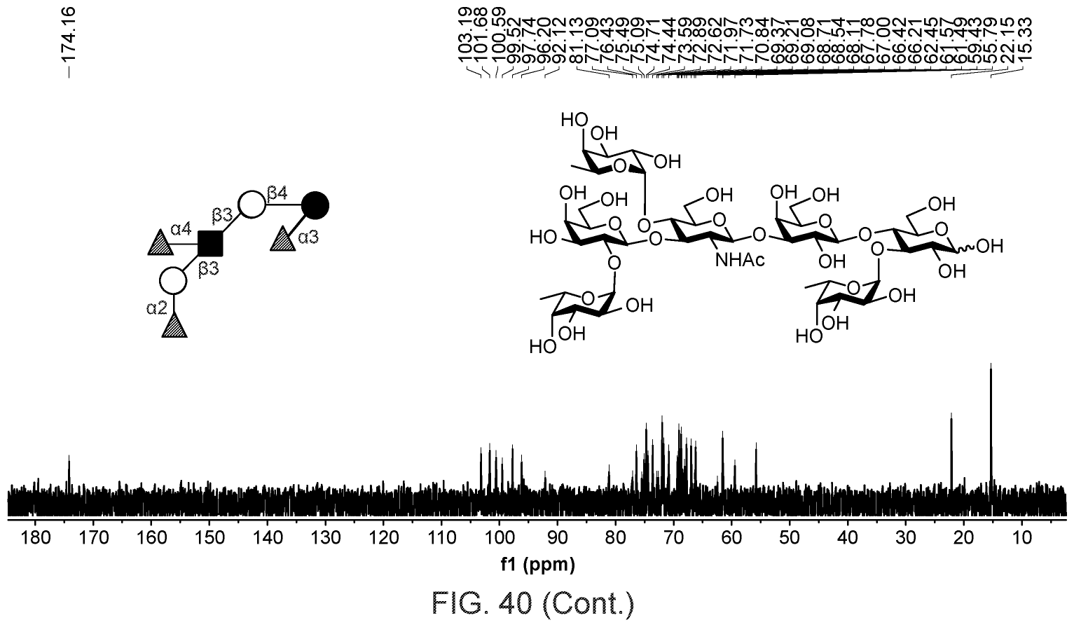

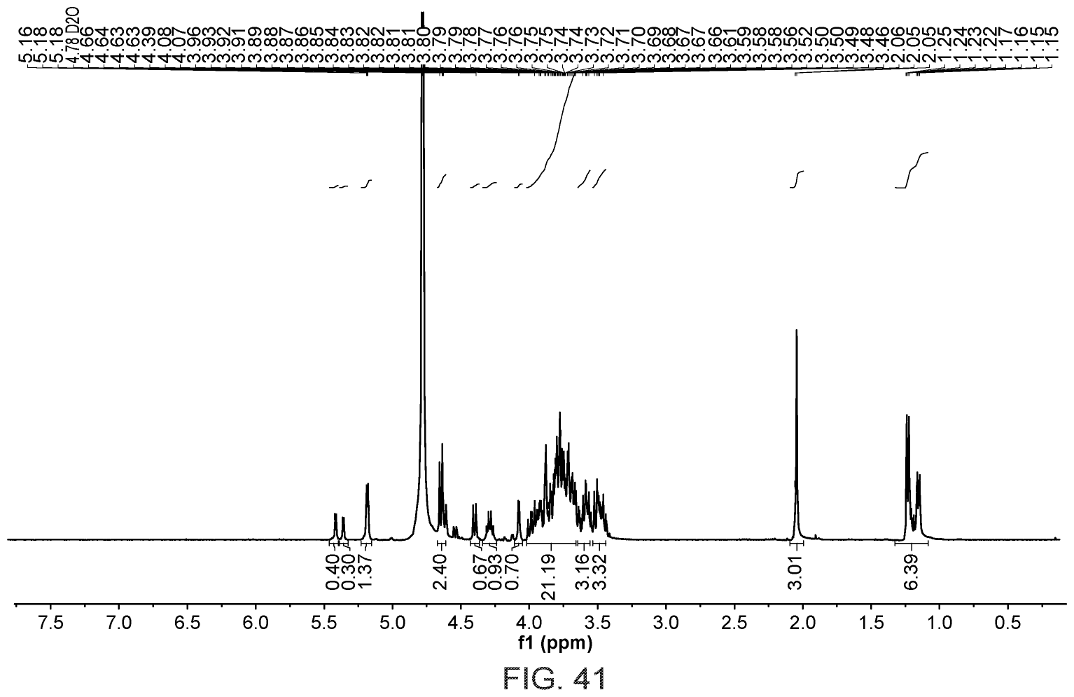

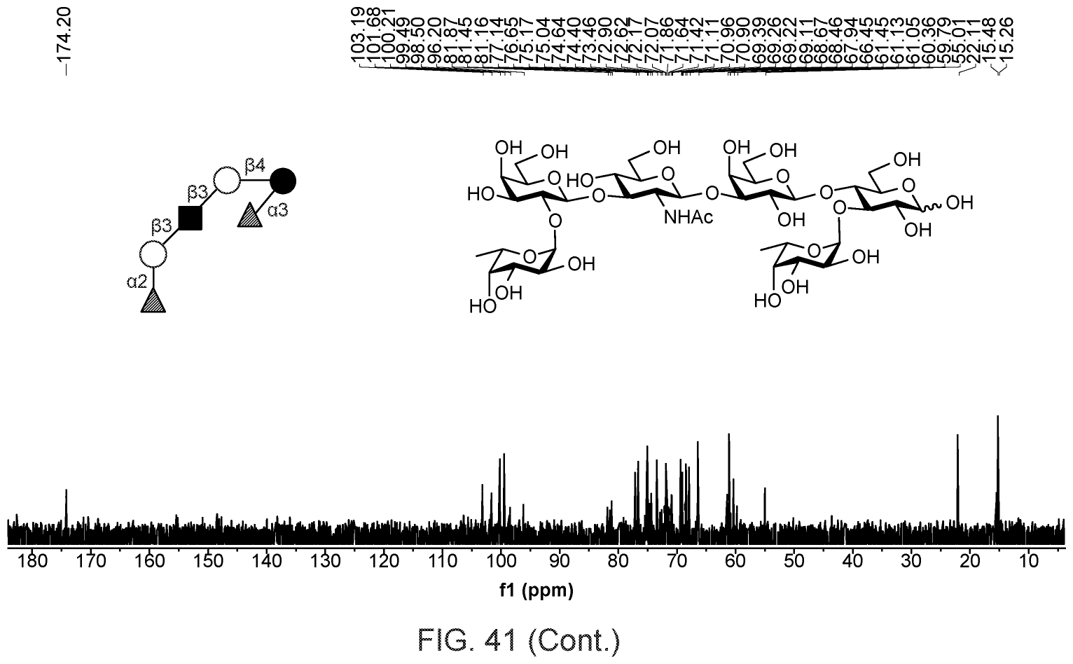

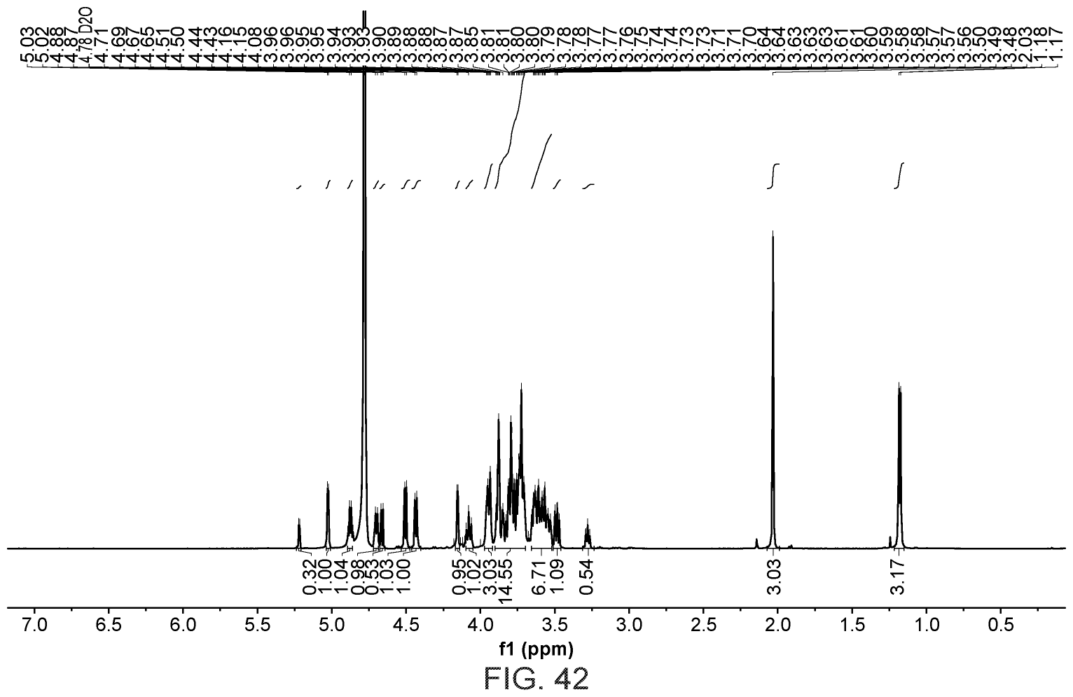

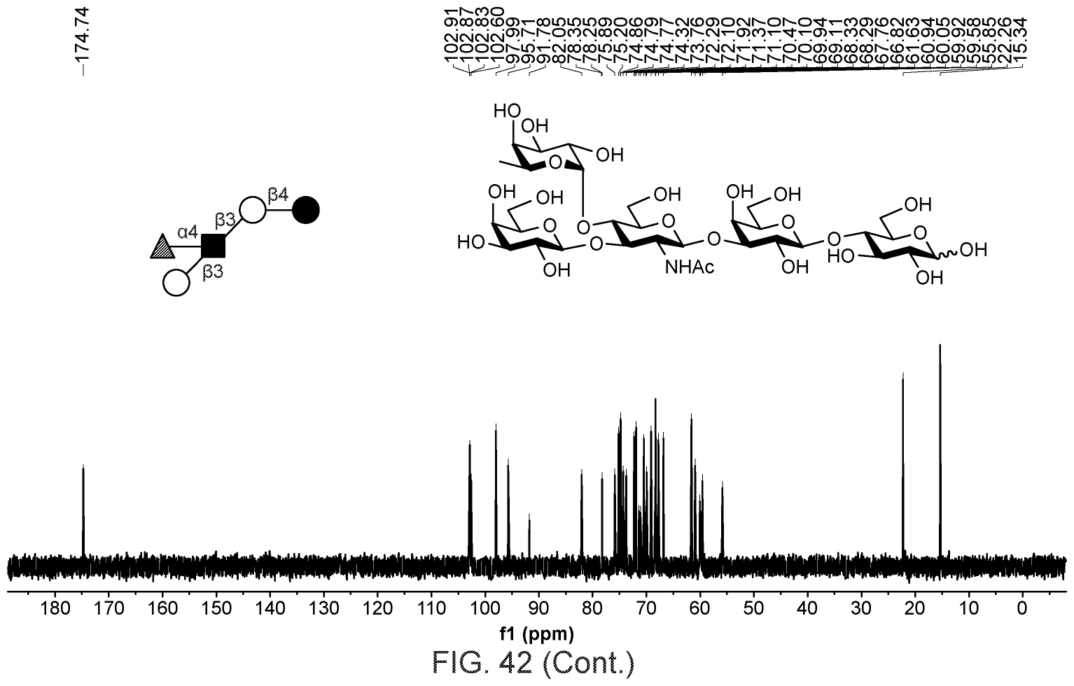

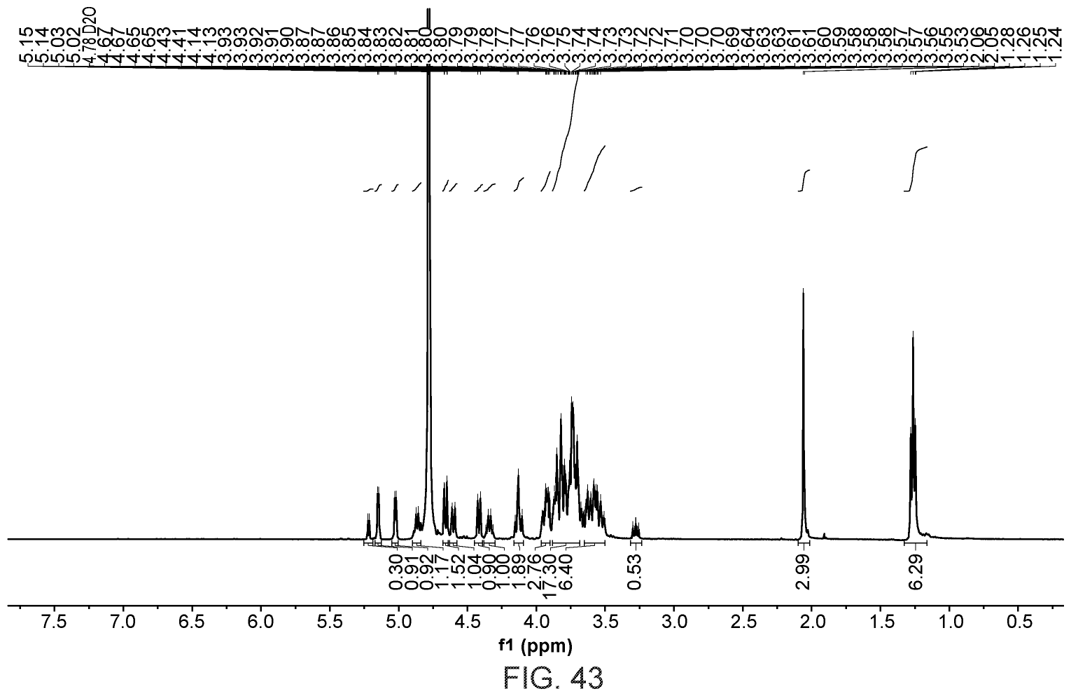

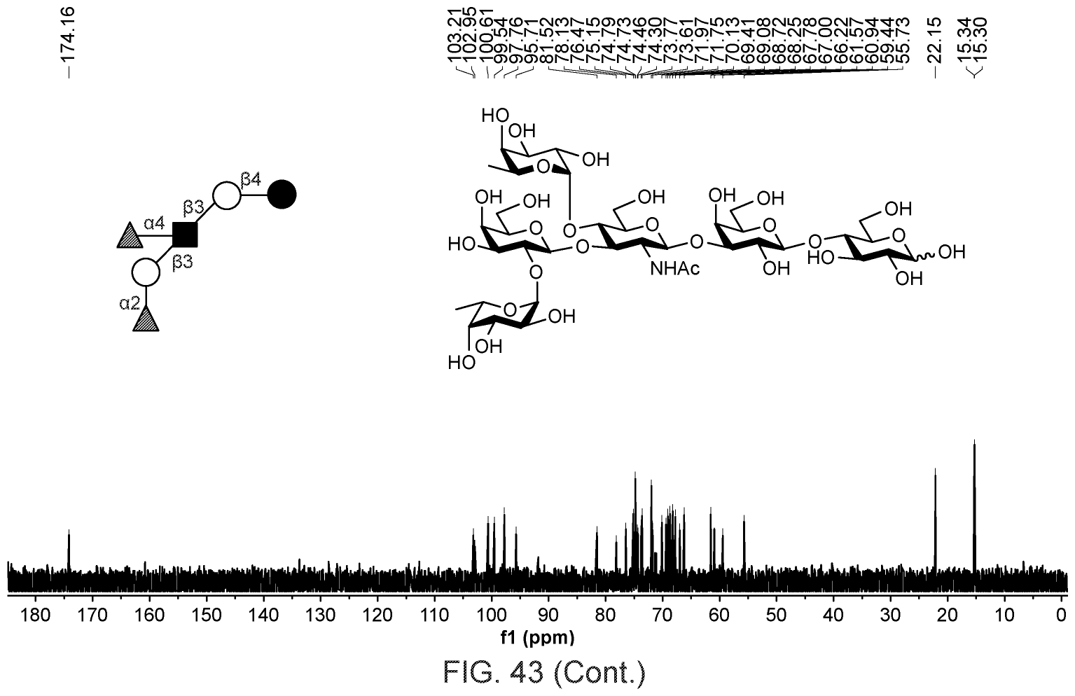

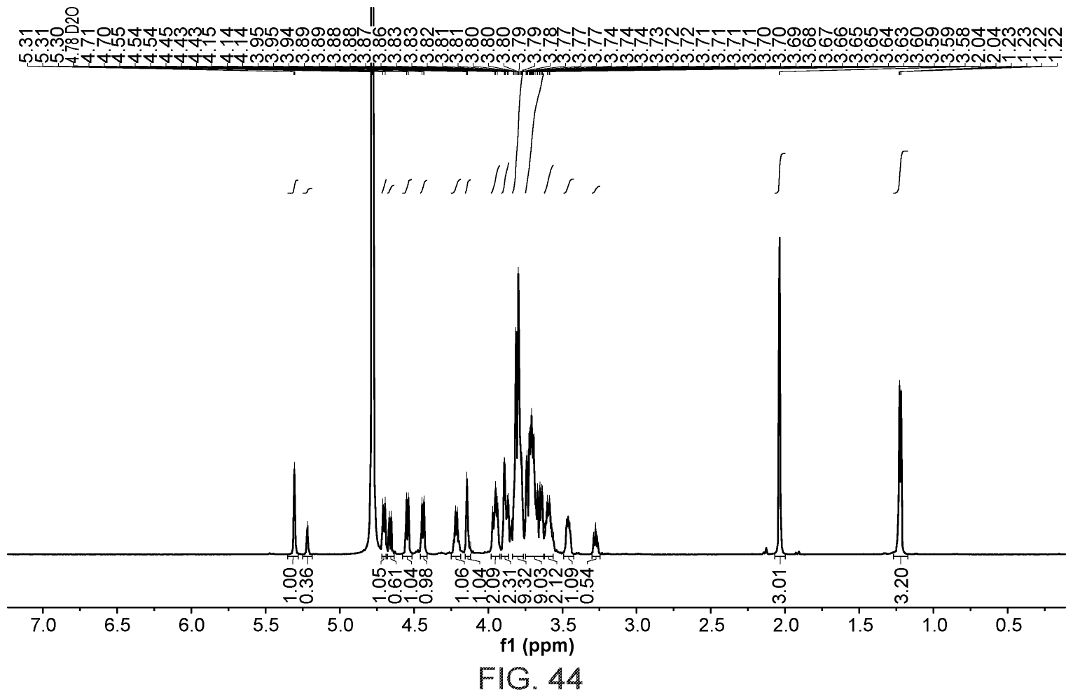

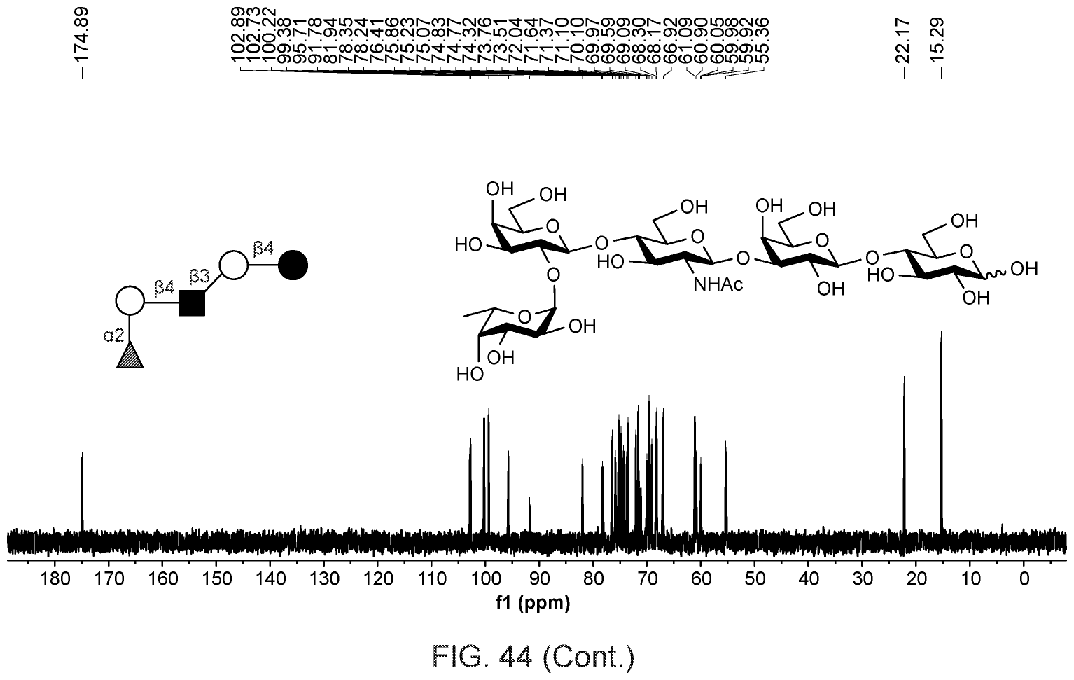

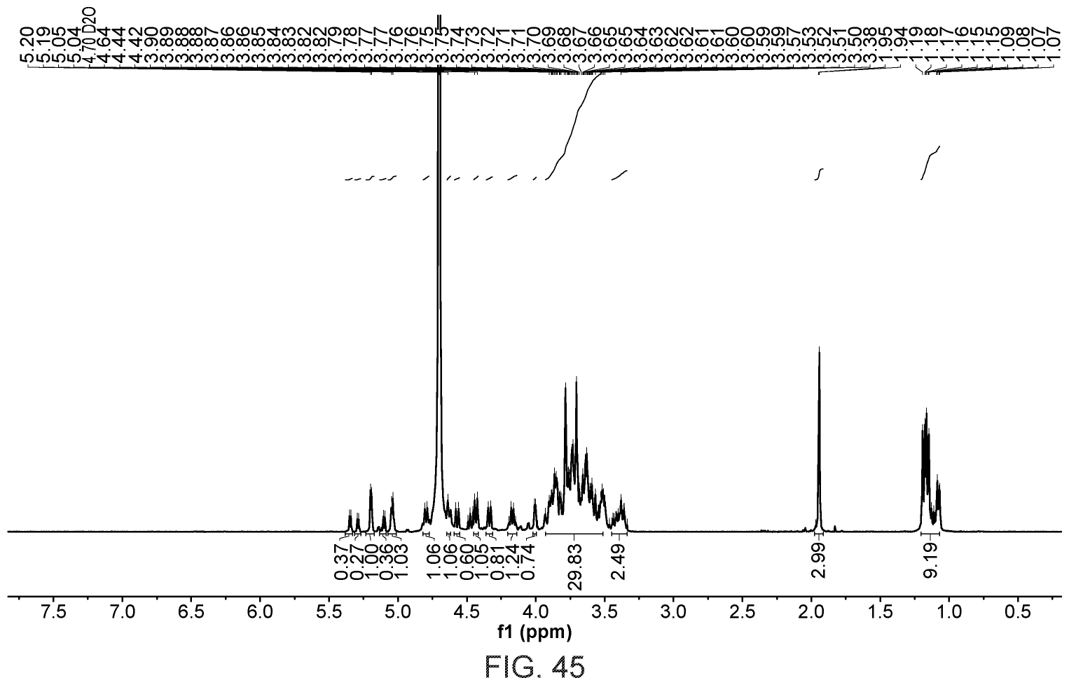

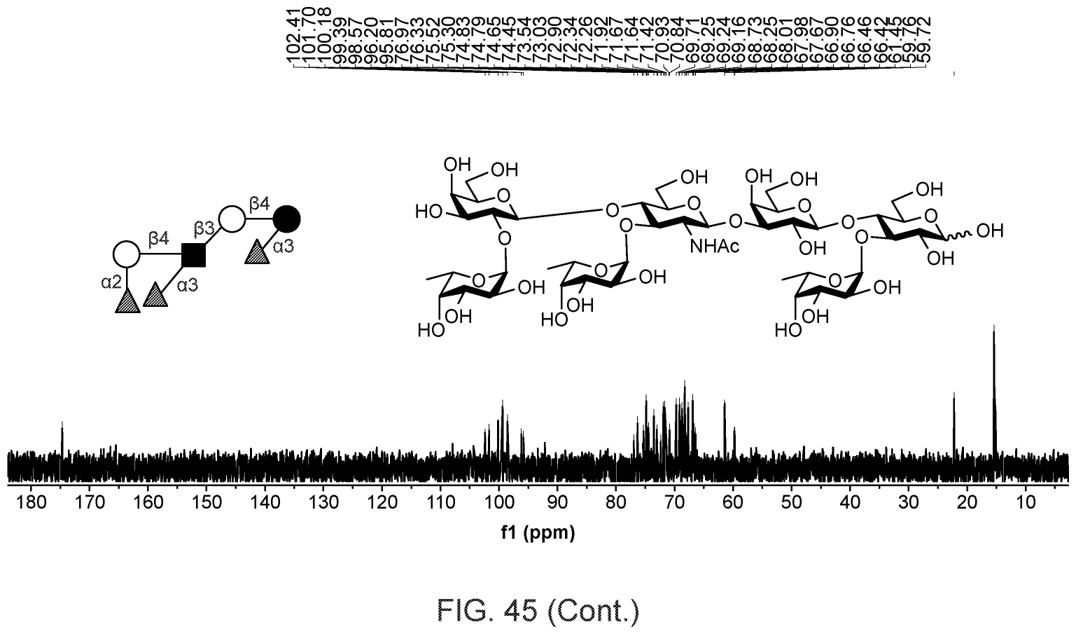

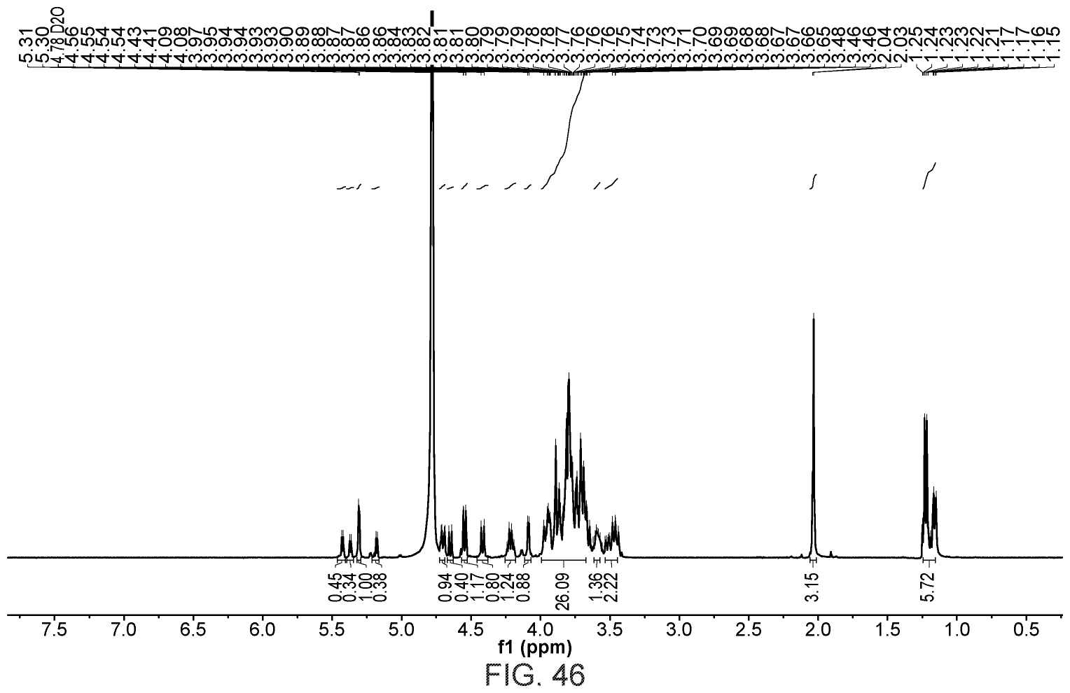

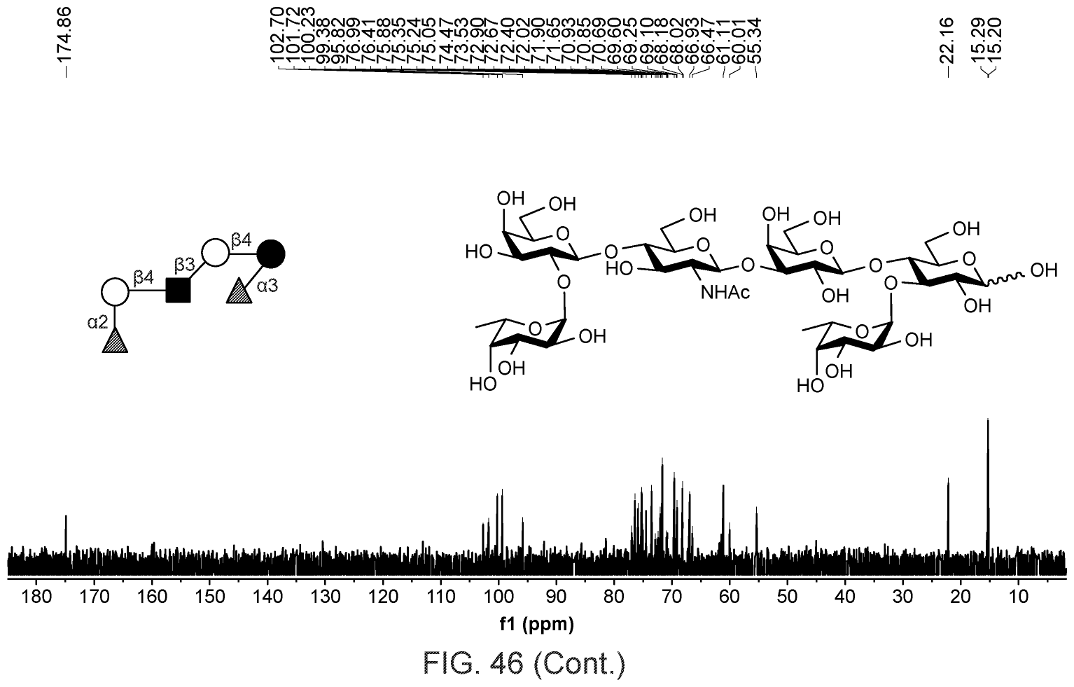

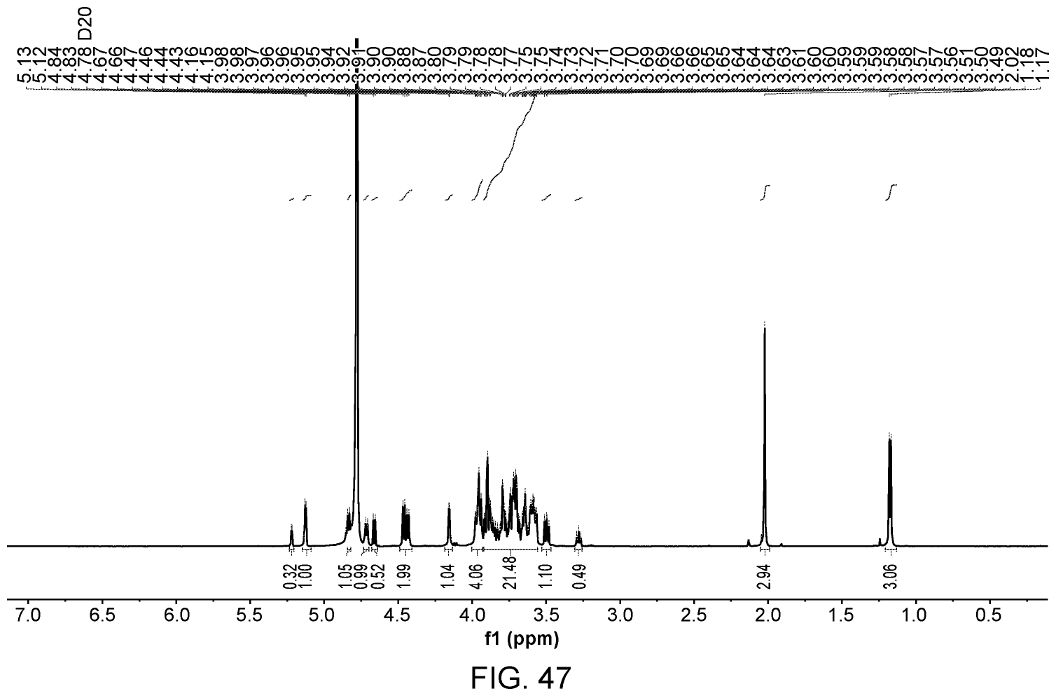

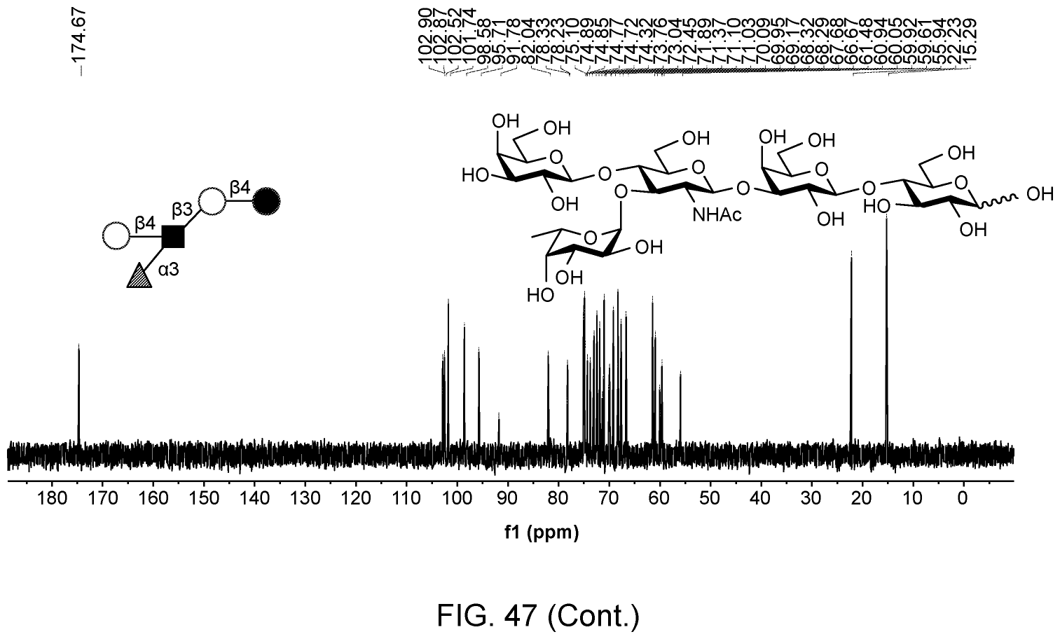

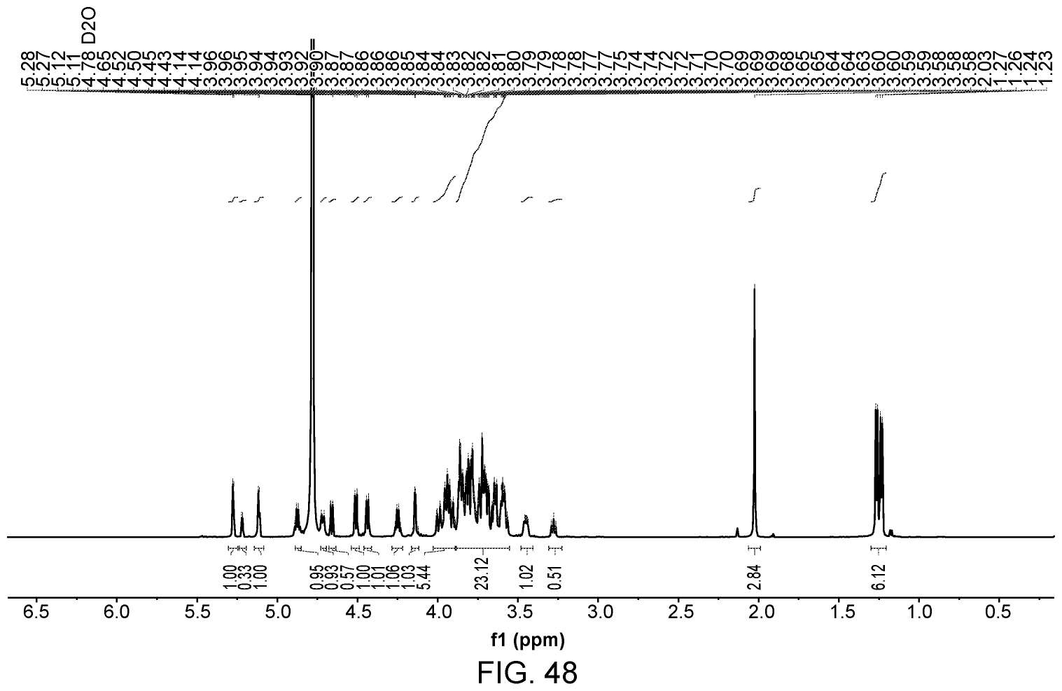

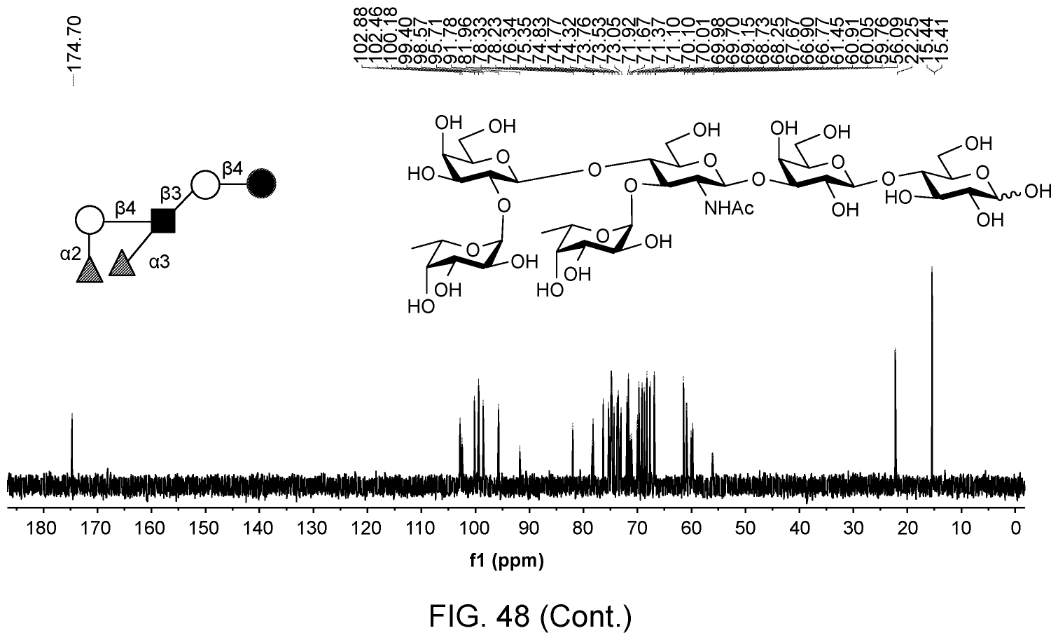

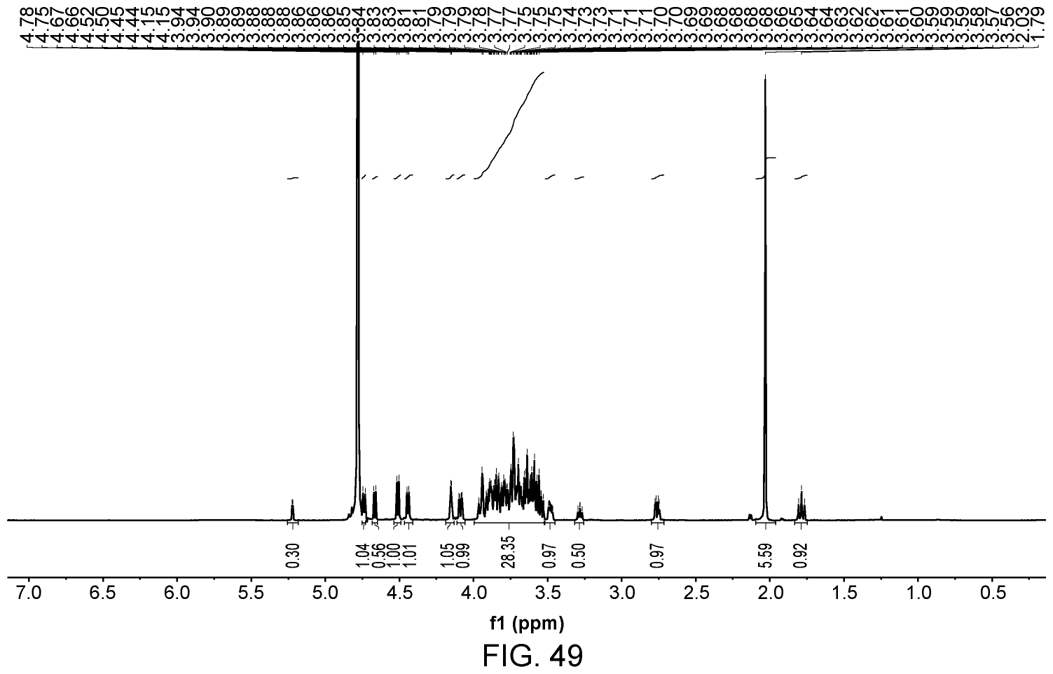

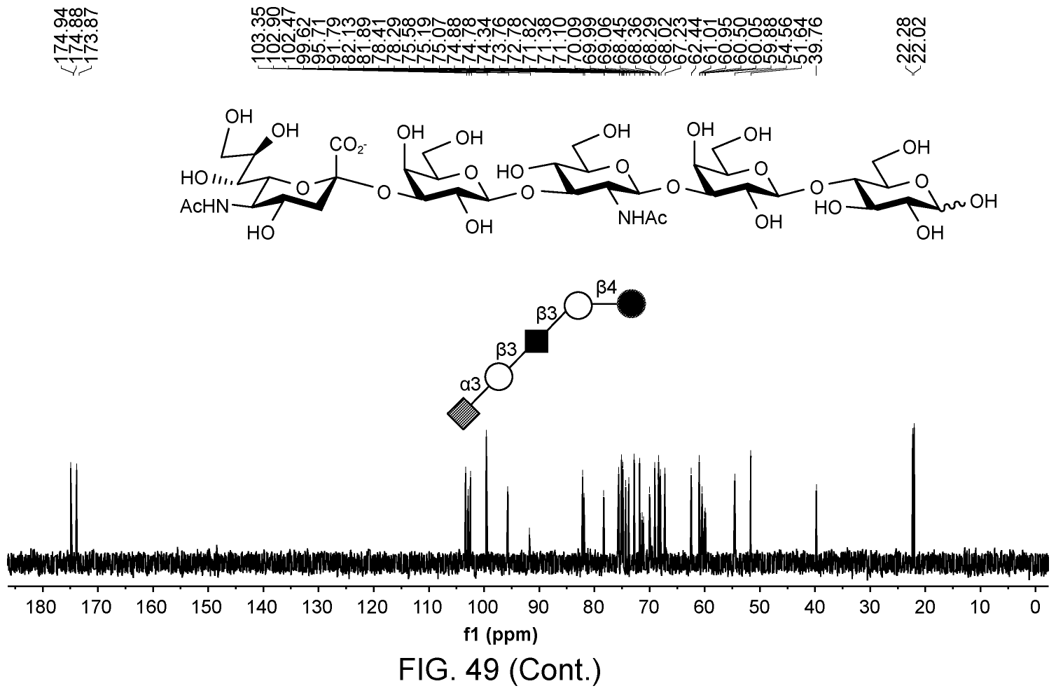

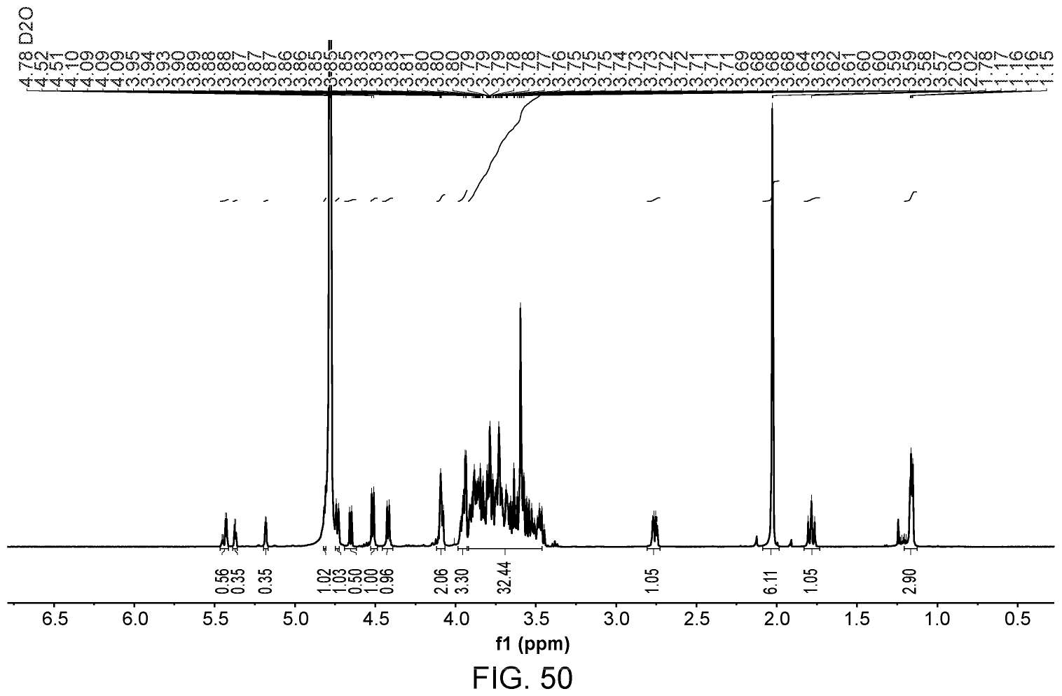

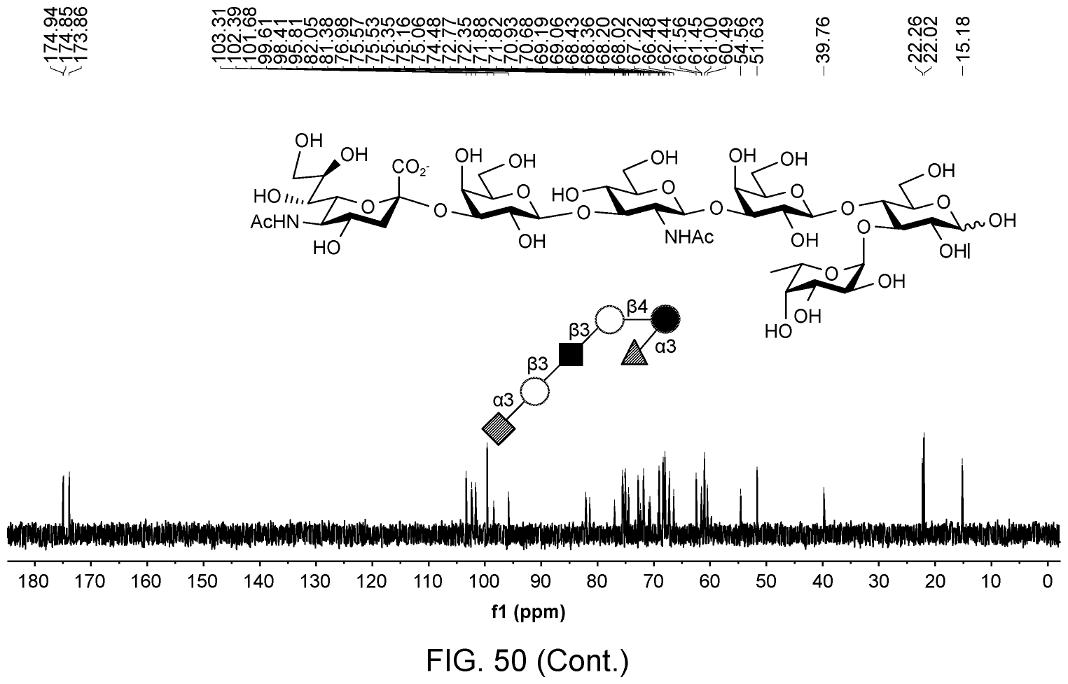

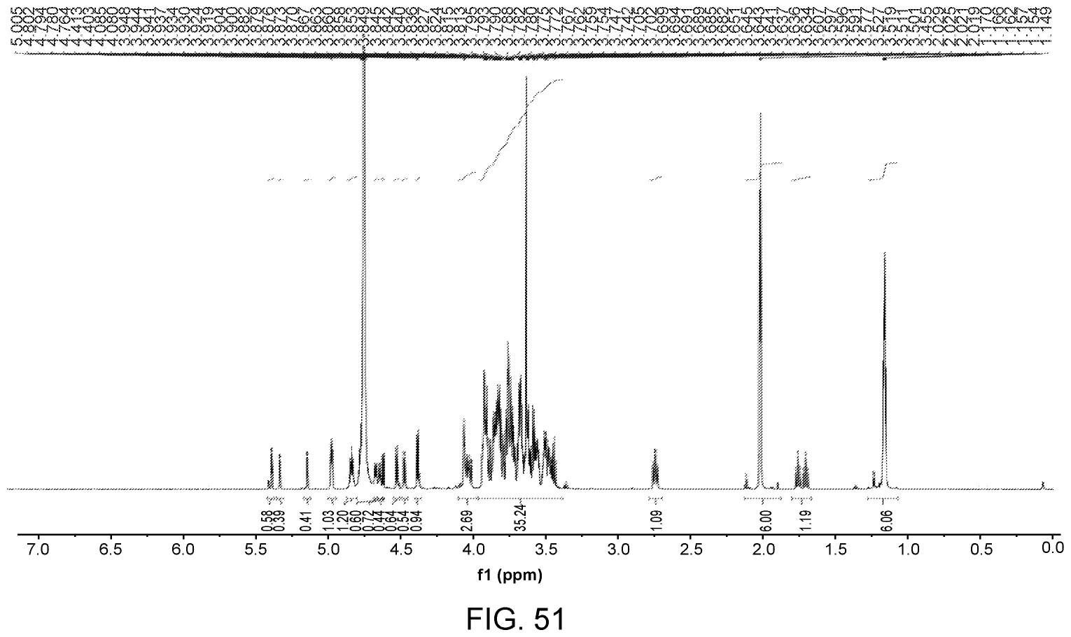

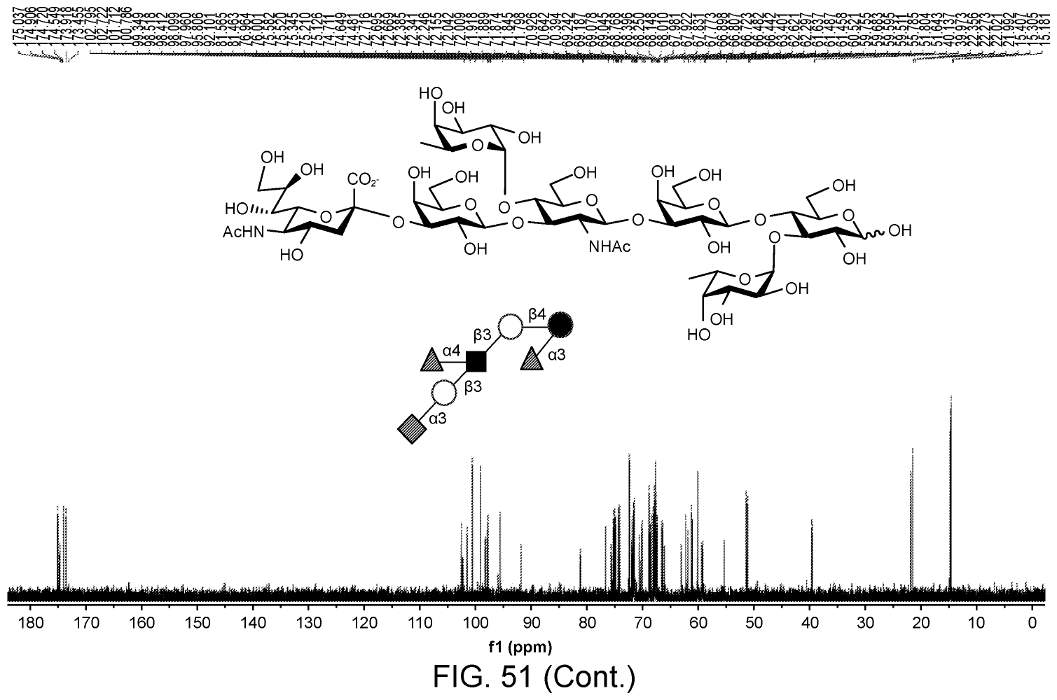

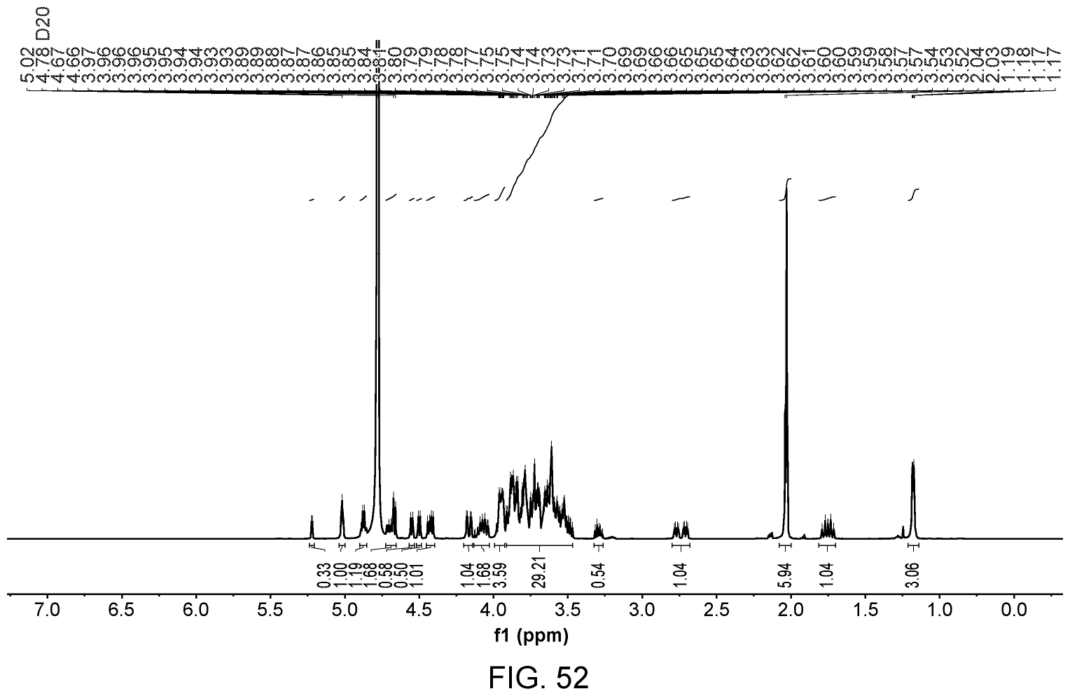

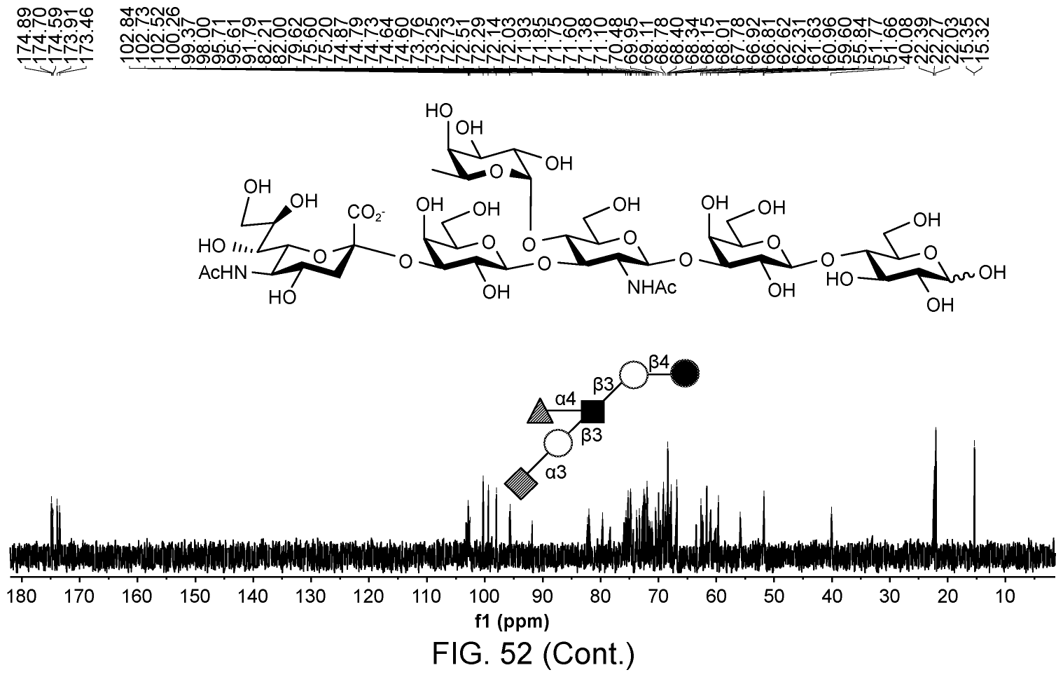

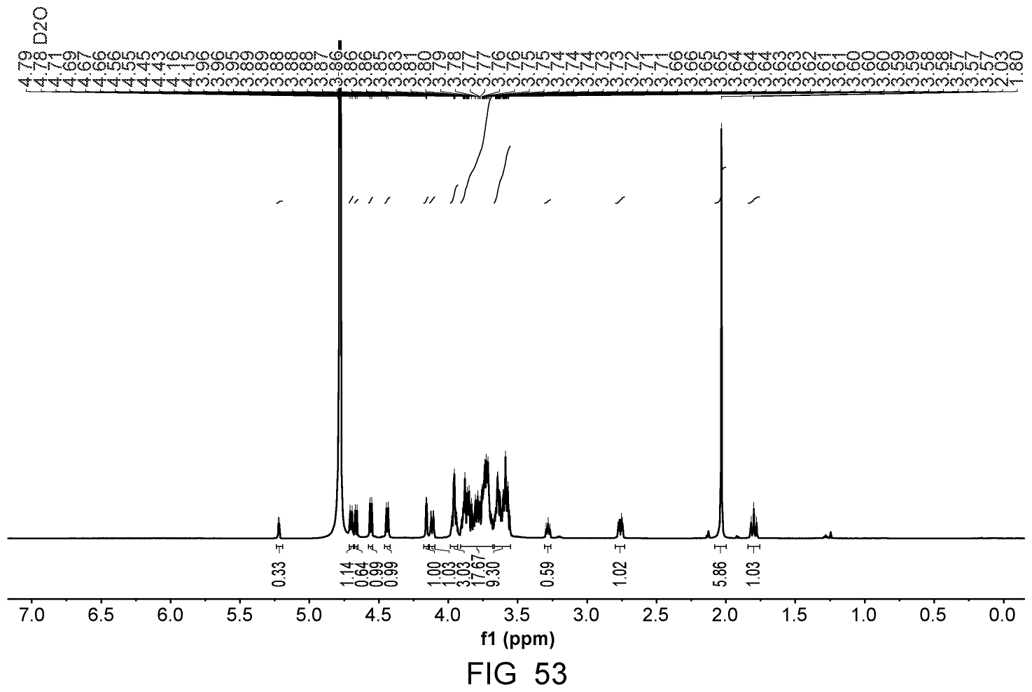

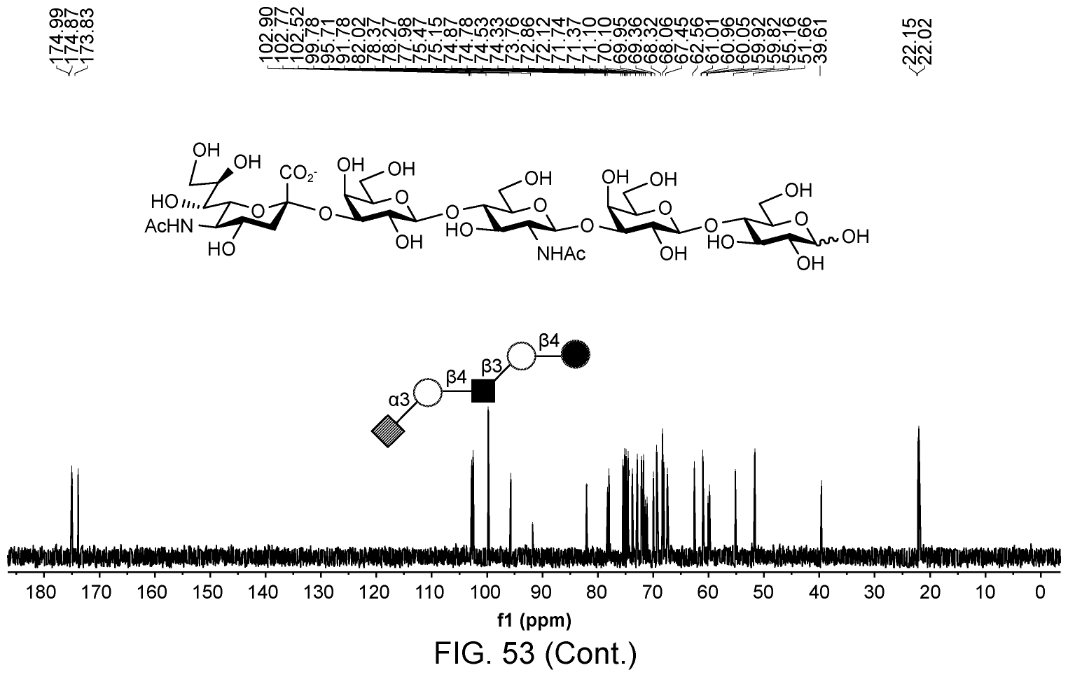

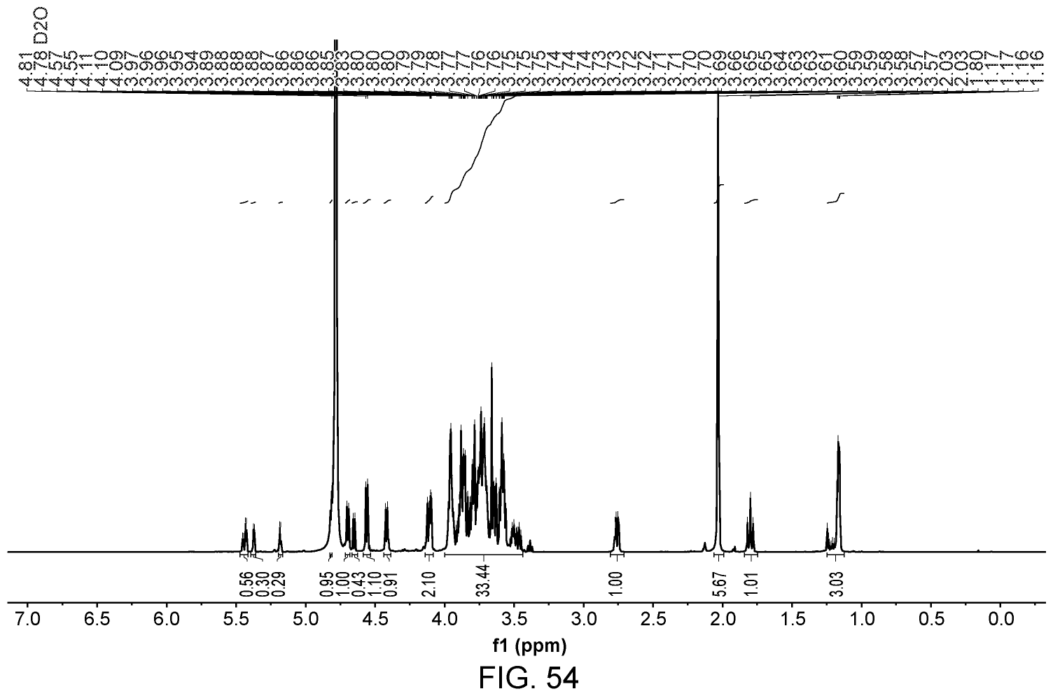

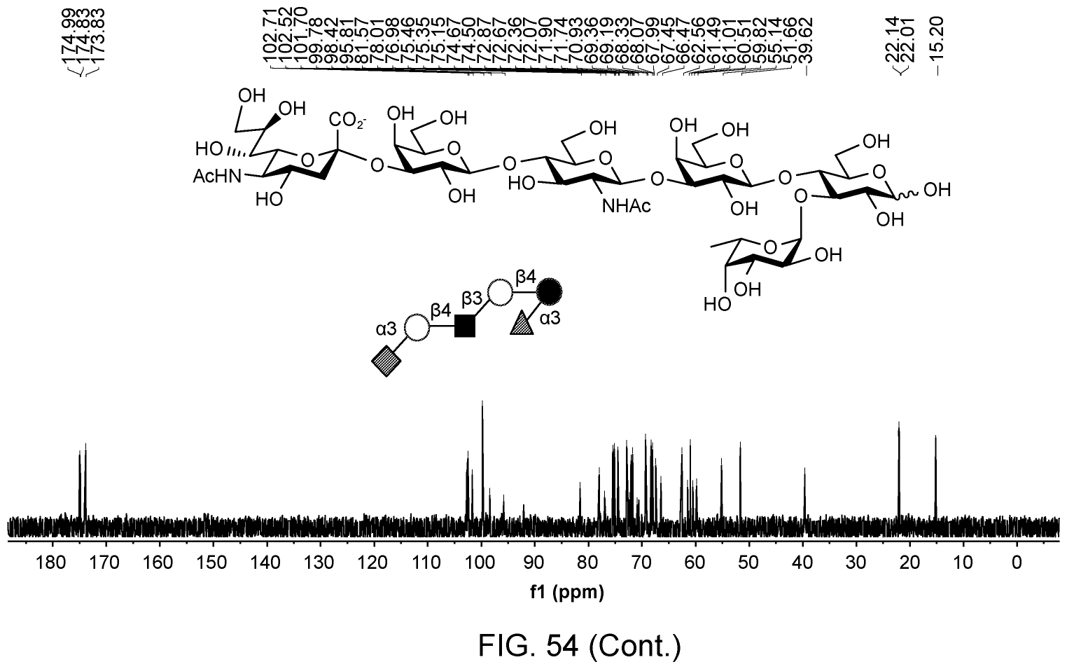

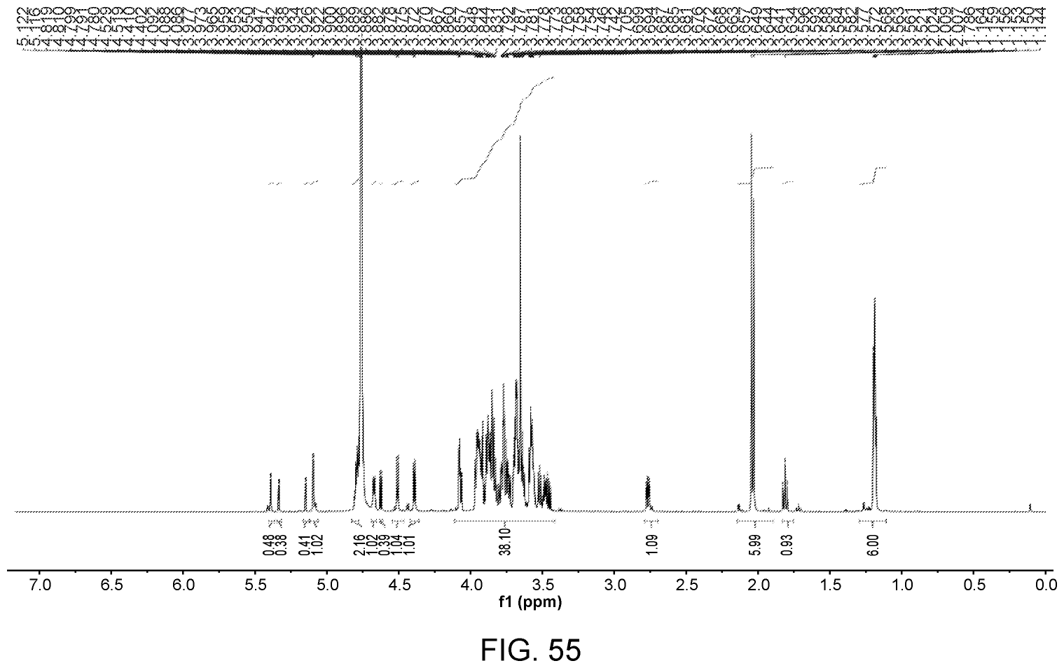

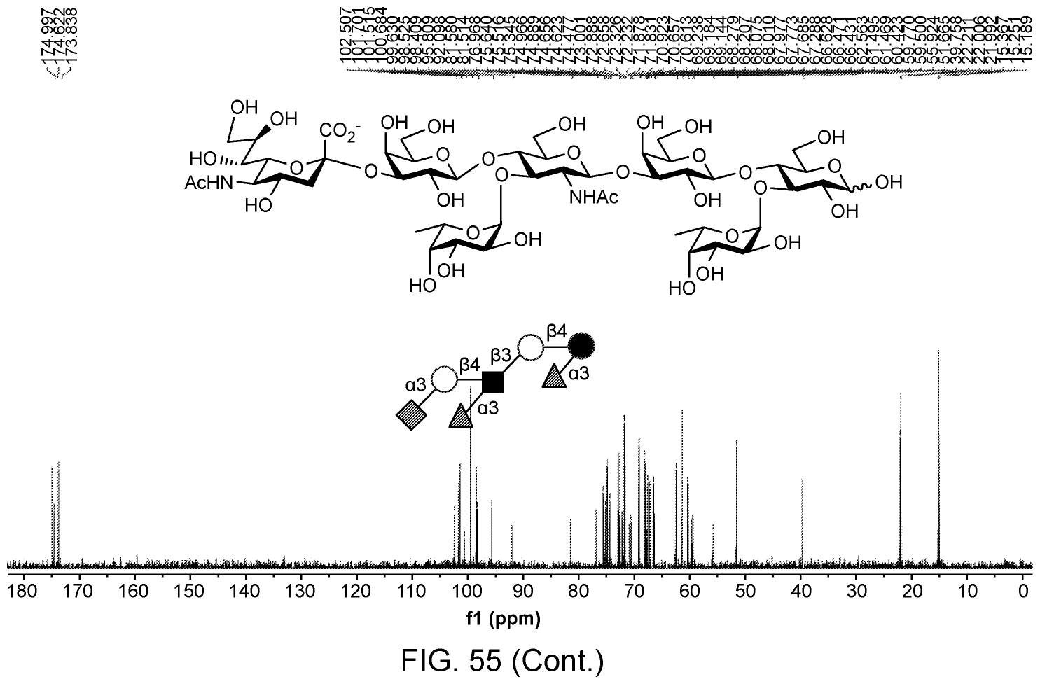

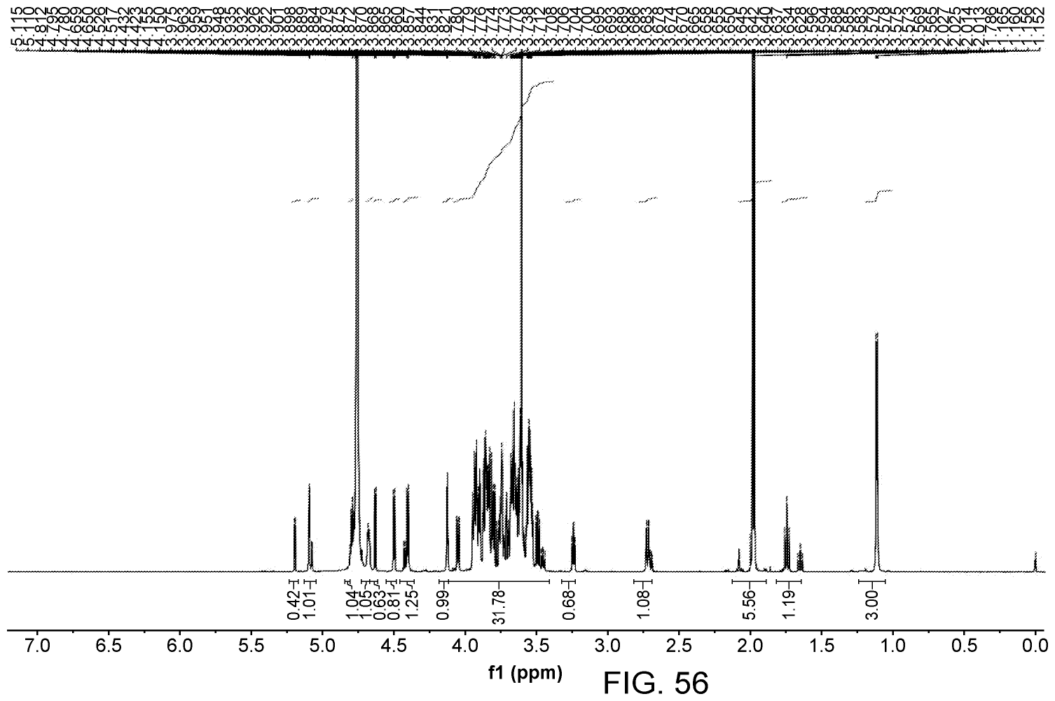

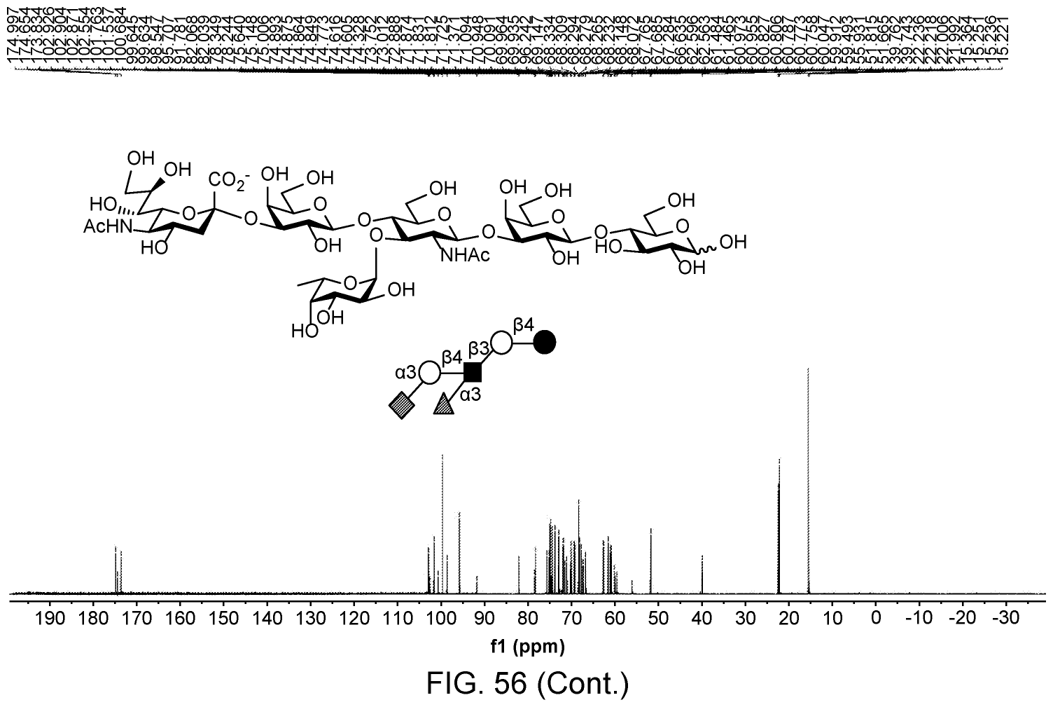

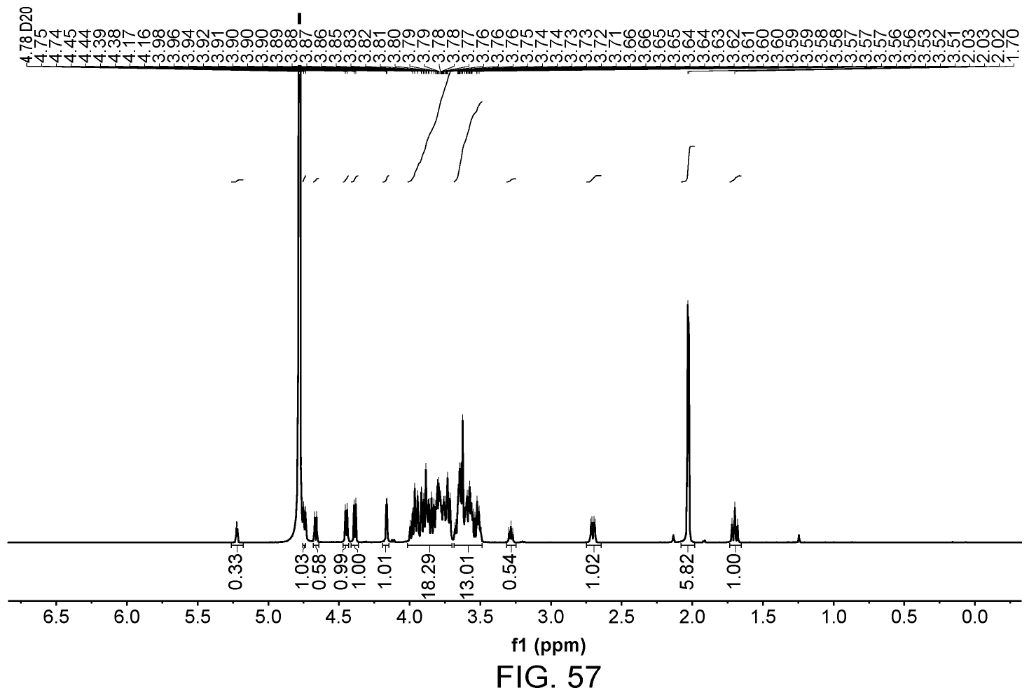

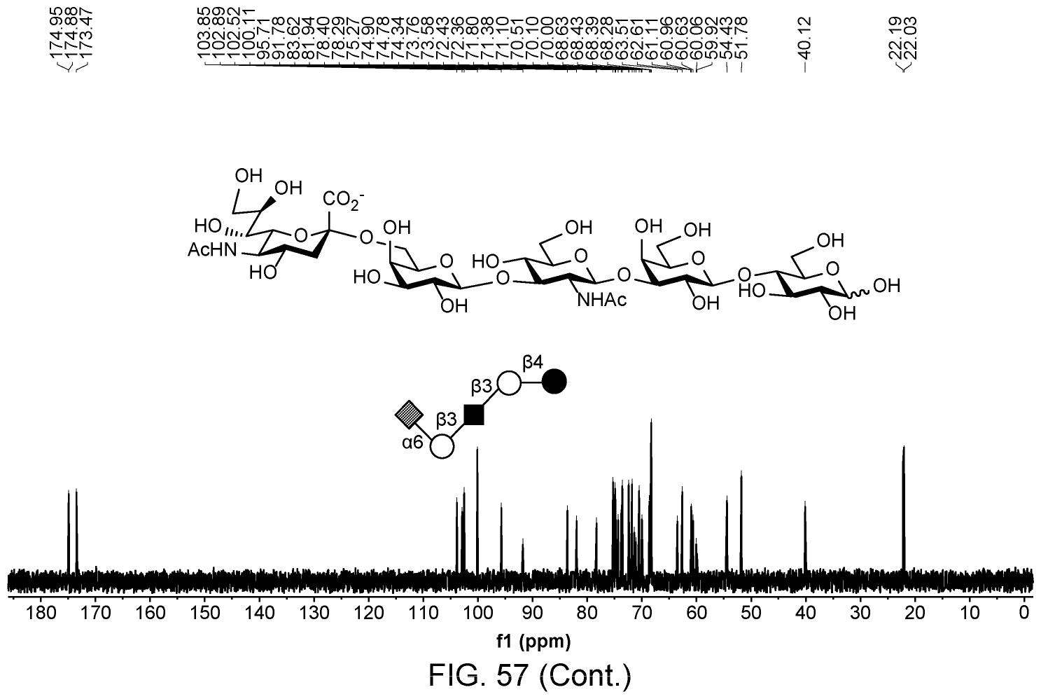

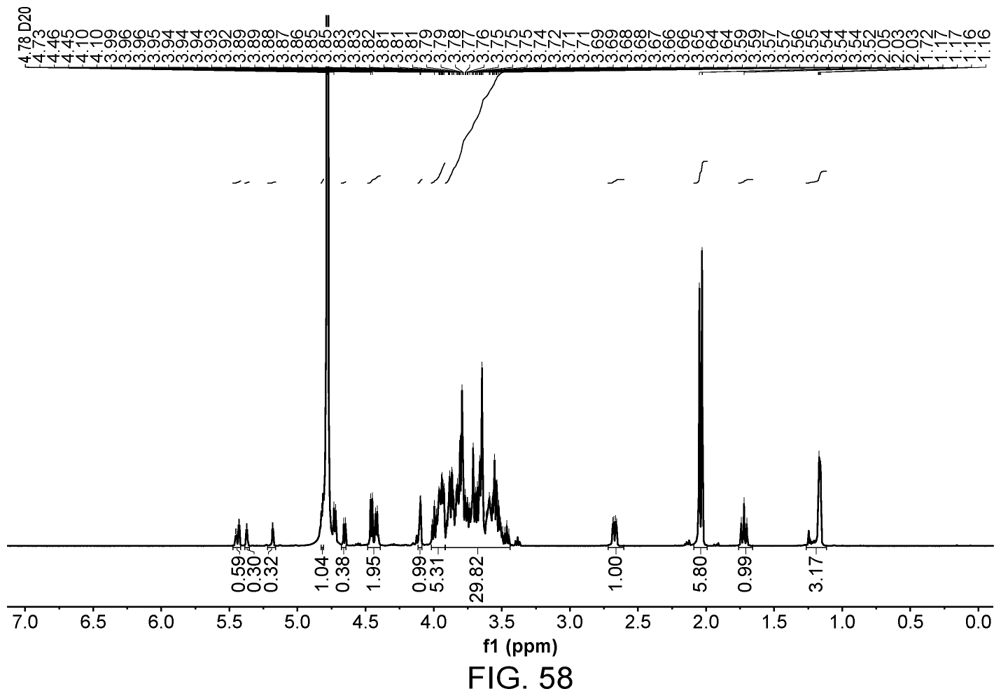

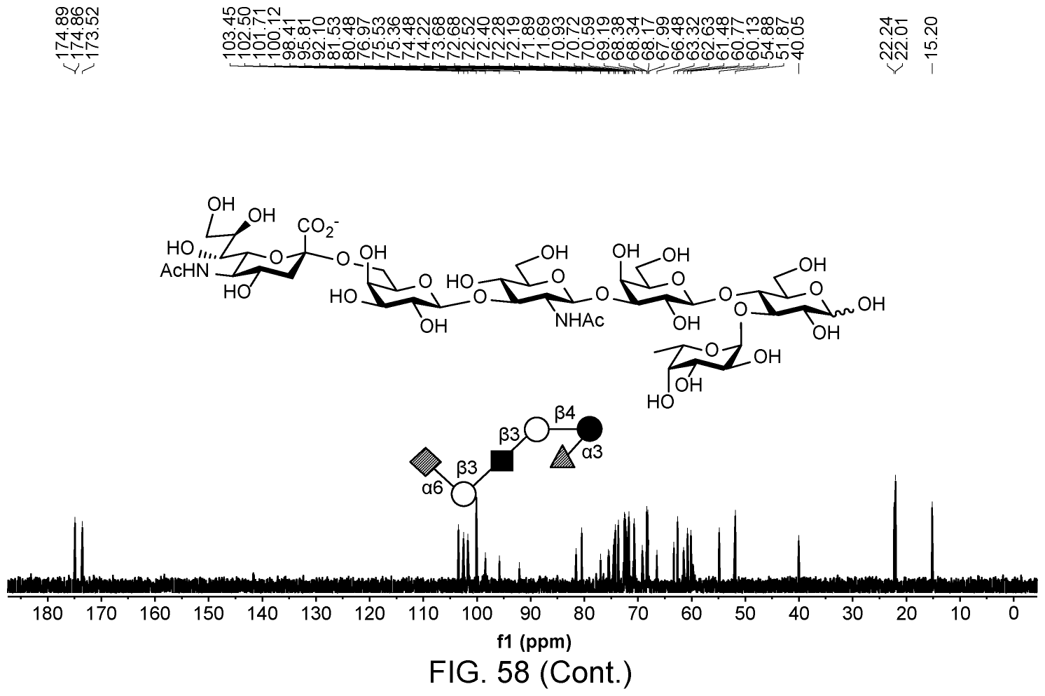

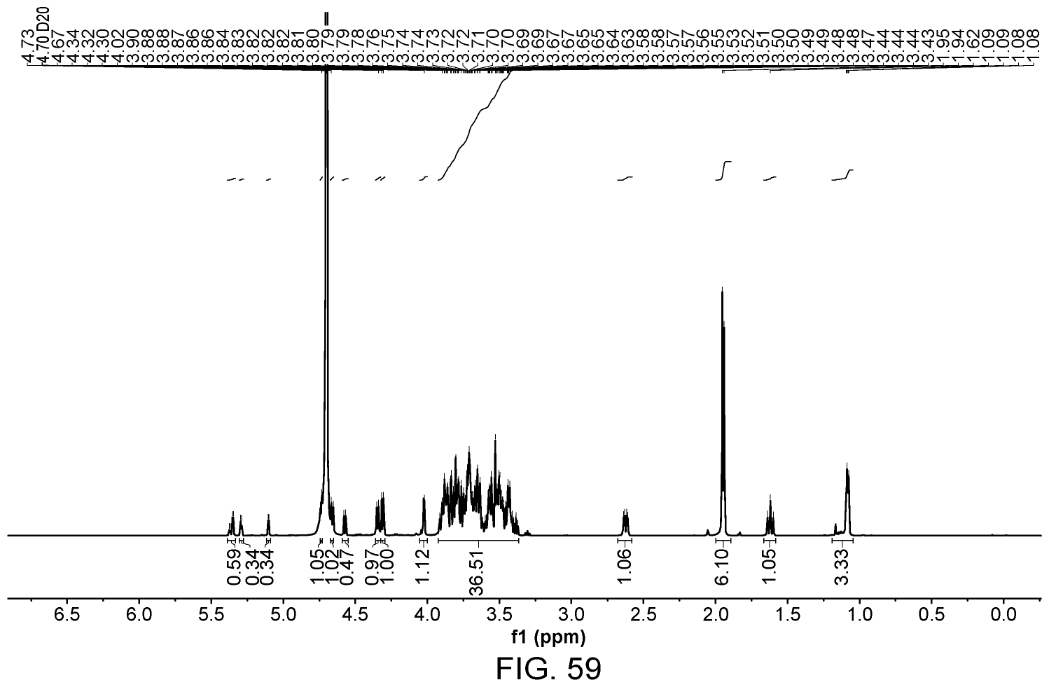

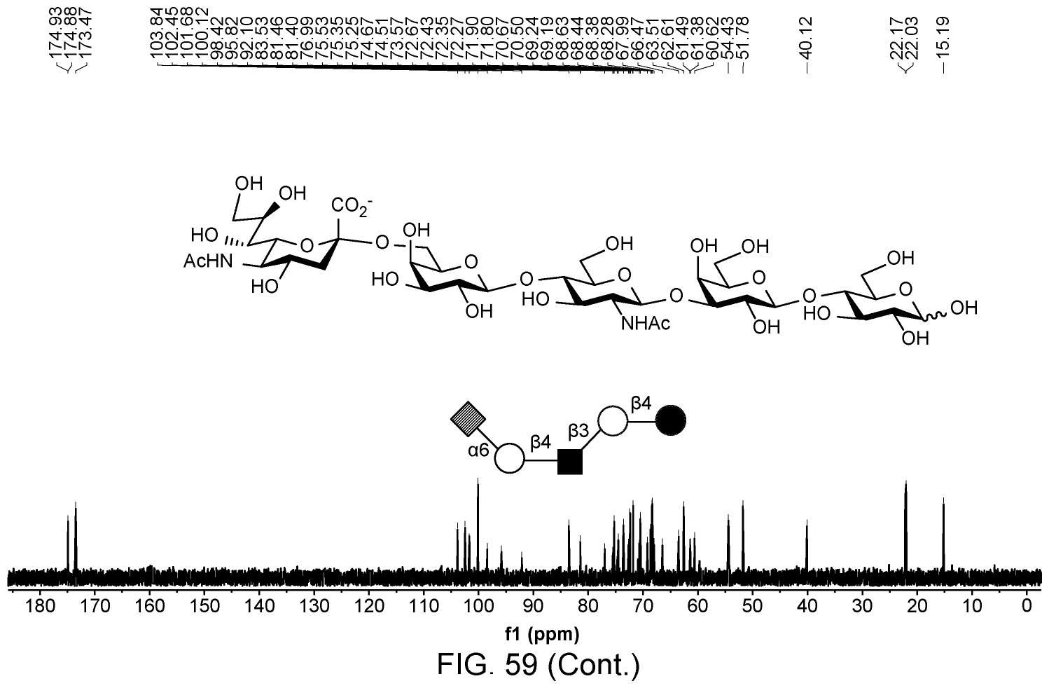

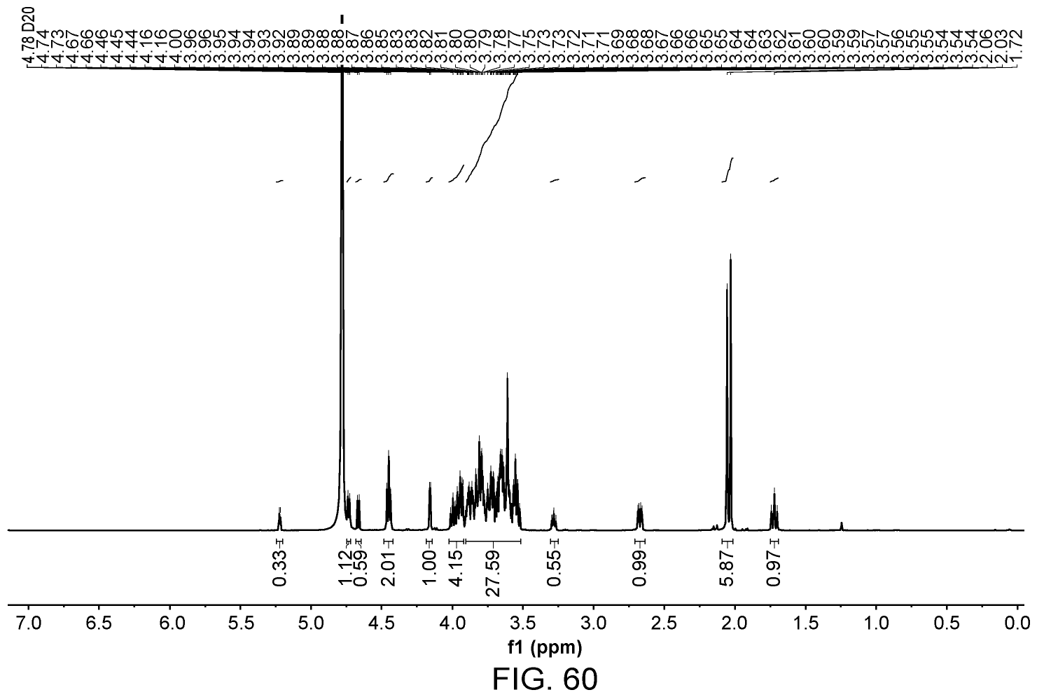

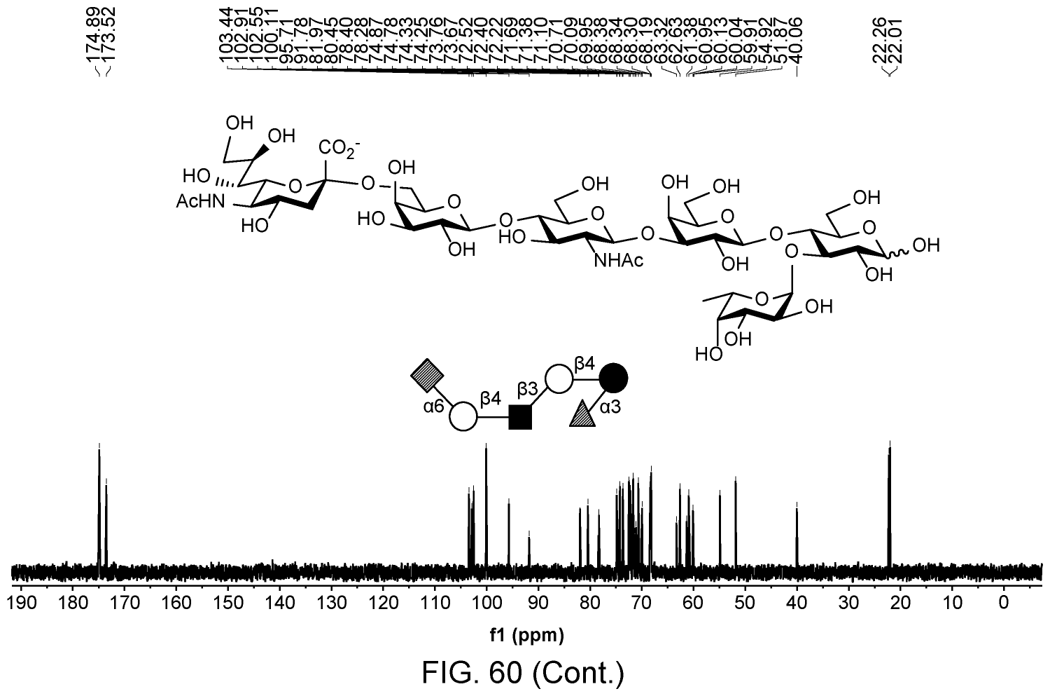

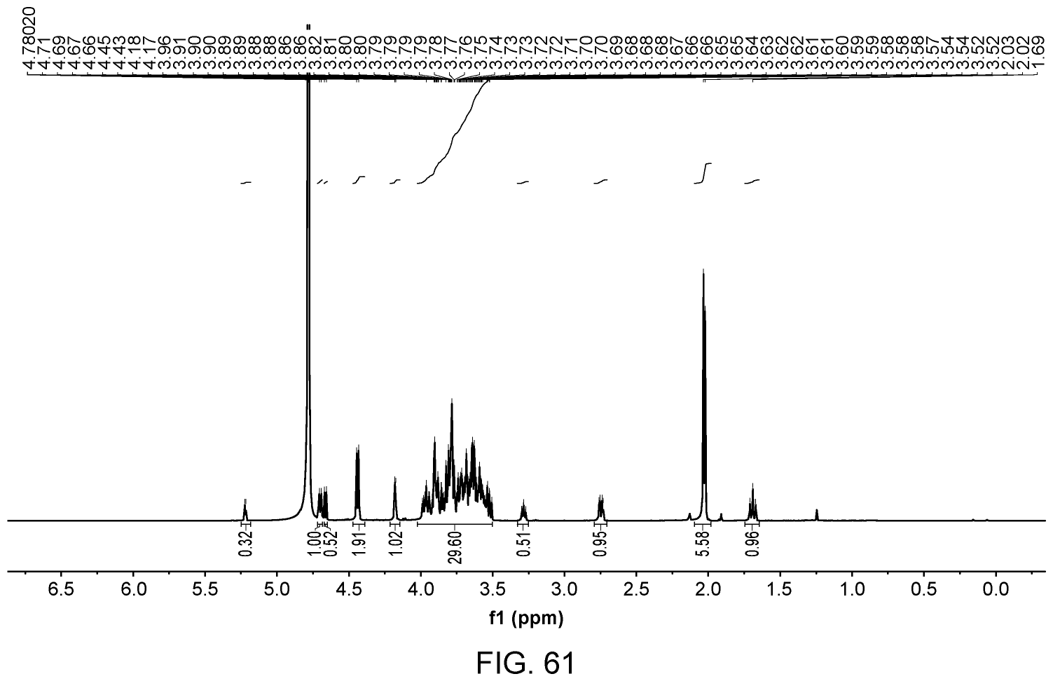

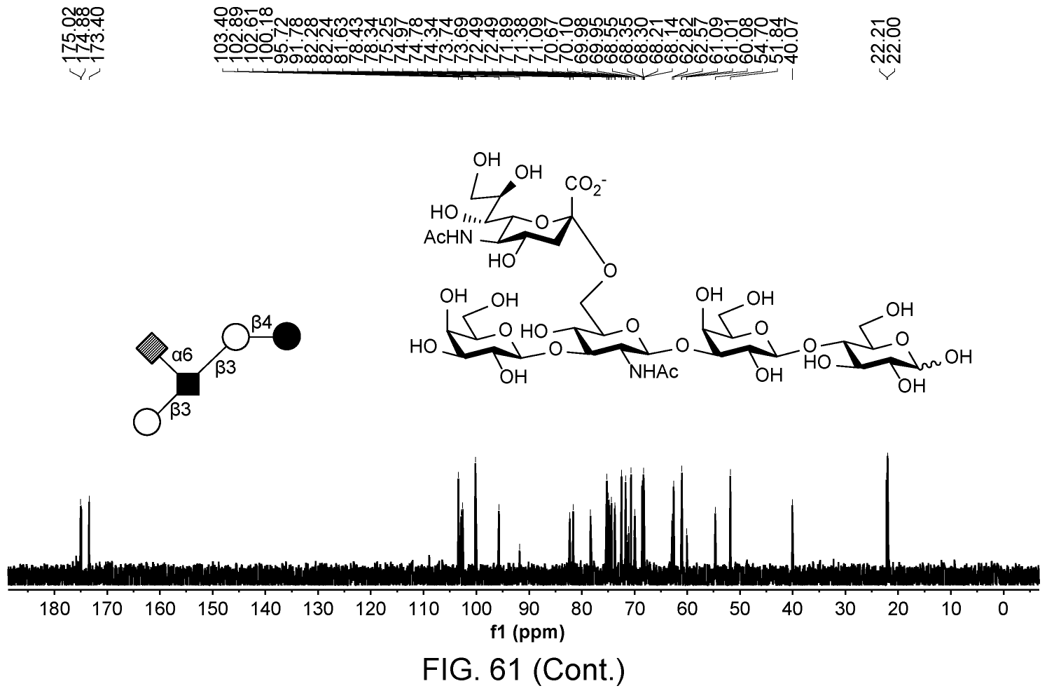

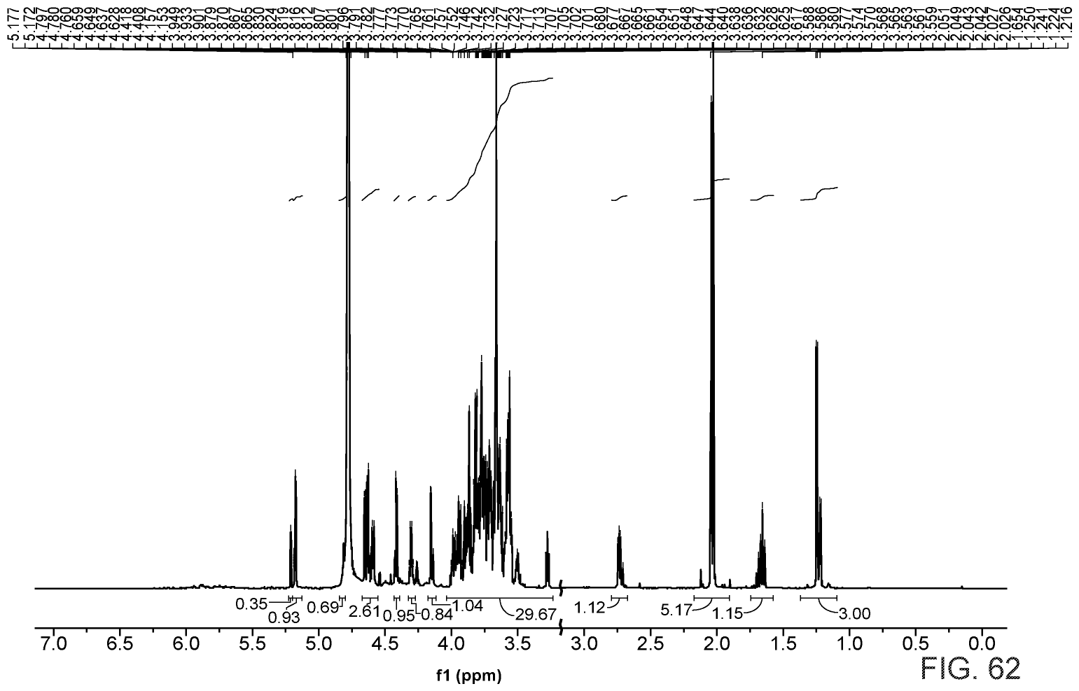

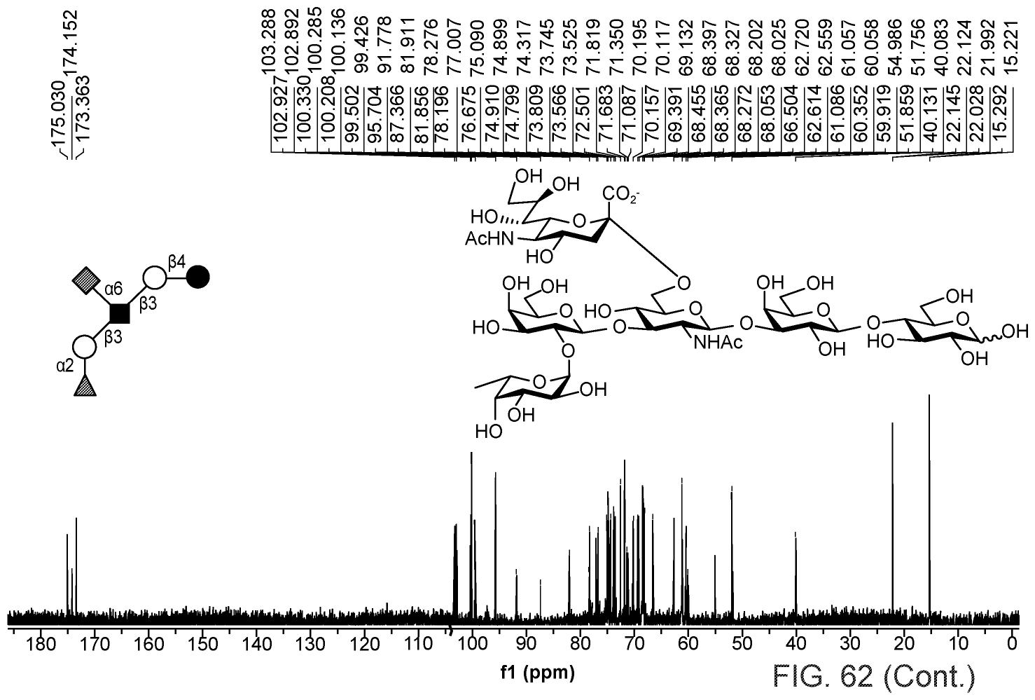

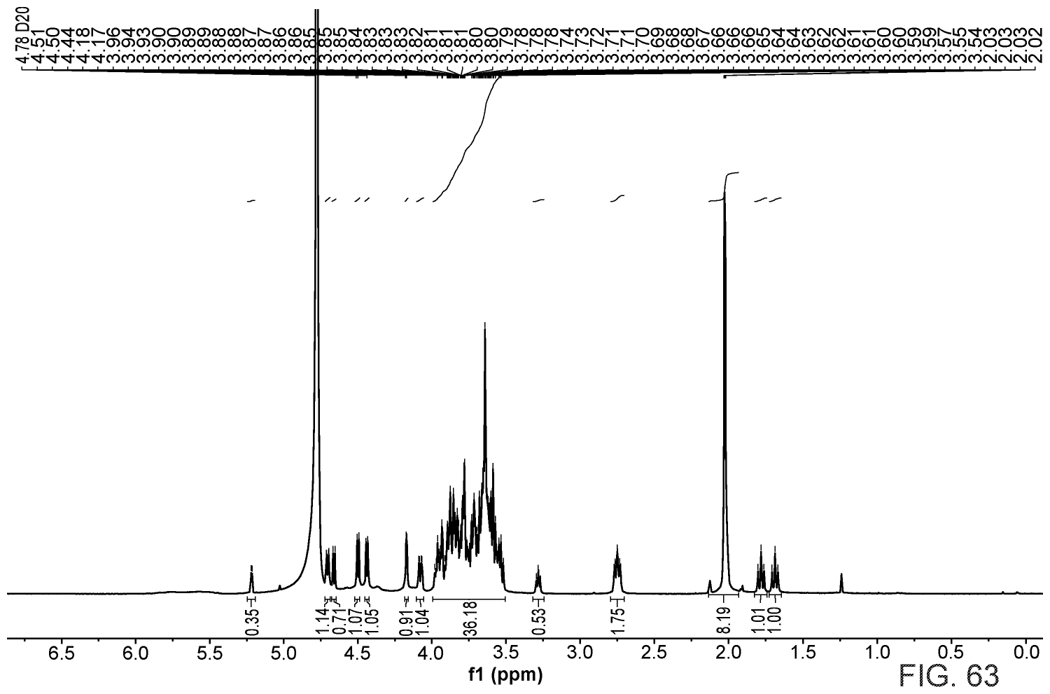

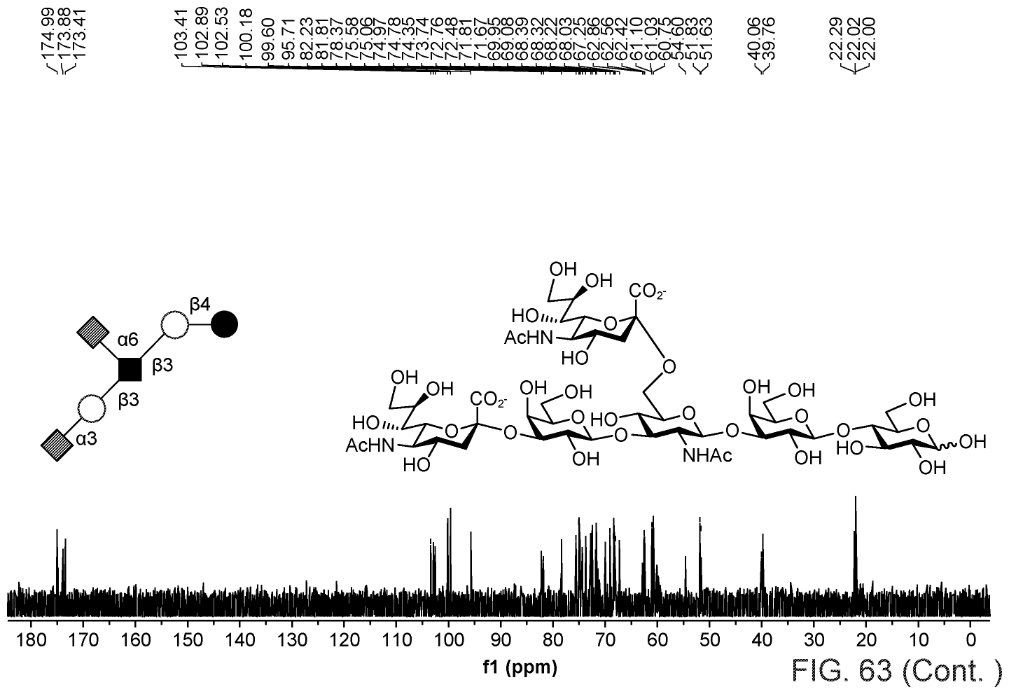

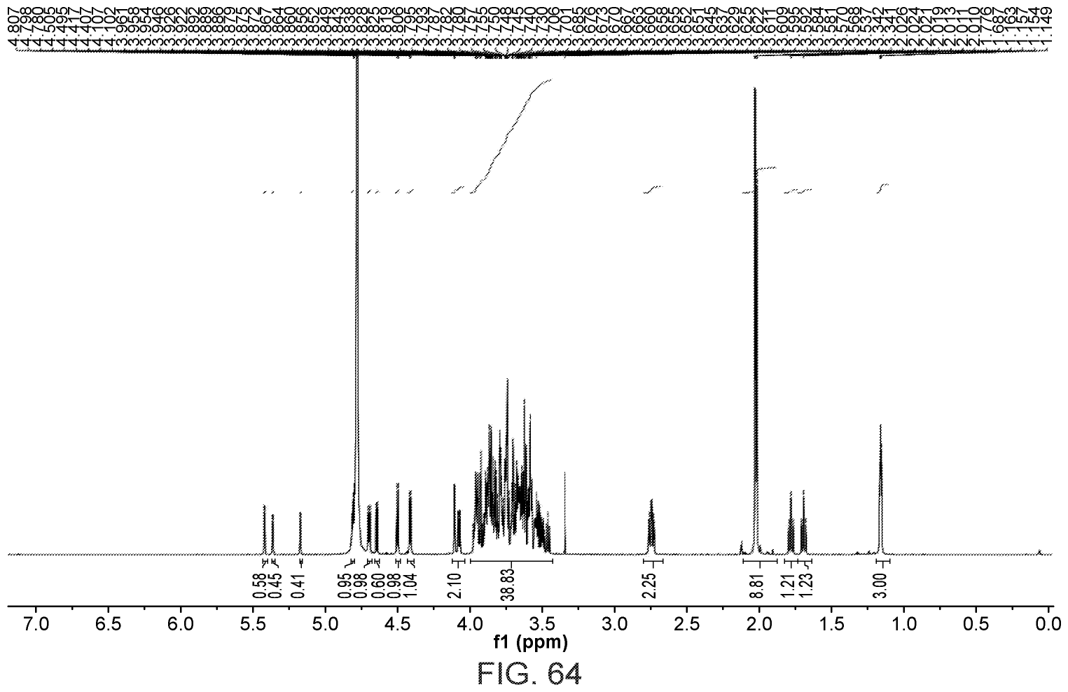

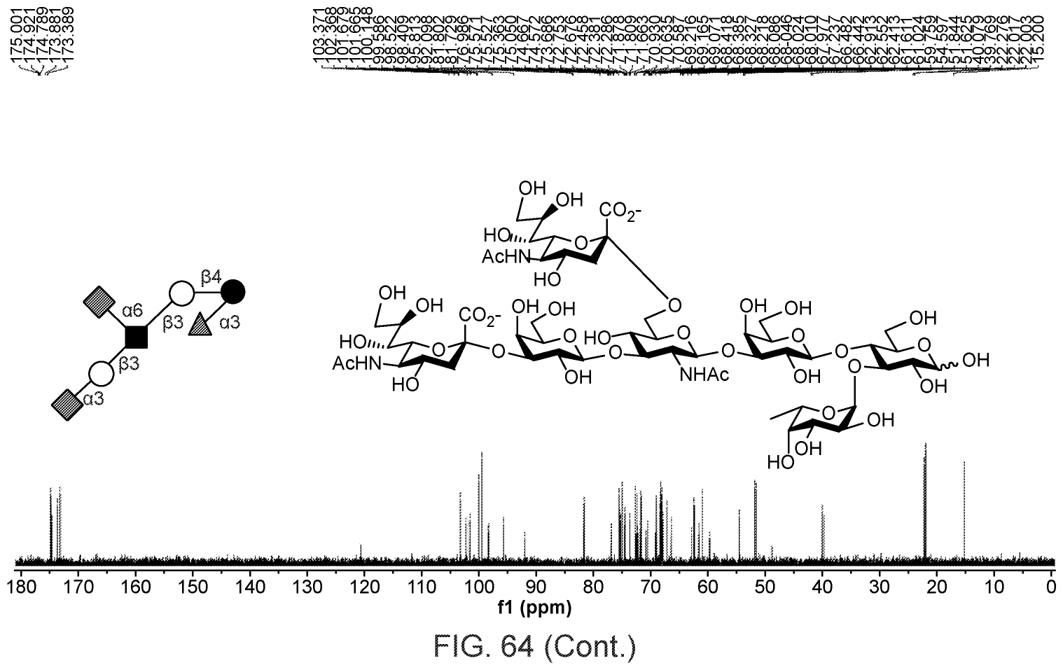

Furthermore, both V99M and stop-loss (which lead to the addition of 20 amino acids at the C-terminus) mutations of ST6GALNAC V have shown to enhance its catalytic activity and to correlate to the genetic cause of coronary artery disease (CAD). Both mutants and a combination mutant containing both mutations were constructed. They were expressed in E. coli and their expression levels and activities were compared. The soluble expression levels of both MBP-Δ50hST6GALNAC V_V99M-His₆ and MBP-Δ50hST6GALNAC V_ext20-His₆ were higher than that of the wild-type recombinant enzyme. The combination mutant MBP-Δ50hST6GALNAC V_V99M_ext20-His₆ was also successfully expressed as a soluble active enzyme. Furthermore, several PROSS designed mutants, shown in Table 2, expressed in an E. coli host were tested. Among them, MBP-Δ50hST6GALNAC V_Design7_ext20-His₆, shown in Table 2, was shown to have a high soluble expression level and good catalytic activity. It was used for highly efficient synthesis of DSLNTβNHCbz (46) from LSTaβNHCbz (32) using a OP2E sialylation reacton (FIG. 13B) or from LacβNHCbz (1) in a StOPMe strategy with pre-generation of sugar nucleotides.

With active hST6GALNAC V and mutants in hand and an understanding of their acceptor substrate specificities, EASyMap (FIG. 13A) for StOPMe synthesis of four Neu5Acα2-6GlcNAc-containing HMOβNHCbz targets (44-47) was designed.

The preparation of compounds 44-46 shares the common first two glycosylation steps. Again, sugar nucleotides including UDP-GlcNAc, UDP-Gal, and/or GDP-Fuc were generated in the first step followed by NmLgtA (GT1a)-catalyzed extension of LacβNHCbz (1) with a 1-3-linked GlcNAc with UDP-GlcNAc generated from GlcNAc via SA1 in the first step, enzyme deactivation, and Cvβ3GalT (GT2a)-catalyzed extension with a β1-3-linked Gal using UDP-Gal generated from Gal via SA2 in the first step, and enzyme deactivation. From there for the top route, hST6GALNAC V (GT4e)-catalyzed addition of Neu5Ac α2-6-linked to the internal GlcNAc with in situ generation of CMP-Neu5Ac formed LSTbβNHCbz (44). On one hand, deactivating the enzymes in the resulting reaction mixture followed by a single C18-cartridge purification produced pure LSTbβNHCbz (44). On the other hand, the reaction mixture after deactivation of enzymes was used directly for the next step to add a terminal α1-2-linked L-fucose by Hm2FT (GT3a)-catalyzed reaction with GDP-Fuc generated from L-fucose via SA3 in the first step formed F-LSTbβNHCbz (45) which was readily purified by deactivation of enzymes followed by a C18-cartridge purification. For the middle route, after the first two glycosylation steps for the formation of the LNTβNHCbz core, PmST1_M144D (GT4a)-catalyzed α2-3-sialylation of the terminal Gal with in situ generation of CMP-Neu5Ac via SA4, enzyme deactivation, followed by hST6GALNAC V (GT4e)-catalyzed addition of Neu5Ac α2-6-linked to the internal GlcNAc with in situ generation of CMP-Neu5Ac via SA4 formed DSLNTβNHCbz (46) which was readily purified by deactivation of enzymes followed by a C18-cartridge purification.

The production of FDS-LNT-IIβNHCbz (47) with a fucose α1-3-linked to the inner most glucose residue shared the same first step glycosylation reaction by NmLgtA (GT1a)-catalyzed extension of LacβNHCbz (1) with a β1-3-linked GlcNAc with in situ generation of UDP-GlcNAc from GlcNAc via SA1 and the generation of other sugar nucleotides needed in the future steps. After deactivation of the enzymes in the reaction mixture, the bottom route in the EASyMap FIG. 13A was followed. Briefly, Hp3/4FT (GT3b)-catalyzed reaction with GDP-Fuc generated from L-Fuc via SA3 in the first step, enzyme deactivation, Cvβ3GalT (GT2a)-catalyzed extension with a β1-3-linked Gal with UDP-Gal generated from Gal via SA2 in the first step, enzyme deactivation, PmST1_M144D (GT4a)-catalyzed α2-3-sialylation of the terminal Gal with in situ generation of CMP-Neu5Ac via SA4, enzyme deactivation, and finally hST6GALNAC V (GT4e)-catalyzed addition of Neu5Ac α2-6-linked to the internal GlcNAc with in situ generation of CMP-Neu5Ac, enzyme deactivation, and a single C18-cartridge purification produced pure FDS-LNT-IIβNHCbz (47).

For the synthesis of DSLNTβNHCbz (46), it was found that synthesizing it from LSTaβNHCbz (32) using OP2E sialylation reacton (FIG. 13B) was more efficient compared to the StOPMe strategy.

MBP-Δ50hST6GALNAC V_V99M-His₆, MBP-Δ50hST6GALNAC V_ext20-His₆, and the combination mutants MBP-Δ50hST6GALNAC V_V99M_ext20-His₆ were shown to be able to catalyze the sialylation of monosialyl Lewis a structure Neu5Acα3LNFP-IIβNHCbz (S-LNF II or F-LSTa) (37) to form the complex disialyl Lewis a structure FDS-LNT IβNHCbz (48) (FIG. 13C).

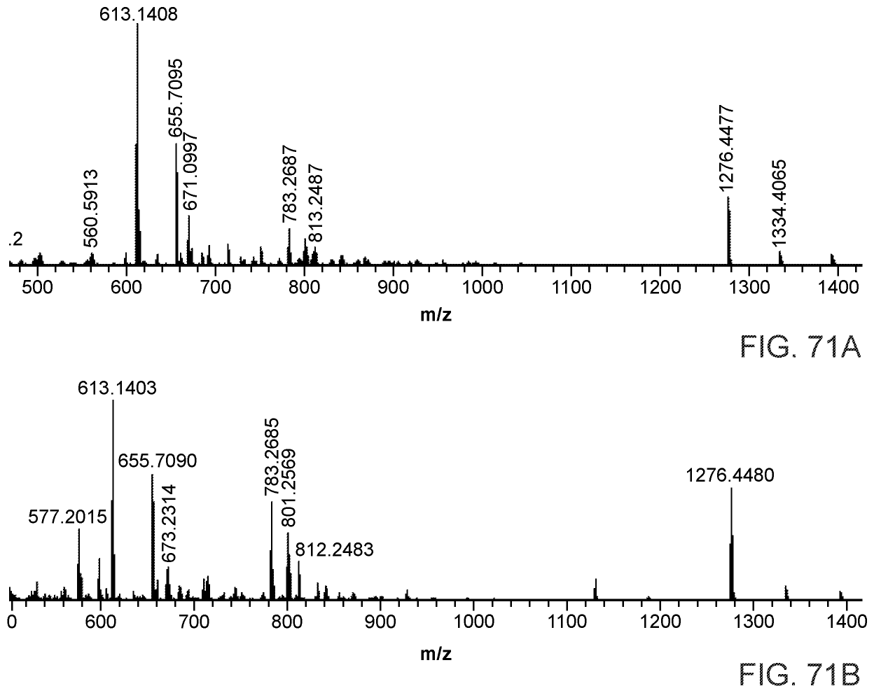

Assays for synthesizing FDS-LNTβNHCbz (48) from S-LNF-IIβNHCbz (37). Assays were carried out at 30° C. in a total volume of 10 μL in Tris-HCl buffer (100 mM, pH 7.5) containing (10 mM), CMP-Neu5Ac (15 mM), MgCl₂ (20 mM), MBP-Δ50hST6GALNAC V_ext20-Hiss (16 g), MBP-Δ50hST6GALNAC V_V99M-His₆, or MBP-Δ50hST6GALNAC V_V99M_ext20-His₆. The reactions were analyzed by HRMS at 24 hours and 72 hours. The formation of FDS-LNTβNHCbz (48) was observed (FIGS. 71A and 71B), and the conversion was around 40-50% after 72 h incubation using MBP-Δ50hST6GALNAC V_ext20-His₆. The formation of the target product was also observed in the reactions using MBP-Δ50hST6GALNAC V_V99M-His₆ or MBP-Δ50hST6GALNAC V_V99M_ext20-His₆.

### Example 14. Cloning, Expression, and Synthetic Application of β1-6-GlcNAcTs

The gene encoding full length human GCNT2-B (hGCNT2-B, GenBank ID: NP_001482), a β1-6-GlcNAcT, was codon optimized for E. coli expression, synthesized, and cloned into pET28a(+) by Twist Bioscience to obtain pET28a(+)-hGCNT2-B plasmid. The gene was subcloned into pMAL-c2X to obtain plasmid pMAL-c2X-hGCNT2-His₆. In addition, the plasmid containing the N-terminal 25 amino acid-truncated version of hGCNT2-B was also constructed for expressing MBP-Δ25hGCNT2-B-His₆ with an N-terminal MBP fusion component and a C-terminal His₆ tag. Origami B (DE3) transformed with pGro7 harboring chaperon GroEL-GroES was used as host cell for expressing MBP-Δ25hGCNT2-B-His₆. Its activity was confirmed using LNnTβNHCbz as the acceptor substrate and UDP-GlcNAc as the donor substrate in both small-scale (10 μL) reactions and preparative-scale synthesis with NMR characterization.

Enzyme cloning and truncation. The synthetic gene hGCNT2-B in the pET28a(+)-hGCNT2-B plasmid was amplified via polymer chain reactions (PCR) using forward primer: 5′- GACCGAATTCATGCCCTTATCGATGAGATATCTC-3′ with EcoRI restriction site (underlined) and reverse primer: 5′-CAGCAAGCTTTAGTGGTGGTGATGATGATGGAAGTACCATGATGGCTGAATG-3′ with HindIII restriction site (underlined). The PCR was performed in a reaction mixture (50 μL) containing the template plasmid DNA (10 ng), forward and reverse primer (0.2 M each), 1× Phusion HF buffer, dNTP mixture (0.2 mM each), and 1 U (0.5 L) of Phusion® High-Fidelity DNA Polymerase. The reaction mixture was subjected to 30 cycles of amplification at an annealing temperature of 62° C. The resulting PCR product was gel purified and double digested with EcoRI and HindIII restriction enzymes. The digested and purified PCR product was inserted into pMAL-c2X vector predigested with the same restriction enzymes via T4 ligase and transformed into E. coli DH5α Z-competent cells. Selected clones were grown for plasmid minipreps, and the nucleic acid sequence was confirmed by customer sequencing by Genewiz.

The truncation was carried out using Q5® Site-Directed Mutagenesis kit according to the procedures provided by the manufactory. The plasmid pMAL-c2X-hGCNT2-B-His₆ was used as the PCR template and the primers used were forward primer: 5′-TTCGGCGGAGATCCTTCT-3′ and reverse primer: 5′-CATGAATTCTGAAATCCTTCCC-3′. Briefly, a reaction mixture (25 L) containing 12.5 μL of Q5 Hot Start High-Fidelity 2× Master Mix, 10 ng (1 μL) plasmid as the template, 0.5 μM (1.25 μL) for each of forward and reverse primers, Nuclease-free Water (9 μL) was subjected to 30 cycles of amplification with 30 seconds of annealing (61° C.), 3.5 minutes of extension at 72° C. for each cycle, and 2 more minutes extension after the amplification. The standard KLD reaction (10 μL) containing 5 μL of 2×KLD Reaction Buffer, 1 μL of 10×KLD Enzyme Mix and 3 μL Nuclease-free Water was carried out by incubating the mixture at room temperature for 5 minutes. The products were then transformed to DH5α competent cell prepared by Mix and Go!™ Competent Cells (Zymo Research). The plasmids with target mutants were confirmed by DNA sequencing by Genewiz. The nucleic acid and the amino acid sequences of MBP-Δ25hGCNT2-B-His₆ are shown in SEQ ID NO: 9 and SEQ ID NO: 10, respectively.

Enzyme expression and purification. E. coli Origami B(DE3) cells with pGro7 harboring the recombinant plasmid containing the target gene were cultured at 37° C. in 50 mL LB media containing 0.05 mg mL⁻¹ ampicillin, 0.025 mg mL⁻¹ kanamycin, 0.0175 mg mL⁻¹ chloramphenicol, 0.005 mg mL⁻¹ tetracycline with rapid shaking at 220 rpm. Then 15 mL of the overnight cell culture was transferred to 1 L of LB media containing the same antibiotics and 0.5 g L⁻¹ L-arabinose and incubated at 37° C. When the OD600 nm of the cell culture reached 0.6-0.8, IPTG (0.1 mM) was added to induce the expression of the recombinant enzyme followed by incubation at 15° C. for 24 h with shaking at 220 rpm in a C25KC incubator shaker (New Brunswick Scientific, Edison, NJ).

Bacterial cells were harvested by centrifugation at 4° C. in a Thermo Lynx 6000 centrifuge with Rotor Lynx F9-6×1000 at 4,392×g for 30 min. The cell pellet was re-suspended with lysis buffer (Tris-HCl buffer, 100 mM, pH 8.0 containing 0.1% Triton X-100) (20 mL for cells collected from 1 L cell culture). The cells were lysed by homogenization using an Avestin EmulsiFlex-C₃ homogenizer or sonication using a Sonics Vibra-Cell system with the following protocol: 10 s (sonication)/59 s (rest) for a total of 7 min on ice. Cell lysate was obtained by centrifugation at 4° C. with Sorvall ST16R, HIGHConic II 6×94 mL fixed angle rotor at 9016×g for 1 hr and the supernatant (lysate) was collected. Protein purification was carried out using 5 mL Bio-Scale Mini Profinity IMAC Cartridges in a Bio-Rad NGC 100 Medium-Pressure Chromatography System with a flow rate of 5 mL min⁻¹. The supernatant was loaded to the Ni²⁺-NTA column pre-equilibrated with 10 column volumes of a binding buffer (5 mM imidazole, 0.5 mM NaCl, 50 mM Tris-HCl buffer, pH 7.5). The column was washed with 10 column volumes of the binding buffer followed by 10 column volumes of a washing buffer (10 mM imidazole, 0.5 mM NaCl, 50 mM Tris-HCl buffer, pH 7.5). The target protein was eluted with 10 column volumes of a elution buffer (200 mM imidazole, 0.5 mM NaCl, 50 mM Tris-HCl buffer, pH 7.5). Fractions containing the target enzyme were collected and concentrated via MilliporeSigma™ Amicon™ Ultra-15 Centrifugal Filter Units with MWCOs of 10 kDa, 10% glycerol was added before stored at −20° C.

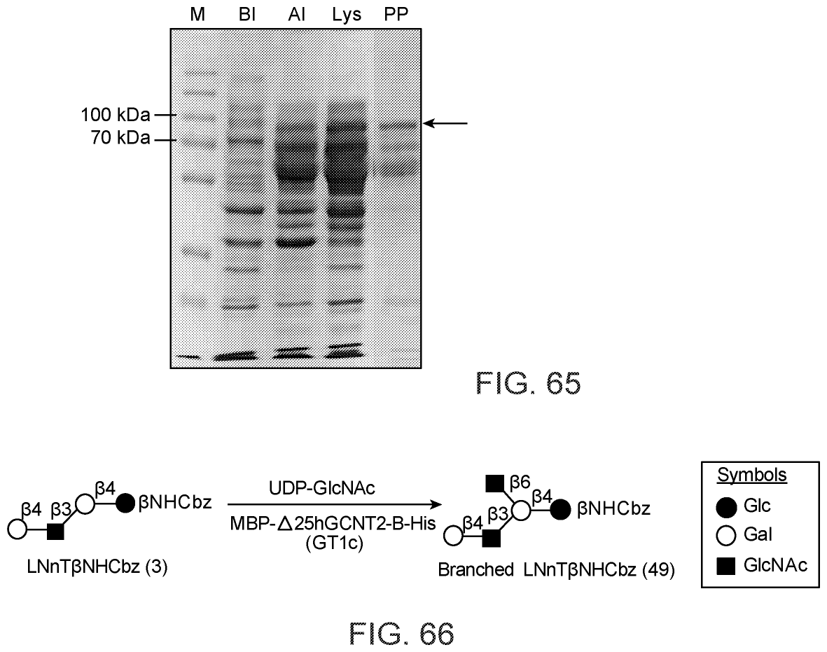

SDS-PAGE analysis results for enzyme expression and purification of MBP-Δ25hGCNT2-B-His₆ are shown in FIG. 65.

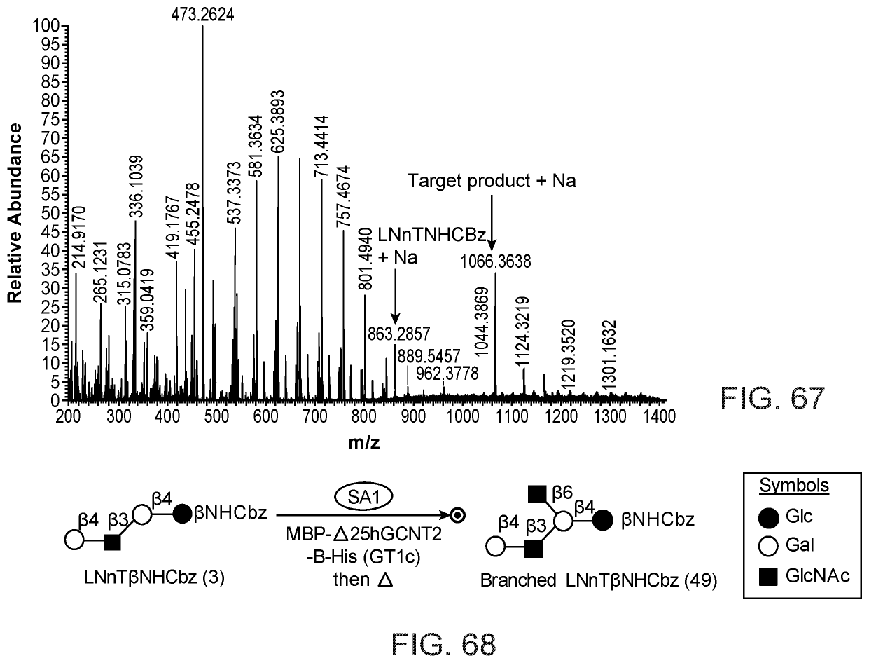

Enzyme activity test and synthesis. The MBP-Δ25hGCNT2-B-His₆ enzyme showed good activity with incubated at 30° C. for overnight in a reaction mixture (10 L) (FIG. 66) containing Tris-HCl (100 mM, pH 7.4), EDTA (10 mM), LNnTβNHCbz (2 mM), UDP-GlcNAc (3 mM), and 3 μL of concentrated enzyme (out of ˜300 μL total purified from 1 L LB culture). The product was identified by HRMS analysis (FIG. 67).

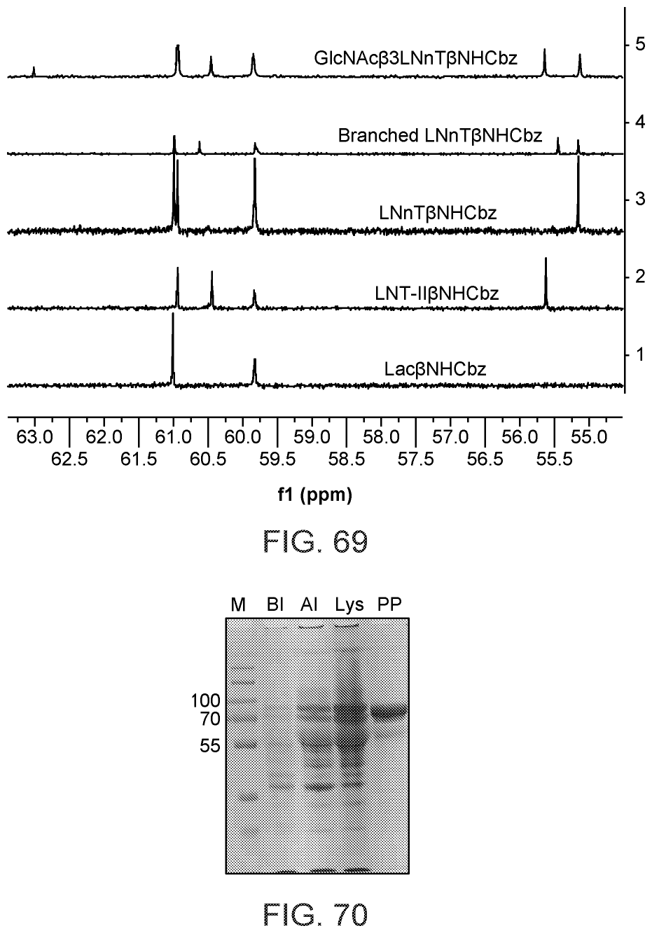

Preparative-scale synthesis was also successfully carried out using LNnTβNHCbz as the acceptor substrate using a one-pot multienzyme (OPME) process (FIG. 68). Briefly, LNnTβNHCbz (3) (10 mg, 0.012 mmol), ATP (0.018 mmol), UTP (0.018 mmol), and GlcNAc (0.018 mmol) were dissolved in water and combined to form a solution containing 20 mM MgCl₂ and 100 mM Tris-HCl pH 7.5, BLNahK (0.4 mg), PmGlmU (0.25 mg), PmPpA (0.1 mg), and MBP-Δ25hGCNT2-B-His₆ purified from 2 L culture. The reaction was completed in two days with incubation at 30° C. After purification with a C18 column on a CombiFlash system to obtain the pure product as a white powder. The yield was 95%. ¹³C NMR spectra comparison was carried out (FIG. 69).

### Example 15. Synthesis of HMOβNHCbz Containing the Hexoase pLNnH-Core Using StOPMe Strategy with Pre-Generation of Sugar Nucleotides

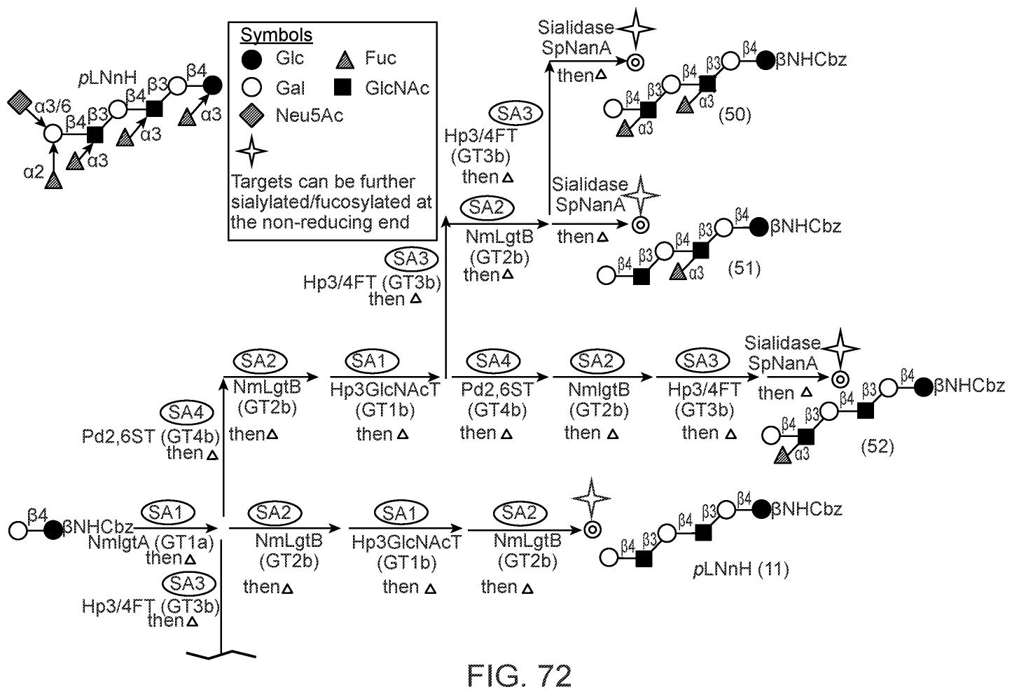

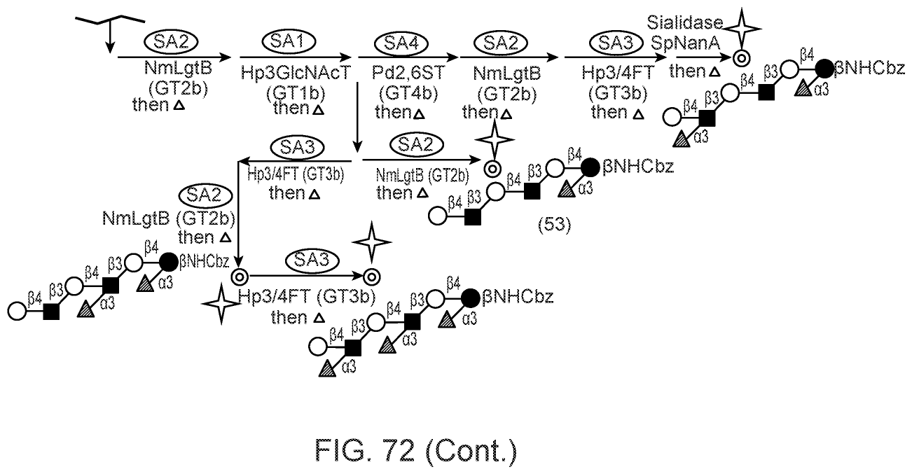

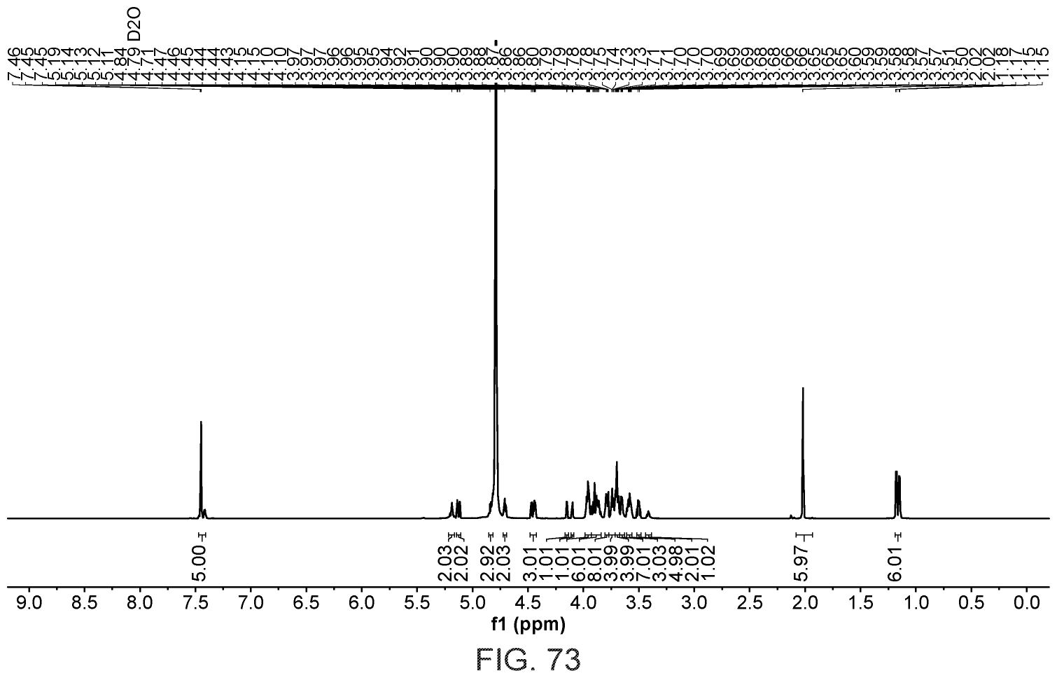

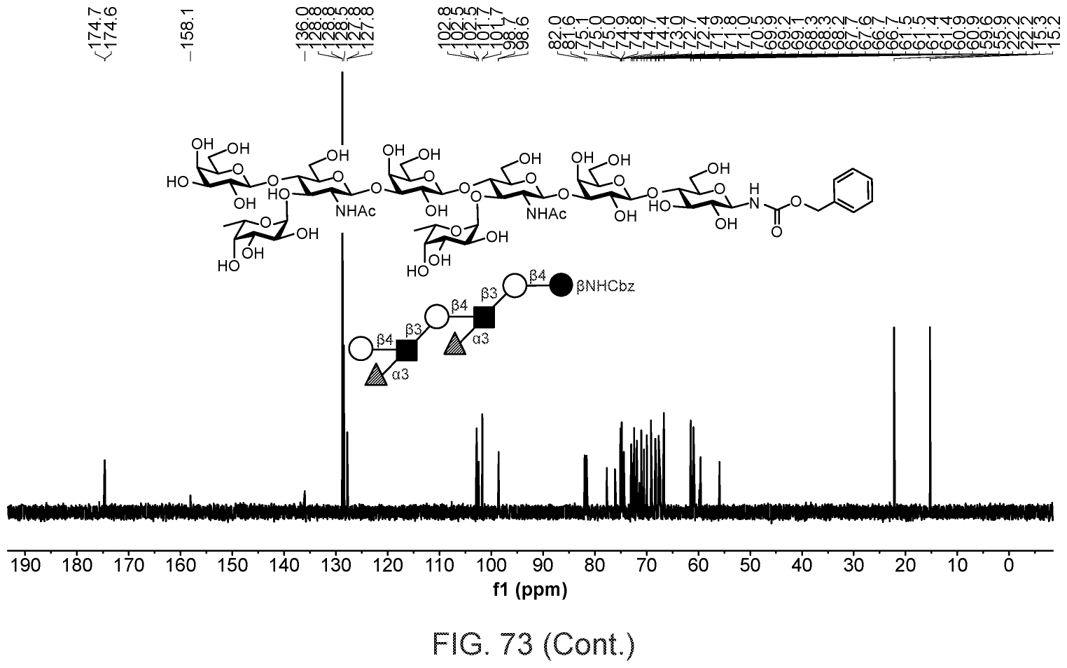

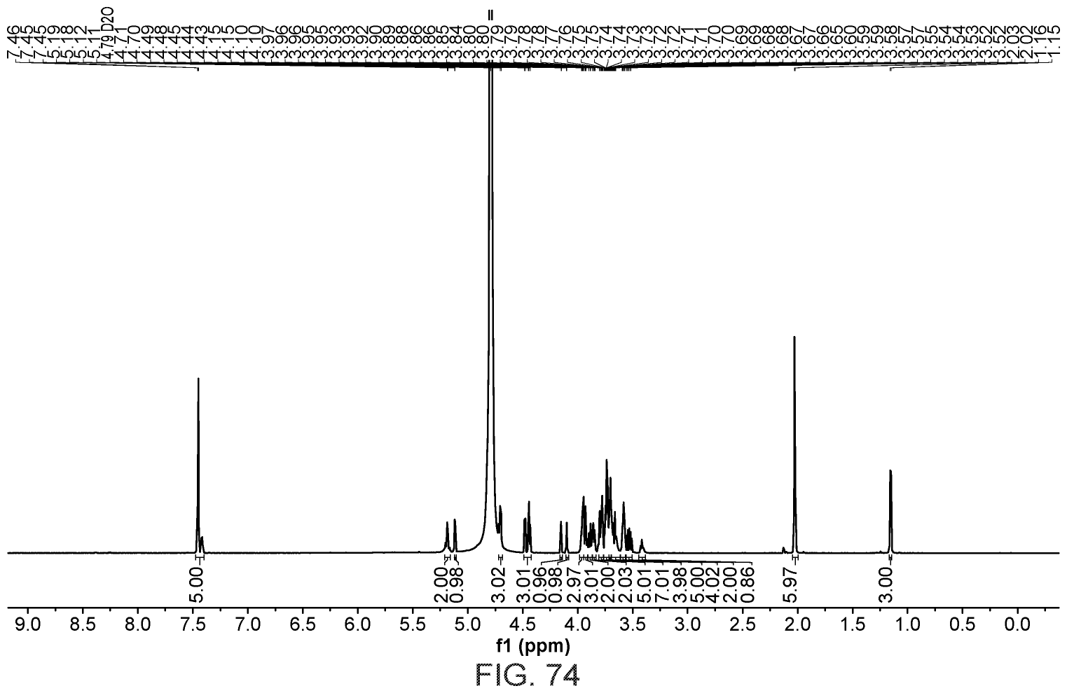

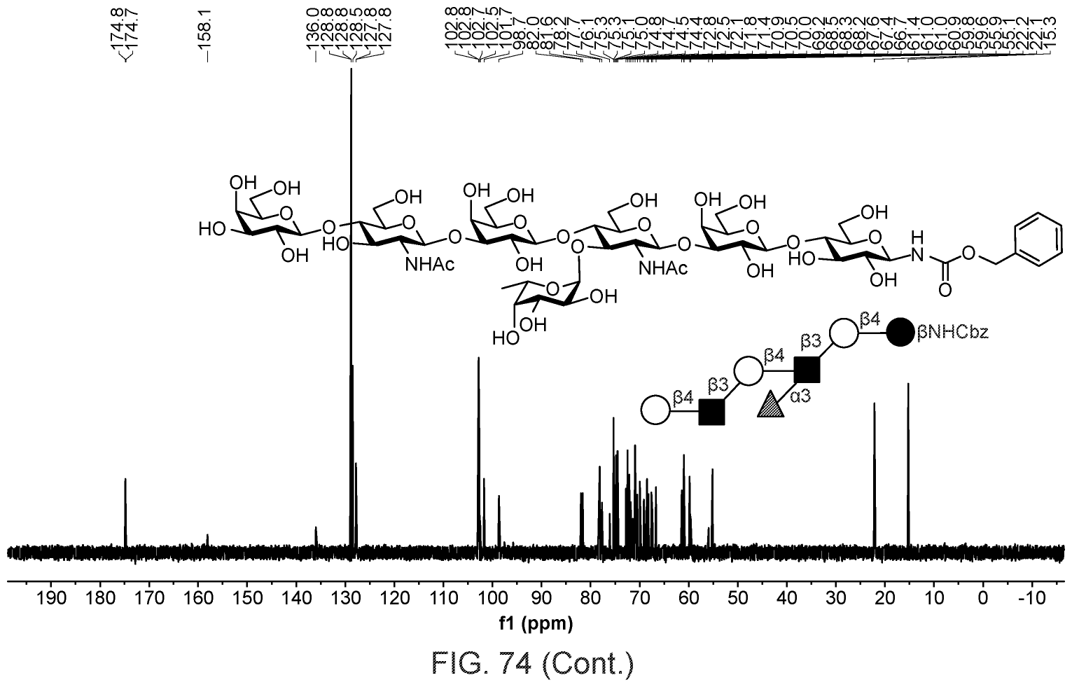

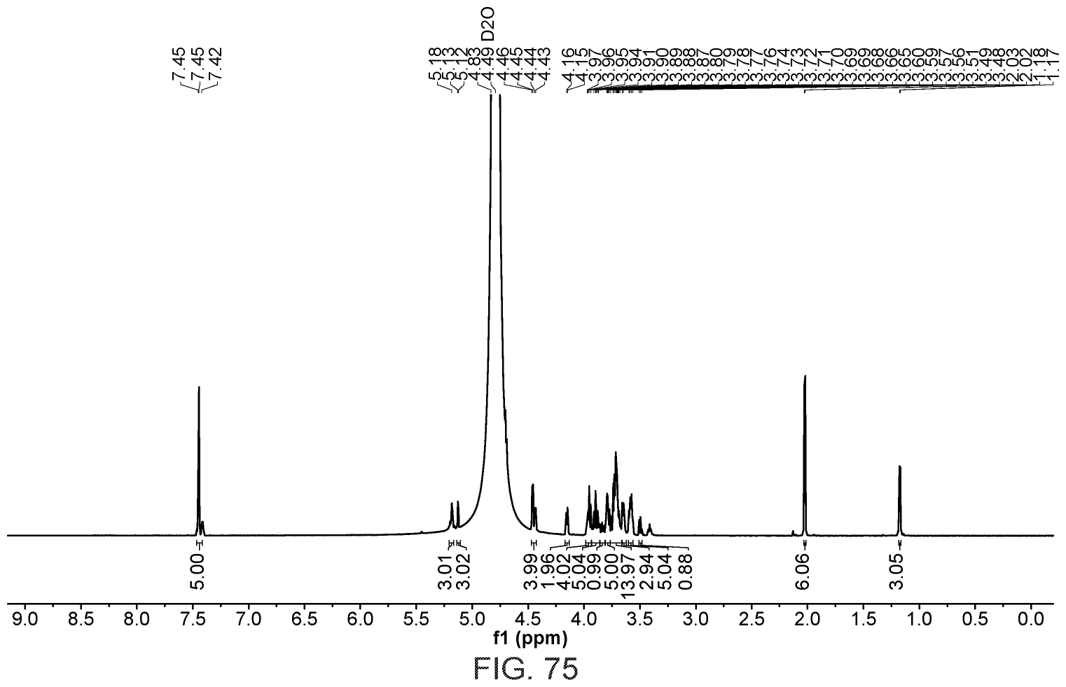

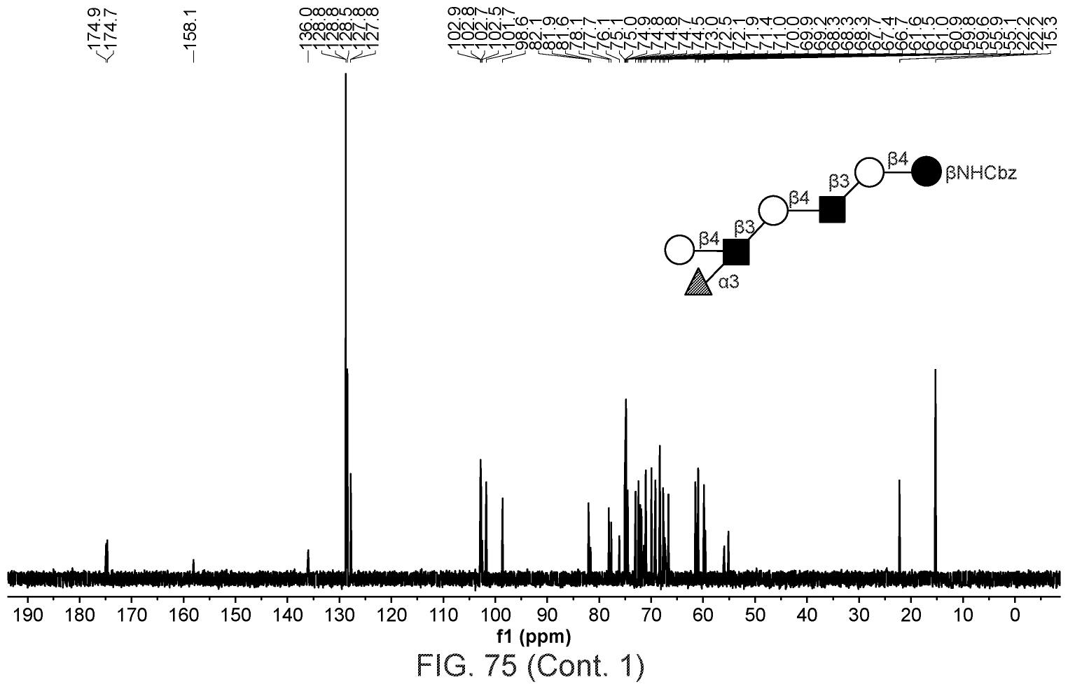

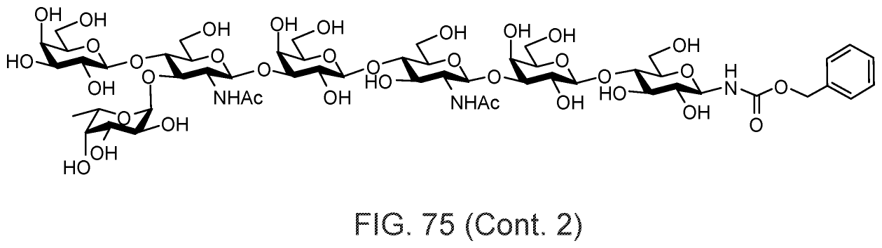

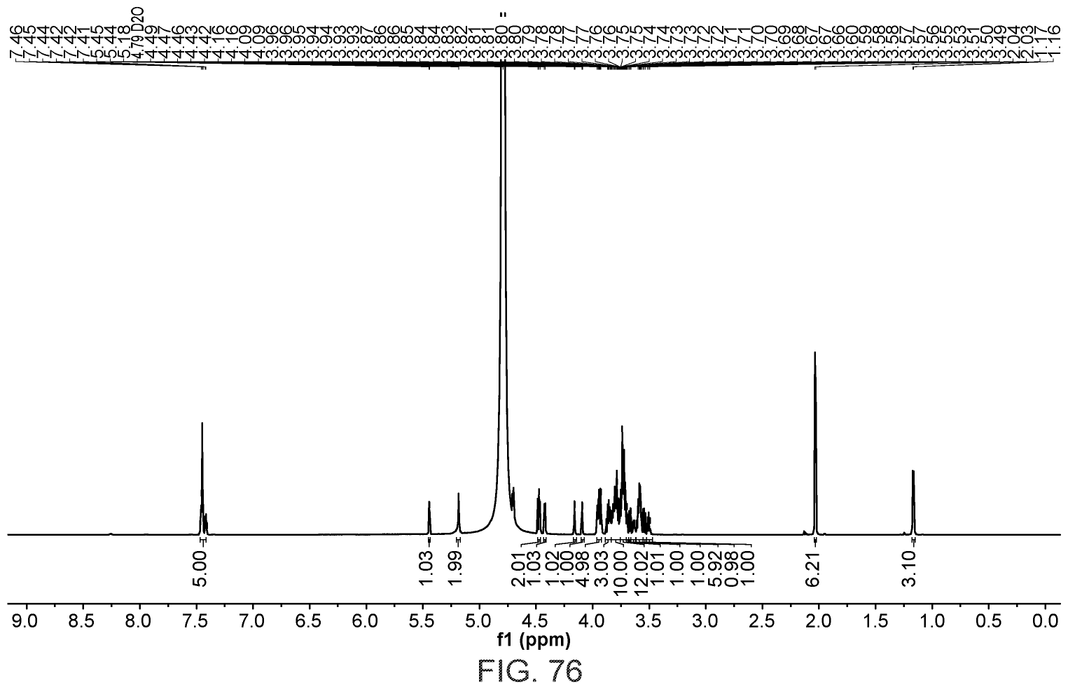

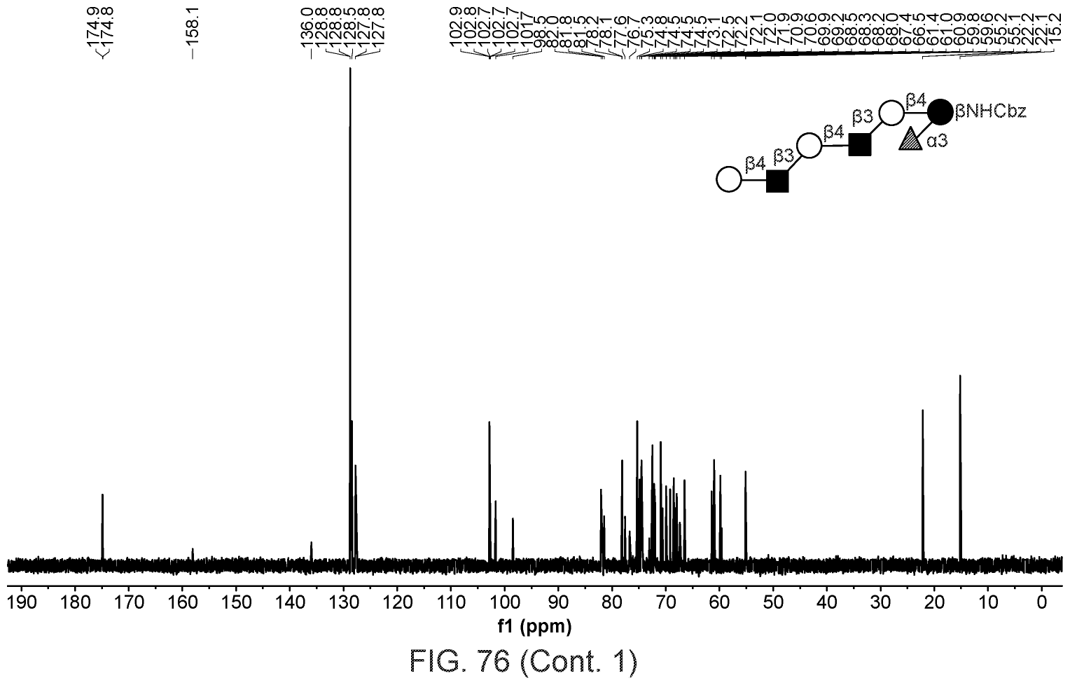

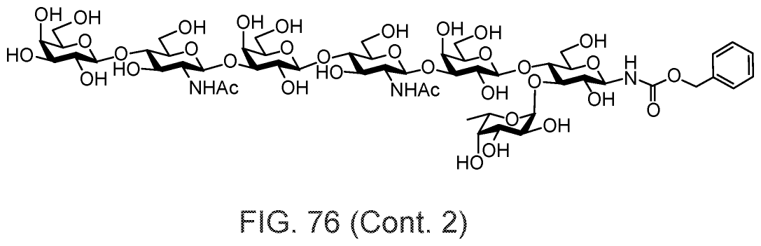

The StOPMe strategy with pre-generation of sugar nucleotides has also been successfully applied for synthesizing other HMOβNHCbz compounds including those containing the hexoase pLNnH-core. Some examples are described below with the guidance of the EASyMap designed (FIG. 72).

StOPMe synthesis of Galβ4GlcNAcβ3Galβ4GlcNAcβ3Galβ4GlcβNHCbz (pLNnHβNHCbz) (11). Galactose (0.504 mmol), GlcNAc (0.504 mmol), ATP (1.26 mmol), and UTP (1.26 mmol) were dissolved in water in a 50 mL centrifuge tube containing Tris-HCl buffer (100 mM, pH 7.5) and MgCl₂ (20 mM). The pH of the mixture was adjusted to 7.5 by adding 5 M NaOH. After the addition of BLNahK (4 mg), PmGlmU (4 mg), SpGalK (3.5 mg), BLUSP (3 mg), and PmPpA (2 mg), water was added to bring the final volume to 5 mL. The reaction mixture was incubated at 30° C. in an incubator shaker for 20 h with agitation at 180 rpm. Reaction progress was monitored by HRMS analysis. After incubating the reaction mixture in a boiling water bath for 5 min then cooled down, LacβNHCbz (100 mg, 0.21 mmol) and NmLgtA (3 mg) were added to synthesize GlcNAcβ3Galβ4GlcβNHCbz. The reaction mixture was incubated at 30° C. in an incubator shaker for 20 h with agitation at 180 rpm. After the reaction was completed, the reaction mixture was incubated in a boiling water bath for 5 min then cooled down and moved to the next step without purification. For each step, pH was checked and adjusted to 7.5 using 5 M NaOH before adding enzymes. NmLgtB (0.6 mg) was then added into reaction mixture to obtain Galβ4GlcNAcβ3Galβ4GlcβNHCbz. Once the reaction was completed (8 h), the reaction was stopped by the same workup method using boiling water bath. To synthesize GlcNAcβ3Galβ4GlcNAcβ3Galβ4GlcβNHCbz, Hpβ3GlcNAcT (4 mg) was added and the reaction mixture was incubated at 30° C. in an incubator shaker for 20 h with agitation at 180 rpm. After workup, NmLgtB (0.6 mg) was added to synthesize Galβ4GlcNAcβ3Galβ4GlcNAcβ3Galβ4GlcβNHCbz (11). The reaction was incubated at 30° C. in an incubator shaker for 8 h followed by workup with boiling water bath and cooled down to room temperature. The final volume was around 20 mL with final product concentration of around 10 mM. Galβ4GlcNAcβ3Galβ4GlcNAcβ3Galβ4GlcβNHCbz (11) was obtained by single C18-cartridge purification process as a white powder (200 mg, 79% yield for 4 steps from 100 mg LacβNHCbz). The NMR data were consistent with those data described above using the MSOPME strategy.

StOPMe synthesis of Galβ4(Fucα3)GlcNAcβ3Galβ4(Fucα3)GlcNAcβ3Galβ4GlcβNHCbz (50). Galactose (0.504 mmol), GlcNAc (0.504 mmol), L-fucose (0.504 mmol), ATP (1.89 mmol), UTP (1.26 mmol), and GTP (0.63 mmol) were dissolved in water in a 50 mL centrifuge tube containing Tris-HCl buffer (100 mM, pH 7.5) and MgCl₂ (20 mM). The pH of the mixture was adjusted to 7.5 by adding 5 M NaOH. After the addition of BLNahK (4 mg), PmGlmU (4 mg), SpGalK (3.5 mg), BLUSP (3 mg), BfFKP (5 mg), and PmPpA (3 mg), water was added to bring the final volume to 5 mL. The reaction mixture was incubated at 30° C. in an incubator shaker for 20 h with agitation at 180 rpm. Reaction progress was monitored by HRMS. After incubating the reaction mixture in a boiling water bath for 5 min then cooled down to room temperature, LacβNHCbz (100 mg, 0.21 mmol) and NmLgtA (3 mg) were added to synthesize GlcNAcβ3Galβ4GlcβNHCbz. The reaction mixture was incubated at 30° C. in an incubator shaker for 20 h with agitation at 180 rpm. After the reaction was completed, the reaction mixture was incubated in a boiling water bath for 5 min then cooled down to room temperature and moved to the next step without purification. For each step, pH was checked and adjusted to 7.5 by adding 5 M NaOH before adding enzymes. To synthesize GlcNAcβ3(Neu5Acα6)Galβ4GlcβNHCbz, Neu5Ac (0.315 mmol), CTP (0.42 mmol), NmCSS (1 mg), Pd2,6ST (3 mg) were added into the reaction mixture. The reaction was completed in 6 h by incubating the reaction mixture at 30° C. in an incubator shaker with agitation at 180 rpm, followed by incubation in a boiling water bath for 5 min. and cooled down to room temperature. NmLgtB (0.6 mg) was added to produce Galβ4GlcNAcβ3(Neu5Acα6)Galβ4GlcβNHCbz. Once the reaction was completed (8 h), the reaction was stopped by incubating in a boiling water bath for 5 min and cooled down to room temperature. To synthesize GlcNAcβ3Galβ4GlcNAcβ3(Neu5Acα6)Galβ4GlcβNHCbz, Hpβ3GlcNAcT (4 mg) was added and the reaction mixture was incubated at 30° C. in an incubator shaker for 20 h with agitation at 180 rpm. After the reaction was completed. The reaction mixture was incubated in a boiling water bath for 5 min and cooled down to room temperature. Hp3/4FT (1.5 mg) was added and the reaction mixture was incubated at 30° C. in an incubator shaker for 6 h with agitation at 180 rpm to obtain GlcNAcβ3Galβ4(Fucα3)GlcNAcβ3(Neu5Acα6)Galβ4GlcβNHCbz. Then the reaction mixture was incubated in a boiling water bath for 5 min and cooled down to room temperature. NmLgtB (0.6 mg) was added to synthesize Galβ4GlcNAcβ3Galβ4(Fucα3)GlcNAcβ3(Neu5Acα6)Galβ4GlcβNHCbz. The reaction was incubated at 30° C. in an incubator shaker for 8 h followed by incubating in a boiling water bath for 5 min. and cooled down to room temperature. Hp3/4FT (1.5 mg) was added and reaction mixture was incubated at 30° C. in an incubator shaker for 6 h with agitation at 180 rpm to obtain Galβ4(Fucα3)GlcNAcβ3Galβ4(Fucα3)GlcNAcβ3(Neu5Acα6)Galβ4GlcβNHCbz. The reaction mixture was incubated in a boiling water bath for 5 min and cooled down to room temperature. To remove Neu5Ac and obtain the final product Galβ4(Fucα3)GlcNAcβ3Galβ4(Fucα3)GlcNAcβ3Galβ4GlcβNHCbz, SpNanA (1.5 mg) was added and the reaction mixture was incubated at 30° C. in an incubator shaker for 20 h with agitation at 180 rpm. After the reaction was completed, the reaction mixture was incubated in a boiling water bath for 5 min and cooled down to room temperature. The final volume was around 26 mL with a final product concentration of about 7.7 mM. Galβ4(Fucα3)GlcNAcβ3Galβ4(Fucα3)GlcNAcβ3Galβ4GlcβNHCbz (50) was obtained by a single C18-cartridge purification process as a white powder (236 mg, 75% yield for 8 steps from 100 mg LacβNHCbz). ¹H NMR (800 MHz, D₂O) δ 1.16 (dd, J=21.8, 6.6 Hz, 6H), 2.02 (d, J=2.2 Hz, 6H), 3.42 (t, J=9.1 Hz, 1H), 3.50 (dt, J=10.0, 7.2 Hz, 2H), 3.58 (dtd, J=11.9, 6.3, 2.7 Hz, 5H), 3.67-3.63 (m, 3H), 3.71-3.68 (m, 7H), 3.73 (dt, J=12.8, 4.5 Hz, 4H), 3.79 (dt, J=12.5, 3.4 Hz, 4H), 3.88 (dddd, J=32.4, 16.0, 11.3, 3.8 Hz, 8H), 3.99-3.93 (m, 6H), 4.10 (d, J=3.7 Hz, 1H), 4.15 (d, J=3.6 Hz, 1H), 4.48-4.42 (m, 3H), 4.71 (t, J=8.3 Hz, 2H), 4.84 (s, 3H), 5.13 (dd, J=16.7, 4.0 Hz, 2H), 5.19 (p, J=12.9, 11.8 Hz, 2H), 7.45 (d, J=5.7 Hz, 5H); ¹³C NMR (200 MHz, D₂O) δ 15.2, 15.3, 22.2, 22.2, 55.9, 59.6, 59.8, 60.9, 60.9, 61.4, 61.4, 61.5, 61.5, 66.7, 66.7, 67.3, 67.6, 67.7, 68.2, 68.3, 68.3, 69.1, 69.2, 70.0, 70.5, 71.0, 71.4, 71.8, 71.9, 72.4, 72.8, 73.0, 74.4, 74.7, 74.8, 74.9, 74.9, 75.0, 75.0, 75.1, 76.1, 77.7, 81.6, 82.0, 98.6, 98.7, 101.7, 101.7, 102.5, 102.5, 102.8, 127.8, 127.8, 128.5, 128.8, 128.8, 136.0, 158.1, 174.6, 174.7; HRMS (ESI-Orbitrap) m/z: [M+H]⁺ Calcd for C₆₀H₉₆N₃O₄₀ 1498.5565; found 1498.5543.

StOPMe synthesis of Galβ4GlcNAcβ3Galβ4(Fucα3)GlcNAcβ3Galβ4GlcβNHCbz (51). Galactose (0.504 mmol), GlcNAc (0.504 mmol), L-fucose (0.252 mmol), ATP (1.575 mmol), UTP (1.26 mmol), and GTP (0.315 mmol) were dissolved in water in a 50 mL centrifuge tube containing Tris-HCl buffer (100 mM, pH 7.5) and MgCl₂ (20 mM). The pH of the mixture was adjusted to 7.5 by adding 5 M NaOH. After the addition of BLNahK (4 mg), PmGlmU (4 mg), SpGalK (3.5 mg), BLUSP (3 mg), BfFKP (3 mg), and PmPpA (2 mg), water was added to bring the final volume to 5 mL. The reaction mixture was incubated at 30° C. in an incubator shaker for 20 h with agitation at 180 rpm. Reaction progress was monitored by HRMS analysis. After incubating the reaction mixture in a boiling water bath for 5 min then cooled down, LacβNHCbz (100 mg, 0.21 mmol) and NmLgtA (3 mg) were added to synthesize GlcNAcβ3Galβ4GlcβNHCbz. The reaction mixture was incubated at 30° C. in an incubator shaker for 20 h with agitation at 180 rpm. After the reaction was completed, the reaction mixture was incubated in a boiling water bath for 5 min then cooled down and moved to the next step without purification. For each step, pH was checked and adjusted to 7.5 using 5 M NaOH before adding enzymes. To synthesize GlcNAcβ3(Neu5Acα6)Galβ4GlcβNHCbz, Neu5Ac (0.315 mmol), CTP (0.42 mmol), NmCSS (1 mg), Pd2,6ST (3 mg) were added into the reaction mixture. The reaction was completed in 6 h by incubating the reaction mixture at 30° C. in an incubator shaker for 6 h with agitation at 180 rpm, followed by boiling water bath workup and cooling down. NmLgtB (0.6 mg) was added into reaction mixture to obtain Galβ4GlcNAcβ3(Neu5Acα6)Galβ4GlcβNHCbz. Once the reaction was completed (8 h), the reaction was stopped by the same workup method using boiling water bath. To synthesize GlcNAcβ3Galβ4GlcNAcβ3(Neu5Acα6)Galβ4GlcβNHCbz, Hpβ3GlcNAcT (4 mg) was added and the reaction mixture was incubated at 30° C. in an incubator shaker for 20 h with agitation at 180 rpm. After workup, Hp3/4FT (1.5 mg) was added and reaction mixture was incubated at 30° C. in an incubator shaker for 6 h with agitation at 180 rpm to obtain GlcNAcβ3Galβ4(Fucα3)GlcNAcβ3(Neu5Acα6)Galβ4GlcβNHCbz. Then the reaction mixture was incubated in a boiling water bath for 5 min and cooled down. NmLgtB (0.6 mg) was added to synthesize Galβ4GlcNAcβ3Galβ4(Fucα3)GlcNAcβ3(Neu5Acα6)Galβ4GlcβNHCbz. The reaction was incubated at 30° C. in an incubator shaker for 8 h followed by workup with boiling water bath and cooled down. To remove the sialic acid and obtain the final product Galβ4GlcNAcβ3Galβ4(Fucα3)GlcNAcβ3Galβ4GlcβNHCbz, SpNanA (1.5 mg) was added and the reaction mixture was incubated at 30° C. in an incubator shaker for 20 h with agitation at 180 rpm, followed by workup and cooling down. The final volume was around 24 mL with a final product concentration of about 8.3 mM.

Galβ4GlcNAcβ3Galβ4(Fucα3)GlcNAcβ3Galβ4GlcβNHCbz (51) was obtained by single C18-cartridge purification process as a white powder (210 mg, 74% yield for 7 steps from 100 mg LacβNHCbz). ¹H NMR (800 MHz, D₂O) δ 1.15 (d, J=6.6 Hz, 3H), 2.03 (d, J=5.4 Hz, 6H), 3.42 (t, J=9.1 Hz, 1H), 3.53 (ddd, J=19.0, 9.9, 7.8 Hz, 2H), 3.58 (td, J=10.0, 9.3, 3.6 Hz, 4H), 3.67 (ddd, J=16.5, 8.4, 4.9 Hz, 5H), 3.70 (q, J=4.3 Hz, 4H), 3.76-3.71 (m, 7H), 3.81-3.76 (m, 5H), 3.85 (dd, J=12.4, 4.8 Hz, 2H), 3.91-3.87 (m, 2H), 3.95-3.91 (m, 3H), 3.98-3.95 (m, 3H), 4.10 (d, J=3.7 Hz, 1H), 4.15 (d, J=3.6 Hz, 1H), 4.49-4.42 (m, 3H), 4.70 (d, J=8.3 Hz, 3H), 5.12 (d, J=4.1 Hz, 1H), 5.21-5.16 (m, 2H), 7.45 (d, J=5.9 Hz, 5H); ¹³C NMR (200 MHz, D₂O) δ 15.3, 22.1, 22.2, 55.1, 55.9, 59.6, 59.8, 61.0, 61.0, 61.0, 61.4, 66.7, 67.4, 67.6, 68.2, 68.3, 68.5, 69.2, 70.0, 70.5, 70.9, 70.9, 71.4, 71.8, 72.1, 72.5, 72.8, 74.4, 74.7, 74.8, 75.0, 75.1, 75.3, 75.3, 76.1, 77.7, 78.2, 81.6, 82.0, 98.7, 101.7, 102.5, 102.7, 102.8, 102.8, 127.8, 127.8, 128.5, 128.8, 128.8, 136.0, 158.1, 174.7, 174.8. HRMS (ESI-Orbitrap) m/z: [M+H]⁺ Calcd for C₅₄H₈₆N₃O₃₆ 1352.4986; found 1352.4970.

StOPMe synthesis of Galβ4(Fucα3)GlcNAcβ3Galβ4GlcNAcβ3Galβ4GlcβNHCbz (52). Galactose (0.504 mmol), GlcNAc (0.504 mmol), Fucose (0.252 mmol), ATP (1.575 mmol), UTP (1.26 mmol) and GTP (0.315 mmol) were dissolved in water in a 50 mL centrifuge tube containing Tris-HCl buffer (100 mM, pH 7.5) and MgCl₂ (20 mM). The pH of the mixture was adjusted to 7.5 by adding 5 M NaOH. After the addition of BLNahK (4 mg), PmGlmU (4 mg), SpGalK (3.5 mg), BLUSP (3 mg), BfFKP (3 mg) and PmPpA (2 mg), water was added to bring the final volume to 5 ml. The reaction mixture was incubated at 30° C. in an incubator shaker for 20 h with agitation at 180 rpm. Reaction progress was monitored by HRMS analysis. After incubating the reaction mixture in a boiling water bath for 5 min then cooled down, LacβNHCbz (100 mg, 0.21 mmol) and NmLgtA (3 mg) were added to synthesize GlcNAcβ3Galβ34Glcβ3NHCbz. The reaction mixture was incubated at 30° C. in an incubator shaker for 20 h with agitation at 180 rpm. After the reaction was completed, the reaction mixture was incubated in a boiling water bath for 5 min then cooled down and moved to the next step without purification. For each step, pH was checked and adjusted to 7.5 using 5 M NaOH before adding enzymes. To synthesize GlcNAcβ3(Neu5Acα6)Galβ4GlcβNHCbz, Neu5Ac (0.315 mmol), CTP (0.42 mmol), NmCSS (1 mg), Pd2,6ST (3 mg) were added into the reaction mixture. The reaction was completed in 6 h by incubating the reaction mixture at 30° C. in an incubator shaker for 6 h with agitation at 180 rpm, followed by boiling water bath workup and cooling down. NmLgtB (0.6 mg) was added into reaction mixture to obtain Galβ4GlcNAcβ3(Neu5Acα6)Galβ4GlcβNHCbz. Once the reaction was completed (8 h), the reaction was stopped by the same workup method using boiling water bath. To synthesize GlcNAcβ3Galβ4GlcNAcβ3(Neu5Acα6)Galβ4GlcβNHCbz, Hpβ3GlcNAcT (4 mg) was added and the reaction mixture was incubated at 30° C. in an incubator shaker for 20 h with agitation at 180 rpm. After workup, Neu5Ac (0.315 mmol), CTP (0.42 mmol), NmCSS (1 mg), Pd2,6ST (3 mg) were added into the reaction mixture to obtain GlcNAcβ3(Neu5Acα6)Galβ4GlcNAcβ3(Neu5Acα6)Galβ4GlcβNHCbz. The reaction mixture was then incubated in a boiling water bath for 5 min then cooled down. NmLgtB (0.6 mg) was added to synthesize Galβ4GlcNAcβ3(Neu5Acα6)Galβ4GlcNAcβ3(Neu5Acα6)Galβ4GlcβNHCbz. The reaction was incubated at 30° C. in an incubator shaker for 8 h followed by workup with boiling water bath and cooled down.Hp3/4FT (1.5 mg) was added and reaction mixture was incubated at 30° C. in an incubator shaker for 6 h with agitation at 180 rpm to obtain Galβ4(Fucα3)GlcNAcβ3(Neu5Acα6)Galβ4GlcNAcβ3(Neu5Acα6)Galβ4GlcβNHCbz. To remove the sialic acids and obtain the final product Galβ4(Fucα3)GlcNAcβ3Galβ4GlcNAcβ3Galβ4GlcβNHCbz, SpNanA (2 mg) was added and the reaction mixture was incubated at 30° C. in an incubator shaker for 20 h with agitation at 180 rpm, followed by workup and cooling down. The final volume was around 26 mL with a final product concentration of about 7.7 mM.

Galβ4(Fucα3)GlcNAcβ3Galβ4GlcNAcβ3Galβ4GlcβNHCbz (52) was obtained by single C18-cartridge purification process as a white powder (204 mg, 72% yield for 8 steps from 100 mg LacβNHCbz). ¹H NMR (800 MHz, D₂O) δ 1.17 (d, J=6.6 Hz, 3H), 2.02 (d, J=6.4 Hz, 6H), 3.52-3.47 (m, 1H), 3.58 (q, J=9.0 Hz, 5H), 3.65 (d, J=9.3 Hz, 3H), 3.72 (pd, J=19.2, 18.2, 10.3 Hz, 14H), 3.81-3.76 (m, 5H), 3.83 (dd, J=12.5, 5.7 Hz, 1H), 3.94-3.85 (m, 5H), 3.95 (t, J=9.7 Hz, 4H), 4.15 (d, J=8.4 Hz, 2H), 4.45 (dd, J=18.9, 7.8 Hz, 4H), 5.13 (d, J=5.8 Hz, 2H), 5.18 (s, 3H), 7.45 (d, J=5.7 Hz, 5H); ¹³C NMR (200 MHz,) δ 15.3, 22.2, 22.2, 55.1, 55.9, 59.6, 59.8, 60.9, 61.0, 61.5, 61.5, 66.7, 67.4, 67.7, 68.3, 68.3, 68.3, 69.2, 69.9, 70.0, 71.0, 71.4, 71.9, 72.1, 72.5, 73.0, 74.5, 74.7, 74.8, 74.8, 74.9, 75.0, 75.1, 76.1, 77.7, 78.1, 81.6, 82.0, 82.1, 98.6, 101.7, 102.5, 102.7, 102.8, 102.9, 127.7, 127.8, 128.5, 128.8, 128.8, 136.0, 158.1, 174.7, 174.9. HRMS (ESI-Orbitrap) m/z: [M+H]⁺ Calcd for C₅₄H₈₆N₃O₃₆ 1352.4986; found 1352.4960.

StOPMe synthesis of Galβ4GlcNAcβ3Galβ4GlcNAcβ3Galβ4(Fucα3)GlcβNHCbz (53). Galactose (0.504 mmol), GlcNAc (0.504 mmol), Fucose (0.252 mmol), ATP (1.575 mmol), UTP (1.26 mmol) and GTP (0.315 mmol) were dissolved in water in a 50 mL centrifuge tube containing Tris-HCl buffer (100 mM, pH 7.5) and MgCl₂ (20 mM). The pH of the mixture was adjusted to 7.5 by adding 5 M NaOH. After the addition of BLNahK (4 mg), PmGlmU (4 mg), SpGalK (3.5 mg), BLUSP (3 mg), BfFKP (3 mg) and PmPpA (2 mg), water was added to bring the final volume to 5 ml. The reaction mixture was incubated at 30° C. in an incubator shaker for 20 h with agitation at 180 rpm. Reaction progress was monitored by HRMS analysis. After incubating the reaction mixture in a boiling water bath for 5 min then cooled down, LacβNHCbz (100 mg, 0.21 mmol) and NmLgtA (3 mg) were added to synthesize GlcNAcβ33Galβ34Glcβ3NHCbz. The reaction mixture was incubated at 30° C. in an incubator shaker for 20 h with agitation at 180 rpm. After the reaction was completed, the reaction mixture was incubated in a boiling water bath for 5 min then cooled down and moved to the next step without purification. For each step, pH was checked and adjusted to 7.5 using 5 M NaOH before adding enzymes. Hp3/4FT (1.5 mg) was added and reaction mixture was incubated at 30° C. in an incubator shaker for 6 h with agitation at 180 rpm to obtain GlcNAcβ3Galβ4(Fucα3)Glcβ3NHCbz. After incubating the reaction mixture in a boiling water bath for 5 min then cooled down, NmLgtB (0.6 mg) was added into reaction mixture to obtain Galβ4GlcNAcβ3Galβ4(Fucα3)GlcβNHCbz. Once the reaction was completed (8 h), the reaction was stopped by the same workup method using boiling water bath. To synthesize GlcNAcβ3Galβ4GlcNAcβ3Galβ4(Fucα3)GlcβNHCbz, Hpβ3GlcNAcT (4 mg) was added and the reaction mixture was incubated at 30° C. in an incubator shaker for 20 h with agitation at 180 rpm. After workup, NmLgtB (0.6 mg) was added to synthesize Galβ4GlcNAcβ3Galβ4GlcNAcβ3Galβ4(Fucα3)GlcβNHCbz. The reaction was incubated at 30° C. in an incubator shaker for 8 h followed by workup with boiling water bath and cooled down. The final volume was around 22 mL with a final product concentration of about 9.1 mM. Galβ4GlcNAcβ3Galβ4GlcNAcβ3Galβ4(Fucα3)GlcβNHCbz (53) was obtained by single C18-cartridge purification process as a white powder (216 mg, 76% yield for 5 steps from 100 mg LacβNHCbz). ¹H NMR (800.15 MHz, D₂O) δ 1.17 (d, J=6.6 Hz, 3H), 2.03 (d, J=6.0 Hz, 6H), 3.50 (t, J=8.9 Hz, 1H), 3.54 (d, J=10.0 Hz, 1H), 3.62-3.55 (m, 6H), 3.66-3.62 (m, 1H), 3.67 (dd, J=10.0, 3.6 Hz, 1H), 3.70 (d, J=3.2 Hz, 1H), 3.73 (tdd, J=11.8, 8.2, 4.5 Hz, 12H), 3.84-3.75 (m, 10H), 3.89-3.84 (m, 3H), 3.94 (ddd, J=13.5, 9.6, 3.8 Hz, 5H), 4.09 (d, J=3.7 Hz, 1H), 4.16 (d, J=3.6 Hz, 1H), 4.42 (d, J=7.8 Hz, 1H), 4.49-4.46 (m, 2H), 5.18 (s, 2H), 5.44 (d, J=4.2 Hz, 1H), 7.47-7.41 (m, 5H); ¹³C NMR (201.22 MHz, D₂O) δ 15.2, 22.1, 22.2, 55.1, 55.2, 59.6, 59.8, 60.9, 61.0, 61.5, 66.5, 67.4, 68.0, 68.2, 68.3, 68.5, 69.2, 69.9, 70.6, 70.9, 71.9, 72.0, 72.1, 72.2, 72.5, 73.1, 74.5, 74.5, 74.5, 74.8, 75.3, 76.7, 77.6, 78.1, 78.2, 81.5, 81.8, 82.0, 98.5, 101.7, 102.7, 102.7, 102.7, 102.8, 102.9, 127.8, 127.8, 128.5, 128.8, 128.8, 136.0, 158.1, 174.8, 174.9. HRMS (ESI-Orbitrap) m/z: [M+H]⁺ Calcd for C₅₄H₈₆N₃O₃₆ 1352.4986; found 1352.4957.

SEQUENCE LISTING
The nucleic acid and amino acid sequences described herein are
summarized below.
SEQ ID NO: 1
ATGAAAATCGAAGAAGGTAAACTGGTAATCTGGATTAACGGCGATAAAGGCTATAACGGTCT
CGCTGAAGTCGGTAAGAAATTCGAGAAAGATACCGGAATTAAAGTCACCGTTGAGCATCCGG
ATAAACTGGAAGAGAAATTCCCACAGGTTGCGGCAACTGGCGATGGCCCTGACATTATCTTC
TGGGCACACGACCGCTTTGGTGGCTACGCTCAATCTGGCCTGTTGGCTGAAATCACCCCGGA
CAAAGCGTTCCAGGACAAGCTGTATCCGTTTACCTGGGATGCCGTACGTTACAACGGCAAGC
TGATTGCTTACCCGATCGCTGTTGAAGCGTTATCGCTGATTTATAACAAAGATCTGCTGCCG
AACCCGCCAAAAACCTGGGAAGAGATCCCGGCGCTGGATAAAGAACTGAAAGCGAAAGGTAA
GAGCGCGCTGATGTTCAACCTGCAAGAACCGTACTTCACCTGGCCGCTGATTGCTGCTGACG
GGGGTTATGCGTTCAAGTATGAAAACGGCAAGTACGACATTAAAGACGTGGGCGTGGATAAC
GCTGGCGCGAAAGCGGGTCTGACCTTCCTGGTTGACCTGATTAAAAACAAACACATGAATGC
AGACACCGATTACTCCATCGCAGAAGCTGCCTTTAATAAAGGCGAAACAGCGATGACCATCA
ACGGCCCGTGGGCATGGTCCAACATCGACACCAGCAAAGTGAATTATGGTGTAACGGTACTG
CCGACCTTCAAGGGTCAACCATCCAAACCGTTCGTTGGCGTGCTGAGCGCAGGTATTAACGC
CGCCAGTCCGAACAAAGAGCTGGCAAAAGAGTTCCTCGAAAACTATCTGCTGACTGATGAAG
GTCTGGAAGCGGTTAATAAAGACAAACCGCTGGGTGCCGTAGCGCTGAAGTCTTACGAGGAA
GAGTTGGTGAAAGATCCGCGGATTGCCGCCACTATGGAAAACGCCCAGAAAGGTGAAATCAT
GCCGAACATCCCGCAGATGTCCGCTTTCTGGTATGCCGTGCGTACTGCGGTGATCAACGCCG
CCAGCGGTCGTCAGACTGTCGATGAAGCCCTGAAAGACGCGCAGACTAATTCGAGCTCGAAC
AACAACAACAATAACAATAACAACAACCTCGGGATCGAGGGAAGGATTTCAGAATTCGAATT
CCATATGGCCAGTGCCACCGGTAGTAGTCAGCCAGCCGCGGAAAGCAGCACCCAGCAACGCC
CGGGCGTTCCAGCGGGTCCACGTCCGCTGGATGGTTATCTGGGCGTGGCCGATCACAAGCCG
CTGAAAATGCACTGCCGTGATTGCGCGCTGGTGACCAGTAGCGGCCATCTGCTGCACAGCCG
CCAAGGTAGCCAGATTGATCAGACCGAGTGCGTGATCCGCATGAACGACGCGCCAACCCGTG
GTTATGGCCGCGATGTTGGCAACCGTACGAGTCTGCGCGTTATCGCCCACAGTAGCATCCAG
CGCATTCTGCGCAACCGCCACGATCTGCTCAATGTTAGCCAAGGCACCGTGTTCATCTTCTG
GGGCCCGAGCAGCTATATGCGCCGCGATGGCAAGGGCCAAGTTTACAACAACCTCCATCTGC
TCAGCCAAGTTCTGCCGCGTCTGAAAGCGTTCATGATCACCCGCCACAAGATGCTGCAGTTC
GATGAGCTGTTCAAGCAAGAAACGGGTAAGGACCGCAAAATCAGCAACACGTGGCTGAGCAC
GGGCTGGTTTACCATGACGATCGCGCTGGAGCTGTGCGACCGCATCAACGTGTACGGCATGG
TTCCGCCGGATTTTTGCCGCGATCCGAATCATCCAAGCGTGCCGTACCACTACTACGAACCG
TTTGGTCCGGACGAATGTACCATGTATCTGAGCCATGAGCGTGGCCGCAAGGGCAGCCATCA
CCGCTTCATCACGGAGAAGCGCGTGTTCAAGAACTGGGCCCGCACCTTCAACATCCATTTCT
TCCAACCGGATTGGAAGCCGGAAAGTCTGGCCATCAACCACCCAGAAAACAAACCGGTTTTC
GGATCCCATCATCATCATCATCACTGA
SEQ ID NO: 1 shows the nucleic acid sequence of MBP-
Δ50hST6GALNAC V-His₆. The sequences shown in italics and
underlined were from the vector plasmid. The corresponding
codon for V99 residue is bolded.
SEQ ID NO: 2
ATGAAAATCGAAGAAGGTAAACTGGTAATCTGGATTAACGGCGATAAAGGCTATAACGGTCT
CGCTGAAGTCGGTAAGAAATTCGAGAAAGATACCGGAATTAAAGTCACCGTTGAGCATCCGG
ATAAACTGGAAGAGAAATTCCCACAGGTTGCGGCAACTGGCGATGGCCCTGACATTATCTTC
TGGGCACACGACCGCTTTGGTGGCTACGCTCAATCTGGCCTGTTGGCTGAAATCACCCCGGA
CAAAGCGTTCCAGGACAAGCTGTATCCGTTTACCTGGGATGCCGTACGTTACAACGGCAAGC
TGATTGCTTACCCGATCGCTGTTGAAGCGTTATCGCTGATTTATAACAAAGATCTGCTGCCG
AACCCGCCAAAAACCTGGGAAGAGATCCCGGCGCTGGATAAAGAACTGAAAGCGAAAGGTAA
GAGCGCGCTGATGTTCAACCTGCAAGAACCGTACTTCACCTGGCCGCTGATTGCTGCTGACG
GGGGTTATGCGTTCAAGTATGAAAACGGCAAGTACGACATTAAAGACGTGGGCGTGGATAAC
GCTGGCGCGAAAGCGGGTCTGACCTTCCTGGTTGACCTGATTAAAAACAAACACATGAATGC
AGACACCGATTACTCCATCGCAGAAGCTGCCTTTAATAAAGGCGAAACAGCGATGACCATCA
ACGGCCCGTGGGCATGGTCCAACATCGACACCAGCAAAGTGAATTATGGTGTAACGGTACTG
CCGACCTTCAAGGGTCAACCATCCAAACCGTTCGTTGGCGTGCTGAGCGCAGGTATTAACGC
CGCCAGTCCGAACAAAGAGCTGGCAAAAGAGTTCCTCGAAAACTATCTGCTGACTGATGAAG
GTCTGGAAGCGGTTAATAAAGACAAACCGCTGGGTGCCGTAGCGCTGAAGTCTTACGAGGAA
GAGTTGGTGAAAGATCCGCGGATTGCCGCCACTATGGAAAACGCCCAGAAAGGTGAAATCAT
GCCGAACATCCCGCAGATGTCCGCTTTCTGGTATGCCGTGCGTACTGCGGTGATCAACGCCG
CCAGCGGTCGTCAGACTGTCGATGAAGCCCTGAAAGACGCGCAGACTAATTCGAGCTCGAAC
AACAACAACAATAACAATAACAACAACCTCGGGATCGAGGGAAGGATTTCAGAATTCGAATT
CCATATGGCCAGTGCCACCGGTAGTAGTCAGCCAGCCGCGGAAAGCAGCACCCAGCAACGCC
CGGGCGTTCCAGCGGGTCCACGTCCGCTGGATGGTTATCTGGGCGTGGCCGATCACAAGCCG
CTGAAAATGCACTGCCGTGATTGCGCGCTGATGACCAGTAGCGGCCATCTGCTGCACAGCCG
CCAAGGTAGCCAGATTGATCAGACCGAGTGCGTGATCCGCATGAACGACGCGCCAACCCGTG
GTTATGGCCGCGATGTTGGCAACCGTACGAGTCTGCGCGTTATCGCCCACAGTAGCATCCAG
CGCATTCTGCGCAACCGCCACGATCTGCTCAATGTTAGCCAAGGCACCGTGTTCATCTTCTG
GGGCCCGAGCAGCTATATGCGCCGCGATGGCAAGGGCCAAGTTTACAACAACCTCCATCTGC
TCAGCCAAGTTCTGCCGCGTCTGAAAGCGTTCATGATCACCCGCCACAAGATGCTGCAGTTC
GATGAGCTGTTCAAGCAAGAAACGGGTAAGGACCGCAAAATCAGCAACACGTGGCTGAGCAC
GGGCTGGTTTACCATGACGATCGCGCTGGAGCTGTGCGACCGCATCAACGTGTACGGCATGG
TTCCGCCGGATTTTTGCCGCGATCCGAATCATCCAAGCGTGCCGTACCACTACTACGAACCG
TTTGGTCCGGACGAATGTACCATGTATCTGAGCCATGAGCGTGGCCGCAAGGGCAGCCATCA
CCGCTTCATCACGGAGAAGCGCGTGTTCAAGAACTGGGCCCGCACCTTCAACATCCATTTCT
TCCAACCGGATTGGAAGCCGGAAAGTCTGGCCATCAACCACCCAGAAAACAAACCGGTTTTC
GGATCCCATCATCATCATCATCACTGA
SEQ ID NO: 2 shows the nucleic acid sequence of MBP-
Δ50hST6GALNAC V_V99M-His₆. The sequences shown in italics and
underlined were from the vector plasmid. The corresponding
codon for M99 residue is bolded.
SEQ ID NO: 3
ATGAAAATCGAAGAAGGTAAACTGGTAATCTGGATTAACGGCGATAAAGGCTATAACGGTCT
CGCTGAAGTCGGTAAGAAATTCGAGAAAGATACCGGAATTAAAGTCACCGTTGAGCATCCGG
ATAAACTGGAAGAGAAATTCCCACAGGTTGCGGCAACTGGCGATGGCCCTGACATTATCTTC
TGGGCACACGACCGCTTTGGTGGCTACGCTCAATCTGGCCTGTTGGCTGAAATCACCCCGGA
CAAAGCGTTCCAGGACAAGCTGTATCCGTTTACCTGGGATGCCGTACGTTACAACGGCAAGC
TGATTGCTTACCCGATCGCTGTTGAAGCGTTATCGCTGATTTATAACAAAGATCTGCTGCCG
AACCCGCCAAAAACCTGGGAAGAGATCCCGGCGCTGGATAAAGAACTGAAAGCGAAAGGTAA
GAGCGCGCTGATGTTCAACCTGCAAGAACCGTACTTCACCTGGCCGCTGATTGCTGCTGACG
GGGGTTATGCGTTCAAGTATGAAAACGGCAAGTACGACATTAAAGACGTGGGCGTGGATAAC
GCTGGCGCGAAAGCGGGTCTGACCTTCCTGGTTGACCTGATTAAAAACAAACACATGAATGC
AGACACCGATTACTCCATCGCAGAAGCTGCCTTTAATAAAGGCGAAACAGCGATGACCATCA
ACGGCCCGTGGGCATGGTCCAACATCGACACCAGCAAAGTGAATTATGGTGTAACGGTACTG
CCGACCTTCAAGGGTCAACCATCCAAACCGTTCGTTGGCGTGCTGAGCGCAGGTATTAACGC
CGCCAGTCCGAACAAAGAGCTGGCAAAAGAGTTCCTCGAAAACTATCTGCTGACTGATGAAG
GTCTGGAAGCGGTTAATAAAGACAAACCGCTGGGTGCCGTAGCGCTGAAGTCTTACGAGGAA
GAGTTGGTGAAAGATCCGCGGATTGCCGCCACTATGGAAAACGCCCAGAAAGGTGAAATCAT
GCCGAACATCCCGCAGATGTCCGCTTTCTGGTATGCCGTGCGTACTGCGGTGATCAACGCCG
CCAGCGGTCGTCAGACTGTCGATGAAGCCCTGAAAGACGCGCAGACTAATTCGAGCTCGAAC
AACAACAACAATAACAATAACAACAACCTCGGGATCGAGGGAAGGATTTCAGAATTCGAATT
CCATATGGCCAGTGCCACCGGTAGTAGTCAGCCAGCCGCGGAAAGCAGCACCCAGCAACGCC
CGGGCGTTCCAGCGGGTCCACGTCCGCTGGATGGTTATCTGGGCGTGGCCGATCACAAGCCG
CTGAAAATGCACTGCCGTGATTGCGCGCTGGTGACCAGTAGCGGCCATCTGCTGCACAGCCG
CCAAGGTAGCCAGATTGATCAGACCGAGTGCGTGATCCGCATGAACGACGCGCCAACCCGTG
GTTATGGCCGCGATGTTGGCAACCGTACGAGTCTGCGCGTTATCGCCCACAGTAGCATCCAG
CGCATTCTGCGCAACCGCCACGATCTGCTCAATGTTAGCCAAGGCACCGTGTTCATCTTCTG
GGGCCCGAGCAGCTATATGCGCCGCGATGGCAAGGGCCAAGTTTACAACAACCTCCATCTGC
TCAGCCAAGTTCTGCCGCGTCTGAAAGCGTTCATGATCACCCGCCACAAGATGCTGCAGTTC
GATGAGCTGTTCAAGCAAGAAACGGGTAAGGACCGCAAAATCAGCAACACGTGGCTGAGCAC
GGGCTGGTTTACCATGACGATCGCGCTGGAGCTGTGCGACCGCATCAACGTGTACGGCATGG
TTCCGCCGGATTTTTGCCGCGATCCGAATCATCCAAGCGTGCCGTACCACTACTACGAACCG
TTTGGTCCGGACGAATGTACCATGTATCTGAGCCATGAGCGTGGCCGCAAGGGCAGCCATCA
CCGCTTCATCACGGAGAAGCGCGTGTTCAAGAACTGGGCCCGCACCTTCAACATCCATTTCT
TCCAACCGGATTGGAAGCCGGAAAGTCTGGCCATCAACCACCCAGAAAACAAACCGGTTTTC
GGATCCCAAGGAATGTCAATGCCCGACTGTAACCCTCGATACAGCTTGCACCAGACTCCGAG
ACATCATCATCATCATCATCACTGA
SEQ ID NO: 3 shows the nucleic acid sequence of MBP-
Δ50hST6GALNAC V_ext20-His₆. The sequences shown in italics and
underlined were from the vector plasmid. The corresponding
codon for V99 and ext20 residues are bolded.
SEQ ID NO: 4
ATGAAAATCGAAGAAGGTAAACTGGTAATCTGGATTAACGGCGATAAAGGCTATAACGGTCT
CGCTGAAGTCGGTAAGAAATTCGAGAAAGATACCGGAATTAAAGTCACCGTTGAGCATCCGG
ATAAACTGGAAGAGAAATTCCCACAGGTTGCGGCAACTGGCGATGGCCCTGACATTATCTTC
TGGGCACACGACCGCTTTGGTGGCTACGCTCAATCTGGCCTGTTGGCTGAAATCACCCCGGA
CAAAGCGTTCCAGGACAAGCTGTATCCGTTTACCTGGGATGCCGTACGTTACAACGGCAAGC
TGATTGCTTACCCGATCGCTGTTGAAGCGTTATCGCTGATTTATAACAAAGATCTGCTGCCG
AACCCGCCAAAAACCTGGGAAGAGATCCCGGCGCTGGATAAAGAACTGAAAGCGAAAGGTAA
GAGCGCGCTGATGTTCAACCTGCAAGAACCGTACTTCACCTGGCCGCTGATTGCTGCTGACG
GGGGTTATGCGTTCAAGTATGAAAACGGCAAGTACGACATTAAAGACGTGGGCGTGGATAAC
GCTGGCGCGAAAGCGGGTCTGACCTTCCTGGTTGACCTGATTAAAAACAAACACATGAATGC
AGACACCGATTACTCCATCGCAGAAGCTGCCTTTAATAAAGGCGAAACAGCGATGACCATCA
ACGGCCCGTGGGCATGGTCCAACATCGACACCAGCAAAGTGAATTATGGTGTAACGGTACTG
CCGACCTTCAAGGGTCAACCATCCAAACCGTTCGTTGGCGTGCTGAGCGCAGGTATTAACGC
CGCCAGTCCGAACAAAGAGCTGGCAAAAGAGTTCCTCGAAAACTATCTGCTGACTGATGAAG
GTCTGGAAGCGGTTAATAAAGACAAACCGCTGGGTGCCGTAGCGCTGAAGTCTTACGAGGAA
GAGTTGGTGAAAGATCCGCGGATTGCCGCCACTATGGAAAACGCCCAGAAAGGTGAAATCAT
GCCGAACATCCCGCAGATGTCCGCTTTCTGGTATGCCGTGCGTACTGCGGTGATCAACGCCG
CCAGCGGTCGTCAGACTGTCGATGAAGCCCTGAAAGACGCGCAGACTAATTCGAGCTCGAAC
AACAACAACAATAACAATAACAACAACCTCGGGATCGAGGGAAGGATTTCAGAATTCGAATT
CCATATGGCCAGTGCCACCGGTAGTAGTCAGCCAGCCGCGGAAAGCAGCACCCAGCAACGCC
CGGGCGTTCCAGCGGGTCCACGTCCGCTGGATGGTTATCTGGGCGTGGCCGATCACAAGCCG
CTGAAAATGCACTGCCGTGATTGCGCGCTGATGACCAGTAGCGGCCATCTGCTGCACAGCCG
CCAAGGTAGCCAGATTGATCAGACCGAGTGCGTGATCCGCATGAACGACGCGCCAACCCGTG
GTTATGGCCGCGATGTTGGCAACCGTACGAGTCTGCGCGTTATCGCCCACAGTAGCATCCAG
CGCATTCTGCGCAACCGCCACGATCTGCTCAATGTTAGCCAAGGCACCGTGTTCATCTTCTG
GGGCCCGAGCAGCTATATGCGCCGCGATGGCAAGGGCCAAGTTTACAACAACCTCCATCTGC
TCAGCCAAGTTCTGCCGCGTCTGAAAGCGTTCATGATCACCCGCCACAAGATGCTGCAGTTC
GATGAGCTGTTCAAGCAAGAAACGGGTAAGGACCGCAAAATCAGCAACACGTGGCTGAGCAC
GGGCTGGTTTACCATGACGATCGCGCTGGAGCTGTGCGACCGCATCAACGTGTACGGCATGG
TTCCGCCGGATTTTTGCCGCGATCCGAATCATCCAAGCGTGCCGTACCACTACTACGAACCG
TTTGGTCCGGACGAATGTACCATGTATCTGAGCCATGAGCGTGGCCGCAAGGGCAGCCATCA
CCGCTTCATCACGGAGAAGCGCGTGTTCAAGAACTGGGCCCGCACCTTCAACATCCATTTCT
TCCAACCGGATTGGAAGCCGGAAAGTCTGGCCATCAACCACCCAGAAAACAAACCGGTTTTC
GGATCCCAAGGAATGTCAATGCCCGACTGTAACCCTCGATACAGCTTGCACCAGACTCCGAG
ACATCATCATCATCATCATCACTGA
SEQ ID NO: 4 shows the nucleic acid sequence of MBP-
Δ50hST6GALNAC V_V99M_ext20-His₆. The sequences shown in
italics and underlined were from the vector plasmid. The
corresponding codons for M99 residue and for the ext20
residues are bolded.
SEQ ID NO: 5
HMKIEEGKLVIWINGDKGYNGLAEVGKKFEKDTGIKVTVEHPDKLEEKFPQVAATGDGPDII
FWAHDRFGGYAQSGLLAEITPDKAFQDKLYPFTWDAVRYNGKLIAYPIAVEALSLIYNKDLL
PNPPKTWEEIPALDKELKAKGKSALMFNLQEPYFTWPLIAADGGYAFKYENGKYDIKDVGVD
NAGAKAGLTFLVDLIKNKHMNADTDYSIAEAAFNKGETAMTINGPWAWSNIDTSKVNYGVTV
LPTFKGQPSKPFVGVLSAGINAASPNKELAKEFLENYLLTDEGLEAVNKDKPLGAVALKSYE
EELVKDPRIAATMENAQKGEIMPNIPQMSAFWYAVRTAVINAASGRQTVDEALKDAQTNSSS
NNNNNNNNNNLGIEGRISEFEFHMASATGSSQPAAESSTQQRPGVPAGPRPLDGYLGVADHK
PLKMHCRDCALVTSSGHLLHSRQGSQIDQTECVIRMNDAPTRGYGRDVGNRTSLRVIAHSSI
QRILRNRHDLLNVSQGTVFIFWGPSSYMRRDGKGQVYNNLHLLSQVLPRLKAFMITRHKMLQ
FDELFKQETGKDRKISNTWLSTGWFTMTIALELCDRINVYGMVPPDFCRDPNHPSVPYHYYE
PFGPDECTMYLSHERGRKGSHHRFITEKRVFKNWARTFNIHFFQPDWKPESLAINHPENKPV
FGSHHHHHH*
SEQ ID NO: 5 shows the amino acid sequence of MBP-
Δ50hST6GALNAC V-His₆. The sequences shown in italics and
underlined were from the vector plasmid. The V99 residue is
bolded.
SEQ ID NO: 6
MKIEEGKLVIWINGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVEALSLIYNKDLLP
NPPKTWEEIPALDKELKAKGKSALMFNLQEPYFTWPLIAADGGYAFKYENGKYDIKDVGVDN
AGAKAGLTFLVDLIKNKHMNADTDYSIAEAAFNKGETAMTINGPWAWSNIDTSKVNYGVTVL
PTFKGQPSKPFVGVLSAGINAASPNKELAKEFLENYLLTDEGLEAVNKDKPLGAVALKSYEE
ELVKDPRIAATMENAQKGEIMPNIPQMSAFWYAVRTAVINAASGRQTVDEALKDAQTNSSSN
NNNNNNNNNLGIEGRISEFEFHMASATGSSQPAAESSTQQRPGVPAGPRPLDGYLGVADHKP
LKMHCRDCALMTSSGHLLHSRQGSQIDQTECVIRMNDAPTRGYGRDVGNRTSLRVIAHSSIQ
RILRNRHDLLNVSQGTVFIFWGPSSYMRRDGKGQVYNNLHLLSQVLPRLKAFMITRHKMLQF
DELFKQETGKDRKISNTWLSTGWFTMTIALELCDRINVYGMVPPDFCRDPNHPSVPYHYYEP
FGPDECTMYLSHERGRKGSHHRFITEKRVFKNWARTFNIHFFQPDWKPESLAINHPENKPVF
GSHHHHHH*
SEQ ID NO: 6 shows the amino acid sequence of MBP-
Δ50hST6GALNAC V_V99M-His₆. The sequences shown in italics and
underlined were from the vector plasmid. The M99 residue is
bolded.
SEQ ID NO: 7
MKIEEGKLVIWINGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVEALSLIYNKDLLP
NPPKTWEEIPALDKELKAKGKSALMFNLQEPYFTWPLIAADGGYAFKYENGKYDIKDVGVDN
AGAKAGLTFLVDLIKNKHMNADTDYSIAEAAFNKGETAMTINGPWAWSNIDTSKVNYGVTVL
PTFKGQPSKPFVGVLSAGINAASPNKELAKEFLENYLLTDEGLEAVNKDKPLGAVALKSYEE
ELVKDPRIAATMENAQKGEIMPNIPQMSAFWYAVRTAVINAASGRQTVDEALKDAQTNSSSN
NNNNNNNNNLGIEGRISEFEFHMASATGSSQPAAESSTQQRPGVPAGPRPLDGYLGVADHKP
LKMHCRDCALVTSSGHLLHSRQGSQIDQTECVIRMNDAPTRGYGRDVGNRTSLRVIAHSSIQ
RILRNRHDLLNVSQGTVFIFWGPSSYMRRDGKGQVYNNLHLLSQVLPRLKAFMITRHKMLQF
DELFKQETGKDRKISNTWLSTGWFTMTIALELCDRINVYGMVPPDFCRDPNHPSVPYHYYEP
FGPDECTMYLSHERGRKGSHHRFITEKRVFKNWARTFNIHFFQPDWKPESLAINHPENKPVF
GSQGMSMPDCNPRYSLHQTPRHHHHHHH*
SEQ ID NO: 7 shows the amino acid sequence of MBP-
Δ50hST6GALNAC V_ext20-His₆. The sequences shown in italics and
underlined were from the vector plasmid. The V99 residue and
the ext20 residues are bolded.
SEQ ID NO: 8
MKIEEGKLVIWINGDKGYNGLAEVGKKFEKDTGIKVTVEHPDKLEEKFPQVAATGDGPDIIF
WAHDRFGGYAQSGLLAEITPDKAFQDKLYPFTWDAVRYNGKLIAYPIAVEALSLIYNKDLLP
NPPKTWEEIPALDKELKAKGKSALMFNLQEPYFTWPLIAADGGYAFKYENGKYDIKDVGVDN
AGAKAGLTFLVDLIKNKHMNADTDYSIAEAAFNKGETAMTINGPWAWSNIDTSKVNYGVTVL
PTFKGQPSKPFVGVLSAGINAASPNKELAKEFLENYLLTDEGLEAVNKDKPLGAVALKSYEE
ELVKDPRIAATMENAQKGEIMPNIPQMSAFWYAVRTAVINAASGRQTVDEALKDAQTNSSSN
NNNNNNNNNLGIEGRISEFEFHMASATGSSQPAAESSTQQRPGVPAGPRPLDGYLGVADHKP
LKMHCRDCALMTSSGHLLHSRQGSQIDQTECVIRMNDAPTRGYGRDVGNRTSLRVIAHSSIQ
RILRNRHDLLNVSQGTVFIFWGPSSYMRRDGKGQVYNNLHLLSQVLPRLKAFMITRHKMLQF
DELFKQETGKDRKISNTWLSTGWFTMTIALELCDRINVYGMVPPDFCRDPNHPSVPYHYYEP
FGPDECTMYLSHERGRKGSHHRFITEKRVFKNWARTFNIHFFQPDWKPESLAINHPENKPVF
GSQGMSMPDCNPRYSLHQTPRHHHHHHH*
SEQ ID NO: 8 shows the amino acid sequence of MBP-
Δ50hST6GALNAC V_V99M_ext20-His₆. The sequences shown in
italics and underlined were from the vector plasmid. The M99
and the ext20 residues are bolded.

TABLE 2
List of mutations in the PROSS-designed mutants of
MBP-Δ50hST6GALNACV_ext20-His₆. The
mutation site numbers are based on the amino acid
numbers in the amino acid sequence of the
full-length wild-type hST6GALNAC V.

| Design | Mutations within MBP-Δ50hST6GALNAC |
|---|---|
| Number | V_Design#_ext20-His₆ |
| 1 | G82R, V99M, H107G, S140T, L191R, A201L, |
| | H207D, T239V |
| 2 | G82R, A84Y, V99M, H107G, S140T, L191R, |
| | A201L, H207D, Q218R, T239I, N306R |
| 3 | G82R, A84Y, V99M, H107G, G132E, S140T, |
| | Y176K, Q184T, H190L, L191R, A201L, H207D, |
| | Q218R, T239I, F275T, N306R |
| 4 | G82R, H107G, S140T, L191R, A201L, H207D, T239V |
| 5 | G82R, H107G, S140T, L191R, A201L, H207D, T239V, |
| | add QGMSMPDCNPRYSLHQTPR at C terminal |
| 6 | G82R, A84Y, H107G, S140T, L191R, A201L, |
| | H207D, Q218R, T239I, N306R |
| 7* | G82R, A84Y, H107G, S140T, L191R, A201L, |
| | H207D, Q218R, T239I, N306R, add |
| | QGMSMPDCNPRYSLHQTPR at C terminal |
| 8 | G82R, A84Y, H107G, G132E, S140T, Y176K, |
| | Q184T, H190L, L191R, A201L, H207D, Q218R, |
| | T239I, F275T, N306R, add |
| | QGMSMPDCNPRYSLHQTPR at C terminal |
| 9 | G82R, A84Y, H107G, G132E, S140T, Y176K, |
| | Q184T, H190L, L191R, A201L, H207D, Q218R, |
| | T239I, F275T, N306R, |
| 10 | G82R, V99M, A84Y, H107G, S140T, L191R, |
| | A201L, H207D, Q218R, T239I, N306R, add |
| | QGMSMPDCNPRYSLHQTPR at C terminal |
| 11 | G82R, V99L, A84Y, H107G, S140T, L191R, |
| | A201L, H207D, Q218R, T239I, N306R, add |
| | QGMSMPDCNPRYSLHQTPR at C terminal |
| 12 | G82R, V99A, A84Y, H107G, S140T, L191R, |
| | A201L, H207D, Q218R, T239I, N306R, add |
| | QGMSMPDCNPRYSLHQTPR at C terminal |
| 13 | G82R, V99G, A84Y, H107G, S140T, L191R, |
| | A201L, H207D, Q218R, T239I, N306R, add |
| | QGMSMPDCNPRYSLHQTPR at C terminal |
| 14 | G82R, V99F, A84Y, H107G, S140T, L191R, |
| | A201L, H207D, Q218R, T239I, N306R, add |
| | QGMSMPDCNPRYSLHQTPR at C terminal |
| 15 | G82R, V99W, A84Y, H107G, S140T, L191R, |
| | A201L, H207D, Q218R, T239I, N306R, add |
| | QGMSMPDCNPRYSLHQTPR at C terminal |

*Design 7 (MBP-Δ50hST6GALNAC V_Design7_ext20-His₆) was shown to have a good expression level and was used for synthesizing DSLNTβNHCbz (46) using both OPME and StOPMe strategies.

SEQ ID NO: 9
ATGAAAATCGAAGAAGGTAAACTGGTAATCTGGATTAACGGCGAT
AAAGGCTATAACGGTCTCGCTGAAGTCGGTAAGAAATTCGAGAAA
GATACCGGAATTAAAGTCACCGTTGAGCATCCGGATAAACTGGAA
GAGAAATTCCCACAGGTTGCGGCAACTGGCGATGGCCCTGACATT
ATCTTCTGGGCACACGACCGCTTTGGTGGCTACGCTCAATCTGGC
CTGTTGGCTGAAATCACCCCGGACAAAGCGTTCCAGGACAAGCTG
TATCCGTTTACCTGGGATGCCGTACGTTACAACGGCAAGCTGATT
GCTTACCCGATCGCTGTTGAAGCGTTATCGCTGATTTATAACAAA
GATCTGCTGCCGAACCCGCCAAAAACCTGGGAAGAGATCCCGGCG
CTGGATAAAGAACTGAAAGCGAAAGGTAAGAGCGCGCTGATGTTC
AACCTGCAAGAACCGTACTTCACCTGGCCGCTGATTGCTGCTGAC
GGGGGTTATGCGTTCAAGTATGAAAACGGCAAGTACGACATTAAA
GACGTGGGCGTGGATAACGCTGGCGCGAAAGCGGGTCTGACCTTC
CTGGTTGACCTGATTAAAAACAAACACATGAATGCAGACACCGAT
TACTCCATCGCAGAAGCTGCCTTTAATAAAGGCGAAACAGCGATG
ACCATCAACGGCCCGTGGGCATGGTCCAACATCGACACCAGCAAA
GTGAATTATGGTGTAACGGTACTGCCGACCTTCAAGGGTCAACCA
TCCAAACCGTTCGTTGGCGTGCTGAGCGCAGGTATTAACGCCGCC
AGTCCGAACAAAGAGCTGGCAAAAGAGTTCCTCGAAAACTATCTG
CTGACTGATGAAGGTCTGGAAGCGGTTAATAAAGACAAACCGCTG
GGTGCCGTAGCGCTGAAGTCTTACGAGGAAGAGTTGGCGAAAGAT
CCACGTATTGCCGCCACTATGGAAAACGCCCAGAAAGGTGAAATC
ATGCCGAACATCCCGCAGATGTCCGCTTTCTGGTATGCCGTGCGT
ACTGCGGTGATCAACGCCGCCAGCGGTCGTCAGACTGTCGATGAA
GCCCTGAAAGACGCGCAGACTAATTCGAGCTCGAACAACAACAAC
AATAACAATAACAACAACCTCGGGATCGAGGGAAGGATTTCAGAA
TTCATGTTCGGCGGAGATCCTTCTTTCCAACGCTTAAATATTAGC
GACCCGTTACGTCTTACACAAGTGTGTACATCATTTATTAATGGG
AAAACTCGCTTCTTATGGAAGAACAAGCTCATGATCCACGAGAAA
AGCAGCTGCAAGGAATACCTGACACAAAGTCATTATATTACCGCA
CCATTGAGTAAAGAAGAGGCCGATTTCCCCTTAGCGTACATTATG
GTCATACACCACCATTTCGACACCTTCGCCCGGCTGTTTCGCGCT
ATATACATGCCGCAGAATATTTACTGCGTTCACGTTGACGAAAAG
GCAACAACAGAGTTCAAGGACGCTGTTGAACAACTTCTGTCCTGT
TTCCCCAACGCATTTCTTGCATCTAAGATGGAGCCGGTGGTGTAC
GGCGGTATTTCACGCCTTCAAGCAGACCTCAATTGCATACGTGAC
TTATCAGCCTTCGAGGTTTCTTGGAAATACGTTATAAACACGTGC
GGACAAGACTTCCCACTGAAGACAAACAAGGAGATTGTGCAATAC
CTGAAGGGCTTTAAGGGAAAGAATATTACGCCCGGGGTATTACCA
CCGGCACATGCGATTGGGCGAACGAAATACGTCCACCAAGAGCAC
TTAGGTAAGGAATTGTCCTATGTCATACGTACTACAGCTCTGAAG
CCGCCACCCCCTCACAATTTAACTATTTATTTTGGTTCCGCTTAC
GTTGCTTTGAGTCGCGAGTTCGCTAATTTCGTCTTACATGATCCT
CGGGCGGTGGACTTACTGCAATGGTCCAAAGACACTTTCAGCCCC
GACGAACACTTTTGGGTAACACTGAACCGAATACCAGGTGTACCT
GGCTCCATGCCTAATGCCTCTTGGACCGGCAACCTGAGAGCAATT
AAATGGTCCGATATGGAAGACCGCCACGGTGGATGTCATGGGCAT
TACGTGCACGGAATATGTATATACGGTAACGGTGACCTTAAGTGG
CTGGTGAATAGTCCGAGCCTGTTTGCTAACAAGTTCGAGCTGAAC
ACATATCCTCTTACTGTGGAGTGTCTCGAACTTCGTCATCGCGAA
AGAACGCTGAACCAGTCCGAGACGGCCATTCAGCCATCATGGTAC
TTCCATCATCATCACCACCACTAA

SEQ ID NO: 9 shows the nucleic acid sequence of MBP-Δ25hGCNT2-B-His₆. The sequences shown in italics and underlined were from the pMAL-c2X vector.

SEQ ID NO: 10
MKIEEGKLVIWINGDKGYNGLAEVGKKFEKDTGIKVTVEHPDKLE
EKFPQVAATGDGPDIIFWAHDRFGGYAQSGLLAEITPDKAFQDKL
YPFTWDAVRYNGKLIAYPIAVEALSLIYNKDLLPNPPKTWEEIPA
LDKELKAKGKSALMFNLQEPYFTWPLIAADGGYAFKYENGKYDIK
DVGVDNAGAKAGLTFLVDLIKNKHMNADTDYSIAEAAFNKGETAM
TINGPWAWSNIDTSKVNYGVTVLPTFKGQPSKPFVGVLSAGINAA
SPNKELAKEFLENYLLTDEGLEAVNKDKPLGAVALKSYEEELAKD
PRIAATMENAQKGEIMPNIPQMSAFWYAVRTAVINAASGRQTVDE
ALKDAQTNSSSNNNNNNNNNNLGIEGRISEFMFGGDPSFQRLNIS
DPLRLTQVCTSFINGKTRFLWKNKLMIHEKSSCKEYLTQSHYITA
PLSKEEADFPLAYIMVIHHHFDTFARLFRAIYMPQNIYCVHVDEK
ATTEFKDAVEQLLSCFPNAFLASKMEPVVYGGISRLQADLNCIRD
LSAFEVSWKYVINTCGQDFPLKTNKEIVQYLKGFKGKNITPGVLP
PAHAIGRTKYVHQEHLGKELSYVIRTTALKPPPPHNLTIYFGSAY
VALSREFANFVLHDPRAVDLLQWSKDTFSPDEHFWVTLNRIPGVP
GSMPNASWTGNLRAIKWSDMEDRHGGCHGHYVHGICIYGNGDLKW
LVNSPSLFANKFELNTYPLTVECLELRHRERTLNQSETAIQPSWY
FHHHHHH*

SEQ ID NO: 10 shows the amino acid sequence of MBP-Δ25hGCNT2-B-His₆. The sequences shown in italics and underlined were from the pMAL-c2X vector.

A200Y forward
SEQ ID NO: 11
5′-TATACAAATACATATCATGTTTTTAATAATTTACCACCT-3′

SEQ ID NO: 11 is the forward primer sequence for generating the A200Y mutation as described herein.

A200Y reverse
SEQ ID NO: 12
5′-ATTAAAAACATGATATGTATTTGTATAGAACTCTATATT-3′

SEQ ID NO: 12 is the reverse primer sequence for generating the A200Y mutation as described herein.

S232Y forward
SEQ ID NO: 13
5′-GATGATGGTTCTTATGAATATGTAAGTT-TATATCAATGG-3′

SEQ ID NO: 13 is the forward primer sequence for generating the S232Y mutation as described herein.

S232Y reverse
SEQ ID NO: 14
5′-ACTTACATATTCATAAGAACCATCATCATACAAAC-TAAT-3′

SEQ ID NO: 14 is the reverse primer sequence for generating the S232Y mutation as described herein.

forward:
SEQ ID NO: 15
5′-TTGCGCGCTGGGCACCAGTAGCG-3′

SEQ ID NO: 15 is the forward primer sequence for generating the V99M mutant as described herein.

reverse:
SEQ ID NO: 16
5′- TCACGGCAGTGCATTTTCAGC-3′

SEQ ID NO: 16 is the reverse primer sequence for generating the V99M mutant as described herein.

forward
SEQ ID NO: 17
5′-CGATACAGCTTGCACCAGACTCCGAGACATCATCA
TCATCATCATCACTGATC-3′

SEQ ID NO: 17 is the forward primer sequence for generating the ext20 mutant as described herein.

reverse
SEQ ID NO: 18
5′-AGGGTTACAGTCGGGCATTGACATTCCTTGGGATC
CGAAAACCGGTTTG-3′

SEQ ID NO: 18 is the reverse primer sequence for generating the ext20 mutant as described herein.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A compound according to Formula I: 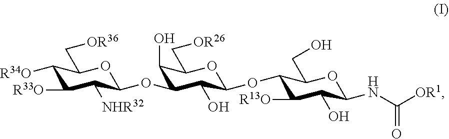
or a salt thereof, wherein:
R¹ is selected from the group consisting of benzyl and fluorenylmethyl;
R¹³ is selected from the group consisting of H and fucosyl;
R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R³² is selected from the group consisting of H, acetyl, trifluoroacetyl, Boc, Fmoc;
R³⁶ is selected from the group consisting of H and N-acetylneuraminic acid;
R³³ and R³⁴ are independently selected from the group consisting of H, fucosyl, and a galactosyl moiety A: 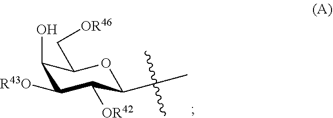
R⁴² is selected from the group consisting of H and fucosyl;
R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R⁴³ is selected from the group consisting of a H, a monosaccharide, and an oligosaccharide comprising 2-10 monosaccharides; and
each monosaccharide is independently selected from the group consisting of glucose, galactose, _{L}-fucose, N-acetylglucosamine, N-trifluoroacetylglucosamine, glucosamine, N-Fmoc-glucosamine, N-Boc-glucosamine, and N-acetylneuraminic acid;
provided that:
(i) at least one of R³³ and R³⁴ is fucosyl or galactosyl moiety A;
(ii) at least one of R⁴² and R⁴³ is a monosaccharide or an oligosaccharide
when (a) R²⁶, R³⁶, and R⁴⁶ are H, and
(b) R³³ is galactosyl moiety A and R³⁴ is H or fucosyl, or
R³⁴ is galactosyl moiety A and R³³ is H or fucosyl; and
(iii) R⁴³ is a monosaccharide or an oligosaccharide when R²⁶, R³⁶, and R⁴⁶ are H,
R⁴² is fucosyl, R³³ is galactosyl moiety A, and R³⁴ is H.

2. A compound according to Formula II: 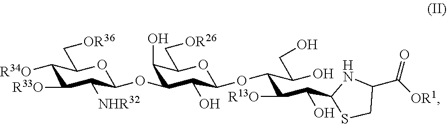
or a salt thereof, wherein:
R¹ is selected from the group consisting of benzyl and fluorenylmethyl;
R¹³ is selected from the group consisting of H and fucosyl;
R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R³² is selected from the group consisting of H, acetyl, trifluoroacetyl, Boc, Fmoc;
R³⁶ is selected from the group consisting of H and N-acetylneuraminic acid;
R³³ and R³⁴ are independently selected from the group consisting of H, fucosyl, and a galactosyl moiety A: 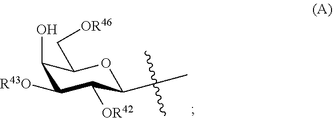
R⁴² is selected from the group consisting of H and fucosyl;
R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R⁴³ is selected from the group consisting of a H, a monosaccharide, and an oligosaccharide comprising 2-10 monosaccharides; and
each monosaccharide is independently selected from the group consisting of glucose, galactose, _{L}-fucose, N-acetylglucosamine, N-trifluoroacetylglucosamine, glucosamine, N-Fmoc-glucosamine, N-Boc-glucosamine, and N-acetylneuraminic acid.

3. The compound of claim 2 or a salt thereof, wherein:
(i) at least one of R³³ and R³⁴ is fucosyl or galactosyl moiety A;
(ii) at least one of R⁴² and R⁴³ is a monosaccharide or an oligosaccharide when
(a) R²⁶, R³⁶, and R⁴⁶ are H, and
(b) R³³ is galactosyl moiety A and R³⁴ is H or fucosyl, or
R³⁴ is galactosyl moiety A and R³³ is H or fucosyl; and
(iii) R⁴³ is a monosaccharide or an oligosaccharide when R⁴² is fucosyl, R³³ is galactosyl moiety A, and R³⁴ is H.

4. A compound according to Formula III: 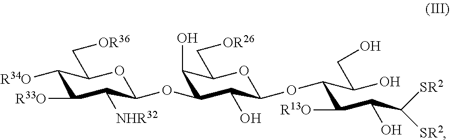
or a salt thereof, wherein:
each R² is is independently selected from the group consisting of C₁₋₆ alkyl and optionally substituted C₆₋₁₄ aryl, or two R² are taken together to form a C₁₋₆ alkylene diradical;
R¹³ is selected from the group consisting of H and fucosyl;
R²⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R³² is selected from the group consisting of H, acetyl, trifluoroacetyl, Boc, Fmoc;
R³⁶ is selected from the group consisting of H and N-acetylneuraminic acid;
R³³ and R³⁴ are independently selected from the group consisting of H, fucosyl, and a galactosyl moiety A: 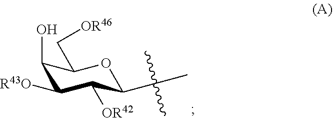
R⁴² is selected from the group consisting of H and fucosyl;
R⁴⁶ is selected from the group consisting of H, N-acetylglucosamine, N-acetylneuraminic acid, and an oligosaccharide comprising 2-10 monosaccharides; and each monosaccharide is independently selected from the group consisting of glucose, galactose, L-fucose, N-acetylglucosamine, N-trifluoroacetylglucosamine, glucosamine, N-Fmoc-glucosamine, N-Boc-glucosamine, and N-acetylneuraminic acid;
R⁴³ is selected from the group consisting of a H, a monosaccharide, and an oligosaccharide comprising 2-10 monosaccharides; and
each monosaccharide is independently selected from the group consisting of glucose, galactose, _{L}-fucose, N-acetylglucosamine, N-trifluoroacetylglucosamine, glucosamine, N-Fmoc-glucosamine, N-Boc-glucosamine, and N-acetylneuraminic acid.

5. The compound of claim 4, or a salt thereof, wherein:
(i) at least one of R³³ and R³⁴ is fucosyl or galactosyl moiety A;
(ii) at least one of R⁴² and R⁴³ is a monosaccharide or an oligosaccharide when
(a) R²⁶, R³⁶, and R⁴⁶ are H, and
(b) R³³ is galactosyl moiety A and R³⁴ is H or fucosyl, or
R³⁴ is galactosyl moiety A and R³³ is H or fucosyl; and
(iii) R⁴³ is a monosaccharide or an oligosaccharide when R⁴² is fucosyl, R³³ is galactosyl moiety A, and R³⁴ is H.

6. The compound of claim 1, or a salt thereof, wherein R¹ is benzyl.

7. The compound of claim 1, or a salt thereof, wherein R³³ is galactosyl moiety A.

8. The compound of claim 1, or a salt thereof, wherein R³⁴ is galactosyl moiety A.

9. The compound of claim 1, or a salt thereof, wherein R⁴² is H and R⁴³ is a monosaccharide or an oligosaccharide.

10. The compound of claim 1, or a salt thereof, wherein R¹³ is H.

11. The compound of claim 1, or a salt thereof, wherein R¹³ is fucosyl.

12. The compound of claim 1, which is selected from the group consisting of:
Galβ4GlcβNHCbz, GlcNAcβ3Galβ4GlcβNHCbz, Galβ4GlcNAcβ3Galβ4GlcβNHCbz, GlcNAcβ3Galβ4GlcNAcβ3Galβ4GlcβNHCbz, Galβ4GlcNAcβ3Galβ4(Fucα3)GlcβNHCbz, Galβ4(Fucα3)GlcNAcβ3Galβ4(Fucα3)GlcβNHCbz, GalβGlcNAcβ3Galβ4GlcβNHCbz, Fucα2Galβ33GlcNAcβ3Galβ4GlcβNHCbz, GalβGlcNAcβ3Galβ4(Fucα3)GlcβNHCbz, Galβ(Fucα4)GlcNAcβ3Galβ4(Fucα3)GlcβNHCbz, Galβ4GlcNAcβ3Galβ4GlcNAcβ3Galβ4GlcβNHCbz, GlcNAcβ3Galβ4GlcNAcβ3Galβ4GlcNAcβ3Galβ4GlcβNHCbz, Fucα2Galβ4GlcNAcβ3Galβ4GlcNAcβ3Galβ4GlcβNHCbz, Galβ4(Fucα3)GlcNAcβ3Galβ4(Fucα3)GlcNAcβ3Galβ4(Fucα3)GlcβNHCbz, Neu5Acα3Gal β4GlcNAcβ33Galβ4GlcNAcβ3Galβ4GlcβNHCbz, Neu5Acα6Galβ4GlcNAcβ3Galβ4GlcNAcβ3Galβ4GlcβNHCbz, Gal β3GlcNAcβ3Galβ4GlcNAcβ3Galβ4GlcβNHCbz, Fucα2Galβ33GlcNAcβ3Galβ4GlcNAcβ3Galβ4GlcβNHCbz, Galβ(Fucα4)GlcNAcβ3Galβ4(Fucα3)GlcNAcβ3Galβ4(Fucα3)GlcβNHCbz, Galβ4GlcNAcβ3Galβ4GlcNAcβ3Galβ4GlcNAcβ3Galβ4GlcβNHCbz, GalβGlcNAcβ3Galβ4GlcNAcβ3Galβ4GlcNAcβ3Galβ4GlcβNHCbz, Fucα2Galβ33GlcNAcβ3Galβ4GlcNAcβ3Galβ4GlcNAcβ3Galβ4GlcβNHCbz, Fucα2Galβ3(Fucα4)GlcNAcβ3Galβ4(Fucα3)GlcβNHCbz, Fucα2Galβ33GlcNAcβ3Galβ4(Fucα3)GlcβNHCbz, Galβ(Fucα4)GlcNAcβ3Galβ4GlcβNHCbz, Fucα2Galβ3(Fucα4)GlcNAcβ3Galβ4GlcβNHCbz, Fucα2Galβ4GlcNAcβ3Galβ4GlcβNHCbz, Fucα2Galβ4(Fucα3)GlcNAcβ3Galβ4(Fucα3)GlcβNHCbz, Fucα2Galβ4GlcNAcβ3Galβ4(Fucα3)GlcβNHCbz, Galβ4(Fucα3)GlcNAcβ3Galβ4GlcβNHCbz, Fucα2Galβ4(Fucα3)GlcNAcβ3Galβ4GlcβNHCbz, Neu5Acα3GalβGlcNAcβ3Galβ4GlcβNHCbz, Neu5Acα6Galβ3GlcNAcβ3Galβ4GlcβNHCbz, Neu5Acα3Gal β3GlcNAcβ3Galβ4(Fucα3)GlcβNHCbz, Neu5Acα6GalβGlcNAcβ3Galβ4(Fucα3)GlcβNHCbz, Neu5Acα3Galβ33(Fucα4)GlcNAcβ3 Galβ4(Fucα3)GlcβNHCbz, Neu5Acα3Galβ33(Fucα4)GlcNAcβ3Galβ4GlcβNHCbz, Neu5Acα3Galβ4GlcNAcβ3Galβ4GlcβNHCbz, Neu5Acα6Galβ4GlcNAcβ3Galβ4GlcβNHCbz, Neu5Acα3Galβ4GlcNAcβ3Galβ4(Fucα3)GlcβNHCbz, Neu5Acα6Galβ4GlcNAcβ3Galβ4(Fucα3)GlcβNHCbz, Neu5Acα3Galβ4(Fucα3)GlcNAcβ3Galβ4(Fucα3)GlcβNHCbz, Neu5Acα3Gal β4(Fucα3)GlcNAcβ33Galβ4GlcβNHCbz, Galβ33(Neu5Acα6)GlcNAcβ33Galβ4GlcβNHCbz, Fucα2Galβ33(Neu5Acα,6)GlcNAcβ3Galβ4GlcβNHCbz, Neu5Acα3Galβ33(Neu5Acα6)GlcNAcβ3Galβ4GlcβNHCbz, Neu5Acα3Galβ33(Neu5Acα6)GlcNAcβ3Galβ4(Fucα3)GlcβNHCbz, Neu5Acα3Galβ(Neu5Acα6) (Fucα4)GlcNAcβ3Galβ4GlcβNHCbz, Galβ4GlcNAcβ3(GlcNAcβ6)Galβ4GlcβNHCbz, Galβ4(Fucα3)GlcNAcβ3Galβ4(Fucα3)GlcNAcβ3Galβ4GlcβNHCbz, Galβ4GlcNAcβ3Galβ4(Fucα3)GlcNAcβ3Galβ4GlcβNHCbz, Galβ4(Fucα3)GlcNAcβ3Galβ4GlcNAcβ3Galβ4GlcβNHCbz, Galβ4GlcNAcβ3Galβ4GlcNAcβ3Galβ4(Fucα3)GlcβNHCbz, GlcNHTFAβ3Galβ4GlcβNHCbz, GlcNH₂Galβ4GlcβNHCbz, Galβ4GlcNAcβ3Galβ4GlcNH₂β3Galβ4GlcβNHCbz, Galβ4GlcNAcβ3Galβ4GlcNHBocβ3Galβ4GlcβNHCbz, Galβ4GlcNAcβ3Galβ4GlcNHFmocβ3Galβ4GlcβNHCbz, Galβ4(Fucα3)GlcNAcβ3Galβ4GlcNHBocβ3Galβ4(Fucα3)GlcβNHCbz, Galβ4(Fucα3)GlcNAcβ3Galβ4GlcNHFmocβ3Galβ4(Fucα3)GlcβNHCbz, Galβ4(Fucα3)GlcNAcβ3Galβ4GlcNAcβ3Galβ4(Fucα3)GlcβNHCbz, GalβGlcNAcβ3Galβ4GlcNH₂β3Galβ4GlcβNHCbz, GalβGlcNAcβ3Galβ4GlcNHBocβ3Galβ4GlcβNHCbz, GalβGlcNAcβ3Galβ4GlcNHFmocβ3Galβ4GlcβNHCbz, Galβ(Fucα4)GlcNAcβ3Galβ4GlcNHBocβ3Galβ4(Fucα3)GlcβNHCbz, Galβ(Fucα4)GlcNAcβ3Galβ4GlcNHFmocβ3Galβ4(Fucα3)GlcβNHCbz, Galβ(Fucα4)GlcNAcβ3Galβ4GlcNAcβ3Galβ4(Fucα3)GlcβNHCbz, and salts thereof.

13-42. (canceled)

43. The compound of claim 2, or a salt thereof, wherein R¹ is benzyl.

44. The compound of claim 2, or a salt thereof, wherein R³³ is galactosyl moiety A.

45. The compound of claim 2, or a salt thereof, wherein R³⁴ is galactosyl moiety A.

46. The compound of claim 2, or a salt thereof, wherein R⁴² is H and R⁴³ is a monosaccharide or an oligosaccharide.

47. The compound of claim 2, or a salt thereof, wherein R¹³ is H.

48. The compound of claim 2, or a salt thereof, wherein R¹³ is fucosyl.

49. The compound of claim 4, or a salt thereof, wherein R¹³ is H.

50. The compound of claim 4, or a salt thereof, wherein R¹³ is fucosyl.


SEQUENCE LISTING (18 sequences)

SEQ ID NO: 1 - DNA, 2073 bp, Synthetic construct
  source 1..2073: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcggattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaattccatatggccagtgccaccggtagtagtcagccagccgcggaaagcagcacccagcaacgcccgggcgttccagcgggtccacgtccgctggatggttatctgggcgtggccgatcacaagccgctgaaaatgcactgccgtgattgcgcgctggtgaccagtagcggccatctgctgcacagccgccaaggtagccagattgatcagaccgagtgcgtgatccgcatgaacgacgcgccaacccgtggttatggccgcgatgttggcaaccgtacgagtctgcgcgttatcgcccacagtagcatccagcgcattctgcgcaaccgccacgatctgctcaatgttagccaaggcaccgtgttcatcttctggggcccgagcagctatatgcgccgcgatggcaagggccaagtttacaacaacctccatctgctcagccaagttctgccgcgtctgaaagcgttcatgatcacccgccacaagatgctgcagttcgatgagctgttcaagcaagaaacgggtaaggaccgcaaaatcagcaacacgtggctgagcacgggctggtttaccatgacgatcgcgctggagctgtgcgaccgcatcaacgtgtacggcatggttccgccggatttttgccgcgatccgaatcatccaagcgtgccgtaccactactacgaaccgtttggtccggacgaatgtaccatgtatctgagccatgagcgtggccgcaagggcagccatcaccgcttcatcacggagaagcgcgtgttcaagaactgggcccgcaccttcaacatccatttcttccaaccggattggaagccggaaagtctggccatcaaccacccagaaaacaaaccggttttcggatcccatcatcatcatcatcactga

SEQ ID NO: 2 - DNA, 2073 bp, Synthetic construct
  source 1..2073: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcggattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaattccatatggccagtgccaccggtagtagtcagccagccgcggaaagcagcacccagcaacgcccgggcgttccagcgggtccacgtccgctggatggttatctgggcgtggccgatcacaagccgctgaaaatgcactgccgtgattgcgcgctgatgaccagtagcggccatctgctgcacagccgccaaggtagccagattgatcagaccgagtgcgtgatccgcatgaacgacgcgccaacccgtggttatggccgcgatgttggcaaccgtacgagtctgcgcgttatcgcccacagtagcatccagcgcattctgcgcaaccgccacgatctgctcaatgttagccaaggcaccgtgttcatcttctggggcccgagcagctatatgcgccgcgatggcaagggccaagtttacaacaacctccatctgctcagccaagttctgccgcgtctgaaagcgttcatgatcacccgccacaagatgctgcagttcgatgagctgttcaagcaagaaacgggtaaggaccgcaaaatcagcaacacgtggctgagcacgggctggtttaccatgacgatcgcgctggagctgtgcgaccgcatcaacgtgtacggcatggttccgccggatttttgccgcgatccgaatcatccaagcgtgccgtaccactactacgaaccgtttggtccggacgaatgtaccatgtatctgagccatgagcgtggccgcaagggcagccatcaccgcttcatcacggagaagcgcgtgttcaagaactgggcccgcaccttcaacatccatttcttccaaccggattggaagccggaaagtctggccatcaaccacccagaaaacaaaccggttttcggatcccatcatcatcatcatcactga

SEQ ID NO: 3 - DNA, 2133 bp, Synthetic construct
  source 1..2133: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcggattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaattccatatggccagtgccaccggtagtagtcagccagccgcggaaagcagcacccagcaacgcccgggcgttccagcgggtccacgtccgctggatggttatctgggcgtggccgatcacaagccgctgaaaatgcactgccgtgattgcgcgctggtgaccagtagcggccatctgctgcacagccgccaaggtagccagattgatcagaccgagtgcgtgatccgcatgaacgacgcgccaacccgtggttatggccgcgatgttggcaaccgtacgagtctgcgcgttatcgcccacagtagcatccagcgcattctgcgcaaccgccacgatctgctcaatgttagccaaggcaccgtgttcatcttctggggcccgagcagctatatgcgccgcgatggcaagggccaagtttacaacaacctccatctgctcagccaagttctgccgcgtctgaaagcgttcatgatcacccgccacaagatgctgcagttcgatgagctgttcaagcaagaaacgggtaaggaccgcaaaatcagcaacacgtggctgagcacgggctggtttaccatgacgatcgcgctggagctgtgcgaccgcatcaacgtgtacggcatggttccgccggatttttgccgcgatccgaatcatccaagcgtgccgtaccactactacgaaccgtttggtccggacgaatgtaccatgtatctgagccatgagcgtggccgcaagggcagccatcaccgcttcatcacggagaagcgcgtgttcaagaactgggcccgcaccttcaacatccatttcttccaaccggattggaagccggaaagtctggccatcaaccacccagaaaacaaaccggttttcggatcccaaggaatgtcaatgcccgactgtaaccctcgatacagcttgcaccagactccgagacatcatcatcatcatcatcactga

SEQ ID NO: 4 - DNA, 2133 bp, Synthetic construct
  source 1..2133: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcggattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcgaattccatatggccagtgccaccggtagtagtcagccagccgcggaaagcagcacccagcaacgcccgggcgttccagcgggtccacgtccgctggatggttatctgggcgtggccgatcacaagccgctgaaaatgcactgccgtgattgcgcgctgatgaccagtagcggccatctgctgcacagccgccaaggtagccagattgatcagaccgagtgcgtgatccgcatgaacgacgcgccaacccgtggttatggccgcgatgttggcaaccgtacgagtctgcgcgttatcgcccacagtagcatccagcgcattctgcgcaaccgccacgatctgctcaatgttagccaaggcaccgtgttcatcttctggggcccgagcagctatatgcgccgcgatggcaagggccaagtttacaacaacctccatctgctcagccaagttctgccgcgtctgaaagcgttcatgatcacccgccacaagatgctgcagttcgatgagctgttcaagcaagaaacgggtaaggaccgcaaaatcagcaacacgtggctgagcacgggctggtttaccatgacgatcgcgctggagctgtgcgaccgcatcaacgtgtacggcatggttccgccggatttttgccgcgatccgaatcatccaagcgtgccgtaccactactacgaaccgtttggtccggacgaatgtaccatgtatctgagccatgagcgtggccgcaagggcagccatcaccgcttcatcacggagaagcgcgtgttcaagaactgggcccgcaccttcaacatccatttcttccaaccggattggaagccggaaagtctggccatcaaccacccagaaaacaaaccggttttcggatcccaaggaatgtcaatgcccgactgtaaccctcgatacagcttgcaccagactccgagacatcatcatcatcatcatcactga

SEQ ID NO: 5 - protein, 691 aa, Synthetic construct
HMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRISEFEFHMASATGSSQPAAESSTQQRPGVPAGPRPLDGYLGVADHKPLKMHCRDCALVTSSGHLLHSRQGSQIDQTECVIRMNDAPTRGYGRDVGNRTSLRVIAHSSIQRILRNRHDLLNVSQGTVFIFWGPSSYMRRDGKGQVYNNLHLLSQVLPRLKAFMITRHKMLQFDELFKQETGKDRKISNTWLSTGWFTMTIALELCDRINVYGMVPPDFCRDPNHPSVPYHYYEPFGPDECTMYLSHERGRKGSHHRFITEKRVFKNWARTFNIHFFQPDWKPESLAINHPENKPVFGSHHHHHH

SEQ ID NO: 6 - protein, 690 aa, Synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRISEFEFHMASATGSSQPAAESSTQQRPGVPAGPRPLDGYLGVADHKPLKMHCRDCALMTSSGHLLHSRQGSQIDQTECVIRMNDAPTRGYGRDVGNRTSLRVIAHSSIQRILRNRHDLLNVSQGTVFIFWGPSSYMRRDGKGQVYNNLHLLSQVLPRLKAFMITRHKMLQFDELFKQETGKDRKISNTWLSTGWFTMTIALELCDRINVYGMVPPDFCRDPNHPSVPYHYYEPFGPDECTMYLSHERGRKGSHHRFITEKRVFKNWARTFNIHFFQPDWKPESLAINHPENKPVFGSHHHHHH

SEQ ID NO: 7 - protein, 710 aa, Synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRISEFEFHMASATGSSQPAAESSTQQRPGVPAGPRPLDGYLGVADHKPLKMHCRDCALVTSSGHLLHSRQGSQIDQTECVIRMNDAPTRGYGRDVGNRTSLRVIAHSSIQRILRNRHDLLNVSQGTVFIFWGPSSYMRRDGKGQVYNNLHLLSQVLPRLKAFMITRHKMLQFDELFKQETGKDRKISNTWLSTGWFTMTIALELCDRINVYGMVPPDFCRDPNHPSVPYHYYEPFGPDECTMYLSHERGRKGSHHRFITEKRVFKNWARTFNIHFFQPDWKPESLAINHPENKPVFGSQGMSMPDCNPRYSLHQTPRHHHHHHH

SEQ ID NO: 8 - protein, 710 aa, Synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRISEFEFHMASATGSSQPAAESSTQQRPGVPAGPRPLDGYLGVADHKPLKMHCRDCALMTSSGHLLHSRQGSQIDQTECVIRMNDAPTRGYGRDVGNRTSLRVIAHSSIQRILRNRHDLLNVSQGTVFIFWGPSSYMRRDGKGQVYNNLHLLSQVLPRLKAFMITRHKMLQFDELFKQETGKDRKISNTWLSTGWFTMTIALELCDRINVYGMVPPDFCRDPNHPSVPYHYYEPFGPDECTMYLSHERGRKGSHHRFITEKRVFKNWARTFNIHFFQPDWKPESLAINHPENKPVFGSQGMSMPDCNPRYSLHQTPRHHHHHHH

SEQ ID NO: 9 - DNA, 2319 bp, Synthetic construct
  source 1..2319: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcatgttcggcggagatccttctttccaacgcttaaatattagcgacccgttacgtcttacacaagtgtgtacatcatttattaatgggaaaactcgcttcttatggaagaacaagctcatgatccacgagaaaagcagctgcaaggaatacctgacacaaagtcattatattaccgcaccattgagtaaagaagaggccgatttccccttagcgtacattatggtcatacaccaccatttcgacaccttcgcccggctgtttcgcgctatatacatgccgcagaatatttactgcgttcacgttgacgaaaaggcaacaacagagttcaaggacgctgttgaacaacttctgtcctgtttccccaacgcatttcttgcatctaagatggagccggtggtgtacggcggtatttcacgccttcaagcagacctcaattgcatacgtgacttatcagccttcgaggtttcttggaaatacgttataaacacgtgcggacaagacttcccactgaagacaaacaaggagattgtgcaatacctgaagggctttaagggaaagaatattacgcccggggtattaccaccggcacatgcgattgggcgaacgaaatacgtccaccaagagcacttaggtaaggaattgtcctatgtcatacgtactacagctctgaagccgccaccccctcacaatttaactatttattttggttccgcttacgttgctttgagtcgcgagttcgctaatttcgtcttacatgatcctcgggcggtggacttactgcaatggtccaaagacactttcagccccgacgaacacttttgggtaacactgaaccgaataccaggtgtacctggctccatgcctaatgcctcttggaccggcaacctgagagcaattaaatggtccgatatggaagaccgccacggtggatgtcatgggcattacgtgcacggaatatgtatatacggtaacggtgaccttaagtggctggtgaatagtccgagcctgtttgctaacaagttcgagctgaacacatatcctcttactgtggagtgtctcgaacttcgtcatcgcgaaagaacgctgaaccagtccgagacggccattcagccatcatggtacttccatcatcatcaccaccactaa

SEQ ID NO: 10 - protein, 772 aa, Synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFMFGGDPSFQRLNISDPLRLTQVCTSFINGKTRFLWKNKLMIHEKSSCKEYLTQSHYITAPLSKEEADFPLAYIMVIHHHFDTFARLFRAIYMPQNIYCVHVDEKATTEFKDAVEQLLSCFPNAFLASKMEPVVYGGISRLQADLNCIRDLSAFEVSWKYVINTCGQDFPLKTNKEIVQYLKGFKGKNITPGVLPPAHAIGRTKYVHQEHLGKELSYVIRTTALKPPPPHNLTIYFGSAYVALSREFANFVLHDPRAVDLLQWSKDTFSPDEHFWVTLNRIPGVPGSMPNASWTGNLRAIKWSDMEDRHGGCHGHYVHGICIYGNGDLKWLVNSPSLFANKFELNTYPLTVECLELRHRERTLNQSETAIQPSWYFHHHHHH

SEQ ID NO: 11 - DNA, 39 bp, Synthetic construct
  source 1..39: mol_type = other DNA
tatacaaatacatatcatgtttttaataatttaccacct

SEQ ID NO: 12 - DNA, 39 bp, Synthetic construct
  source 1..39: mol_type = other DNA
attaaaaacatgatatgtatttgtatagaactctatatt

SEQ ID NO: 13 - DNA, 39 bp, Synthetic construct
  source 1..39: mol_type = other DNA
gatgatggttcttatgaatatgtaagtttatatcaatgg

SEQ ID NO: 14 - DNA, 39 bp, Synthetic construct
  source 1..39: mol_type = other DNA
acttacatattcataagaaccatcatcatacaaactaat

SEQ ID NO: 15 - DNA, 23 bp, Synthetic construct
  source 1..23: mol_type = other DNA
ttgcgcgctgggcaccagtagcg

SEQ ID NO: 16 - DNA, 21 bp, Synthetic construct
  source 1..21: mol_type = other DNA
tcacggcagtgcattttcagc

SEQ ID NO: 17 - DNA, 53 bp, Synthetic construct
  source 1..53: mol_type = other DNA
cgatacagcttgcaccagactccgagacatcatcatcatcatcatcactgatc

SEQ ID NO: 18 - DNA, 49 bp, Synthetic construct
  source 1..49: mol_type = other DNA
agggttacagtcgggcattgacattccttgggatccgaaaaccggtttg